(19) United States
(12) Patent Application Publication
(10) Pub. No.: US 2026/0035689 A1
(43) Pub. Date: Feb. 5, 2026

(54) REDIRECTION OF TROPISM OF AAV CAPSIDS

(71) Applicant: VOYAGER THERAPEUTICS, INC., Lexington, MA (US)

(72) Inventors: Mathieu E. Nonnenmacher, Boston, MA (US); Jinzhao Hou, Lexington, MA (US); Wei Wang, Lexington, MA (US)

(21) Appl. No.: 19/297,841

(22) Filed: Aug. 12, 2025

Related U.S. Application Data
(62) Division of application No. 17/282,479, now Pat. No. 12,467,046.
(60) Provisional application No. 62/740,310, filed on Oct. 2, 2018.
(60) Provisional application No. 62/839,883, filed on Apr. 29, 2019.

(51) Int. Cl. C12N 15/10; C07K 14/005

(52) U.S. Cl. CPC C12N 15/1037; C07K 14/005; C12N 2750/14122; C12N 2750/14145

(57) ABSTRACT

The disclosure relates to compositions, methods, and processes for the preparation, use, and/or formulation of adeno-associated virus capsid proteins, wherein the capsid proteins comprise targeting peptide inserts for enhanced tropism to a target tissue.

### CROSS REFERENCE TO RELATED APPLICATIONS

This application is a Divisional of U.S. patent application Ser. No. 17/282,479, filed Apr. 2, 2021, which is a 35 U.S.C. § 371 U.S. National Stage Entry of International Application No. PCT/US2019/054345, filed Oct. 2, 2019 and entitled REDIRECTION OF TROPISM OF AAV CAPSIDS; which claims the benefit of: U.S. Provisional Patent Application No. 62/740,310, filed Oct. 2, 2018, entitled AAV CAPSID LIBRARIES AND TISSUE TARGETING PEPTIDE INSERTS; U.S. Provisional Patent Application No. 62/839,883, filed Apr. 29, 2019 entitled REDIRECTION OF TROPISM OF AAV CAPSIDS; the contents of which are each incorporated herein by reference in their entirety.

### REFERENCE TO THE SEQUENCE LISTING

The present application is being filed along with a Sequence Listing submitted electronically in XML format and is hereby incorporated by reference in its entirety. Said XML copy, created on Aug. 11, 2025, is named V2071-1060USDIVI_SL.xml and is 1,679,340 bytes in size.

### FIELD OF THE DISCLOSURE

The disclosure relates to compositions, methods, and processes for the preparation, use, and/or formulation of adeno-associated virus capsid proteins, wherein the capsid proteins comprise targeting peptide inserts for enhanced tropism to a target tissue.

### BACKGROUND

Gene delivery to the adult central nervous system (CNS) remains a major challenge in gene therapy, and engineered AAV capsids with improved brain tropism represent an attractive solution.

Adeno-associated virus (AAV)-derived vectors are promising tools for clinical gene transfer because of their non-pathogenic nature, their low immunogenic profile, low rate of integration into the host genome and long-term transgene expression in non-dividing cells. However, the transduction efficiency of AAV natural variants in certain organs is too low for clinical applications, and capsid neutralization by pre-existing neutralizing antibodies may prevent treatment of a large proportion of patients. For these reasons, major efforts have been devoted to obtaining novel capsid variants with enhanced properties. Of many approaches tested so far, the most significant advances have resulted from directed evolution of AAV capsids using in vitro or in vivo selection of capsid variants created by capsid sequence randomization using either error-prone PCR, shuffling of various parent serotypes or insertion of fully randomized short peptides at defined positions.

In order to perform directed evolution of AAV capsids, the sequence encoding the viral capsid is itself flanked by inverted terminal repeats (ITR) so it can be packaged into its own capsid shell. Following infection of cultured cells or animals by the mixed population of capsids, the DNA encoding capsids variants that have successfully homed into the tissue of interest is recovered by PCR for further rounds of selection. In this approach, all viral DNA species present in a given tissue are recovered, with no discrimination for specific cell types or for vectors able to perform complete transduction (cell surface binding, endocytosis, trafficking, nuclear import, uncoating, second-strand synthesis, transcription). For example, in the case of highly complex tissues containing multiple cell types, such as the central nervous system (CNS), it would be highly preferable to apply a more stringent selective pressure aimed at recovering capsid variants capable of transducing neuron and/or astrocyte rather than microglia or blood vessel endothelial cells.

Attempts at improving the CNS tropism of AAV capsids upon systemic administration have been met with limited success.

Two previous approaches have been used to address this issue. The first strategy used co-infection of cultured cells (Grimm et al., 2008) or in situ animal tissue (Lisowski et al., 2014) with adenovirus, in order to trigger exponential replication of infectious AAV DNA. Another successful approach involved the use of cell-specific CRE transgenic mice (Deverman et al., 2016) allowing viral DNA recombination specifically in astrocytes, followed by recovery of CRE-recombined capsid variants. Both approaches proved successful, allowing the isolation of several capsid variants with enhanced transduction of target cell populations.

This finding suggested that cell type-specific library selection could improve the outcome of directed evolution. However, the transgenic CRE system used by Deverman et al. is not tractable in other animal species and AAV variants selected by directed evolution in mouse tissue do not show similar properties in large animals. Therefore, it would be necessary to perform the entire directed evolution process directly in non-human primates to increase the probability of translatability in human subjects. None of the previously described transduction-specific approaches are amenable to large animal studies because: 1) many tissues of interest (e.g. CNS) are not readily accessible to adenovirus co-infection, 2) the specific Ad tropism itself would bias the library distribution, and 3) large animals are typically not amenable to transgenesis and cannot be genetically engineered to express CRE recombinase in defined cell types.

To address this problem, we have developed a broadly-applicable functional AAV capsid library screening platform for cell type-specific biopanning in non-transgenic animals. In the TRACER (Tropism Redirection of AAV by Cell type-specific Expression of RNA) platform system, the capsid gene is placed under the control of a cell type-specific promoter to drive capsid mRNA expression in the absence of helper virus co-infection. This RNA-driven screen increases the selective pressure in favor of capsid variants which transduce a specific cell type.

The TRACER platform allows generation of AAV capsid libraries whereby specific recovery and subcloning of capsid mRNA expressed in transduced cells is achieved with no need for transgenic animals or helper virus co-infection. Since mRNA transcription is a hallmark of full transduction, these methods will allow identification of fully infectious AAV capsid mutants. In addition to its higher stringency, this method allows identification of capsids with high tropism for particular cell types using libraries designed to express CAP mRNA under the control of any cell-specific promoter such as, but not limited to, synapsin-1 promoter (neurons), GFAP promoter (astrocytes), TBG promoter (liver), CAMK promoter (skeletal muscle), MYH6 promoter (cardiomyocytes).

### SUMMARY OF THE DISCLOSURE

The present disclosure provides compositions and methods for the engineering and/or redirecting the tropism of AAV capsids. Also provided herein are peptides which may be inserted into AAV capsid sequences to increase the tropism of the capsid for a particular tissue. In one aspect, the peptides may be used to target the capsids to brain or regions of the brain or the spinal cord.

The present disclosure presents methods for generating one or more variant AAV capsid polypeptides. In certain embodiments, the variant AAV capsid polypeptides exhibit at least one of improved transduction or increased cell or tissue specificity, relative to a parental AAV capsid polypeptide. In certain embodiments, the method includes: (a) generating a library of variant AAV capsid polypeptides, wherein said library includes (i) a plurality of capsid polypeptides having a region of randomized sequence of 2, 3, 4, 5, 6, 7, 8, or 9 consecutive amino acids, or (ii) a plurality of capsid polypeptides from more than one parental AAV capsid polypeptide; (b) generating an AAV vector library by cloning the capsid polypeptides of libraries (a) (i) or (a) (ii) into AAV vectors, wherein the AAV vectors include a first promoter and a second promoter, wherein said second promoter drives capsid mRNA expression in the absence of helper virus co-infection.

In certain embodiments, the first promoter is AAV2 P40. In certain embodiments, the second promoter is a ubiquitous promoter. In certain embodiments, the first promoter is AAV2 P40 and the second promoter is a ubiquitous promoter.

In certain embodiments, the first promoter is AAV2 P40. In certain embodiments, the second promoter is a cell-type-specific promoter. In certain embodiments, the first promoter is AAV2 P40 and the second promoter is a cell-type-specific promoter.

In certain embodiments, the promoter is selected from any promoter listed in Table 3. In certain embodiments, the ubiquitous or cell-specific promoter allows the expression of RNA encoding the capsid polypeptides.

In certain embodiments, the method includes recovery of the RNA encoding the capsid polypeptides. In certain embodiments, the method includes determining the sequence of the capsid polypeptides. In certain embodiments, the capsid polypeptides recovered exhibit increased target cell transduction or target cell specificity (tropism) as compared to a parental capsid polypeptide.

In certain embodiments, the target cell is a neuronal cell, a neural stem cell, an astrocyte, an oligodendrocyte, a microglia cell, a retinal cell, a tumor cell, a hematopoietic stem cell, an insulin producing beta cell, a lung epithelium cell, an endothelial cell, a liver cell, a skeletal muscle cell, a muscle stem cell, a muscle satellite cell, or a cardiac muscle cell.

In certain embodiments, the AAV vectors comprise a first promoter and a second promoter, wherein the second promoter is located the downstream of the capsid gene and drives its anti-sense RNA expression in the absence of helper virus co-infection.

In certain embodiments, the first promoter is AAV2 P40 and the second promoter is a ubiquitous promoter. In certain embodiments, the first promoter is AAV2 P40 and the second promoter is a cell-specific promoter. In certain embodiments, the ubiquitous or cell-specific promoter allows the expression of gene encoding the capsid polypeptide of variant AAV in an anti-sense direction, resulting in the anti-sense RNA. In certain embodiments, the method included the recovery of the anti-sense RNA that can be converted to RNA encoding the variant AAV capsid polypeptide that is used to determine the sequence of the variant AAV capsid polypeptides.

In certain embodiments, the variant AAV capsid polypeptide exhibits increased target cell transduction or target cell specificity (tropism) as compared to a parental capsid polypeptide.

### BRIEF DESCRIPTION OF THE DRAWINGS

The foregoing and other objects, features, and advantages will be apparent from the following description of particular embodiments of the disclosure, as illustrated in the accompanying drawings. The drawings are not necessarily to scale, emphasis instead being placed upon illustrating the principles of various embodiments of the disclosure.

FIG. 1A and FIG. 1B are maps of wild-type AAV capsid gene transcription and CMV-CAP vectors. FIG. 1A shows transcription of VP1, VP2 and VP3 AAV transcripts from wildtype AAV genome. Transcription start sites of each viral promoter are indicated. SD, splice donor, SA, splice acceptor. Sequence of start codons for each reading frame is indicated. Translation of AAP and VP3 is performed by leaky scanning of the major mRNA. FIG. 1B shows the structure of the CMV-p40 dual promoter vectors used to determine the minimal regulatory sequences necessary for efficient virus production. The pREP2ACAP vector shown at the bottom is obtained by deletion of most CAP reading frame and is used to provide the REP protein in trans.

FIG. 2A and FIG. 2B are histogram representations of the data and show the effect of CMV promoter position on virus yield and CAP mRNA splicing. FIG. 2A shows average yield of AAV9 produced in HEK-293T cells using the constructs described in FIG. 1, co-transfected with an Ad Helper vector. Wild-type AAV9 plasmid (pAV9) is used as a positive control. Y-axis values indicate AAV DNA copies per ul from each 15-cm plate (˜1000 ul total, left panel) or the percentage of wtAAV9 (right panel). FIG. 2B shows evidence for expression of CAP transcripts in transfected cells. mRNA from transfected 293T cells was subjected to RT-PCR using primers specific for the major spliced CAP transcript. Note the lack of p40-driven transcription in the absence of Ad Helper vector (lane 2).

FIG. 3A, FIG. 3B and FIG. 3C show the effect of REP helper plasmid optimization on virus yield. FIG. 3A shows the design of improved pREP helper vectors. The MscI fragment deletion removes the C-terminal part of VP proteins, which is necessary for capsid formation. Asterisks represent early stop codons introduced to disrupt the coding potential of VP1, VP2 and VP3 reading frames. FIG. 3B shows the yield of Synapsin-p40-CAP9 AAV produced with various REP plasmid architectures. Values on the Y-axis represent the percentage of VG relative to wild-type AAV9. FIG. 3C shows the quantification of recombination and/or illegitimate packaging of full-length REP from the pREP plasmids. Virus stocks produced were subjected to qPCR using Taqman probes located in the N-terminal part of REP absent from the ITR-containing vectors.

FIG. 4A, FIG. 4B, FIG. 4C and FIG. 4D describe the in vivo analysis of the second-generation vectors. FIG. 4A shows the design of Pro9 vectors. Architecture of all three vectors is based on the BstEII construct. AAV9 capsid RNA is placed under control of P40 and CMV, hSyn1 or GFAP promoters, respectively. FIG. 4B shows the silver stain of SDS-PAGE gel obtained by running 1e10 VG of each vector, after double iodixanol purification. FIG. 4C shows the biodistribution of viral DNA in mouse brain (cortex), liver and heart following tail-vein injection of 1e12 VG per mouse. AAV9 VP3 DNA is quantified by Taqman PCR and normalized to mouse transferrin receptor gene. FIG. 4D shows the capsid RNA recovery from mouse tissues. Total RNA was reverse transcribed and Taqman PCR was performed with capsid-specific Taqman primers and probe. Values represent VP3 cDNA copies normalized to TBP housekeeping gene.

FIG. 5A, FIG. 5B, FIG. 5C, FIG. 5D and FIG. 5E describes in vitro analysis of intronic second generation vectors. FIG. 5A shows the design of intronic Pro9 vectors harboring a hybrid CMV/Globin intron. AAV9 capsid RNA is placed under control of P40 and CBA, hSyn1 or GFAP promoters in a tandem configuration (top) or in an inverted configuration (bottom). In the inverted promoter vectors, an extra SV40 polyadenylation site (orange) is added at the 3′ extremity to allow polyadenylation of antisense CAP9 transcripts. FIG. 5B shows the AAV9 CAP cDNA amplification. All vectors depicted were produced using triple transfection with pHelper and pREP-3stops and resulting viruses were used to infect HEK-293T cells at a MOI of 1e4 VG per cell. RNA was extracted 48 hours post-infection and subjected to RT-PCR with primers amplifying full capsid (top) or a C-terminal fragment (bottom). FIG. 5C shows the AAV9 VP3 cDNA from cells infected with intronless or intronic viruses with tandem promoters in forward orientation was quantified by Taqman PCR and normalized to GAPDH housekeeping gene. Values indicate the ratio of VP3 to GAPDH cDNA. FIG. 5D shows the mapping of capsid RNA recovery from cells infected with tandem or inverted constructs. Total RNA was reverse transcribed and PCR was performed with primers flanking the entire capsid gene. White arrowheads represent VP3 size variants resulting from aberrant splicing of antisense CAP mRNA. FIG. 5E shows the analysis of Globin intron splicing. CAG9 plasmid (left) or cDNA from HEK-293T cells transduced by CAG9 virus was submitted to PCR with forward primers located before (Glo ex1) or within (GloSpliceF4 (SEQ ID NO: 26) and GloSpliceF6 (SEQ ID NO: 13)) the Globin exon-exon junction. Primers spanning junction between exon 1 (no underline) and exon 2 (underline) are described at the bottom.

FIG. 6 provides in vitro evidence that the presence of the P40 promoter downstream of Synapsin or Gfabc1D promoters does not relieve the repression of either promoter in HEK-293T cells.

FIG. 7 illustrates the basic tenets of the TRACER platform.

FIG. 8 illustrates features of the TRACER platform including the use of a tissue specific promoter and RNA recovery.

FIG. 9 provides one embodiment of the TRACER production architecture.

FIG. 10 provides a comparison between traditional vDNA recovery and 2nd generation vRNA recovery.

FIG. 11 provides an overview of the use of cell-specific RNA expression for targeted evolution.

FIG. 12A and FIG. 12B provide diagrams representing capsid gene transcription of natural AAV (FIG. 12A) and TRACER libraries (FIG. 12B).

FIG. 13 is a diagram of the AAV6, AAV5 and AAV-DJ capsid peptide display libraries used for in vivo evolution (SEQ ID NOS 27-32, respectively, in order of appearance).

FIG. 14 is a diagram of the AAV9 capsid peptide display libraries used for in vivo evolution (SEQ ID NOS 33-42, respectively, in order of appearance).

FIG. 15A and FIG. 15B present the method used for library construction. FIG. 15A shows the sequence of the insertion site used to introduce random libraries (SEQ ID NOS 43-46, respectively, in order of appearance). FIG. 15B provides a description of the assembly procedure.

FIG. 16 provides an exemplary diagram of cloning-free rolling circle procedure used for library amplification (SEQ ID NO 47; NNK₇).

FIG. 17 provides the sequence of the codon-mutant AAV9 library shuttle designed to minimize wild-type contamination (SEQ ID NOS 33-34 and 48-52, respectively, in order of appearance).

FIG. 18 provides a description of AAV9 peptide libraries biopanning.

FIG. 19 illustrates the recovery process from an initial pool with recovery at 50%.

FIG. 20 provides an example of the cDNA recovery and amplification from GFAP-driven libraries (B group and F group).

FIG. 21A, FIG. 21B and FIG. 21C show the progression of AAV9 peptide library diversity throughout the biopanning process. FIG. 21A describes RNA library evolution. FIG. 21B and FIG. 21C show the amino acid distribution of NNK machine mix preparations for P0 and P1 virus.

FIG. 22 provides neuron (SYN)-AAV9 Peptide Libraries Composition at P2.

FIG. 23 provides astrocyte (GFAP)-AAV9 Peptide Libraries Composition at P2.

FIG. 24 provides an estimation of brain/liver specificity in GFAP-AAV9 peptide library candidates.

FIG. 25 provides an estimation of brain/liver specificity in GFAP-AAV9 peptide library candidates.

FIG. 26 provide an example subpopulation selection of variants.

FIG. 27 provides an exemplary design of a library generation and cloning procedure.

FIG. 28 provides the NNK/NNM codon distribution (covariance of codon mutants) of AAV produced with a synthetic library of 666 sequence variants (GFAP promoter).

FIG. 29 provides the NNK/NNM codon distribution (covariance of codon mutants) of AAV produced with a synthetic library of 666 sequence variants (SYN9 promoter).

FIG. 30 provides the data from the tissue recovery, one-month post injection, from brain and a liver punch.

FIG. 31A, FIG. 31B, FIG. 31C and FIG. 31D provide results of control capsids from the Syn-driven synthetic library NGS analysis. FIG. 31A shows the enrichment analysis of internal AAV9, PHP.B and PHP.eB controls (SEQ ID NOS 53-58 and 53-58, respectively, in order of appearance). FIG. 31B, FIG. 31C and FIG. 31D show the NNK/NNM codon distribution in mRNA from mouse brain tissue.

FIG. 32A and FIG. 32B provide the results of the neuron synthetic library NGS analysis (SEQ ID NOS 59-60, 59-61, 61-63, 62, 64, 64, 63, 65-67, 67, 65, 68, 66, 69, 70-71, and 70-74, respectively, in order of appearance).

FIG. 33 provides the results of the astrocyte synthetic library NGS analysis (SEQ ID NOS 53-58, 53-58, and 53-58, respectively, in order of appearance).

FIG. 34A and FIG. 34B provide astrocyte synthetic library codon mutants covariance.

FIG. 35 provides the results of the astrocyte synthetic library NGS analysis (SEQ ID NOS 75, 75-78, 76-77, 79-83, 65, 78, 84, 80, 85, 70, 86, 82, 81, 79, 87, 65, 85, 84, 70, 86, 88-90, 87, 91, 83, 88, 63, 89-90, 92-93, 91, 94-97, 93, 95, 98, 98, 97, 63, 92, 94, 99-101, 75, 75-78, 76-77, 79-83, 65, 78, 84, 80, 85, 70, 86, 82, 81, 79, 87, 65, 85, 84, 70, 86, 88-90, 87, 91, 83, 88, 63, 89-90, 92-93, 91, 94-97, 93, 95, 98, 98, 97, 63, 92, 94, 99-102, 99, 103, 103-104, 96, 105-106, 101, 100, 102, 107, 104-105, 108-113, 106, 60, 66, 114-117, 109, 113, 72, 108, 110, 67, 118-119, 116, 120, 120, 107, 112, 121-123, 66, 124-125, 115, 118, 126, 121, 127-128, 60, 129, 119, 130-132, 72, 133, 123, 125, 69, 134-139, 62, 124, 67, 111, 114, 126, 140-141, 122, 142, 128-129, 143, 138, 144, 134, 62, 136, 145, 141, 146-153, 127, 154, 69, 144, 155, 71, 156, 133, 132, 137, 147, 157-158, 135, 159, 140, 117, 160, 139, 161-162, 130, 163, 143, 164, 152, 151, 165-167, 155, 168, 71, 169, and 146, respectively, in order of appearance).

FIG. 36 provides the GFAP synthetic library NGS analysis.

FIG. 37A and FIG. 37B provides the top 38 variants from the synthetic library screen. FIG. 37A shows the phylogenetic analysis of 9-mer peptide sequences, and also shows the sequence of the peptide variants (SEQ ID NOS 67, 59, 64, 61, 77, 84, 96, 60, 80, 82, 66, 62, 83, 85, 106, 131, 94, 90, 76, 68-69, 79, 75, 81, 88, 139, 78, 155, 102, 63, 140, 87, 70, 105, 120, 89, 65, and 109, respectively, in order of appearance). Highlighted sequences represent the peptides that were selected for individual transduction assay. FIG. 37B shows the graphic representation of the neuron and astrocyte tropism of each peptide, both axis indicate the inverted rank in Synapsin and GFAP screen.

FIG. 38 provides the top consensus sequences as compared to PHP.N and PHP.B (SEQ ID NOS 168 and 71, respectively, in order of appearance).

FIG. 39 is a diagram of the Gibson assembly library cloning procedure.

FIG. 40 provides an example of TRIM/NNK peptide prevalence (SEQ ID NOS 170-171, respectively, in order of appearance).

FIG. 41 provides peptide diversity statistics from a study using the Illumina adapter having 42 million bacterial transformants, 81 million sequence reads and 12 million sequence variants (SEQ ID NOS 172-173, 48-49, and 174-175, respectively, in order of appearance).

FIG. 42 provides an exemplary diagram of cloning-free DNA amplification by rolling circle amplification.

FIG. 43 provides a diagram of protelomerase monomer processing (SEQ ID NOS 176-178, respectively, in order of appearance).

FIG. 44 provides a diagram comparing the traditional and cloning-free methods.

FIG. 45A and FIG. 45C provide the full ranking of Syn-driven (FIG. 45A) and GFAP-driven (FIG. 45B) 333 variants in the brain, spinal cord, liver and heart tissues. Capsid variants are ranked by their average brain RNA enrichment score (average of NNK and NNM codons). The rank of internal control capsids PHP.B, PHP.eB and AAV9 is indicated (FIG. 45A and FIG. 45B). A comparison of combined Syn-driven results and GFAP-driven results is provided (FIG. 45C). Only 4 animals were represented for the GFAP-driven libraries because 2/6 mice showed a very different ranking profile and were considered as outliers.

FIG. 46A and FIG. 46B provide the comparison of results of the neuron and astrocyte synthetic library NGS analysis. FIG. 46A shows the ranking of capsids using SYN or GFAP promoters; FIG. 46B shows the scatter plot showing the correlation of Syn-versus GFAP-driven libraries.

FIG. 47 illustrates one embodiment of a multi-species (e.g., rodent) study followed by next generation sequencing (NGS).

FIG. 48A, FIG. 48B and FIG. 48C provide results from a multi-strain/species comparison of 333 capsid variants. FIG. 48A shows the ranking of 333 capsids by brain RNA enrichment score in C57BL/6 mice, BALB/C mice and rats. Capsids are ranked according to Syn-driven brain enrichment score in C57BL/6 mice. FIG. 48B shows the scatter plots showing the correlation between C57BL/6 and BALB/C enrichment scores from Syn- and GFAP-driven pools. FIG. 48C shows the Venn diagram showing the intersection and consensus sequence of capsids with a brain enrichment score >10-fold higher than AAV9 (either Syn- or GFAP-driven) in C57BL/6 and BALB/C strains. In rats, no capsid showed an enrichment score >10-fold versus AAV9.

FIG. 49A, FIG. 49B, FIG. 49C and FIG. 49D provide transduction (RNA) and biodistribution (DNA) analysis of 10 capsid variants indicated in FIG. 49A (SEQ ID NOS 179-188, respectively, in order of appearance). Individual capsids were used to package self-complementary CBA-EGFP genomes (FIG. 49B) and injected intravenously to C57BL/6 mice. FIG. 49C shows the RNA expression in brain and spinal cord samples. FIG. 49D shows the DNA distribution in brain and spinal cord samples.

FIG. 50A, FIG. 50B and FIG. 50C provide the results of testing of individual capsids and their mRNA expression in brain, spinal cord and liver. EGFP mRNA expression results are shown for the brain (FIG. 50A), the spinal cord (FIG. 50B) and the liver (FIG. 50C).

FIG. 51 provides results for NGS screening using neuronal NeuN marker (FIG. 51) for both GFAP screening and SYN screening.

FIG. 52 provides the results of testing of individual capsids in whole brain.

FIG. 53 provides the results of testing of additional individual capsids in whole brain.

FIG. 54 provides the results of testing of individual capsids in cerebellum.

FIG. 55 provides the results of testing of individual capsids in cortex.

FIG. 56 provides the results of testing of individual capsids in hippocampus.

FIG. 57A and FIG. 57B provide transduction data of 10 capsid variants in mouse liver (FIG. 57B), analyzed by EGFP RNA expression and whole tissue fluorescence (FIG. 57A).

FIG. 58A and FIG. 58B provide results for comparison studies on the efficacy of the 333 capsid variants to transduce CNS for C57BL/6 mice BMVEC (FIG. 58A) and Human BMVEC (FIG. 58B).

FIG. 59A, FIG. 59B and FIG. 57C provide diagrams of external barcoding for NGS analysis and recovery of full-length capsid variants. A general barcode pair is shown (FIG. 59C). Full ITR-to-ITR constructs are shown with the barcode pair 5′ of the CAP sequence (FIG. 59A) and 3′ of the CAP sequence (FIG. 59B).

FIG. 60A, FIG. 60B and FIG. 60C provide detailed analysis of virus production and RNA splicing with several configurations of intronic barcoded platforms. A general ITR-to-ITR construct is shown in FIG. 60A (SEQ ID NOS 189-193, respectively, in order of appearance), with intronic barcode yields (FIG. 60B) and gel columns showing AAV intron splicing and Globin intron splicing results (FIG. 60C).

### DETAILED DESCRIPTION OF THE DISCLOSURE

The details of one or more embodiments of the disclosure are set forth in the accompanying description below. Although any materials and methods similar or equivalent to those described herein can be used in the practice or testing of the present disclosure, the preferred materials and methods are now described. Other features, objects and advantages of the disclosure will be apparent from the description. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

According to the present disclosure, AAV particles with enhanced tropism for a target tissue (e.g., CNS) are provided, as well as associated processes for their targeting, preparation, formulation and use. Targeting peptides and nucleic acid sequences encoding the targeting peptides are provided. These targeting peptides may be inserted into an AAV capsid protein sequence to alter tropism to a particular cell-type, tissue, organ or organism, in vivo, ex vivo or in vitro.

As used herein, an “AAV particle” or “AAV vector” comprises a capsid protein and a viral genome, wherein the viral genome comprises at least one payload region and at least one inverted terminal repeat (ITR). The AAV particle and/or its component capsid and viral genome may be engineered to alter tropism to a particular cell-type, tissue, organ or organism.

As used herein, “viral genome” or “vector genome” refers to the nucleic acid sequence(s) encapsulated in an AAV particle. A viral genome comprises a nucleic acid sequence with at least one payload region encoding a payload and at least one ITR.

As used herein, a “payload region” is any nucleic acid molecule which encodes one or more “payloads” of the disclosure. As non-limiting examples, a payload region may be a nucleic acid sequence encoding a payload comprising an RNAi agent or a polypeptide.

As used herein, a “targeting peptide” refers to a peptide of 3-20 amino acids in length. These targeting peptides may be inserted into, or attached to, a parent amino acid sequence to alter the characteristics (e.g., tropism) of the parent protein. As a non-limiting example, the targeting peptide can be inserted into an AAV capsid sequence for enhanced targeting to a desired cell-type, tissue, organ or organism.

The AAV particles and payloads of the disclosure may be delivered to one or more target cells, tissues, organs, or organisms. In a preferred embodiment, the AAV particles of the disclosure demonstrate enhanced tropism for a target cell type, tissue or organ. As a non-limiting example, the AAV particle may have enhanced tropism for cells and tissues of the central or peripheral nervous systems (CNS and PNS, respectively). The AAV particles of the disclosure may, in addition, or alternatively, have decreased tropism for an undesired target cell-type, tissue or organ.

Adeno-associated viruses (AAV) are small non-enveloped icosahedral capsid viruses of the Parvoviridae family characterized by a single stranded DNA viral genome. Parvoviridae family viruses consist of two subfamilies: Parvovirinae, which infect vertebrates, and Densovirinae, which infect invertebrates. The Parvoviridae family comprises the Dependovirus genus which includes AAV, capable of replication in vertebrate hosts including, but not limited to, human, primate, bovine, canine, equine, and ovine species.

The parvoviruses and other members of the Parvoviridae family are generally described in Kenneth I. Berns, “Parvoviridae: The Viruses and Their Replication,” Chapter 69 in FIELDS VIROLOGY (3d Ed. 1996), the contents of which are incorporated by reference in their entirety.

AAV have proven to be useful as a biological tool due to their relatively simple structure, their ability to infect a wide range of cells (including quiescent and dividing cells) without integration into the host genome and without replicating, and their relatively benign immunogenic profile. The genome of the virus may be manipulated to contain a minimum of components for the assembly of a functional recombinant virus, or viral particle, which is loaded with or engineered to target a particular tissue and express or deliver a desired payload.

The wild-type AAV vector genome is a linear, single-stranded DNA (ssDNA) molecule approximately 5,000 nucleotides (nt) in length. Inverted terminal repeats (ITRs) traditionally cap the viral genome at both the 5′ and the 3′ end, providing origins of replication for the viral genome. While not wishing to be bound by theory, an AAV viral genome typically comprises two ITR sequences. These ITRs have a characteristic T-shaped hairpin structure defined by a self-complementary region (145 nt in wild-type AAV) at the 5′ and 3′ ends of the ssDNA which form an energetically stable double stranded region. The double stranded hairpin structures comprise multiple functions including, but not limited to, acting as an origin for DNA replication by functioning as primers for the endogenous DNA polymerase complex of the host viral replication cell.

The wild-type AAV viral genome further comprises nucleotide sequences for two open reading frames, one for the four non-structural Rep proteins (Rep78, Rep68, Rep52, Rep40, encoded by Rep genes) and one for the three capsid, or structural, proteins (VP1, VP2, VP3, encoded by capsid genes or Cap genes). The Rep proteins are important for replication and packaging, while the capsid proteins are assembled to create the protein shell of the AAV, or AAV capsid. Alternative splicing and alternate initiation codons and promoters result in the generation of four different Rep proteins from a single open reading frame and the generation of three capsid proteins from a single open reading frame. Though it varies by AAV serotype, as a non-limiting example, for AAV9/hu.14 (SEQ ID NO: 123 of U.S. Pat. No. 7,906,111, the contents of which are herein incorporated by reference in their entirety) VP1 refers to amino acids 1-736, VP2 refers to amino acids 138-736, and VP3 refers to amino acids 203-736. In other words, VP1 is the full-length capsid sequence, while VP2 and VP3 are shorter components of the whole. As a result, changes in the sequence in the VP3 region, are also changes to VP1 and VP2, however, the percent difference as compared to the parent sequence will be greatest for VP3 since it is the shortest sequence of the three. Though described here in relation to the amino acid sequence, the nucleic acid sequence encoding these proteins can be similarly described. Together, the three capsid proteins assemble to create the AAV capsid protein. While not wishing to be bound by theory, the AAV capsid protein typically comprises a molar ratio of 1:1:10 of VP1:VP2:VP3. As used herein, an “AAV serotype” is defined primarily by the AAV capsid. In some instances, the ITRs are also specifically described by the AAV serotype (e.g., AAV2/9).

AAV vectors of the present disclosure may be produced recombinantly and may be based on adeno-associated virus (AAV) parent or reference sequences. As used herein, a “vector” is any molecule or moiety which transports, transduces, or otherwise acts as a carrier of a heterologous molecule such as the nucleic acids described herein.

In addition to single stranded AAV viral genomes (e.g., ssAAVs), the present disclosure also provides for self-complementary AAV (scAAVs) viral genomes. scAAV vector genomes contain DNA strands which anneal together to form double stranded DNA. By skipping second strand synthesis, scAAVs allow for rapid expression in the transduced cell.

In one embodiment, the AAV particle of the present disclosure is an scAAV.

In one embodiment, the AAV particle of the present disclosure is an ssAAV.

Methods for producing and/or modifying AAV particles are disclosed in the art such as pseudotyped AAV vectors (PCT Patent Publication Nos. WO200028004; WO200123001; WO2004112727; WO2005005610; and WO2005072364, the content of each of which is incorporated herein by reference in its entirety).

In one embodiment, the AAV particles of the disclosure comprising a capsid with an inserted targeting peptide and a viral genome, may have enhanced tropism for a cell-type or tissue of the human CNS.

### AAV Capsids

AAV particles of the present disclosure may comprise or be derived from any natural or recombinant AAV serotype. AAV serotypes may differ in characteristics such as, but not limited to, packaging, tropism, transduction and immunogenic profiles. While not wishing to be bound by theory, the AAV capsid protein is often considered to be the driver of AAV particle tropism to a particular tissue.

In one embodiment, an AAV particle may have a capsid protein and ITR sequences derived from the same parent serotype (e.g., AAV2 capsid and AAV2 ITRs). In another embodiment, the AAV particle may be a pseudo-typed AAV particle, wherein the capsid protein and ITR sequences are derived from different parent serotypes (e.g., AAV9 capsid and AAV2 ITRs; AAV2/9).

The AAV particles of the present disclosure may comprise an AAV capsid protein with a targeting peptide inserted into the parent sequence. The parent capsid or serotype may comprise or be derived from any natural or recombinant AAV serotype. As used herein, a “parent” sequence is a nucleotide or amino acid sequence into which a targeting sequence is inserted (i.e., nucleotide insertion into nucleic acid sequence or amino acid sequence insertion into amino acid sequence).

In a preferred embodiment, the parent AAV capsid nucleotide sequence is as set forth in SEQ ID NO: 1.

In another embodiment, the parent AAV capsid nucleotide sequence is a K449R variant of SEQ ID NO: 1, wherein the codon encoding a lysine (e.g., AAA or AAG) at position 449 in the amino acid sequence (nucleotides 1345-1347) is exchanged for one encoding an arginine (CGT, CGC, CGA, CGG, AGA, AGG). The K449R variant has the same function as wild-type AAV9.

In one embodiment, the parent AAV capsid amino acid sequence is as set forth in SEQ ID NO: 2.

In another embodiment, the parent AAV capsid amino acid sequence is as set forth in SEQ ID NO: 3.

In one embodiment the parent AAV capsid sequence is any of those shown in Table 1.

TABLE 1
AAV Capsid Sequences

| | SEQ | |
|---|---|---|
| Serotype | ID NO | Reference Information |
| AAV9/hu.14 (nt) | 1 | U.S. Pat. No. 7,906,111 SEQ ID NO: |
| | | 3; WO2015038958 SEQ ID NO: 11 |
| AAV9/hu.14 (aa) | 2 | U.S. Pat. No. 7,906,111 SEQ ID NO: |
| | | 123; WO2015038958 SEQ ID NO: 2 |
| AAV9/hu.14 K449R (aa) | 3 | WO2017100671 SEQ ID NO: 45 |

Each of the patents, applications and or publications listed in Table 1 are hereby incorporated by reference in their entirety.

The parent AAV serotype and associated capsid sequence may be any of those known in the art. Non-limiting examples of such AAV serotypes include, AAV9, AAV9 K449R (or K449R AAV9), AAV1, AAVrh10, AAV-DJ, AAV-DJ8, AAV5, AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP (3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2A15/G2A3 (G2A3), AAVG2B4 (G2B4), AAVG2B5 (G2B5), PHP.S, AAV2, AAV2G9, AAV3, AAV3a, AAV3b, AAV3-3, AAV4, AAV4-4, AAV6, AAV6.1, AAV6.2, AAV6.1.2, AAV7, AAV7.2, AAV8, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AA Vhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AA Vrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, and/or AAVF9/HSC9 and variants thereof.

In some embodiments, the serotype may be AAVDJ or a variant thereof, such as AAVDJ8 (or AAV-DJ8), as described by Grimm et al. (Journal of Virology 82(12): 5887-5911 (2008), US Publication US20140359799 and U.S. Pat. No. 7,588,772, each of which is herein incorporated by reference in its entirety). The amino acid sequence of AAVDJ8 may comprise two or more mutations in order to remove the heparin binding domain (HBD). As a non-limiting example, the AAV-DJ sequence is as described by SEQ ID NO: 1 in U.S. Pat. No. 7,588,772, the contents of which are herein incorporated by reference in their entirety, and the AAVDJ8 sequence may comprise two mutations: (1) R587Q where arginine (R; Arg) at amino acid 587 is changed to glutamine (Q; Gln) and (2) R590T where arginine (R; Arg) at amino acid 590 is changed to threonine (T; Thr). As another non-limiting example, the AAVDJ8 sequence may comprise three mutations: (1) K406R where lysine (K; Lys) at amino acid 406 is changed to arginine (R; Arg), (2) R587Q where arginine (R; Arg) at amino acid 587 is changed to glutamine (Q; Gln) and (3) R590T where arginine (R; Arg) at amino acid 590 is changed to threonine (T; Thr).

In one embodiment, the parent AAV capsid sequence comprises an AAV9 sequence.

In one embodiment, the parent AAV capsid sequence comprises an K449R AAV9 sequence.

In one embodiment, the parent AAV capsid sequence comprises an AAVDJ sequence.

In one embodiment, the parent AAV capsid sequence comprises an AAVDJ8 sequence.

In one embodiment, the parent AAV capsid sequence comprises an AAVrh10 sequence.

In one embodiment, the parent AAV capsid sequence comprises an AAV1 sequence.

In one embodiment, the parent AAV capsid sequence comprises an AAV5 sequence.

While not wishing to be bound by theory, it is understood that a parent AAV capsid sequence comprises a VP1 region. In one embodiment, a parent AAV capsid sequence comprises a VP1, VP2 and/or VP3 region, or any combination thereof. A parent VP1 sequence may be considered synonymous with a parent AAV capsid sequence.

The present disclosure refers to structural capsid proteins (including VP1, VP2 and VP3) which are encoded by capsid (Cap) genes. These capsid proteins form an outer protein structural shell (i.e. capsid) of a viral vector such as AAV. VP capsid proteins synthesized from Cap polynucleotides generally include a methionine as the first amino acid in the peptide sequence (Met1), which is associated with the start codon (AUG or ATG) in the corresponding Cap nucleotide sequence. However, it is common for a first-methionine (Met1) residue or generally any first amino acid (AA1) to be cleaved off after or during polypeptide synthesis by protein processing enzymes such as Met-aminopeptidases. This “Met/AA-clipping” process often correlates with a corresponding acetylation of the second amino acid in the polypeptide sequence (e.g., alanine, valine, serine, threonine, etc.). Met-clipping commonly occurs with VP1 and VP3 capsid proteins but can also occur with VP2 capsid proteins.

Where the Met/AA-clipping is incomplete, a mixture of one or more (one, two or three) VP capsid proteins comprising the viral capsid may be produced, some of which may include a Met1/AA1 amino acid (Met+/AA+) and some of which may lack a Met1/AA1 amino acid as a result of Met/AA-clipping (Met−/AA−). For further discussion regarding Met/AA-clipping in capsid proteins, see Jin, et al. Direct Liquid Chromatography/Mass Spectrometry Analysis for Complete Characterization of Recombinant Adeno-Associated Virus Capsid Proteins. Hum Gene Ther Methods. 2017 Oct. 28(5):255-267; Hwang, et al. N-Terminal Acetylation of Cellular Proteins Creates Specific Degradation Signals. Science. 2010 Feb. 19. 327(5968):973-977; the contents of which are each incorporated herein by reference in its entirety.

According to the present disclosure, references to capsid proteins is not limited to either clipped (Met−/AA−) or unclipped (Met+/AA+) and may, in context, refer to independent capsid proteins, viral capsids comprised of a mixture of capsid proteins, and/or polynucleotide sequences (or fragments thereof) which encode, describe, produce or result in capsid proteins of the present disclosure. A direct reference to a “capsid protein” or “capsid polypeptide” (such as VP1, VP2 or VP2) may also comprise VP capsid proteins which include a Met1/AA1 amino acid (Met+/AA+) as well as corresponding VP capsid proteins which lack the Met1/AA1 amino acid as a result of Met/AA-clipping (Met−/AA−).

Further according to the present disclosure, a reference to a specific SEQ ID NO: (whether a protein or nucleic acid) which comprises or encodes, respectively, one or more capsid proteins which include a Met1/AA1 amino acid (Met+/AA+) should be understood to teach the VP capsid proteins which lack the Met1/AA1 amino acid as upon review of the sequence, it is readily apparent any sequence which merely lacks the first listed amino acid (whether or not Met1/AA1).

As a non-limiting example, reference to a VP1 polypeptide sequence which is 736 amino acids in length and which includes a “Met1” amino acid (Met+) encoded by the AUG/ATG start codon may also be understood to teach a VP1 polypeptide sequence which is 735 amino acids in length and which does not include the “Met1” amino acid (Met−) of the 736 amino acid Met+ sequence. As a second non-limiting example, reference to a VP1 polypeptide sequence which is 736 amino acids in length and which includes an “AA1” amino acid (AA1+) encoded by any NNN initiator codon may also be understood to teach a VP1 polypeptide sequence which is 735 amino acids in length and which does not include the “AA1” amino acid (AA1−) of the 736 amino acid AA1+ sequence.

References to viral capsids formed from VP capsid proteins (such as reference to specific AAV capsid serotypes), can incorporate VP capsid proteins which include a Met1/AA1 amino acid (Met+/AA1+), corresponding VP capsid proteins which lack the Met1/AA1 amino acid as a result of Met/AA1-clipping (Met−/AA1−), and combinations thereof (Met+/AA1+ and Met−/AA1−).

As a non-limiting example, an AAV capsid serotype can include VP1 (Met+/AA1+), VP1 (Met−/AA1−), or a combination of VP1 (Met+/AA1+) and VP1 (Met−/AA1−). An AAV capsid serotype can also include VP3 (Met+/AA1+), VP3 (Met−/AA1−), or a combination of VP3 (Met+/AA1+) and VP3 (Met−/AA1−); and can also include similar optional combinations of VP2 (Met+/AA1) and VP2 (Met−/AA1−).

In one embodiment, the parent AAV capsid sequence may comprise an amino acid sequence with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any of the those described above.

In one embodiment, the parent AAV capsid sequence may be encoded by a nucleotide sequence with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any of those described above.

In one embodiment, the parent sequence is not an AAV capsid sequence and is instead a different vector (e.g., lentivirus, plasmid, etc.). In another embodiment, the parent sequence is a delivery vehicle (e.g., a nanoparticle) and the targeting peptide is attached thereto.

### Targeting Peptides

Disclosed herein are targeting peptides and associated AAV particles comprising a capsid protein with one or more targeting peptide inserts, for enhanced or improved transduction of a target tissue (e.g., cells of the CNS or PNS).

In one embodiment, the targeting peptide may direct an AAV particle to a cell or tissue of the CNS. The cell of the CNS may be, but is not limited to, neurons (e.g., excitatory, inhibitory, motor, sensory, autonomic, sympathetic, parasympathetic, Purkinje, Betz, etc.), glial cells (e.g., microglia, astrocytes, oligodendrocytes) and/or supporting cells of the brain such as immune cells (e.g., T cells). The tissue of the CNS may be, but is not limited to, the cortex (e.g., frontal, parietal, occipital, temporal), thalamus, hypothalamus, striatum, putamen, caudate nucleus, hippocampus, entorhinal cortex, basal ganglia, or deep cerebellar nuclei.

In one embodiment, the targeting peptide may direct an AAV particle to a cell or tissue of the PNS. The cell or tissue of the PNS may be, but is not limited to, a dorsal root ganglion (DRG).

The targeting peptide may direct an AAV particle to the CNS (e.g., the cortex) after intravenous administration.

The targeting peptide may direct and AAV particle to the PNS (e.g., DRG) after intravenous administration.

A targeting peptide may vary in length. In one embodiment, the targeting peptide is 3-20 amino acids in length. As non-limiting examples, the targeting peptide may be 3, 4, 5, 6, 7, 8, 9, 10, 11, 12, 13, 14, 15, 16, 17, 18, 19, 20 or 3-5, 3-8, 3-10, 3-12, 3-15, 3-18, 3-20, 5-10, 5-15, 5-20, 10-12, 10-15, 10-20, 12-20, or 15-20 amino acids in length.

Targeting peptides of the present disclosure may be identified and/or designed by any method known in the art. As a non-limiting example, the CREATE system as described in Deverman et al., (Nature Biotechnology 34(2):204-209 (2016)), Chan et al., (Nature Neuroscience 20(8):1172-1179 (2017)), and in International Patent Application Publication Nos. WO2015038958 and WO2017100671, the contents of each of which are herein incorporated by reference in their entirety, may be used as a means of identifying targeting peptides, in either mice or other research animals, such as, but not limited to, non-human primates.

Targeting peptides and associated AAV particles may be identified from libraries of AAV capsids comprised of targeting peptide variants. In one embodiment, the targeting peptides may be 7 amino acid sequences (7-mers). In another embodiment, the targeting peptides may be 9 amino acid sequences (9-mers). The targeting peptides may also differ in their method of creation or design, with non-limiting examples including, random peptide selection, site saturation mutagenesis, and/or optimization of a particular region of the peptide (e.g., flanking regions or central core).

In one embodiment, a targeting peptide library comprises targeting peptides of 7 amino acids (7-mer) in length randomly generated by PCR.

In one embodiment, a targeting peptide library comprises targeting peptides with 3 mutated amino acids. In one embodiment, these 3 mutated amino acids are consecutive amino acids. In another embodiment, these 3 mutated amino acids are not consecutive amino acids. In one embodiment, the parent targeting peptide is a 7-mer. In another embodiment, the parent peptide is a 9-mer.

In one embodiment, a targeting peptide library comprises 7-mer targeting peptides, wherein the amino acids of the targeting peptide and/or the flanking sequences are evolved through site saturation mutagenesis of 3 consecutive amino acids. In one embodiment, NNK (N=any base; K=G or T) codons are used to generate the site saturated mutation sequences.

AAV particles comprising capsid proteins with targeting peptide inserts are generated and viral genomes encoding a reporter (e.g., GFP) encapsulated within. These AAV particles (or AAV capsid library) are then administered to a transgenic mouse by intravenous delivery to the tail vein. Administration of these capsid libraries to cre-expressing mice results in expression of the reporter payload in the target tissue, due to the expression of Cre.

AAV particles and/or viral genomes may be recovered from the target tissue for identification of targeting peptides and associated AAV particles that are enriched, indicating enhanced transduction of target tissue. Standard methods in the art, such as, but not limited to next generation sequencing (NGS), viral genome quantification, biochemical assays, immunohistochemistry and/or imaging of target tissue samples may be used to determine enrichment.

A target tissue may be any cell, tissue or organ of a subject. As non-limiting examples, samples may be collected from brain, spinal cord, dorsal root ganglia and associated roots, liver, heart, gastrocnemius muscle, soleus muscle, pancreas, kidney, spleen, lung, adrenal glands, stomach, sciatic nerve, saphenous nerve, thyroid gland, eyes (with or without optic nerve), pituitary gland, skeletal muscle (rectus femoris), colon, duodenum, ileum, jejunum, skin of the leg, superior cervical ganglia, urinary bladder, ovaries, uterus, prostate gland, testes, and/or any sites identified as having a lesion, or being of interest.

### Targeting Peptide Sequences

In one embodiment the targeting peptide may comprise a sequence as set forth in Table 2. In Table 2, “_1” refers to NNM codons where A or C is in the third position and “_2” refers to NNK codons where G or T is in the third position. Additionally, the NNM codons cannot cover the entire repertoire of amino acids since Met or Trp can only be encoded by codons ATG and TGG, respectively. Therefore, some “NNM” sequences also contain some codons ending in G.

TABLE 2
Peptides

| Peptide | SEQ | Peptide | SEQ |
|---|---|---|---|
| Sequence_ID | ID NO: | Sequence_ID | ID NO: |
| AQAGAGSER_1 | 194 | DGTGQVTGW_1 | 68 |
| AQAGAGSER_2 | 194 | DGTGQVTGW_2 | 68 |
| AQDQNPGRW_1 | 195 | DGTGRLTGW_1 | 159 |
| AQDQNPGRW_2 | 195 | DGTGRLTGW_2 | 159 |
| AQELTRPFL_1 | 144 | DGTGRTVGW_1 | 117 |
| AQELTRPFL_2 | 144 | DGTGRTVGW_2 | 117 |
| AQEVPGYRW_1 | 196 | DGTGSGMMT_1 | 306 |
| AQEVPGYRW_2 | 196 | DGTGSGMMT_2 | 306 |
| AQFPTNYDS_1 | 66 | DGTGSISGW_1 | 307 |
| AQFPTNYDS_2 | 66 | DGTGSISGW_2 | 307 |
| AQFVVGQQY_1 | 95 | DGTGSLAGW_1 | 308 |
| AQFVVGQQY_2 | 95 | DGTGSLAGW_2 | 308 |
| AQGASPGRW_1 | 149 | DGTGSLNGW_1 | 309 |
| AQGASPGRW_2 | 149 | DGTGSLNGW_2 | 309 |
| AQGENPGRW_1 | 96 | DGTGSLQGW_1 | 310 |
| AQGENPGRW_2 | 96 | DGTGSLQGW_2 | 310 |
| AQGGNPGRW_1 | 91 | DGTGSLSGW_1 | 311 |
| AQGGNPGRW_2 | 91 | DGTGSLSGW_2 | 311 |
| AQGGSTGSN_1 | 197 | DGTGSLVGW_1 | 312 |
| AQGGSTGSN_2 | 197 | DGTGSLVGW_2 | 312 |
| AQGPTRPFL_1 | 125 | DGTGSTHGW_1 | 119 |
| AQGPTRPFL_2 | 125 | DGTGSTHGW_2 | 119 |
| AQGRDGWAA_1 | 198 | DGTGSTKGW_1 | 313 |
| AQGRDGWAA_2 | 198 | DGTGSTKGW_2 | 313 |
| AQGRMTDSQ_1 | 199 | DGTGSTMGW_1 | 314 |
| AQGRMTDSQ_2 | 199 | DGTGSTMGW_2 | 314 |
| AQGSDVGRW_1 | 128 | DGTGSTQGW_1 | 315 |
| AQGSDVGRW_2 | 128 | DGTGSTQGW_2 | 315 |
| AQGSNPGRW_1 | 103 | DGTGSTSGW_1 | 316 |
| AQGSNPGRW_2 | 103 | DGTGSTSGW_2 | 316 |
| AQGSNSPQV_1 | 200 | DGTGSTTGW_1 | 134 |
| AQGSNSPQV_2 | 200 | DGTGSTTGW_2 | 134 |
| AQGSWNPPA_1 | 80 | DGTGSVMGW_1 | 317 |
| AQGSWNPPA_2 | 80 | DGTGSVMGW_2 | 317 |
| AQGTWNPPA_1 | 82 | DGTGSVTGW_1 | 318 |
| AQGTWNPPA_2 | 82 | DGTGSVTGW_2 | 318 |
| AQGVFIPPK_1 | 201 | DGTGTLAGW_1 | 319 |
| AQGVFIPPK_2 | 201 | DGTGTLAGW_2 | 319 |
| AQHVNASQS_1 | 202 | DGTGTLHGW_1 | 320 |
| AQHVNASQS_2 | 202 | DGTGTLHGW_2 | 320 |
| AQIKAGWAQ_1 | 203 | DGTGTLKGW_1 | 321 |
| AQIKAGWAQ_2 | 203 | DGTGTLKGW_2 | 321 |
| AQIMSGYAQ_1 | 204 | DGTGTLSGW_1 | 322 |
| AQIMSGYAQ_2 | 204 | DGTGTLSGW_2 | 322 |
| AQKSVGSVY_1 | 205 | DGTGTTLGW_1 | 323 |
| AQKSVGSVY_2 | 205 | DGTGTTLGW_2 | 323 |
| AQLEHGFAQ_1 | 206 | DGTGTTMGW_1 | 324 |
| AQLEHGFAQ_2 | 206 | DGTGTTMGW_2 | 324 |
| AQLGGVLSA_1 | 207 | DGTGTTTGW_1 | 130 |
| AQLGGVLSA_2 | 207 | DGTGTTTGW_2 | 130 |
| AQLGLSQGR_1 | 208 | DGTGTTVGW_1 | 74 |
| AQLGLSQGR_2 | 208 | DGTGTTVGW_2 | 74 |
| AQLGYGFAQ_1 | 209 | DGTGTTYGW_1 | 325 |
| AQLGYGFAQ_2 | 209 | DGTGTTYGW_2 | 325 |
| AQLKYGLAQ_1 | 115 | DGTGTVHGW_1 | 326 |
| AQLKYGLAQ_2 | 115 | DGTGTVHGW_2 | 326 |
| AQLRIGFAQ_1 | 210 | DGTGTVQGW_1 | 327 |
| AQLRIGFAQ_2 | 210 | DGTGTVQGW_2 | 327 |
| AQLRMGYSQ_1 | 211 | DGTGTVSGW_1 | 328 |
| AQLRMGYSQ_2 | 211 | DGTGTVSGW_2 | 328 |
| AQLRQGYAQ_1 | 212 | DGTGTVTGW_1 | 329 |
| AQLRQGYAQ_2 | 212 | DGTGTVTGW_2 | 329 |
| AQLRVGFAQ_1 | 123 | DGTHARLSS_1 | 330 |
| AQLRVGFAQ_2 | 123 | DGTHARLSS_2 | 330 |
| AQLSCRSQM_1 | 213 | DGTHAYMAS_1 | 153 |
| AQLSCRSQM_2 | 213 | DGTHAYMAS_2 | 153 |
| AQLTYSQSL_1 | 214 | DGTHFAPPR_1 | 112 |
| AQLTYSQSL_2 | 214 | DGTHFAPPR_2 | 112 |
| AQLYKGYSQ_1 | 215 | DGTHIHLSS_1 | 162 |
| AQLYKGYSQ_2 | 215 | DGTHIHLSS_2 | 162 |
| AQMPQRPFL_1 | 216 | DGTHIRALS_1 | 331 |
| AQMPQRPFL_2 | 216 | DGTHIRALS_2 | 331 |
| AQNGNPGRW_1 | 84 | DGTHIRLAS_1 | 332 |
| AQNGNPGRW_2 | 84 | DGTHIRLAS_2 | 332 |
| AQPEGSARW_1 | 60 | DGTHLQPFR_1 | 333 |
| AQPEGSARW_2 | 60 | DGTHLQPFR_2 | 333 |
| AQPLAVYGA_1 | 217 | DGTHSFYDA_1 | 334 |
| AQPLAVYGA_2 | 217 | DGTHSFYDA_2 | 334 |
| AQPQSSSMS_1 | 218 | DGTHSTTGW_1 | 145 |
| AQPQSSSMS_2 | 218 | DGTHSTTGW_2 | 145 |
| AQPSVGGYW_1 | 219 | DGTHTRTGW_1 | 90 |
| AQPSVGGYW_2 | 219 | DGTHTRTGW_2 | 90 |
| AQQAVGQSW_1 | 220 | DGTHVRALS_1 | 335 |
| AQQAVGQSW_2 | 220 | DGTHVRALS_2 | 335 |
| AQQRSLASG_1 | 221 | DGTHVYMAS_1 | 336 |
| AQQRSLASG_2 | 221 | DGTHVYMAS_2 | 336 |
| AQQVMNSQG_1 | 222 | DGTHVYMSS_1 | 337 |
| AQQVMNSQG_2 | 222 | DGTHVYMSS_2 | 337 |
| AQRGVGLSQ_1 | 223 | DGTIALPFK_1 | 338 |
| AQRGVGLSQ_2 | 223 | DGTIALPFK_2 | 338 |
| AQRHDAEGS_1 | 224 | DGTIALPFR_1 | 339 |
| AQRHDAEGS_2 | 224 | DGTIALPFR_2 | 339 |
| AQRKGEPHY_1 | 225 | DGTIATRYV_1 | 340 |
| AQRKGEPHY_2 | 225 | DGTIATRYV_2 | 340 |
| AQRYTGDSS_1 | 138 | DGTIERPFR_1 | 87 |
| AQRYTGDSS_2 | 138 | DGTIERPFR_2 | 87 |
| AQSAMAAKG_1 | 226 | DGTIGYAYV_1 | 341 |
| AQSAMAAKG_2 | 226 | DGTIGYAYV_2 | 341 |
| AQSGGLTGS_1 | 227 | DGTIQAPFK_1 | 342 |
| AQSGGLTGS_2 | 227 | DGTIQAPFK_2 | 342 |
| AQSGGVGQV_1 | 228 | DGTIRLPFK_1 | 343 |
| AQSGGVGQV_2 | 228 | DGTIRLPFK_2 | 343 |
| AQSLATPFR_1 | 169 | DGTISKEVG_1 | 344 |
| AQSLATPFR_2 | 169 | DGTISKEVG_2 | 344 |
| AQSMSRPFL_1 | 229 | DGTISQPFK_1 | 105 |
| AQSMSRPFL_2 | 229 | DGTISQPFK_2 | 105 |
| AQSQLRPFL_1 | 230 | DGTKIQLSS_1 | 146 |
| AQSQLRPFL_2 | 230 | DGTKIQLSS_2 | 146 |
| AQSVAKPFL_1 | 231 | DGTKIRLSS_1 | 111 |
| AQSVAKPFL_2 | 231 | DGTKIRLSS_2 | 111 |
| AQSVSQPFR_1 | 232 | DGTKLMLSS_1 | 157 |
| AQSVSQPFR_2 | 232 | DGTKLMLSS_2 | 157 |
| AQSVVRPFL_1 | 233 | DGTKLRLSS_1 | 118 |
| AQSVVRPFL_2 | 233 | DGTKLRLSS_2 | 118 |
| AQTALSSST_1 | 234 | DGTKMVLQL_1 | 142 |
| AQTALSSST_2 | 234 | DGTKMVLQL_2 | 142 |
| AQTEMGGRC_1 | 235 | DGTKSLVQL_1 | 345 |
| AQTEMGGRC_2 | 235 | DGTKSLVQL_2 | 345 |
| AQTGFAPPR_1 | 161 | DGTKVLVQL_1 | 122 |
| AQTGFAPPR_2 | 161 | DGTKVLVQL_2 | 122 |
| AQTIRGYSS_1 | 236 | DGTLAAPFK_1 | 120 |
| AQTIRGYSS_2 | 236 | DGTLAAPFK_2 | 120 |
| AQTISNYHT_1 | 237 | DGTLAVNFK_1 | 346 |
| AQTISNYHT_2 | 237 | DGTLAVNFK_2 | 346 |
| AQTLARPFV_1 | 98 | DGTLAVPFK_1 | 71 |
| AQTLARPFV_2 | 98 | DGTLAVPFK_2 | 71 |
| AQTLAVPFK_1 | 168 | DGTLAYPFK_1 | 347 |
| AQTLAVPFK_2 | 168 | DGTLAYPFK_2 | 347 |
| AQTPDRPWL_1 | 238 | DGTLERPFR_1 | 156 |
| AQTPDRPWL_2 | 238 | DGTLERPFR_2 | 156 |
| AQTRAGYAQ_1 | 126 | DGTLEVHFK_1 | 348 |
| AQTRAGYAQ_2 | 126 | DGTLEVHFK_2 | 348 |
| AQTRAGYSQ_1 | 141 | DGTLLRLSS_1 | 121 |
| AQTRAGYSQ_2 | 141 | DGTLLRLSS_2 | 121 |
| AQTREYLLG_1 | 93 | DGTLNNPFR_1 | 109 |
| AQTREYLLG_2 | 93 | DGTLNNPFR_2 | 109 |
| AQTSAKPFL_1 | 163 | DGTLQQPFR_1 | 89 |
| AQTSAKPFL_2 | 163 | DGTLQQPFR_2 | 89 |
| AQTSARPFL_1 | 100 | DGTLSQPFR_1 | 65 |
| AQTSARPFL_2 | 100 | DGTLSQPFR_2 | 65 |
| AQTTDRPFL_1 | 85 | DGTLSRTLW_1 | 349 |
| AQTTDRPFL_2 | 85 | DGTLSRTLW_2 | 349 |
| AQTTEKPWL_1 | 83 | DGTLSSPFR_1 | 350 |
| AQTTEKPWL_2 | 83 | DGTLSSPFR_2 | 350 |
| AQTVARPFY_1 | 239 | DGTLTVPFR_1 | 351 |
| AQTVARPFY_2 | 239 | DGTLTVPFR_2 | 351 |
| AQTVATPFR_1 | 240 | DGTLVAPFR_1 | 352 |
| AQTVATPFR_2 | 240 | DGTLVAPFR_2 | 352 |
| AQTVTQLFK_1 | 241 | DGTMDKPFR_1 | 70 |
| AQTVTQLFK_2 | 241 | DGTMDKPFR_2 | 70 |
| AQVHVGSVY_1 | 165 | DGTMDRPFK_1 | 102 |
| AQVHVGSVY_2 | 165 | DGTMDRPFK_2 | 102 |
| AQVLAGYNM_1 | 242 | DGTMLRLSS_1 | 148 |
| AQVLAGYNM_2 | 242 | DGTMLRLSS_2 | 148 |
| AQVSEARVR_1 | 243 | DGTMQLTGW_1 | 353 |
| AQVSEARVR_2 | 243 | DGTMQLTGW_2 | 353 |
| AQVVVGYSQ_1 | 244 | DGTNGLKGW_1 | 76 |
| AQVVVGYSQ_2 | 244 | DGTNGLKGW_2 | 76 |
| AQWAAGYNV_1 | 245 | DGTNSISGW_1 | 354 |
| AQWAAGYNV_2 | 245 | DGTNSISGW_2 | 354 |
| AQWELSNGY_1 | 246 | DGTNSLSGW_1 | 355 |
| AQWELSNGY_2 | 246 | DGTNSLSGW_2 | 355 |
| AQWEVKGGY_1 | 247 | DGTNSTTGW_1 | 143 |
| AQWEVKGGY_2 | 247 | DGTNSTTGW_2 | 143 |
| AQWEVKRGY_1 | 248 | DGTNSVTGW_1 | 356 |
| AQWEVKRGY_2 | 248 | DGTNSVTGW_2 | 356 |
| AQWEVQSGF_1 | 249 | DGTNTINGW_1 | 124 |
| AQWEVQSGF_2 | 249 | DGTNTINGW_2 | 124 |
| AQWEVRGGY_1 | 250 | DGTNTLGGW_1 | 357 |
| AQWEVRGGY_2 | 250 | DGTNTLGGW_2 | 357 |
| AQWEVTSGW_1 | 251 | DGTNTTHGW_1 | 113 |
| AQWEVTSGW_2 | 251 | DGTNTTHGW_2 | 113 |
| AQWGAPSHG_1 | 252 | DGTNYRLSS_1 | 358 |
| AQWGAPSHG_2 | 252 | DGTNYRLSS_2 | 358 |
| AQWMELGSS_1 | 253 | DGTQALSGW_1 | 359 |
| AQWMELGSS_2 | 253 | DGTQALSGW_2 | 359 |
| AQWMFGGSG_1 | 254 | DGTQFRLSS_1 | 129 |
| AQWMFGGSG_2 | 254 | DGTQFRLSS_2 | 129 |
| AQWMLGGAQ_1 | 255 | DGTQFSPPR_1 | 108 |
| AQWMLGGAQ_2 | 255 | DGTQFSPPR_2 | 108 |
| AQWPTAYDA_1 | 256 | DGTQGLKGW_1 | 158 |
| AQWPTAYDA_2 | 256 | DGTQGLKGW_2 | 158 |
| AQWPTSYDA_1 | 62 | DGTQTTSGW_1 | 360 |
| AQWPTSYDA_2 | 62 | DGTQTTSGW_2 | 360 |
| AQWQVQTGF_1 | 257 | DGTRALTGW_1 | 361 |
| AQWQVQTGF_2 | 257 | DGTRALTGW_2 | 361 |
| AQWSTEGGY_1 | 258 | DGTRFSLSS_1 | 362 |
| AQWSTEGGY_2 | 258 | DGTRFSLSS_2 | 362 |
| AQWTAAGGY_1 | 259 | DGTRGLSGW_1 | 363 |
| AQWTAAGGY_2 | 259 | DGTRGLSGW_2 | 363 |
| AQWTTESGY_1 | 260 | DGTRIGLSS_1 | 364 |
| AQWTTESGY_2 | 260 | DGTRIGLSS_2 | 364 |
| AQWVYGSSH_1 | 261 | DGTRLHLAS_1 | 365 |
| AQWVYGSSH_2 | 261 | DGTRLHLAS_2 | 365 |
| AQYLAGYTV_1 | 262 | DGTRLHLSS_1 | 366 |
| AQYLAGYTV_2 | 262 | DGTRLHLSS_2 | 366 |
| AQYLKGYSV_1 | 152 | DGTRLLLSS_1 | 367 |
| AQYLKGYSV_2 | 152 | DGTRLLLSS_2 | 367 |
| AQYLSGYNT_1 | 263 | DGTRLMLSS_1 | 368 |
| AQYLSGYNT_2 | 263 | DGTRLMLSS_2 | 368 |
| DGAAATTGW_1 | 264 | DGTRLNLSS_1 | 369 |
| DGAAATTGW_2 | 264 | DGTRLNLSS_2 | 369 |
| DGAGGTSGW_1 | 151 | DGTRMVVQL_1 | 370 |
| DGAGGTSGW_2 | 151 | DGTRMVVQL_2 | 370 |
| DGAGTTSGW_1 | 265 | DGTRNMYEG_1 | 135 |
| DGAGTTSGW_2 | 265 | DGTRNMYEG_2 | 135 |
| DGAHGLSGW_1 | 266 | DGTRSITGW_1 | 371 |
| DGAHGLSGW_2 | 266 | DGTRSITGW_2 | 371 |
| DGAHVGLSS_1 | 267 | DGTRSLHGW_1 | 372 |
| DGAHVGLSS_2 | 267 | DGTRSLHGW_2 | 372 |
| DGARTVLQL_1 | 268 | DGTRSTTGW_1 | 373 |
| DGARTVLQL_2 | 268 | DGTRSTTGW_2 | 373 |
| DGEYQKPFR_1 | 269 | DGTRTTTGW_1 | 106 |
| DGEYQKPFR_2 | 269 | DGTRTTTGW_2 | 106 |
| DGGGTTTGW_1 | 270 | DGTRTVTGW_1 | 374 |
| DGGGTTTGW_2 | 270 | DGTRTVTGW_2 | 374 |
| DGHATSMGW_1 | 271 | DGTRTVVQL_1 | 375 |
| DGHATSMGW_2 | 271 | DGTRTVVQL_2 | 375 |
| DGKGSTQGW_1 | 272 | DGTRVHLSS_1 | 376 |
| DGKGSTQGW_2 | 272 | DGTRVHLSS_2 | 376 |
| DGKQYQLSS_1 | 92 | DGTSFPYAR_1 | 86 |
| DGKQYQLSS_2 | 92 | DGTSFPYAR_2 | 86 |
| DGNGGLKGW_1 | 167 | DGTSFTPPK_1 | 81 |
| DGNGGLKGW_2 | 167 | DGTSFTPPK_2 | 81 |
| DGQGGLSGW_1 | 273 | DGTSFTPPR_1 | 88 |
| DGQGGLSGW_2 | 273 | DGTSFTPPR_2 | 88 |
| DGQHFAPPR_1 | 110 | DGTSGLHGW_1 | 377 |
| DGQHFAPPR_2 | 110 | DGTSGLHGW_2 | 377 |
| DGRATKTLY_1 | 274 | DGTSGLKGW_1 | 101 |
| DGRATKTLY_2 | 274 | DGTSGLKGW_2 | 101 |
| DGRNALTGW_1 | 275 | DGTSIHLSS_1 | 378 |
| DGRNALTGW_2 | 275 | DGTSIHLSS_2 | 378 |
| DGRRQVIQL_1 | 276 | DGTSIMLSS_1 | 379 |
| DGRRQVIQL_2 | 276 | DGTSIMLSS_2 | 379 |
| DGRVYGLSS_1 | 277 | DGTSLRLSS_1 | 166 |
| DGRVYGLSS_2 | 277 | DGTSLRLSS_2 | 166 |
| DGSGRTTGW_1 | 147 | DGTSNYGAR_1 | 380 |
| DGSGRTTGW_2 | 147 | DGTSNYGAR_2 | 380 |
| DGSGTTRGW_1 | 114 | DGTSSYYDA_1 | 381 |
| DGSGTTRGW_2 | 114 | DGTSSYYDA_2 | 381 |
| DGSGTVSGW_1 | 278 | DGTSSYYDS_1 | 59 |
| DGSGTVSGW_2 | 278 | DGTSSYYDS_2 | 59 |
| DGSPEKPFR_1 | 160 | DGTSTISGW_1 | 382 |
| DGSPEKPFR_2 | 160 | DGTSTISGW_2 | 382 |
| DGSQSTTGW_1 | 136 | DGTSTITGW_1 | 383 |
| DGSQSTTGW_2 | 136 | DGTSTITGW_2 | 383 |
| DGSSFYPPK_1 | 127 | DGTSTLHGW_1 | 384 |
| DGSSFYPPK_2 | 127 | DGTSTLHGW_2 | 384 |
| DGSSSYYDA_1 | 64 | DGTSTLRGW_1 | 385 |
| DGSSSYYDA_2 | 64 | DGTSTLRGW_2 | 385 |
| DGSIERPFR_1 | 99 | DGTSTLSGW_1 | 386 |
| DGSIERPFR_2 | 99 | DGTSTLSGW_2 | 386 |
| DGTAARLSS_1 | 132 | DGTSYVPPK_1 | 97 |
| DGTAARLSS_2 | 132 | DGTSYVPPK_2 | 97 |
| DGTADKPFR_1 | 63 | DGTSYVPPR_1 | 78 |
| DGTADKPFR_2 | 63 | DGTSYVPPR_2 | 78 |
| DGTADRPFR_1 | 155 | DGTTATYYK_1 | 387 |
| DGTADRPFR_2 | 155 | DGTTATYYK_2 | 387 |
| DGTAERPFR_1 | 140 | DGTTFTPPR_1 | 79 |
| DGTAERPFR_2 | 140 | DGTTFTPPR_2 | 79 |
| DGTAIHLSS_1 | 67 | DGTTLAPFR_1 | 388 |
| DGTAIHLSS_2 | 67 | DGTTLAPFR_2 | 388 |
| DGTAIYLSS_1 | 279 | DGTTLVPPR_1 | 116 |
| DGTAIYLSS_2 | 279 | DGTTLVPPR_2 | 116 |
| DGTALMLSS_1 | 280 | DGTTSKTLW_1 | 389 |
| DGTALMLSS_2 | 280 | DGTTSKTLW_2 | 389 |
| DGTASISGW_1 | 281 | DGTTSRTLW_1 | 390 |
| DGTASISGW_2 | 281 | DGTTSRTLW_2 | 390 |
| DGTASTSGW_1 | 282 | DGTTTRSLY_1 | 391 |
| DGTASTSGW_2 | 282 | DGTTTRSLY_2 | 391 |
| DGTASVTGW_1 | 283 | DGTTTTTGW_1 | 392 |
| DGTASVTGW_2 | 283 | DGTTTTTGW_2 | 392 |
| DGTASYYDS_1 | 61 | DGTTTYGAR_1 | 77 |
| DGTASYYDS_2 | 61 | DGTTTYGAR_2 | 77 |
| DGTATTMGW_1 | 284 | DGTTWTPPR_1 | 139 |
| DGTATTMGW_2 | 284 | DGTTWTPPR_2 | 139 |
| DGTATTTGW_1 | 285 | DGTTYMLSS_1 | 393 |
| DGTATTTGW_2 | 285 | DGTTYMLSS_2 | 393 |
| DGTAYRLSS_1 | 286 | DGTTYVPPR_1 | 75 |
| DGTAYRLSS_2 | 286 | DGTTYVPPR_2 | 75 |
| DGTDKMWSL_1 | 287 | DGTVANPFR_1 | 394 |
| DGTDKMWSL_2 | 287 | DGTVANPFR_2 | 394 |
| DGTGGIKGW_1 | 131 | DGTVDRPFK_1 | 395 |
| DGTGGIKGW_2 | 131 | DGTVDRPFK_2 | 395 |
| DGTGGIMGW_1 | 288 | DGTVIHLSS_1 | 73 |
| DGTGGIMGW_2 | 288 | DGTVIHLSS_2 | 73 |
| DGTGGISGW_1 | 289 | DGTVILLSS_1 | 396 |
| DGTGGISGW_2 | 289 | DGTVILLSS_2 | 396 |
| DGTGGLAGW_1 | 290 | DGTVIMLSS_1 | 397 |
| DGTGGLAGW_2 | 290 | DGTVIMLSS_2 | 397 |
| DGTGGLHGW_1 | 291 | DGTVLHLSS_1 | 398 |
| DGTGGLHGW_2 | 291 | DGTVLHLSS_2 | 398 |
| DGTGGLQGW_1 | 292 | DGTVLMLSS_1 | 399 |
| DGTGGLQGW_2 | 292 | DGTVLMLSS_2 | 399 |
| DGTGGLRGW_1 | 154 | DGTVLVPFR_1 | 150 |
| DGTGGLRGW_2 | 154 | DGTVLVPFR_2 | 150 |
| DGTGGLSGW_1 | 293 | DGTVPYLAS_1 | 400 |
| DGTGGLSGW_2 | 293 | DGTVPYLAS_2 | 400 |
| DGTGGLTGW_1 | 294 | DGTVPYLSS_1 | 401 |
| DGTGGLTGW_2 | 294 | DGTVPYLSS_2 | 401 |
| DGTGGTKGW_1 | 107 | DGTVRVPFR_1 | 164 |
| DGTGGTKGW_2 | 107 | DGTVRVPFR_2 | 164 |
| DGTGGTSGW_1 | 295 | DGTVSMPFK_1 | 402 |
| DGTGGTSGW_2 | 295 | DGTVSMPFK_2 | 402 |
| DGTGGVHGW_1 | 296 | DGTVSNPFR_1 | 403 |
| DGTGGVHGW_2 | 296 | DGTVSNPFR_2 | 403 |
| DGTGGVMGW_1 | 297 | DGTVSTRWV_1 | 404 |
| DGTGGVMGW_2 | 297 | DGTVSTRWV_2 | 404 |
| DGTGGVSGW_1 | 298 | DGTVTTTGW_1 | 405 |
| DGTGGVSGW_2 | 298 | DGTVTTTGW_2 | 405 |
| DGTGGVTGW_1 | 299 | DGTVTVTGW_1 | 406 |
| DGTGGVTGW_2 | 299 | DGTVTVTGW_2 | 406 |
| DGTGGVYGW_1 | 300 | DGTVWVPPR_1 | 407 |
| DGTGGVYGW_2 | 300 | DGTVWVPPR_2 | 407 |
| DGTGNLQGW_1 | 301 | DGTVYRLSS_1 | 408 |
| DGTGNLQGW_2 | 301 | DGTVYRLSS_2 | 408 |
| DGTGNLRGW_1 | 133 | DGTYARLSS_1 | 409 |
| DGTGNLRGW_2 | 133 | DGTYARLSS_2 | 409 |
| DGTGNLSGW_1 | 302 | DGTYGNKLW_1 | 410 |
| DGTGNLSGW_2 | 302 | DGTYGNKLW_2 | 410 |
| DGTGNTHGW_1 | 72 | DGTYIHLSS_1 | 411 |
| DGTGNTHGW_2 | 72 | DGTYIHLSS_2 | 411 |
| DGTGNTRGW_1 | 94 | DGTYSTSGW_1 | 412 |
| DGTGNTRGW_2 | 94 | DGTYSTSGW_2 | 412 |
| DGTGNTSGW_1 | 137 | DGVHPGLSS_1 | 104 |
| DGTGNTSGW_2 | 137 | DGVHPGLSS_2 | 104 |
| DGTGNVSGW_1 | 303 | DGVVALLAS_1 | 413 |
| DGTGNVSGW_2 | 303 | DGVVALLAS_2 | 413 |
| DGTGNVTGW_1 | 69 | DGYVGVGSL_1 | 414 |
| DGTGNVTGW_2 | 69 | DGYVGVGSL_2 | 414 |
| DGTGQLVGW_1 | 304 | control | |
| | | (wtAAV9- | |
| | | NNM) | |
| DGTGQLVGW_2 | 304 | control | |
| | | (wtAAV9- | |
| | | NNK) | |
| DGTGQTIGW_1 | 305 | | |
| DGTGQTIGW_2 | 305 | | |

In one embodiment, the targeting peptide may comprise an amino acid sequence with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any of the sequences shown in Table 2.

In one embodiment, a targeting peptide may comprise 4 or more contiguous amino acids of any of the targeting peptides disclosed herein. In one embodiment the targeting peptide may comprise 4 contiguous amino acids of any of the sequences as set forth in Table 2. In one embodiment the targeting peptide may comprise 5 contiguous amino acids of any of the sequences as set forth in Table 2. In one embodiment the targeting peptide may comprise 6 contiguous amino acids of any of the sequences as set forth in Table 2.

In one embodiment, the AAV particle of the disclosure comprises an AAV capsid with a targeting peptide insert, wherein the targeting peptide has an amino acid sequence as set forth in any of Table 2.

In one embodiment, the AAV particle of the disclosure comprises an AAV capsid with a targeting peptide insert, wherein the targeting peptide has an amino acid sequence comprising at least 4 contiguous amino acids of any of the sequences as set forth in any of Table 2.

In one embodiment, the AAV particle of the disclosure comprises an AAV capsid with a targeting peptide insert, wherein the targeting peptide has an amino acid sequence substantially comprising any of the sequences as set forth in any of Table 2.

In one embodiment, the AAV particle of the disclosure comprises an AAV capsid polynucleotide with a targeting nucleic acid insert, wherein the targeting nucleic acid insert has a nucleotide sequence substantially comprising any of those set forth as Table 2.

The AAV particle of the disclosure comprising a targeting nucleic acid insert, may have a polynucleotide sequence with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more, identity to the parent capsid sequence.

The AAV particle of the disclosure comprising a targeting peptide insert, may have an amino acid sequence with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more, identity to the parent capsid sequence.

In any of the DNA and RNA sequences referenced and/or described herein, the single letter symbol has the following description: A for adenine; C for cytosine; G for guanine; T for thymine; U for Uracil; W for weak bases such as adenine or thymine; S for strong nucleotides such as cytosine and guanine; M for amino nucleotides such as adenine and cytosine; K for keto nucleotides such as guanine and thymine; R for purines adenine and guanine; Y for pyrimidine cytosine and thymine; B for any base that is not A (e.g., cytosine, guanine, and thymine); D for any base that is not C (e.g., adenine, guanine, and thymine); H for any base that is not G (e.g., adenine, cytosine, and thymine); V for any base that is not T (e.g., adenine, cytosine, and guanine); N for any nucleotide (which is not a gap); and Z is for zero.

In any of the amino acid sequences referenced and/or described herein, the single letter symbol has the following description: G (Gly) for Glycine; A (Ala) for Alanine; L (Leu) for Leucine; M (Met) for Methionine; F (Phe) for Phenylalanine; W (Trp) for Tryptophan; K (Lys) for Lysine; Q (Gln) for Glutamine; E (Glu) for Glutamic Acid; S (Ser) for Serine; P (Pro) for Proline; V (Val) for Valine; I (Ile) for Isoleucine; C (Cys) for Cysteine; Y (Tyr) for Tyrosine; H (His) for Histidine; R (Arg) for Arginine; N (Asn) for Asparagine; D (Asp) for Aspartic Acid; T (Thr) for Threonine; B (Asx) for Aspartic acid or Asparagine; J (Xle) for Leucine or Isoleucine; O (Pyl) for Pyrrolysine; U (Sec) for Selenocysteine; X (Xaa) for any amino acid; and Z (Glx) for Glutamine or Glutamic acid.

### Use of Targeting Peptides in AAV Particles

Targeting peptides may be stand-alone peptides or may be inserted into or conjugated to a parent sequence. In one embodiment, the targeting peptides are inserted into the capsid protein of an AAV particle.

One or more targeting peptides may be inserted into a parent AAV capsid sequence to generate the AAV particles of the disclosure.

Targeting peptides may be inserted into a parent AAV capsid sequence in any location that results in fully functional AAV particles. The targeting peptide may be inserted in VP1, VP2 and/or VP3. Numbering of the amino acid residues differs across AAV serotypes, and so the exact amino acid position of the targeting peptide insertion may not be critical. As used herein, amino acid positions of the parent AAV capsid sequence are described using AAV9 (SEQ ID NO: 2) as reference.

In one embodiment, the targeting peptides are inserted in a hypervariable region of the AAV capsid sequence. Non-limiting examples of such hypervariable regions include Loop IV and Loop VIII of the parent AAV capsid. While not wishing to be bound by theory, these surface exposed loops are unstructured and poorly conserved, making them ideal regions for insertion of targeting peptides.

In one embodiment, the targeting peptide is inserted into Loop IV. In another embodiment, the targeting peptide is used to replace a portion, or all of Loop IV. As a non-limiting example, addition of the targeting peptide to the parent AAV capsid sequence may result in the replacement or mutation of at least one amino acid of the parent AAV capsid.

In one embodiment, the targeting peptide is inserted into Loop VIII. In another embodiment, the targeting peptide is used to replace a portion, or all of Loop VIII. As a non-limiting example, addition of the targeting peptide to the parent AAV capsid sequence may result in the replacement or mutation of at least one amino acid of the parent AAV capsid.

In one embodiment, more than one targeting peptide is inserted into a parent AAV capsid sequence. As a non-limiting example, targeting peptides may be inserted at both Loop IV and Loop VIII in the same parent AAV capsid sequence.

Targeting peptides may be inserted at any amino acid position of the parent AAV capsid sequence, such as, but not limited to, between amino acids at positions 586-592, 588-589, 586-589, 452-458, 262-269, 464-473, 491-495, 546-557 and/or 659-668.

In a preferred embodiment, the targeting peptides are inserted into a parent AAV capsid sequence between amino acids at positions 588 and 589 (Loop VIII). In one embodiment, the parent AAV capsid is AAV9 (SEQ ID NO: 2). In a second embodiment, the parent AAV capsid is K449R AAV9 (SEQ ID NO: 3).

The targeting peptides described herein may increase the transduction of the AAV particles of the disclosure to a target tissue as compared to the parent AAV particle lacking a targeting peptide insert. In one embodiment, the targeting peptide increases the transduction of an AAV particle to a target tissue by at least about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 200%, 300%, 400%, 500%, or more as compared to a parent AAV particle lacking a targeting peptide insert.

In one embodiment, the targeting peptide increases the transduction of an AAV particle to a cell or tissue of the CNS by at least about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 200%, 300%, 400%, 500%, or more as compared to a parent AAV particle lacking a targeting peptide insert.

In one embodiment, the targeting peptide increases the transduction of an AAV particle to a cell or tissue of the PNS by at least about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 200%, 300%, 400%, 500%, or more as compared to a parent AAV particle lacking a targeting peptide insert.

In one embodiment, the targeting peptide increases the transduction of an AAV particle to a cell or tissue of the DRG by at least about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 200%, 300%, 400%, 500%, or more as compared to a parent AAV particle lacking a targeting peptide insert.

### AAV Production

Viral production disclosed herein describes processes and methods for producing AAV particles (with enhanced, improved and/or increased tropism for a target tissue) that may be used to contact a target cell to deliver a payload.

The present disclosure provides methods for the generation of AAV particles comprising targeting peptides. In one embodiment, the AAV particles are prepared by viral genome replication in a viral replication cell. Any method known in the art may be used for the preparation of AAV particles. In one embodiment, AAV particles are produced in mammalian cells (e.g., HEK293). In another embodiment, AAV particles are produced in insect cells (e.g., Sf9)

Methods of making AAV particles are well known in the art and are described in e.g., U.S. Pat. Nos. 6,204,059, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508, 5,064,764, 6,194,191, 6,566,118, 8,137,948; or International Publication Nos. WO1996039530, WO1998010088, WO1999014354, WO1999015685, WO1999047691, WO2000055342, WO2000075353 and WO2001023597; Methods In Molecular Biology, ed. Richard, Humana Press, NJ (1995); O'Reilly et al., Baculovirus Expression Vectors, A Laboratory Manual, Oxford Univ. Press (1994); Samulski et al., J. Vir. 63:3822-8 (1989); Kajigaya et al., Proc. Nat'l. Acad. Sci. USA 88:4646-50 (1991); Ruffing et al., J. Vir. 66:6922-30 (1992); Kimbauer et al., Vir., 219:37-44 (1996); Zhao et al., Vir. 272:382-93 (2000); the contents of each of which are herein incorporated by reference in their entirety. In one embodiment, the AAV particles are made using the methods described in International Patent Publication WO2015191508, the contents of which are herein incorporated by reference in their entirety.

### Therapeutic Applications

The present disclosure provides a method for treating a disease, disorder and/or condition in a mammalian subject, including a human subject, comprising administering to the subject an AAV particle described herein where the AAV particle comprises the novel capsids (“TRACER AAV particles”) defined by the present disclosure or administering to the subject any of the described compositions, including pharmaceutical compositions, described herein.

In one embodiment, the TRACER AAV particles of the present disclosure are administered to a subject prophylactically, to prevent on-set of disease. In another embodiment, the TRACER AAV particles of the present disclosure are administered to treat (lessen the effects of) a disease or symptoms thereof. In yet another embodiment, the TRACER AAV particles of the present disclosure are administered to cure (eliminate) a disease. In another embodiment, the TRACER AAV particles of the present disclosure are administered to prevent or slow progression of disease. In yet another embodiment, the TRACER AAV particles of the present disclosure are used to reverse the deleterious effects of a disease. Disease status and/or progression may be determined or monitored by standard methods known in the art.

In some embodiments, the TRACER AAV particles of the disclosure are useful in the field of medicine for the treatment, prophylaxis, palliation or amelioration of neurological diseases and/or disorders.

In some embodiments, the TRACER AAV particles of the disclosure are useful in the field of medicine for the treatment, prophylaxis, palliation or amelioration of tauopathy.

In some embodiments, the TRACER AAV particles of the disclosure are useful in the field of medicine for the treatment, prophylaxis, palliation or amelioration of Alzheimer's Disease.

In some embodiments, the TRACER AAV particles of the disclosure are useful in the field of medicine for the treatment, prophylaxis, palliation or amelioration of Friedreich's ataxia, or any disease stemming from a loss or partial loss of frataxin protein.

In some embodiments, the TRACER AAV particles of the disclosure are useful in the field of medicine for the treatment, prophylaxis, palliation or amelioration of Parkinson's Disease.

In some embodiments, the TRACER AAV particles of the disclosure are useful in the field of medicine for the treatment, prophylaxis, palliation or amelioration of Amyotrophic lateral sclerosis.

In some embodiments, the TRACER AAV particles of the disclosure are useful in the field of medicine for the treatment, prophylaxis, palliation or amelioration of Huntington's Disease.

In some embodiments, the TRACER AAV particles of the disclosure are useful in the field of medicine for the treatment, prophylaxis, palliation or amelioration of chronic or neuropathic pain.

In some embodiments, the TRACER AAV particles of the disclosure are useful in the field of medicine for treatment, prophylaxis, palliation or amelioration of a disease associated with the central nervous system.

In some embodiments, the TRACER AAV particles of the disclosure are useful in the field of medicine for treatment, prophylaxis, palliation or amelioration of a disease associated with the peripheral nervous system.

In one embodiment, the TRACER AAV particles of the present disclosure are administered to a subject having at least one of the diseases or symptoms described herein.

As used herein, any disease associated with the central or peripheral nervous system and components thereof (e.g., neurons) may be considered a “neurological disease”.

Any neurological disease may be treated with the TRACER AAV particles of the disclosure, or pharmaceutical compositions thereof, including but not limited to, Absence of the Septum Pellucidum, Acid Lipase Disease, Acid Maltase Deficiency, Acquired Epileptiform Aphasia, Acute Disseminated Encephalomyelitis, Attention Deficit-Hyperactivity Disorder (ADHD), Adie's Pupil, Adie's Syndrome, Adrenoleukodystrophy, Agenesis of the Corpus Callosum, Agnosia, Aicardi Syndrome, Aicardi-Goutieres Syndrome Disorder, AIDS-Neurological Complications, Alexander Disease, Alpers' Disease, Alternating Hemiplegia, Alzheimer's Disease, Amyotrophic Lateral Sclerosis (ALS), Anencephaly, Aneurysm, Angelman Syndrome, Angiomatosis, Anoxia, Antiphospholipid Syndrome, Aphasia, Apraxia, Arachnoid Cysts, Arachnoiditis, Arnold-Chiari Malformation, Arteriovenous Malformation, Asperger Syndrome, Ataxia, Ataxia Telangiectasia, Ataxias and Cerebellar or Spinocerebellar Degeneration, Atrial Fibrillation and Stroke, Attention Deficit-Hyperactivity Disorder, Autism Spectrum Disorder, Autonomic Dysfunction, Back Pain, Barth Syndrome, Batten Disease, Becker's Myotonia, Bechet's Disease, Bell's Palsy, Benign Essential Blepharospasm, Benign Focal Amyotrophy, Benign Intracranial Hypertension, Bernhardt-Roth Syndrome, Binswanger's Disease, Blepharospasm, Bloch-Sulzberger Syndrome, Brachial Plexus Birth Injuries, Brachial Plexus Injuries, Bradbury-Eggleston Syndrome, Brain and Spinal Tumors, Brain Aneurysm, Brain Injury, Brown-Sequard Syndrome, Bulbar palsy, Bulbospinal Muscular Atrophy, Cerebral Autosomal Dominant Arteriopathy with Sub-cortical Infarcts and Leukoencephalopathy (CADASIL), Canavan Disease, Carpal Tunnel Syndrome, Causalgia, Cavernomas, Cavernous Angioma, Cavernous Malformation, Central Cervical Cord Syndrome, Central Cord Syndrome, Central Pain Syndrome, Central Pontine Myelinolysis, Cephalic Disorders, Ceramidase Deficiency, Cerebellar Degeneration, Cerebellar Hypoplasia, Cerebral Aneurysms, Cerebral Arteriosclerosis, Cerebral Atrophy, Cerebral Beriberi, Cerebral Cavernous Malformation, Cerebral Gigantism, Cerebral Hypoxia, Cerebral Palsy, Cerebro-Oculo-Facio-Skeletal Syndrome (COFS), Charcot-Marie-Tooth Disease, Chiari Malformation, Cholesterol Ester Storage Disease, Chorea, Choreoacanthocytosis, Chronic Inflammatory Demyelinating Polyneuropathy (CIDP), Chronic Orthostatic Intolerance, Chronic Pain, Cockayne Syndrome Type II, Coffin Lowry Syndrome, Colpocephaly, Coma, Complex Regional Pain Syndrome, Concentric sclerosis (Baló's sclerosis), Congenital Facial Diplegia, Congenital Myasthenia, Congenital Myopathy, Congenital Vascular Cavernous Malformations, Corticobasal Degeneration, Cranial Arteritis, Craniosynostosis, Cree encephalitis, Creutzfeldt-Jakob Disease, Chronic progressive external ophtalmoplegia, Cumulative Trauma Disorders, Cushing's Syndrome, Cytomegalic Inclusion Body Disease, Cytomegalovirus Infection, Dancing Eyes-Dancing Feet Syndrome, Dandy-Walker Syndrome, Dawson Disease, De Morsier's Syndrome, Dejerine-Klumpke Palsy, Dementia, Dementia-Multi-Infarct, Dementia-Semantic, Dementia-Subcortical, Dementia With Lewy Bodies, Demyelination diseases, Dentate Cerebellar Ataxia, Dentatorubral Atrophy, Dermatomyositis, Developmental Dyspraxia, Devic's Syndrome, Diabetic Neuropathy, Diffuse Sclerosis, Distal hereditary motor neuronopathies, Dravet Syndrome, Dysautonomia, Dysgraphia, Dyslexia, Dysphagia, Dyspraxia, Dyssynergia Cerebellaris Myoclonica, Dyssynergia Cerebellaris Progressiva, Dystonias, Early Infantile Epileptic Encephalopathy, Empty Sella Syndrome, Encephalitis, Encephalitis Lethargica, Encephaloceles, Encephalomyelitis, Encephalopathy, Encephalopathy (familial infantile), Encephalotrigeminal Angiomatosis, Epilepsy, Epileptic Hemiplegia, Episodic ataxia, Erb's Palsy, Erb-Duchenne and Dejerine-Klumpke Palsies, Essential Tremor, Extrapontine Myelinolysis, Faber's disease, Fabry Disease, Fahr's Syndrome, Fainting, Familial Dysautonomia, Familial Hemangioma, Familial Idiopathic Basal Ganglia Calcification, Familial Periodic Paralyses, Familial Spastic Paralysis, Farber's Disease, Febrile Seizures, Fibromuscular Dysplasia, Fisher Syndrome, Floppy Infant Syndrome, Foot Drop, Friedreich's Ataxia, Frontotemporal Dementia, Gaucher Disease, Generalized Gangliosidoses (GM1, GM2), Gerstmann's Syndrome, Gerstmann-Straussler-Scheinker Disease, Giant Axonal Neuropathy, Giant Cell Arteritis, Giant Cell Inclusion Disease, Globoid Cell Leukodystrophy, Glossopharyngeal Neuralgia, Glycogen Storage Disease, Guillain-Barre Syndrome, Hallervorden-Spatz Disease, Head Injury, Headache, Hemicrania Continua, Hemifacial Spasm, Hemiplegia Alterans, Hereditary Neuropathies, Hereditary Spastic Paraplegia, Heredopathia Atactica Polyneuritiformis, Herpes Zoster, Herpes Zoster Oticus, Hirayama Syndrome, Holmes-Adie syndrome, Holoprosencephaly, HTLV-1 Associated Myelopathy, Hughes Syndrome, Huntington's Disease, Hurler syndrome, Hydranencephaly, Hydrocephalus, Hydrocephalus-Normal Pressure, Hydromyelia, Hypercortisolism, Hypersomnia, Hypertonia, Hypotonia, Hypoxia, Immune-Mediated Encephalomyelitis, Inclusion Body Myositis, Incontinentia Pigmenti, Infantile Hypotonia, Infantile Neuroaxonal Dystrophy, Infantile Phytanic Acid Storage Disease, Infantile Refsum Disease, Infantile Spasms, Inflammatory Myopathies, Iniencephaly, Intestinal Lipodystrophy, Intracranial Cysts, Intracranial Hypertension, Isaacs' Syndrome, Joubert Syndrome, Kearns-Sayre Syndrome, Kennedy's Disease, Kinsbourne syndrome, Kleine-Levin Syndrome, Klippel-Feil Syndrome, Klippel-Trenaunay Syndrome (KTS), Klüver-Bucy Syndrome, Korsakoff's Amnesic Syndrome, Krabbe Disease, Kugelberg-Welander Disease, Kuru, Lambert-Eaton Myasthenic Syndrome, Landau-Kleffner Syndrome, Lateral Femoral Cutaneous Nerve Entrapment, Lateral Medullary Syndrome, Learning Disabilities, Leigh's Disease, Lennox-Gastaut Syndrome, Lesch-Nyhan Syndrome, Leukodystrophy, Levine-Critchley Syndrome, Lewy Body Dementia, Lichtheim's disease, Lipid Storage Diseases, Lipoid Proteinosis, Lissencephaly, Locked-In Syndrome, Lou Gehrig's Disease, Lupus-Neurological Sequelae, Lyme Disease-Neurological Complications, Lysosomal storage disorders, Machado-Joseph Disease, Macrencephaly, Megalencephaly, Melkersson-Rosenthal Syndrome, Meningitis, Meningitis and Encephalitis, Menkes Disease, Meralgia Paresthetica, Metachromatic Leukodystrophy, Microcephaly, Migraine, Miller Fisher Syndrome, Mini Stroke, Mitochondrial Myopathy, Mitochondrial DNA depletion syndromes, Moebius Syndrome, Monomelic Amyotrophy, Morvan Syndrome, Motor Neuron Diseases, Moyamoya Disease, Mucolipidoses, Mucopolysaccharidoses, Multi-Infarct Dementia, Multifocal Motor Neuropathy, Multiple Sclerosis, Multiple System Atrophy, Multiple System Atrophy with Orthostatic Hypotension, Muscular Dystrophy, Myasthenia-Congenital, Myasthenia Gravis, Myelinoclastic Diffuse Sclerosis, Myelitis, Myoclonic Encephalopathy of Infants, Myoclonus, Myoclonus epilepsy, Myopathy, Myopathy-Congenital, Myopathy-Thyrotoxic, Myotonia, Myotonia Congenita, Narcolepsy, NARP (neuropathy, ataxia and retinitis pigmentosa), Neuroacanthocytosis, Neurodegeneration with Brain Iron Accumulation, Neurodegenerative disease, Neurofibromatosis, Neuroleptic Malignant Syndrome, Neurological Complications of AIDS, Neurological Complications of Lyme Disease, Neurological Consequences of Cytomegalovirus Infection, Neurological Manifestations of Pompe Disease, Neurological Sequelae Of Lupus, Neuromyelitis Optica, Neuromyotonia, Neuronal Ceroid Lipofuscinosis, Neuronal Migration Disorders, Neuropathic pain, Neuropathy-Hereditary, Neuropathy, Neurosarcoidosis, Neurosyphilis, Neurotoxicity, Nevus Cavernosus, Niemann-Pick Disease, O'Sullivan-McLeod Syndrome, Occipital Neuralgia, Ohtahara Syndrome, Olivopontocerebellar Atrophy, Opsoclonus Myoclonus, Orthostatic Hypotension, Overuse Syndrome, Pain-Chronic, Pantothenate Kinase-Associated Neurodegeneration, Paraneoplastic Syndromes, Paresthesia, Parkinson's Disease, Paroxysmal Choreoathetosis, Paroxysmal Hemicrania, Parry-Romberg, Pelizaeus-Merzbacher Disease, Pena Shokeir II Syndrome, Perineural Cysts, Peroneal muscular atrophy, Periodic Paralyses, Peripheral Neuropathy, Periventricular Leukomalacia, Persistent Vegetative State, Pervasive Developmental Disorders, Phytanic Acid Storage Disease, Pick's Disease, Pinched Nerve, Piriformis Syndrome, Pituitary Tumors, Polymyositis, Pompe Disease, Porencephaly, Post-Polio Syndrome, Postherpetic Neuralgia, Postinfectious Encephalomyelitis, Postural Hypotension, Postural Orthostatic Tachycardia Syndrome, Postural Tachycardia Syndrome, Primary Dentatum Atrophy, Primary Lateral Sclerosis, Primary Progressive Aphasia, Prion Diseases, Progressive bulbar palsy, Progressive Hemifacial Atrophy, Progressive Locomotor Ataxia, Progressive Multifocal Leukoencephalopathy, Progressive Muscular Atrophy, Progressive Sclerosing Poliodystrophy, Progressive Supranuclear Palsy, Prosopagnosia, Pseudobulbar palsy, Pseudo-Torch syndrome, Pseudotoxoplasmosis syndrome, Pseudotumor Cerebri, Psychogenic Movement, Ramsay Hunt Syndrome I, Ramsay Hunt Syndrome II, Rasmussen's Encephalitis, Reflex Sympathetic Dystrophy Syndrome, Refsum Disease, Refsum Disease-Infantile, Repetitive Motion Disorders, Repetitive Stress Injuries, Restless Legs Syndrome, Retrovirus-Associated Myelopathy, Rett Syndrome, Reye's Syndrome, Rheumatic Encephalitis, Riley-Day Syndrome, Sacral Nerve Root Cysts, Saint Vitus Dance, Salivary Gland Disease, Sandhoff Disease, Schilder's Disease, Schizencephaly, Seitelberger Disease, Seizure Disorder, Semantic Dementia, Septo-Optic Dysplasia, Severe Myoclonic Epilepsy of Infancy (SMEI), Shaken Baby Syndrome, Shingles, Shy-Drager Syndrome, Sjögren's Syndrome, Sleep Apnea, Sleeping Sickness, Sotos Syndrome, Spasticity, Spina Bifida, Spinal Cord Infarction, Spinal Cord Injury, Spinal Cord Tumors, Spinal Muscular Atrophy, Spinocerebellar Ataxia, Spinocerebellar Atrophy, Spinocerebellar Degeneration, Sporadic ataxia, Steele-Richardson-Olszewski Syndrome, Stiff-Person Syndrome, Striatonigral Degeneration, Stroke, Sturge-Weber Syndrome, Subacute Sclerosing Panencephalitis, Subcortical Arteriosclerotic Encephalopathy, Short-lasting, Unilateral, Neuralgiform (SUNCT) Headache, Swallowing Disorders, Sydenham Chorea, Syncope, Syphilitic Spinal Sclerosis, Syringohydromyelia, Syringomyelia, Systemic Lupus Erythematosus, Tabes Dorsalis, Tardive Dyskinesia, Tarlov Cysts, Tay-Sachs Disease, Temporal Arteritis, Tethered Spinal Cord Syndrome, Thomsen's Myotonia, Thoracic Outlet Syndrome, Thyrotoxic Myopathy, Tic Douloureux, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Troyer Syndrome, Tuberous Sclerosis, Vascular Erectile Tumor, Vasculitis Syndromes of the Central and Peripheral Nervous Systems, Vitamin B12 deficiency, Von Economo's Disease, Von Hippel-Lindau Disease (VHL), Von Recklinghausen's Disease, Wallenberg's Syndrome, Werdnig-Hoffman Disease, Wernicke-Korsakoff Syndrome, West Syndrome, Whiplash, Whipple's Disease, Williams Syndrome, Wilson Disease, Wolman's Disease, X-Linked Spinal and Bulbar Muscular Atrophy.

### Methods of Treatment of Neurological Disease

### TRACER AAV Particles Encoding Protein Payloads

Provided in the present disclosure are methods for introducing the TRACER AAV particles of the present disclosure into cells, the method comprising introducing into said cells any of the vectors in an amount sufficient for an increase in the production of target mRNA and protein to occur. In some aspects, the cells may be neurons such as but not limited to, motor, hippocampal, entorhinal, thalamic, cortical, sensory, sympathetic, or parasympathetic neurons, and glial cells such as astrocytes, microglia, and/or oligodendrocytes.

Disclosed in the present disclosure are methods for treating neurological disease associated with insufficient function/presence of a target protein (e.g., ApoE, FXN) in a subject in need of treatment. The method optionally comprises administering to the subject a therapeutically effective amount of a composition comprising TRACER AAV particles of the present disclosure. As a non-limiting example, the TRACER AAV particles can increase target gene expression, increase target protein production, and thus reduce one or more symptoms of neurological disease in the subject such that the subject is therapeutically treated.

In one embodiment, the composition comprising the TRACER AAV particles of the present disclosure is administered to the central nervous system of the subject via systemic administration. In one embodiment, the systemic administration is intravenous injection.

In some embodiments, the composition comprising the TRACER AAV particles of the present disclosure is administered to the central nervous system of the subject. In other embodiments, the composition comprising the TRACER AAV particles of the present disclosure is administered to a CNS tissue of a subject (e.g., putamen, thalamus or cortex of the subject).

In one embodiment, the composition comprising the TRACER AAV particles of the present disclosure is administered to the central nervous system of the subject via intraparenchymal injection. Non-limiting examples of intraparenchymal injections include intraputamenal, intracortical, intrathalamic, intrastriatal, intrahippocampal or into the entorhinal cortex.

In one embodiment, the composition comprising the TRACER AAV particles of the present disclosure is administered to the central nervous system of the subject via intraparenchymal injection and intravenous injection.

In one embodiment, the TRACER AAV particles of the present disclosure may be delivered into specific types of targeted cells, including, but not limited to, thalamic, hippocampal, entorhinal, cortical, motor, sensory, excitatory, inhibitory, sympathetic, or parasympathetic neurons; glial cells including oligodendrocytes, astrocytes and microglia; and/or other cells surrounding neurons such as T cells.

In one embodiment, the TRACER AAV particles of the present disclosure may be delivered to neurons in the putamen, thalamus and/or cortex.

In some embodiments, the TRACER AAV particles of the present disclosure may be used as a therapy for neurological disease.

In some embodiments, the TRACER AAV particles of the present disclosure may be used as a therapy for tauopathies.

In some embodiments, the TRACER AAV particles of the present disclosure may be used as a therapy for Alzheimer's Disease.

In some embodiments, the TRACER AAV particles of the present disclosure may be used as a therapy for Amyotrophic Lateral Sclerosis.

In some embodiments, the TRACER AAV particles of the present disclosure may be used as a therapy for Huntington's Disease.

In some embodiments, the TRACER AAV particles of the present disclosure may be used as a therapy for Parkinson's Disease.

In some embodiments, the TRACER AAV particles of the present disclosure may be used as a therapy for Friedreich's Ataxia.

In some embodiments, the TRACER AAV particles of the present disclosure may be used as a therapy for chronic or neuropathic pain.

In one embodiment, administration of the TRACER AAV particles described herein to a subject may increase target protein levels in a subject. The target protein levels may be increased by about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in a subject such as, but not limited to, the CNS, a region of the CNS, or a specific cell of the CNS of a subject. As a non-limiting example, the TRACER AAV particles may increase the protein levels of a target protein by at least 50%. As a non-limiting example, the TRACER AAV particles may increase the proteins levels of a target protein by at least 40%. As a non-limiting example, a subject may have an increase of 10% of target protein. As a non-limiting example, the TRACER AAV particles may increase the protein levels of a target protein by fold increases over baseline. In one embodiment, TRACER AAV particles lead to 5-6 times higher levels of a target protein.

In one embodiment, administration of the TRACER AAV particles described herein to a subject may increase the expression of a target protein in a subject. The expression of the target protein may be increased by about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in a subject such as, but not limited to, the CNS, a region of the CNS, or a specific cell of the CNS of a subject. As a non-limiting example, the TRACER AAV particles may increase the expression of a target protein by at least 50%. As a non-limiting example, the TRACER AAV particles may increase the expression of a target protein by at least 40%.

In one embodiment, intravenous administration of the TRACER AAV particles described herein to a subject may increase the CNS expression of a target protein in a subject. The expression of the target protein may be increased by about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in a subject such as, but not limited to, the CNS, a region of the CNS, or a specific cell of the CNS of a subject. As a non-limiting example, the TRACER AAV particles may increase the expression of a target protein in the CNS by at least 50%. As a non-limiting example, the TRACER AAV particles may increase the expression of a target protein in the CNS by at least 40%.

In some embodiments, the TRACER AAV particles of the present disclosure may be used to increase target protein expression in astrocytes in order to treat a neurological disease. Target protein in astrocytes may be increased by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In some embodiments, the TRACER AAV particles may be used to increase target protein in microglia. The increase of target protein in microglia may be, independently, increased by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In some embodiments, the TRACER AAV particles may be used to increase target protein in cortical neurons. The increase of target protein in the cortical neurons may be, independently, increased by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In some embodiments, the TRACER AAV particles may be used to increase target protein in hippocampal neurons. The increase of target protein in the hippocampal neurons may be, independently, increased by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In some embodiments, the TRACER AAV particles may be used to increase target protein in DRG and/or sympathetic neurons. The increase of target protein in the DRG and/or sympathetic neurons may be, independently, increased by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In some embodiments, the TRACER AAV particles of the present disclosure may be used to increase target protein in sensory neurons in order to treat neurological disease. Target protein in sensory neurons may be increased by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In some embodiments, the TRACER AAV particles of the present disclosure may be used to increase target protein and reduce symptoms of neurological disease in a subject. The increase of target protein and/or the reduction of symptoms of neurological disease may be, independently, altered (increased for the production of target protein and reduced for the symptoms of neurological disease) by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In one embodiment, the TRACER AAV particles of the present disclosure may be used to reduce the decline of functional capacity and activities of daily living as measured by a standard evaluation system such as, but not limited to, the total functional capacity (TFC) scale.

In one embodiment, the TRACER AAV particles of the present disclosure may be used to improve performance on any assessment used to measure symptoms of neurological disease. Such assessments include, but are not limited to ADAS-cog (Alzheimer Disease Assessment Scale-cognitive), MMSE (Mini-Mental State Examination), GDS (Geriatric Depression Scale), FAQ (Functional Activities Questionnaire), ADL (Activities of Daily Living), GPCOG (General Practitioner Assessment of Cognition), Mini-Cog, AMTS (Abbreviated Mental Test Score), Clock-drawing test, 6-CIT (6-item Cognitive Impairment Test), TYM (Test Your Memory), MoCa (Montreal Cognitive Assessment), ACE-R (Addenbrookes Cognitive Assessment), MIS (Memory Impairment Screen), BADLS (Bristol Activities of Daily Living Scale), Barthel Index, Functional Independence Measure, Instrumental Activities of Daily Living, IQCODE (Informant Questionnaire on Cognitive Decline in the Elderly), Neuropsychiatric Inventory, The Cohen-Mansfield Agitation Inventory, BEHAVE-AD, EuroQol, Short Form-36 and/or MBR Caregiver Strain Instrument, or any of the other tests as described in Sheehan B (Ther Adv Neurol Disord. 5(6):349-358 (2012)), the contents of which are herein incorporated by reference in their entirety.

In some embodiments, the present composition is administered as a solo therapeutic or as combination therapeutic for the treatment of neurological disease.

The TRACER AAV particles encoding the target protein may be used in combination with one or more other therapeutic agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent.

Therapeutic agents that may be used in combination with the TRACER AAV particles of the present disclosure can be small molecule compounds which are antioxidants, anti-inflammatory agents, anti-apoptosis agents, calcium regulators, antiglutamatergic agents, structural protein inhibitors, compounds involved in muscle function, and compounds involved in metal ion regulation. As a non-limiting example, the combination therapy may be in combination with one or more neuroprotective agents such as small molecule compounds, growth factors and hormones which have been tested for their neuroprotective effect on motor neuron degeneration.

Compounds tested for treating neurological disease which may be used in combination with the TRACER AAV particles described herein include, but are not limited to, cholinesterase inhibitors (donepezil, rivastigmine, galantamine), NMDA receptor antagonists such as memantine, anti-psychotics, anti-depressants, anti-convulsants (e.g., sodium valproate and levetiracetam for myoclonus), secretase inhibitors, amyloid aggregation inhibitors, copper or zinc modulators, BACE inhibitors, inhibitors of tau aggregation, such as Methylene blue, phenothiazines, anthraquinones, n-phenylamines or rhodamines, microtubule stabilizers such as NAP, taxol or paclitaxel, kinase or phosphatase inhibitors such as those targeting GSK3β (lithium) or PP2A, immunization with Aβ peptides or tau phospho-epitopes, anti-tau or anti-amyloid antibodies, dopamine-depleting agents (e.g., tetrabenazine for chorea), benzodiazepines (e.g., clonazepam for myoclonus, chorea, dystonia, rigidity, and/or spasticity), amino acid precursors of dopamine (e.g., levodopa for rigidity), skeletal muscle relaxants (e.g., baclofen, tizanidine for rigidity and/or spasticity), inhibitors for acetylcholine release at the neuromuscular junction to cause muscle paralysis (e.g., botulinum toxin for bruxism and/or dystonia), atypical neuroleptics (e.g., olanzapine and quetiapine for psychosis and/or irritability, risperidone, sulpiride and haloperidol for psychosis, chorea and/or irritability, clozapine for treatment-resistant psychosis, aripiprazole for psychosis with prominent negative symptoms), selective serotonin reuptake inhibitors (SSRIs) (e.g., citalopram, fluoxetine, paroxetine, sertraline, mirtazapine, venlafaxine for depression, anxiety, obsessive compulsive behavior and/or irritability), hypnotics (e.g., xopiclone and/or zolpidem for altered sleep-wake cycle), anticonvulsants (e.g., sodium valproate and carbamazepine for mania or hypomania) and mood stabilizers (e.g., lithium for mania or hypomania).

Neurotrophic factors may be used in combination therapy with the TRACER AAV particles of the present disclosure for treating neurological disease. Generally, a neurotrophic factor is defined as a substance that promotes survival, growth, differentiation, proliferation and/or maturation of a neuron, or stimulates increased activity of a neuron. In some embodiments, the present methods further comprise delivery of one or more trophic factors into the subject in need of treatment. Trophic factors may include, but are not limited to, IGF-I, GDNF, BDNF, CTNF, VEGF, Colivelin, Xaliproden, Thyrotrophin-releasing hormone and ADNF, and variants thereof.

In one aspect, the TRACER AAV particle described herein may be co-administered with TRACER AAV particles expressing neurotrophic factors such as AAV-IGF-I (See e.g., Vincent et al., Neuromolecular medicine, 2004, 6, 79-85; the contents of which are incorporated herein by reference in their entirety) and AAV-GDNF (See e.g., Wang et al., J Neurosci., 2002, 22, 6920-6928; the contents of which are incorporated herein by reference in their entirety).

In one embodiment, administration of the TRACER AAV particles to a subject will increase the expression of a target protein in a subject and the increase of the expression of the target protein will reduce the effects and/or symptoms of neurological disease in a subject.

As a non-limiting example, the target protein may be an antibody, or fragment thereof.

### TRACER AAV Particles Comprising RNAi Agents or Modulatory Polynucleotides

Provided in the present disclosure are methods for introducing the TRACER AAV particles of the disclosure, comprising a viral genome with a nucleic acid sequence encoding one or more siRNA molecules into cells, the method comprising introducing into said cells any of the vectors in an amount sufficient for degradation of a target mRNA to occur, thereby activating target-specific RNAi in the cells. In some aspects, the cells may be neurons such as but not limited to, motor, hippocampal, entorhinal, thalamic, cortical, sensory, sympathetic, or parasympathetic neurons, and glial cells such as astrocytes, microglia, and/or oligodendrocytes.

Disclosed in the present disclosure are methods for treating neurological diseases associated with dysfunction of a target protein in a subject in need of treatment. The method optionally comprises administering to the subject a therapeutically effective amount of a composition comprising TRACER AAV particles comprising a viral genome with a nucleic acid sequence encoding one or more siRNA molecules. As a non-limiting example, the siRNA molecules can silence target gene expression, inhibit target protein production, and reduce one or more symptoms of neurological disease in the subject such that the subject is therapeutically treated.

In some embodiments, the composition comprising the TRACER AAV particles of the present disclosure comprising a viral genome encoding one or more siRNA molecules comprise an AAV capsid that allows for enhanced transduction of CNS and/or PNS cells after intravenous administration.

In some embodiments, the composition comprising the TRACER AAV particles of the present disclosure with a viral genome encoding at least one siRNA molecule is administered to the central nervous system of the subject. In other embodiments, the composition comprising the TRACER AAV particles of the present disclosure is administered to a tissue of a subject (e.g., putamen, thalamus or cortex of the subject).

In one embodiment, the composition comprising the TRACER AAV particles of the disclosure, comprising a viral genome with a nucleic acid sequence encoding one or more siRNA molecules is administered to the central nervous system of the subject via systemic administration. In one embodiment, the systemic administration is intravenous injection.

In one embodiment, the composition comprising the TRACER AAV particles of the disclosure comprising a viral genome with a nucleic acid sequence encoding one or more siRNA molecules is administered to the central nervous system of the subject via intraparenchymal injection. Non-limiting examples of intraparenchymal injections include intraputamenal, intracortical, intrathalamic, intrastriatal, intrahippocampal or into the entorhinal cortex.

In one embodiment, the composition comprising the TRACER AAV particles comprising a viral genome with a nucleic acid sequence encoding one or more siRNA molecules is administered to the central nervous system of the subject via intraparenchymal injection and intravenous injection.

In one embodiment, the TRACER AAV particles comprising a viral genome with a nucleic acid sequence encoding one or more siRNA molecules may be delivered into specific types or targeted cells, including, but not limited to, thalamic, hippocampal, entorhinal, cortical, motor, sensory, excitatory, inhibitory, sympathetic, or parasympathetic neurons; glial cells including oligodendrocytes, astrocytes and microglia; and/or other cells surrounding neurons such as T cells.

In one embodiment, the TRACER AAV particles comprising a viral genome with a nucleic acid sequence encoding one or more siRNA molecules may be delivered to neurons in the putamen, thalamus, and/or cortex.

In one embodiment, the TRACER AAV particles comprising a viral genome with a nucleic acid sequence encoding one or more siRNA molecules may be used as a therapy for neurological disease.

In one embodiment, the TRACER AAV particles comprising a viral genome with a nucleic acid sequence encoding one or more siRNA molecules may be used as a therapy for tauopathies.

In one embodiment, the TRACER AAV particles comprising a viral genome with a nucleic acid sequence encoding one or more siRNA molecules may be used as a therapy for Alzheimer's Disease.

In one embodiment, the TRACER AAV particles comprising a viral genome with a nucleic acid sequence encoding one or more siRNA molecules may be used as a therapy for Amyotrophic Lateral Sclerosis.

In one embodiment, the TRACER AAV particles comprising a viral genome with a nucleic acid sequence encoding one or more siRNA molecules may be used as a therapy for Huntington's Disease.

In one embodiment, the TRACER AAV particles comprising a viral genome with a nucleic acid sequence encoding one or more siRNA molecules may be used as a therapy for Parkinson's Disease.

In one embodiment, the TRACER AAV particles comprising a viral genome with a nucleic acid sequence encoding one or more siRNA molecules may be used as a therapy for Friedreich's Ataxia.

In one embodiment, the administration of TRACER AAV particles comprising a viral genome with a nucleic acid sequence encoding one or more siRNA molecules to a subject may lower target protein levels in a subject. The target protein levels may be lowered by about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in a subject such as, but not limited to, the CNS, a region of the CNS, or a specific cell of the CNS of a subject. As a non-limiting example, the TRACER AAV particles may lower the protein levels of a target protein by at least 50%. As a non-limiting example, the TRACER AAV particles may lower the proteins levels of a target protein by at least 40%.

In one embodiment, the administration of TRACER AAV particles comprising a viral genome with a nucleic acid sequence encoding one or more siRNA molecules to a subject may lower the expression of a target protein in a subject. The expression of a target protein may be lowered by about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in a subject such as, but not limited to, the CNS, a region of the CNS, or a specific cell of the CNS of a subject. As a non-limiting example, the TRACER AAV particles may lower the expression of a target protein by at least 50%. As a non-limiting example, the TRACER AAV particles may lower the expression of a target protein by at least 40%.

In one embodiment, the administration of TRACER AAV particles comprising a viral genome with a nucleic acid sequence encoding one or more siRNA molecules to a subject may lower the expression of a target protein in the CNS of a subject. The expression of a target protein may be lowered by about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in a subject such as, but not limited to, the CNS, a region of the CNS, or a specific cell of the CNS of a subject. As a non-limiting example, the TRACER AAV particles may lower the expression of a target protein by at least 50%. As a non-limiting example, the TRACER AAV particles may lower the expression of a target protein by at least 40%.

In one embodiment, the TRACER AAV particles comprising a viral genome with a nucleic acid sequence encoding one or more siRNA molecules may be used to suppress a target protein in astrocytes in order to treat neurological disease. Target protein in astrocytes may be suppressed by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%. Target protein in astrocytes may be reduced may be 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In one embodiment, the TRACER AAV particles comprising a viral genome with a nucleic acid sequence encoding one or more siRNA molecules may be used to suppress a target protein in microglia. The suppression of the target protein in microglia may be, independently, suppressed by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%. The reduction may be 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In one embodiment, the TRACER AAV particles comprising a viral genome with a nucleic acid sequence encoding one or more siRNA molecules may be used to suppress target protein in cortical neurons. The suppression of a target protein in cortical neurons may be, independently, suppressed by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%. The reduction may be 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In one embodiment, the TRACER AAV particles comprising a viral genome with a nucleic acid sequence encoding one or more siRNA molecules may be used to suppress a target protein in hippocampal neurons. The suppression of a target protein in the hippocampal neurons may be, independently, suppressed by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%. The reduction may be 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In one embodiment, the TRACER AAV particles comprising a viral genome with a nucleic acid sequence encoding one or more siRNA molecules may be used to suppress a target protein in DRG and/or sympathetic neurons. The suppression of a target protein in the DRG and/or sympathetic neurons may be, independently, suppressed by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5- 70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%. The reduction may be 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5- 70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In one embodiment, the TRACER AAV particles comprising a viral genome with a nucleic acid sequence encoding one or more siRNA molecules may be used to suppress a target protein in sensory neurons in order to treat neurological disease. Target protein in sensory neurons may be suppressed by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%. Target protein in the sensory neurons may be reduced may be 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5- 70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In one embodiment, the TRACER AAV particles comprising a viral genome with a nucleic acid sequence encoding one or more siRNA molecules may be used to suppress a target protein and reduce symptoms of neurological disease in a subject. The suppression of target protein and/or the reduction of symptoms of neurological disease may be, independently, reduced or suppressed by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In one embodiment, the TRACER AAV particles comprising a viral genome with a nucleic acid sequence encoding one or more siRNA molecules may be used to reduce the decline of functional capacity and activities of daily living as measured by a standard evaluation system such as, but not limited to, the total functional capacity (TFC) scale.

In some embodiments, the present composition is administered as a solo therapeutic or as combination therapeutic for the treatment of neurological disease.

The TRACER AAV particles comprising a viral genome with a nucleic acid sequence encoding one or more siRNA molecules may be used in combination with one or more other therapeutic agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent.

Therapeutic agents that may be used in combination with the TRACER AAV particles comprising a viral genome with a nucleic acid sequence encoding one or more siRNA molecules can be small molecule compounds which are antioxidants, anti-inflammatory agents, anti-apoptosis agents, calcium regulators, antiglutamatergic agents, structural protein inhibitors, compounds involved in muscle function, and compounds involved in metal ion regulation.

Compounds tested for treating neurological disease which may be used in combination with the TRACER AAV particles comprising a viral genome with a nucleic acid sequence encoding one or more siRNA molecules include, but are not limited to, cholinesterase inhibitors (donepezil, rivastigmine, galantamine), NMDA receptor antagonists such as memantine, anti-psychotics, anti-depressants, anti-convulsants (e.g., sodium valproate and levetiracetam for myoclonus), secretase inhibitors, amyloid aggregation inhibitors, copper or zinc modulators, BACE inhibitors, inhibitors of tau aggregation, such as Methylene blue, phenothiazines, anthraquinones, n-phenylamines or rhodamines, microtubule stabilizers such as NAP, taxol or paclitaxel, kinase or phosphatase inhibitors such as those targeting GSK3β (lithium) or PP2A, immunization with Aβ peptides or tau phospho-epitopes, anti-tau or anti-amyloid antibodies, dopamine-depleting agents (e.g., tetrabenazine for chorea), benzodiazepines (e.g., clonazepam for myoclonus, chorea, dystonia, rigidity, and/or spasticity), amino acid precursors of dopamine (e.g., levodopa for rigidity), skeletal muscle relaxants (e.g., baclofen, tizanidine for rigidity and/or spasticity), inhibitors for acetylcholine release at the neuromuscular junction to cause muscle paralysis (e.g., botulinum toxin for bruxism and/or dystonia), atypical neuroleptics (e.g., olanzapine and quetiapine for psychosis and/or irritability, risperidone, sulpiride and haloperidol for psychosis, chorea and/or irritability, clozapine for treatment-resistant psychosis, aripiprazole for psychosis with prominent negative symptoms), selective serotonin reuptake inhibitors (SSRIs) (e.g., citalopram, fluoxetine, paroxetine, sertraline, mirtazapine, venlafaxine for depression, anxiety, obsessive compulsive behavior and/or irritability), hypnotics (e.g., xopiclone and/or zolpidem for altered sleep-wake cycle), anticonvulsants (e.g., sodium valproate and carbamazepine for mania or hypomania) and mood stabilizers (e.g., lithium for mania or hypomania).

Neurotrophic factors may be used in combination therapy with the TRACER AAV particles comprising a viral genome with a nucleic acid sequence encoding one or more siRNA molecules for treating neurological disease. Generally, a neurotrophic factor is defined as a substance that promotes survival, growth, differentiation, proliferation and/or maturation of a neuron, or stimulates increased activity of a neuron. In some embodiments, the present methods further comprise delivery of one or more trophic factors into the subject in need of treatment. Trophic factors may include, but are not limited to, IGF-I, GDNF, BDNF, CTNF, VEGF, Colivelin, Xaliproden, Thyrotrophin-releasing hormone and ADNF, and variants thereof.

In one aspect, the TRACER AAV particle encoding the nucleic acid sequence for the at least one siRNA duplex targeting the gene of interest may be co-administered with TRACER AAV particles expressing neurotrophic factors such as AAV-IGF-I (See e.g., Vincent et al., Neuromolecular medicine, 2004, 6, 79-85; the content of which is incorporated herein by reference in its entirety) and AAV-GDNF (See e.g., Wang et al., J Neurosci., 2002, 22, 6920-6928; the contents of which are incorporated herein by reference in their entirety).

In one embodiment, administration of the TRACER AAV particles to a subject will reduce the expression of a target protein in a subject and the reduction of expression of the target protein will reduce the effects and/or symptoms of neurological disease in a subject.

### Definitions

Adeno-associated virus: As used herein, the term “adeno-associated virus” or “AAV” refers to members of the dependovirus genus comprising any particle, sequence, gene, protein, or component derived therefrom.

AAV Particle: As used herein, an “AAV particle” is a virus which comprises a capsid and a viral genome with at least one payload region and at least one ITR. As used herein “AAV particles of the disclosure” are AAV particles comprising a parent capsid sequence with at least one targeting peptide insert. AAV particles of the present disclosure may be produced recombinantly and may be based on adeno-associated virus (AAV) parent or reference sequences. AAV particle may be derived from any serotype, described herein or known in the art, including combinations of serotypes (i.e., “pseudotyped” AAV) or from various genomes (e.g., single stranded or self-complementary). In addition, the AAV particle may be replication defective and/or targeted. In one embodiment, the AAV particle may have a targeting peptide inserted into the capsid to enhance tropism for a desired target tissue. It is to be understood that reference to the AAV particles of the disclosure also includes pharmaceutical compositions thereof, even if not explicitly recited.

Administering: As used herein, the term “administering” refers to providing a pharmaceutical agent or composition to a subject.

Amelioration: As used herein, the term “amelioration” or “ameliorating” refers to a lessening of severity of at least one indicator of a condition or disease. For example, in the context of neurodegeneration disorder, amelioration includes the reduction of neuron loss.

Animal: As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically engineered animal, or a clone.

Antisense strand: As used herein, the term “the antisense strand” or “the first strand” or “the guide strand” of a siRNA molecule refers to a strand that is substantially complementary to a section of about 10-50 nucleotides, e.g., about 15-30, 16-25, 18-23 or 19-22 nucleotides of the mRNA of a gene targeted for silencing. The antisense strand or first strand has sequence sufficiently complementary to the desired target mRNA sequence to direct target-specific silencing, e.g., complementarity sufficient to trigger the destruction of the desired target mRNA by the RNAi machinery or process.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Capsid: As used herein, the term “capsid” refers to the protein shell of a virus particle.

Complementary and substantially complementary: As used herein, the term “complementary” refers to the ability of polynucleotides to form base pairs with one another. Base pairs are typically formed by hydrogen bonds between nucleotide units in antiparallel polynucleotide strands. Complementary polynucleotide strands can form base pairs in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenine. However, when a U is denoted in the context of the present disclosure, the ability to substitute a T is implied, unless otherwise stated. Perfect complementarity or 100% complementarity refers to the situation in which each nucleotide unit of one polynucleotide strand can form a hydrogen bond with a nucleotide unit of a second polynucleotide strand. Less than perfect complementarity refers to the situation in which some, but not all, nucleotide units of two strands can form hydrogen bond with each other. For example, for two 20-mers, if only two base pairs on each strand can form a hydrogen bond with each other, the polynucleotide strands exhibit 10% complementarity. In the same example, if 18 base pairs on each strand can form hydrogen bonds with each other, the polynucleotide strands exhibit 90% complementarity. As used herein, the term “substantially complementary” means that the siRNA has a sequence (e.g., in the antisense strand) which is sufficient to bind the desired target mRNA, and to trigger the RNA silencing of the target mRNA.

Control Elements: As used herein, “control elements”, “regulatory control elements” or “regulatory sequences” refers to promoter regions, polyadenylation signals, transcription termination sequences, upstream regulatory domains, origins of replication, internal ribosome entry sites (“IRES”), enhancers, and the like, which provide for the replication, transcription and translation of a coding sequence in a recipient cell. Not all of these control elements need always be present as long as the selected coding sequence is capable of being replicated, transcribed and/or translated in an appropriate host cell.

Delivery: As used herein, “delivery” refers to the act or manner of delivering an AAV particle, a compound, substance, entity, moiety, cargo or payload.

Element: As used herein, the term “element” refers to a distinct portion of an entity. In some embodiments, an element may be a polynucleotide sequence with a specific purpose, incorporated into a longer polynucleotide sequence.

Encapsulate: As used herein, the term “encapsulate” means to enclose, surround or encase. As an example, a capsid protein often encapsulates a viral genome.

Engineered: As used herein, embodiments of the disclosure are “engineered” when they are designed to have a feature or property, whether structural or chemical, that varies from a starting point, wild type or native molecule.

Effective Amount: As used herein, the term “effective amount” of an agent is that amount sufficient to effect beneficial or desired results, for example, clinical results, and, as such, an “effective amount” depends upon the context in which it is being applied. For example, in the context of administering an agent that treats cancer, an effective amount of an agent is, for example, an amount sufficient to achieve treatment, as defined herein, of cancer, as compared to the response obtained without administration of the agent.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Feature: As used herein, a “feature” refers to a characteristic, a property, or a distinctive element.

Formulation: As used herein, a “formulation” includes at least one AAV particle (active ingredient) and an excipient, and/or an inactive ingredient.

Fragment: A “fragment,” as used herein, refers to a portion. For example, an antibody fragment may comprise a CDR, or a heavy chain variable region, or a scFv, etc.

Functional: As used herein, a “functional” biological molecule is a biological molecule in a form in which it exhibits a property and/or activity by which it is characterized.

Gene expression: The term “gene expression” refers to the process by which a nucleic acid sequence undergoes successful transcription and in most instances translation to produce a protein or peptide. For clarity, when reference is made to measurement of “gene expression”, this should be understood to mean that measurements may be of the nucleic acid product of transcription, e.g., RNA or mRNA or of the amino acid product of translation, e.g., polypeptides or peptides. Methods of measuring the amount or levels of RNA, mRNA, polypeptides and peptides are well known in the art.

Homology: As used herein, the term “homology” refers to the overall relatedness between polymeric molecules, e.g. between polynucleotide molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the disclosure, two polynucleotide sequences are considered to be homologous if the polypeptides they encode are at least about 50%, 60%, 70%, 80%, 90%, 95%, or even 99% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In accordance with the disclosure, two protein sequences are considered to be homologous if the proteins are at least about 50%, 60%, 70%, 80%, or 90% identical for at least one stretch of at least about 20 amino acids.

Identity: As used herein, the term “identity” refers to the overall relatedness between polymeric molecules, e.g., between polynucleotide molecules (e.g. DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; the contents of each of which are incorporated herein by reference in their entirety. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

Inhibit expression of a gene: As used herein, the phrase “inhibit expression of a gene” means to cause a reduction in the amount of an expression product of the gene. The expression product can be an RNA transcribed from the gene (e.g., an mRNA) or a polypeptide translated from an mRNA transcribed from the gene. Typically, a reduction in the level of an mRNA results in a reduction in the level of a polypeptide translated therefrom. The level of expression may be determined using standard techniques for measuring mRNA or protein.

Insert: As used herein the term “insert” may refer to the addition of a targeting peptide sequence to a parent AAV capsid sequence. An “insertion” may result in the replacement of one or more amino acids of the parent AAV capsid sequence. Alternatively, an insertion may result in no changes to the parent AAV capsid sequence beyond the addition of the targeting peptide sequence.

Inverted terminal repeat: As used herein, the term “inverted terminal repeat” or “ITR” refers to a cis-regulatory element for the packaging of polynucleotide sequences into viral capsids.

Library: As used herein, the term “library” refers to a diverse collection of linear polypeptides, polynucleotides, viral particles, or viral vectors. As examples, a library may be a DNA library or an AAV capsid library.

Neurological disease: As used herein, a “neurological disease” is any disease associated with the central or peripheral nervous system and components thereof (e.g., neurons).

Naturally Occurring: As used herein, “naturally occurring” or “wild-type” means existing in nature without artificial aid, or involvement of the hand of man.

Open reading frame: As used herein, “open reading frame” or “ORF” refers to a sequence which does not contain a stop codon in a given reading frame.

Parent sequence: As used herein, a “parent sequence” is a nucleic acid or amino acid sequence from which a variant is derived. In one embodiment, a parent sequence is a sequence into which a heterologous sequence is inserted. In other words, a parent sequence may be considered an acceptor or recipient sequence. In one embodiment, a parent sequence is an AAV capsid sequence into which a targeting sequence is inserted.

Particle: As used herein, a “particle” is a virus comprised of at least two components, a protein capsid and a polynucleotide sequence enclosed within the capsid.

Patient: As used herein, “patient” refers to a subject who may seek or be in need of treatment, requires treatment, is receiving treatment, will receive treatment, or a subject who is under care by a trained professional for a particular disease or condition.

Payload region: As used herein, a “payload region” is any nucleic acid sequence (e.g., within the viral genome) which encodes one or more “payloads” of the disclosure. As non-limiting examples, a payload region may be a nucleic acid sequence within the viral genome of an AAV particle, which encodes a payload, wherein the payload is an RNAi agent or a polypeptide. Payloads of the present disclosure may be, but are not limited to, peptides, polypeptides, proteins, antibodies, RNAi agents, etc.

Peptide: As used herein, “peptide” is less than or equal to 50 amino acids long, e.g., about 5, 10, 15, 20, 25, 30, 35, 40, 45, or 50 amino acids long.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Preventing: As used herein, the term “preventing” or “prevention” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Prophylactic: As used herein, “prophylactic” refers to a therapeutic or course of action used to prevent the spread of disease.

Prophylaxis: As used herein, a “prophylaxis” refers to a measure taken to maintain health and prevent the spread of disease.

Region: As used herein, the term “region” refers to a zone or general area. In some embodiments, when referring to a protein or protein module, a region may comprise a linear sequence of amino acids along the protein or protein module or may comprise a three-dimensional area, an epitope and/or a cluster of epitopes. In some embodiments, regions comprise terminal regions. As used herein, the term “terminal region” refers to regions located at the ends or termini of a given agent. When referring to proteins, terminal regions may comprise N- and/or C-termini.

In some embodiments, when referring to a polynucleotide, a region may comprise a linear sequence of nucleic acids along the polynucleotide or may comprise a three-dimensional area, secondary structure, or tertiary structure. In some embodiments, regions comprise terminal regions. As used herein, the term “terminal region” refers to regions located at the ends or termini of a given agent. When referring to polynucleotides, terminal regions may comprise 5′ and/or 3′ termini.

RNA or RNA molecule: As used herein, the term “RNA” or “RNA molecule” or “ribonucleic acid molecule” refers to a polymer of ribonucleotides; the term “DNA” or “DNA molecule” or “deoxyribonucleic acid molecule” refers to a polymer of deoxyribonucleotides. DNA and RNA can be synthesized naturally, e.g., by DNA replication and transcription of DNA, respectively; or be chemically synthesized. DNA and RNA can be single-stranded (i.e., ssRNA or ssDNA, respectively) or multi-stranded (e.g., double stranded, i.e., dsRNA and dsDNA, respectively). The term “mRNA” or “messenger RNA”, as used herein, refers to a single stranded RNA that encodes the amino acid sequence of one or more polypeptide chains.

RNA interfering or RNAi: As used herein, the term “RNA interfering” or “RNAi” refers to a sequence specific regulatory mechanism mediated by RNA molecules which results in the inhibition or interfering or “silencing” of the expression of a corresponding protein-coding gene. RNAi has been observed in many types of organisms, including plants, animals and fungi. RNAi occurs in cells naturally to remove foreign RNAs (e.g., viral RNAs). Natural RNAi proceeds via fragments cleaved from free dsRNA which direct the degradative mechanism to other similar RNA sequences. RNAi is controlled by the RNA-induced silencing complex (RISC) and is initiated by short/small dsRNA molecules in cell cytoplasm, where they interact with the catalytic RISC component argonaute. The dsRNA molecules can be introduced into cells exogenously. Exogenous dsRNA initiates RNAi by activating the ribonuclease protein Dicer, which binds and cleaves dsRNAs to produce double-stranded fragments of 21-25 base pairs with a few unpaired overhang bases on each end. These short double stranded fragments are called small interfering RNAs (siRNAs).

RNAi agent: As used herein, the term “RNAi agent” refers to an RNA molecule, or its derivative, that can induce inhibition, interfering, or “silencing” of the expression of a target gene and/or its protein product. An RNAi agent may knock-out (virtually eliminate or eliminate) expression, or knock-down (lessen or decrease) expression. The RNAi agent may be, but is not limited to, dsRNA, siRNA, shRNA, pre-miRNA, pri-miRNA, miRNA, stRNA, lncRNA, piRNA, or snoRNA.

Sample: As used herein, the term “sample” or “biological sample” refers to a subset of its tissues, cells or component parts (e.g. body fluids, including but not limited to blood, serum, mucus, lymphatic fluid, synovial fluid, cerebrospinal fluid, saliva, amniotic fluid, amniotic cord blood, urine, vaginal fluid and semen). A sample further may include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. A sample further refers to a medium, such as a nutrient broth or gel, which may contain cellular components, such as proteins or nucleic acid molecule.

Self-complementary viral particle: As used herein, a “self-complementary viral particle” is a particle comprised of at least two components, a protein capsid and a self-complementary viral genome enclosed within the capsid.

Sense Strand: As used herein, the term “the sense strand” or “the second strand” or “the passenger strand” of a siRNA molecule refers to a strand that is complementary to the antisense strand or first strand. The antisense and sense strands of a siRNA molecule are hybridized to form a duplex structure. As used herein, a “siRNA duplex” includes a siRNA strand having sufficient complementarity to a section of about 10-50 nucleotides of the mRNA of the gene targeted for silencing and a siRNA strand having sufficient complementarity to form a duplex with the other siRNA strand.

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Short interfering RNA or siRNA: As used herein, the terms “short interfering RNA,” “small interfering RNA” or “siRNA” refer to an RNA molecule (or RNA analog) comprising between about 5-60 nucleotides (or nucleotide analogs) which is capable of directing or mediating RNAi. Preferably, a siRNA molecule comprises between about 15-30 nucleotides or nucleotide analogs, such as between about 16-25 nucleotides (or nucleotide analogs), between about 18-23 nucleotides (or nucleotide analogs), between about 19-22 nucleotides (or nucleotide analogs) (e.g., 19, 20, 21 or 22 nucleotides or nucleotide analogs), between about 19-25 nucleotides (or nucleotide analogs), and between about 19-24 nucleotides (or nucleotide analogs). The term “short” siRNA refers to a siRNA comprising 5-23 nucleotides, preferably 21 nucleotides (or nucleotide analogs), for example, 19, 20, 21 or 22 nucleotides. The term “long” siRNA refers to a siRNA comprising 24-60 nucleotides, preferably about 24-25 nucleotides, for example, 23, 24, 25 or 26 nucleotides. Short siRNAs may, in some instances, include fewer than 19 nucleotides, e.g., 16, 17 or 18 nucleotides, or as few as 5 nucleotides, provided that the shorter siRNA retains the ability to mediate RNAi. Likewise, long siRNAs may, in some instances, include more than 26 nucleotides, e.g., 27, 28, 29, 30, 35, 40, 45, 50, 55, or even 60 nucleotides, provided that the longer siRNA retains the ability to mediate RNAi or translational repression absent further processing, e.g., enzymatic processing, to a short siRNA. siRNAs can be single stranded RNA molecules (ss-siRNAs) or double stranded RNA molecules (ds-siRNAs) comprising a sense strand and an antisense strand which hybridized to form a duplex structure called an siRNA duplex.

Subject: As used herein, the term “subject” or “patient” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Targeting peptide: As used herein, a “targeting peptide” refers to a peptide of 3-20 amino acids in length. These targeting peptides may be inserted into, or attached to, a parent amino acid sequence to alter the characteristics (e.g., tropism) of the parent protein. As a non-limiting example, the targeting peptide can be inserted into an AAV capsid sequence for enhanced targeting to a desired cell-type, tissue, organ or organism. It is to be understood that a targeting peptide is encoded by a targeting polynucleotide which may similarly be inserted into a parent polynucleotide sequence. Therefore, a “targeting sequence” refers to a peptide or polynucleotide sequence for insertion into an appropriate parent sequence (amino acid or polynucleotide, respectively).

Target Cells: As used herein, “target cells” or “target tissue” refers to any one or more cells of interest. The cells may be found in vitro, in vivo, in situ or in the tissue or organ of an organism. The organism may be an animal, preferably a mammal, more preferably a human and most preferably a patient.

Therapeutic Agent: The term “therapeutic agent” refers to any agent that, when administered to a subject, has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In some embodiments, a therapeutically effective amount is provided in a single dose.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Vector: As used herein, the term “vector” refers to any molecule or moiety which transports, transduces or otherwise acts as a carrier of a heterologous molecule. In some embodiments, vectors may be plasmids. In some embodiments, vectors may be viruses. An AAV particle is an example of a vector. Vectors of the present disclosure may be produced recombinantly and may be based on and/or may comprise adeno-associated virus (AAV) parent or reference sequences. The heterologous molecule may be a polynucleotide and/or a polypeptide.

Viral Genome: As used herein, the terms “viral genome” or “vector genome” refer to the nucleic acid sequence(s) encapsulated in an AAV particle. A viral genome comprises a nucleic acid sequence with at least one payload region encoding a payload and at least one ITR.

### EQUIVALENTS AND SCOPE

Those skilled in the art will recognize or be able to ascertain using no more than routine experimentation, many equivalents to the specific embodiments in accordance with the disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

The present disclosure is further illustrated by the following non-limiting examples.

### EXAMPLES

### Example 1. TRACER Proof of Concept: Promoter Selection

Proof-of-concept experiments were conducted by placing the genes encoding an AAV9 peptide display capsid library under the control of either the neuron-specific synapsin promoter (SYN) or the astrocyte-specific GFAP promoter. Following intravenous administration to C57BL/6 mice, RNA was recovered from brain tissue and used for further library evolution. Next-generation sequencing (NGS) showed sequence convergence between animals after only two rounds of selection. Interestingly, several variants highly similar to the PHP.eB capsid were recovered, suggesting that our method allowed a rapid selection of high-performance capsids. A subset of capsids having peptide sequences with high CNS enrichment was selected for further study. It is understood that any promoter may be selected depending on the desired tropism. Examples of such promoters are found in Table 3.

TABLE 3
Promoters, tissue and cell type

| Promoter name | Tissue | Cell type |
|---|---|---|
| B29 promoter | Blood | B cells |
| Immunoglobulin heavy chain | Blood | B cells |
| promoter | | |
| CD45 promoter | Blood | Hematopoietic |
| Mouse INF-β promoter | Blood | Hematopoietic |
| CD45 SV40/CD45 promoter | Blood | Hematopoietic |
| WASP promoter | Blood | Hematopoietic |
| CD43 promoter | Blood | Leuko & Platelets |
| CD43 SV40/CD43 promoter | Blood | Leuko & Platelets |
| CD68 promoter | Blood | Macrophages |
| GPIIb promoter | Blood | Megakaryocyte |
| CD14 promoter | Blood | Monocytes |
| CD2 promoter | Blood | T cells |
| Osteocalcin | Bone | Osteoblasts |
| Bone sialoprotein | Bone | Osteoblasts |
| OG-2 promoter | Bone | Osteoblasts, odontoblasts |
| GFAP promoter | Brain | Astrocytes |
| Vga | Brain | GABAergic neurons |
| Vglut2 | Brain | glutamatergic neurons |
| NSE/RU5′ promoter | Brain | Neurons |
| SYN1 promoter | Brain | Neurons |
| Neurofilament light chain | Brain | Neurons |
| VGF | Brain | Neurons |
| Nestin | Brain | NSC |
| Chx10 | Eye | All retinal neurons |
| PrP | Eye | All retinal neurons |
| Dkk3 | Eye | All retinal neurons |
| Math5 | Eye | Amacrine and horizontal |
| | | cells |
| Ptf1a | Eye | Amacrine and horizontal |
| | | cells |
| Pcp2 | Eye | Bipolar cells |
| Nefh | Eye | Ganglion cells |
| gamma-synuclein gene | Eye | ganglion cells |
| (SNCG) | | |
| Grik4 | Eye | GC |
| Pdgfra | Eye | GC and ONL Müller cells |
| Chat | Eye | GC/Amacrine cells |
| Thy1.2 | Eye | GC/neural retina |
| hVmd2 | Eye | INL Müller cells |
| Thy1 | Eye | INL Müller cells |
| Modified αA-crystallin | Eye | Lens/neural retina |
| hRgp | Eye | M- and S-cone |
| mMo | Eye | M-cone |
| Opn4 | Eye | Melanopsin-expressing GC |
| RLBP1 | Eye | Muller cells |
| Glast | Eye | Müller cells |
| Foxg1 | Eye | Müller cells |
| hVmd2 | Eye | Müller cells/optic nerve/ |
| | | INL |
| Trp1 | Eye | Neural retina |
| Six3 | Eye | Neural retina |
| cx36 | Eye | Neurons |
| Grm6-SV40 eukaryotic | Eye | ON bipolar |
| promoter | | |
| hVmd2 | Eye | Optic nerve |
| Dct | Eye | Pigmented cells |
| Rpc65 | Eye | Retinal pigment epithelium |
| mRho | Eye | Rod |
| Irbp | Eye | Rod |
| hRho | Eye | Rod |
| Pcp2 | Eye | Rod bipolar cells |
| Rhodopsin | Eye | Rod Photoreceptors |
| mSo | Eye | S-cone |
| MLC2v promoter | Heart | Cardiomyocyte |
| αMHC promoter | Heart | Cardiomyocyte |
| rat troponin T (Tnnt2) | Heart | Cardiomyocyte |
| Tie2 | Heart | Endothelial |
| Tcf21 | Heart | Fibroblasts |
| ECAD | Kidney | Collecting duct |
| NKCC2 | Kidney | Loop of Henle |
| KSPC | Kidney | Nephron |
| NPHS1 | Kidney | Podocyte |
| SGLT2 | Kidney | Proximal tubular cells |
| SV40/bAlb promoter | Liver | hepatocytes |
| SV40/hAlb promoter | Liver | hepatocytes |
| Hepatitis B virus core | Liver | hepatocytes |
| promoter | | |
| Alpha fetoprotein | Liver | hepatocytes |
| Surfactant protein B promoter | Lung | AT II cells and Clara cells |
| Surfactant protein C promoter | Lung | AT II cells and Clara cells |
| Desmin | Muscle | Muscle stem cells + |
| | | Myocytes |
| Mb promoter | Muscle | Myocyte |
| Myosin | Muscle | Myocyte |
| Dystrophin | Muscle | Myocyte |
| dMCK and tMCK | Muscle | Myocytes |
| Elastase-1 promoter | Pancreas | Acinar cells |
| PDX1 promoter | Pancreas | Beta cells |
| Insulin promoter | Pancreas | langherans |
| Slco1c1 | Vasculature | BBB Endothelial |
| tie | Vasculature | Endothelial |
| cadherin | Vasculature | Endothelial |
| ICAM-2 | Vasculature | Endothelial |
| claudin 1 | Vasculature | Endothelial |
| Cldn5 | Vasculature | Endothelial |
| Flt-1 promoter | Vasculature | Endothelial |
| Endoglin promoter | Vasculature | Endothelial |

Capsid pools were injected to three rodent species, followed by RNA enrichment analysis for characterization of transduction efficiency in neurons or astrocytes and cross-species performance. Top-ranking capsids were then individually tested and several variants showed CNS transduction similar to or higher than the PHP.eB benchmark. These results suggest that the TRACER platform allows rapid in vivo evolution of AAV capsids in non-transgenic animals with a high degree of tropism improvement. The following examples illustrate the findings in more detail.

### Example 2. Generation of an AAV Vectors Capable of Capsid mRNA Expression in the Absence of Helper Virus

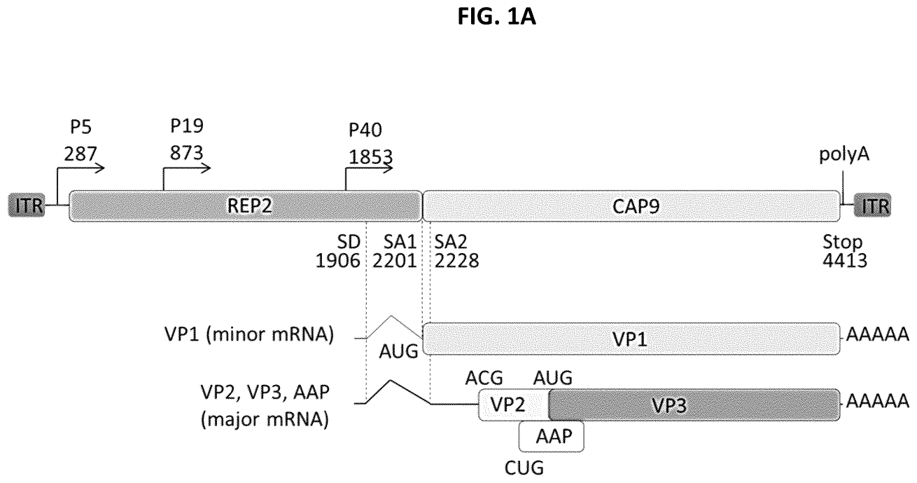

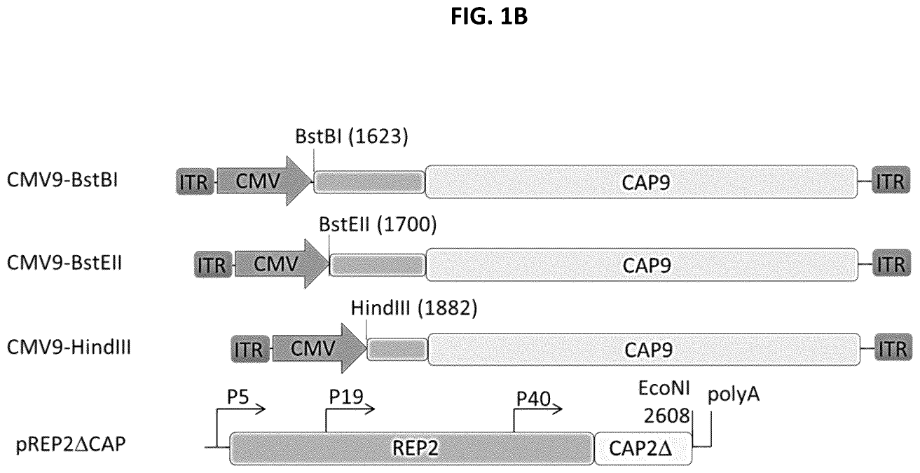

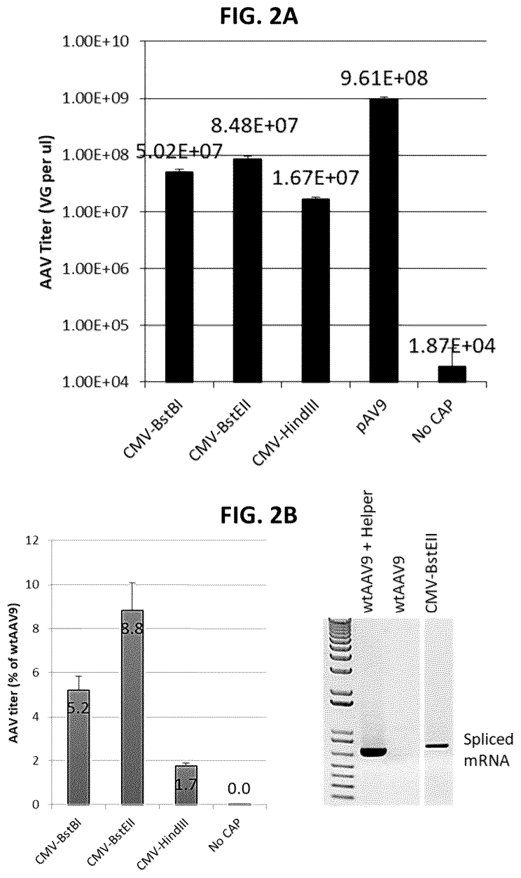

In order to perform cell type- and transduction-restricted in vivo evolution of AAV capsid libraries, a capsid library system was engineered in which the capsid mutant gene can be transcribed in the absence of a helper virus, in a specific cell type. In the wild-type AAV virus, the mRNA encoding the capsid proteins VP1, VP2 and VP3, as well as the AAP accessory protein, are expressed by the P40 promoter located in the 3′ region of the REP gene (FIG. 1A), that is only active in the presence of the REP protein as well as the helper virus functions (Berns et al., 1996). In order to allow expression of the capsid mRNA in animal tissue or in cultured cells, another promoter must be inserted upstream or downstream of the CAP gene. Because of the limited packaging capacity of the AAV capsid, a portion of the REP gene must be deleted to accommodate the extra promoter insertion, and the REP gene has to be provided in trans by another plasmid to allow virus production. The minimal viral sequence required for high titer AAV production was determined by introducing a CMV promoter at various locations upstream of the CAP gene of AAV9 (FIG. 1B). The REP protein was provided in trans by the pREP2 plasmid obtained by deleting the CAP gene from a REP2-CAP2 packaging vector using EcoNI and ClaI (SEQ. ID NO: 4). For small-scale virus production test, HEK-293T cells grown in DMEM supplemented with 5% FBS and 1× pen/strep were plated in 15-cm dishes and co-transfected with 15 ug of pHelper (pFdelta6) plasmid, 10 ug pREP2 plasmid and 1 ug ITR-CMV-CAP plasmid using calcium phosphate transfection. After 72 hours, cells were harvested by scraping, pelleted by a brief centrifugation and suspended in 1 ml of a buffer containing 10 mM Tris and 2 mM MgCl2. Cells were lysed by addition of triton X-100 to 0.1% final concentration and treated with 50U of benzonase for 1 hour. Virus from the supernatants was precipitated with 8% polyethylene glycol and 0.5M NaCl, suspended in 1 ml of 10 mM TRIS-2 mM MgCl2 and combined with the cell lysate. The pooled virus was adjusted to 0.5M NaCl, cleared by centrifugation for 15 minutes at 4,000×g and fractionated on a step iodixanol gradient of 15%, 25%, 40% and 60% for 3 hours at 40,000 prm (Zolotukhin et al., 1999). The 40% fraction containing the purified AAV particles was harvested and viral titers were measured by real-time PCR using a Taqman primer/probe mix specific for the 3′-end of REP, shared by all the constructs. Virus yields were significantly lower than the fully wild-type ITR-REP2-CAP9-ITR used as a reference (1.7% to 8.8%), but the CMV-BstEII construct allowed the highest yields of all three CMV constructs. See FIG. 2. The CMV-HindIII construct, in which most of the P40 promoter sequence is deleted, generated the lowest yield (1.7% of wtAAV9), indicating that even the potent CMV promoter cannot replace the P40 promoter without a severe drop in virus yields. Following these observations, the BstEII architecture (SEQ. ID NO: 5), which preserves the minimal P40 sequence and the CAP mRNA splice donor, was used in all further experiments.

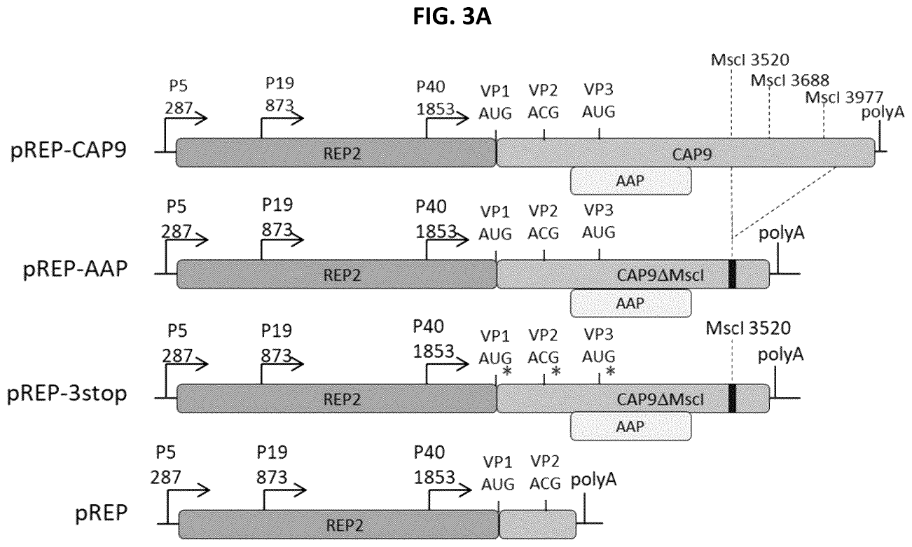

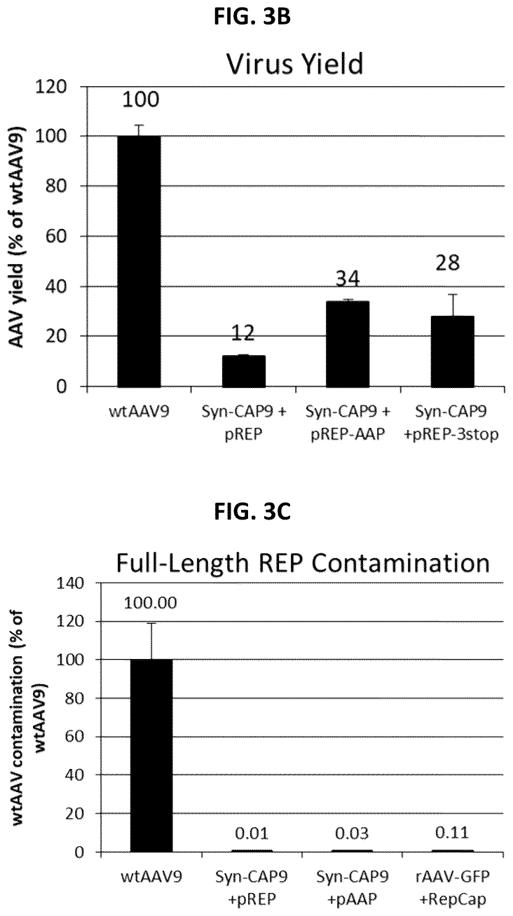

The REP-expressing plasmid was then improved by preserving the AAP reading frame together with a large portion of the capsid gene from the REP2-CAP9 helper vector, which may contain sequences necessary for the regulation of CAP transcription and/or splicing. In order to eliminate the capsid coding potential of the vector, a C-terminus fragment of the capsid gene was deleted by a triple cut with the MscI restriction enzyme followed by self-ligation, in order to obtain the pREP-AAP plasmid (FIG. 3A, SEQ. ID NO: 6).

An iteration of this construct was engineered by introducing premature stop codons immediately after the start codons of VP1, VP2 and VP3, without perturbing the amino acid sequence of the colinear AAP reading frame (FIG. 3A). This construct was named pREP-3stop (SEQ. ID NO: 7). A neuron-specific syn-CAP9 vector (SEQ. ID NO: 8) was derived from the CMV9-BstEII plasmid by swapping the CMV promoter with the neuron-specific human synapsin 1 promoter.

Production efficiency of this Syn-CAP9 was tested as described previously using PREP, pREP-AAP or pREP-3stop plasmid to supply REP in trans. As shown in FIG. 3B, the REP plasmids harboring a longer capsid sequence as well as AAP increased virus yields by approximately 3-fold compared to the pREP plasmid. Virus titers obtained with the pREP-AAP or pREP-3stop vectors reached ˜30% of wild-type AAV9. An important concern with plasmids harboring long homologous regions is the potential for unwanted recombination with the ITR-CAP vector, that would reconstitute a wild-type ITR-REP-CAP vector and contaminate combinatorial libraries.

To evaluate the risk of wild-type virus reconstitution, the viral preparations obtained in FIG. 3B were subjected to real-time PCR with a Taqman probe located in the N terminus of REP. The percentage of capsids containing a detectable full-length REP was less than 0.03% of wild-type virus (FIG. 3C), which was even lower than the routinely detected 0.1% illegitimate REP-CAP packaging occurring in most recombinant AAV preparations obtained from 293T cell transfection (FIG. 3C, our unpublished observations). Because the premature stop codons of the pREP-3stop vector offer an extra layer of safety against potential reconstitution of wild-type capsids and prevents the translation of truncated capsid proteins, the 3stop plasmid was used for all subsequent studies.

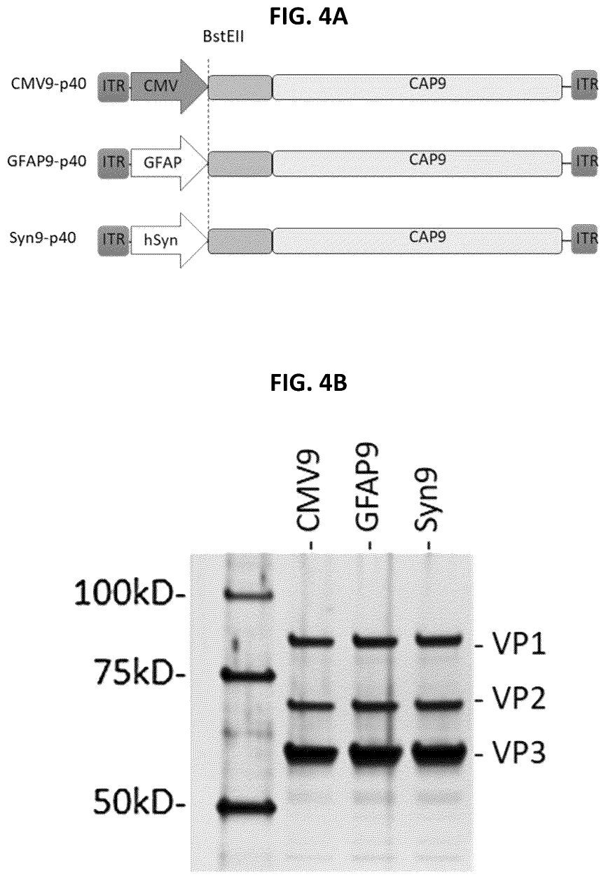

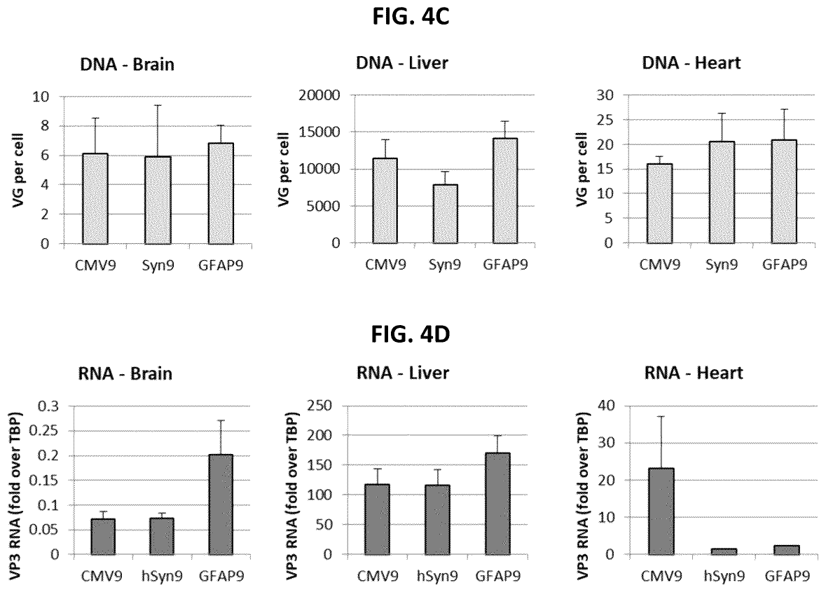

Following this, the feasibility of RNA-driven biopanning in C57BL/6 mice using AAV9-packaged vectors where the AAV9 capsid gene is driven by the CMV promoter, the Synapsin promoter or the astrocyte-specific GFabc1D promoter (SEQ. ID NO: 9), thereafter referred to as GFAP promoter (Brenner et al., 2008) was tested (FIG. 4A). The three vectors were produced in HEK-293T cells as previously described and analyzed by PAGE-silver stain. As shown in FIG. 4B, all vectors showed a normal ratio of VP1, VP2 and VP3 capsid proteins, indicating that the particular promoter architecture does not disrupt the balance of capsid protein expression. Six-week old male C57BL/6 mice were injected intravenously with 1e12 VG per mouse and sacrificed after 28 days. DNA biodistribution and capsid mRNA expression were tested in the brain, liver and heart tissues.

Total DNA was extracted from brain, liver and heart tissues using Qiagen DNeasy Blood and Tissue columns, and viral DNA was quantified by real-time PCR using a Taqman probe located in the VP3 N-terminal region. DNA abundance was normalized using a pre-designed probe detecting the single-copy transferrin receptor gene (Life Technologies ref. 4458366). Viral DNA was highly abundant in the liver and to a lower extent in the heart. The DNA distribution did not show any noticeable difference between the three vectors (FIG. 4C). RNA was extracted with Qiagen RNeasy plus universal kit following manufacturer's instructions, then treated with ezDNAse (Qiagen) to remove residual DNA, and reverse transcribed with Superscript IV (Life technologies).

RNA expression was evaluated using the same VP3 probe used to quantify viral DNA and normalized using TBP as a reference RNA (Life technologies Mm01277042_m1). In the brain, the GFAP promoter allowed the strongest expression level, and the Synapsin promoter allowed a comparable expression as the potent CMV promoter. In the liver, all promoters resulted in a similar expression level, which could be the result of a leaky expression at very high copy number (FIG. 4D). In the heart, the cell type specificity of the Syn and GFAP promoters was evident, since they allowed only ˜3 and 10% of CMV expression, respectively despite of a similar DNA biodistribution.

Overall the experiment showed that mRNA from transduction-competent capsids could be recovered from various animal organs, including weakly transduced tissues such as the brain.

### Example 3. AAV Vector Configuration

Various vector configurations were explored toward increasing RNA expression to maximize library recovery. The CMV promoter was replaced by a hybrid CMV enhancer/Chicken beta-actin promoter sequence (Niwa et al., 1991) and a potent cytomegalovirus-beta-globin hybrid intron derived from the AAV-MCS cloning vector (Stratagene) was inserted between the promoter sequence and the capsid gene, as introns have been shown to increase mRNA processing and stability (Powell et al., 2015). This resulted in the constructs CAG9 (SEQ. ID NO: 10), SYNG9 (SEQ. ID NO: 11) and GFAPG (SEQ. ID NO: 12).

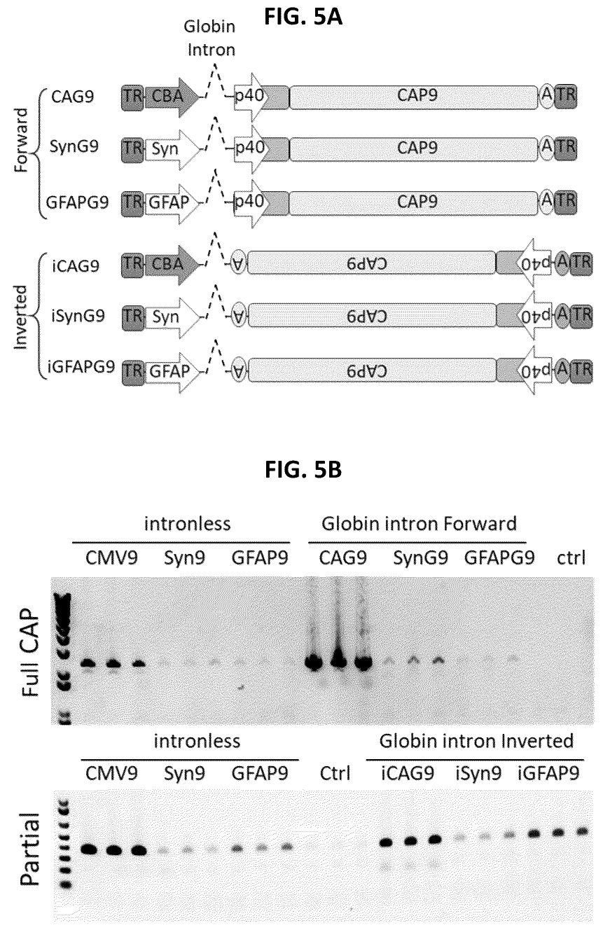

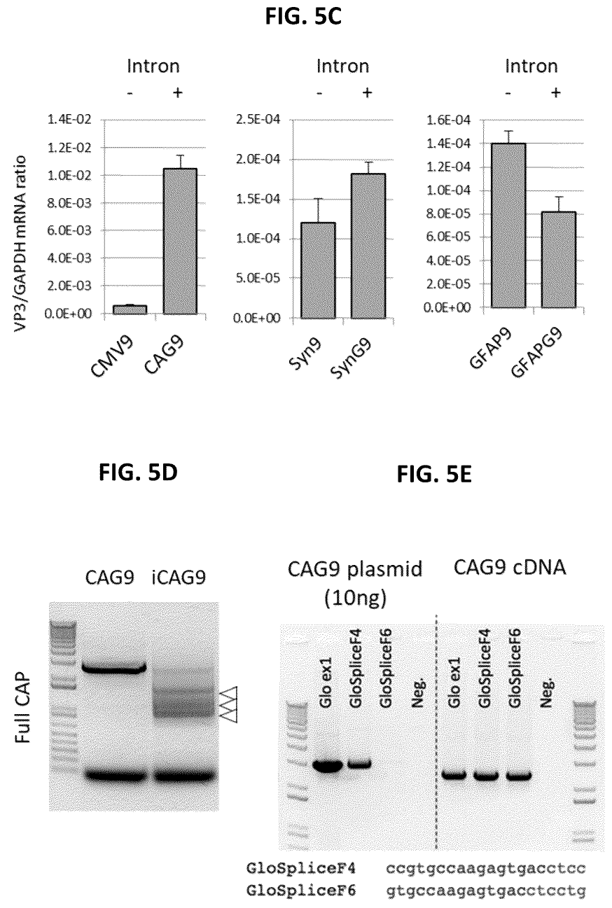

An inverted vector configuration was also tested where the helper-independent promoter was placed downstream of the capsid gene in reverse orientation, in order to avoid potential interference with the P40 promoter (FIG. 5A). This configuration allows the expression of an antisense capsid transcript in animal tissue. Because most polyadenylation signals (AATAAA) are orientation-dependent, it was hypothesized that the natural AAV capsid polyA would not prematurely terminate transcription when placed in reverse orientation. All constructs were co-transfected with pHelper and pREP-3stop plasmids to generate AAV9-packaged virions that were used to transduce HEK-293T cells at a MOI of 1e4 VG per cell. RNA was extracted 48 hours post-transfection and reverse transcribed using the Quantitect kit (Qiagen).

PCR was performed with primers allowing amplification of the full-length capsid or a partial sequence localized close to the C-terminus (FIG. 5B). Overall, the presence of an intron had little influence on the expression from low-activity promoters Syn and GFAP, which indicates that mRNA splicing did not alleviate promoter repression in nonpermissive cells. The combination of the CMV enhancer with a Chicken beta-actin promoter and the hybrid intron allowed a significantly higher (>10-fold) mRNA expression compared to CMV promoter alone (FIG. 5B, C).

When comparing endpoint PCR amplification between forward and inverted intronic vectors, a discrepancy was obvious between full-length and partial capsid amplicons (FIG. 5B, right-hand lanes), which led us to question the integrity of capsid RNA. When cDNA from inverted iCAG9 genome was amplified using primers flanking the full-length capsid, multiple low-molecular weight bands were detected, whereas the forward orientation vector allowed amplification of a single product with the expected length (FIG. 5D). Sanger sequencing of low-molecular weight amplicons showed that each band corresponded to an illegitimate splicing product from the antisense capsid RNA.

In light of these results, the forward tandem promoter architecture for subsequent experiments.

Splice-specific PCR amplification was tested to avoid amplification of residual DNA present in RNA preparations. Two candidate PCR primers overlapping the CMV/Globin exon-exon junction were designed and tested them for amplification of cDNA (spliced) or plasmid DNA (still containing the intron sequence). As shown in FIG. 5E, the GloSpliceF6 primer (SEQ. ID NO: 13) allowed a fully specific amplification from cDNA without producing a detectable amplicon from the plasmid DNA sequence. This primer was used in subsequent assays to ascertain the absence of amplification from contaminating DNA.

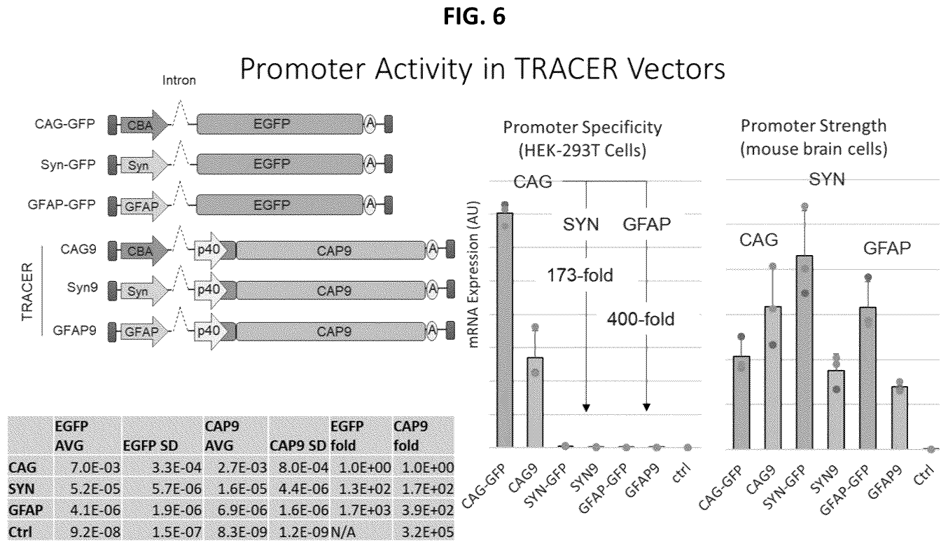

Tandem constructs were then tested for potential interference of the P40 promoter with the cell-specific promoter placed upstream. For this, two series of AAV genomes were tested for transgene mRNA expression in HEK-293T cells. A series of transgenes where the GFP gene was placed immediately downstream of the CAG, SYNG or GFAPG promoter without P40 sequence were tested, and compared to the library constructs where AAV9 capsid was placed downstream of the P40 promoter (FIG. 6A). All genomes were packaged into the AAV9 capsid and used to infect HEK-293T at a MOI of 1e4 VG per cell. RNA was extracted 48 hours post-infection and transgene RNA was quantified by using a Taqman primer/probe mix specific for the spliced globin exon-exon junction. As shown in FIG. 6B, the expression from the CAG promoter was similar between the GFP and the P40-CAP9 constructs (2-fold lower in p40-CAP9, within the error margin of AAV titration). Expression from the synapsin promoter was drastically lower with both constructs and even lower for GFAP-driven mRNA (FIG. 6B). This was expected since HEK-293T cells are not permissive to Synapsin or GFAP promoter expression. Overall, this experiment confirmed that the presence of the P40 sequence did not alter the cell type specificity of synapsin or GFAP promoters.

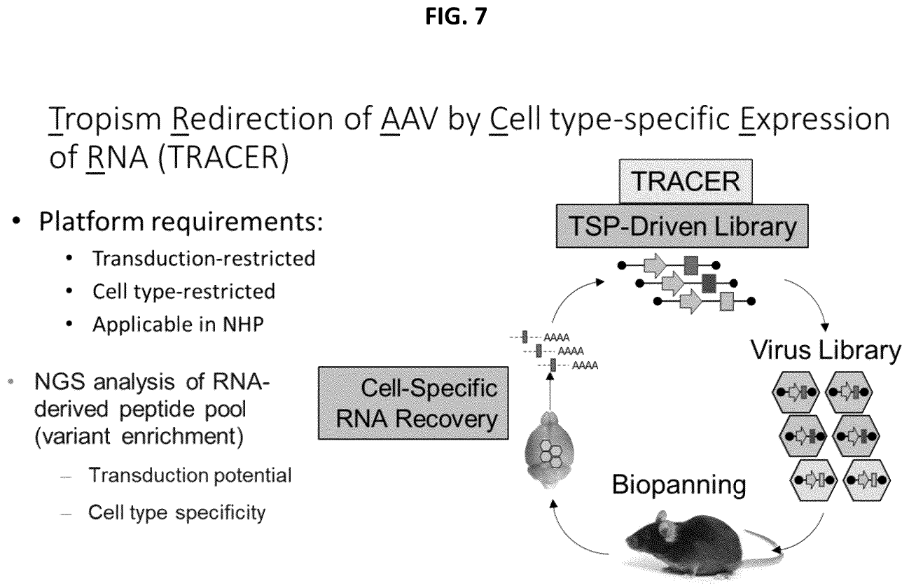

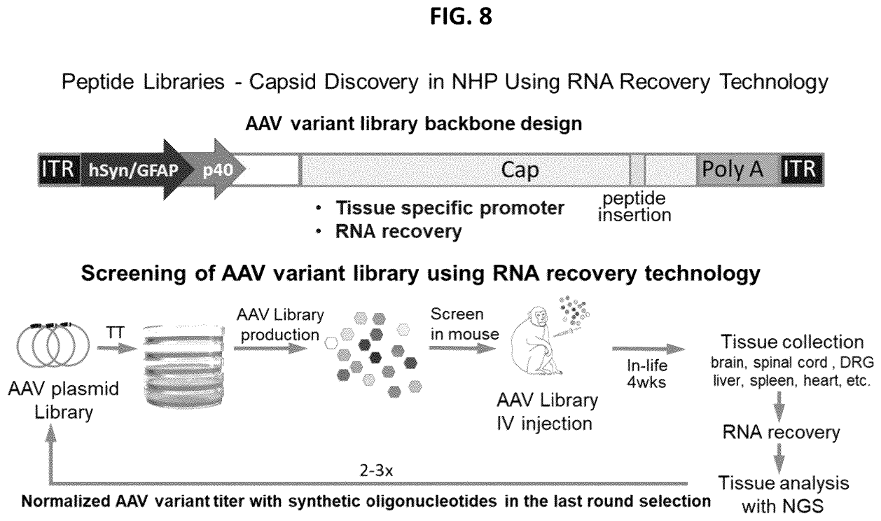

This novel platform was termed TRACER (Tropism Redirection of AAV by Cell type-specific Expression of RNA). The TRACER platform solves the problems of standard methods including transduction and cell-type restrictions. (FIG. 7). Use of the TRACER system is well suited to capsid discovery where targeting peptide libraries are utilized. Screening of such a library may be conducted as outlined in FIG. 8.

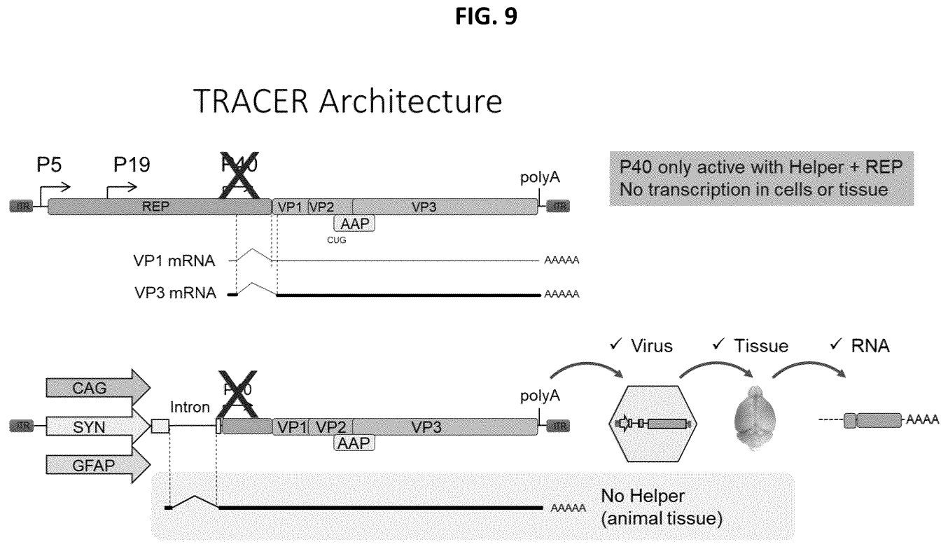

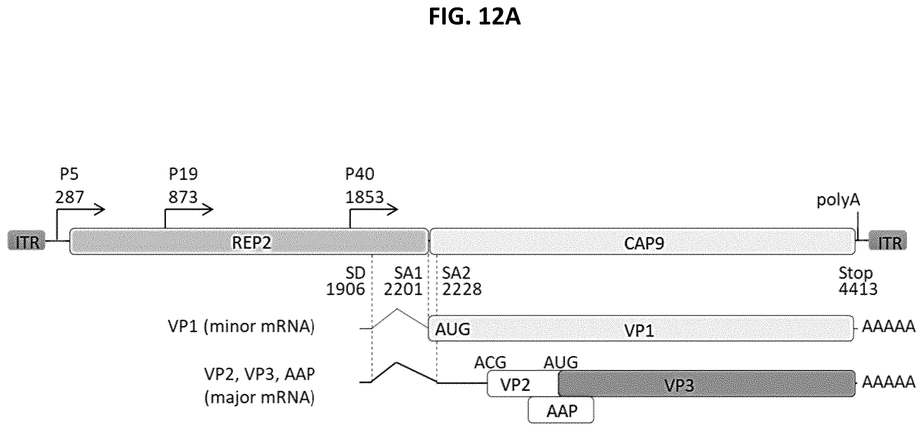

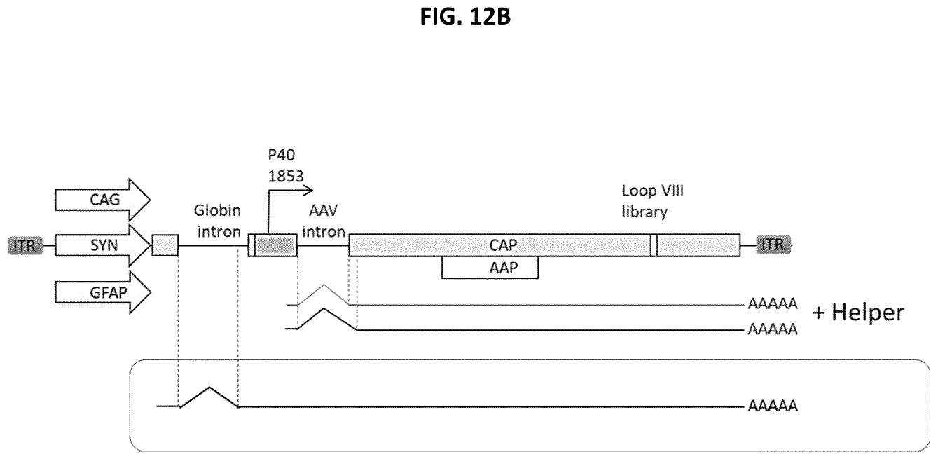

While several variations of the AAV vectors which encode the capsids as payloads are taught herein, one canonical design is shown in FIG. 9B and in FIG. 12A and FIG. 12B.

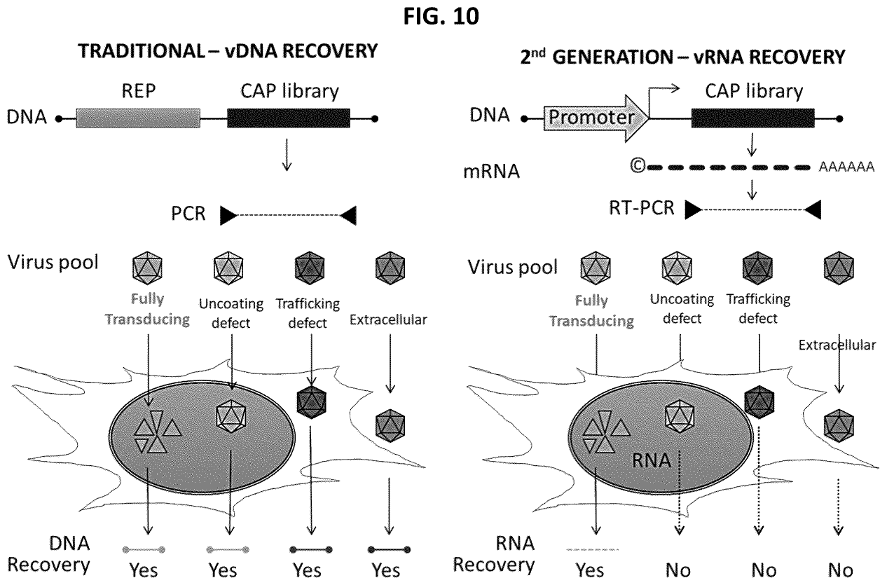

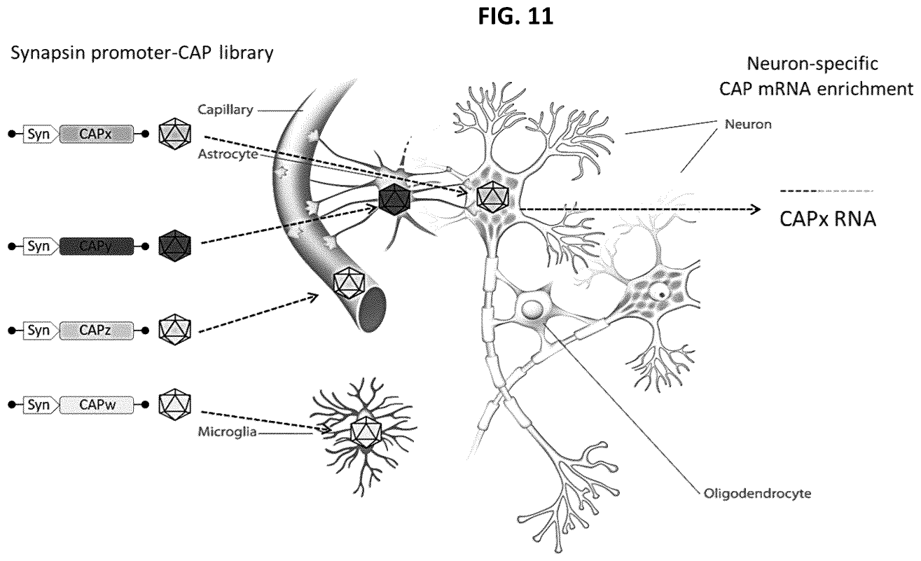

Further advantages of the TRACER platform relate to the nature of the virus pool and the recovery of RNA only from fully transduced cells (FIG. 10). Consequently, capsid discovery can be accelerated in a manner that results in cell and/or tissue specific tropism (FIG. 11).

### Example 4. Generation of Peptide Display Libraries and Cloning-Free Amplification

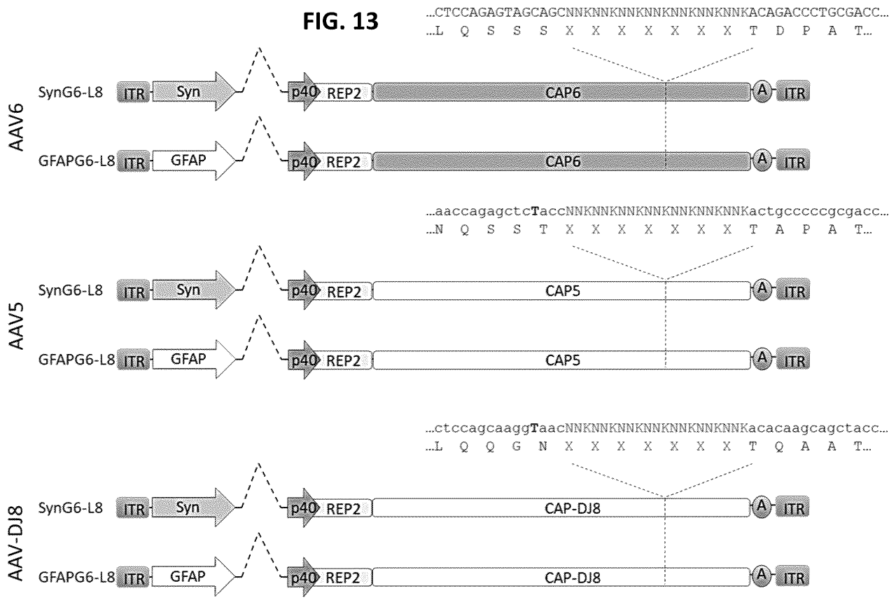

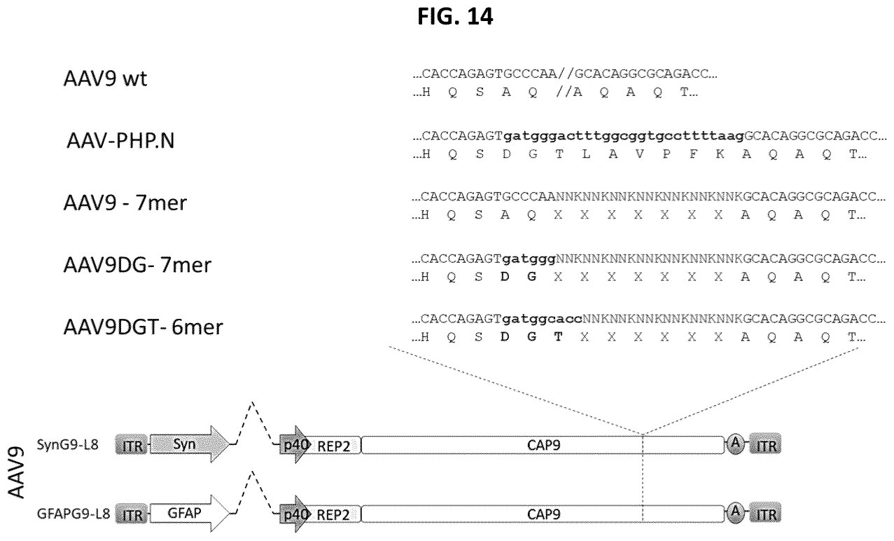

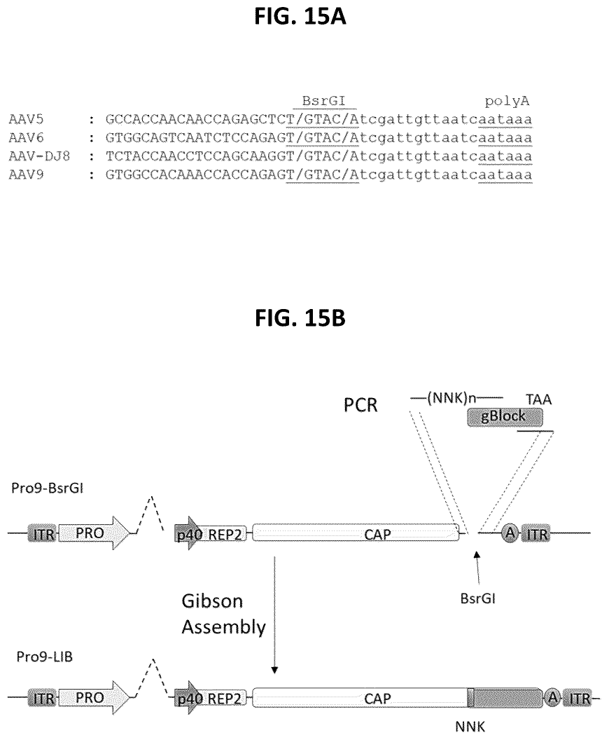

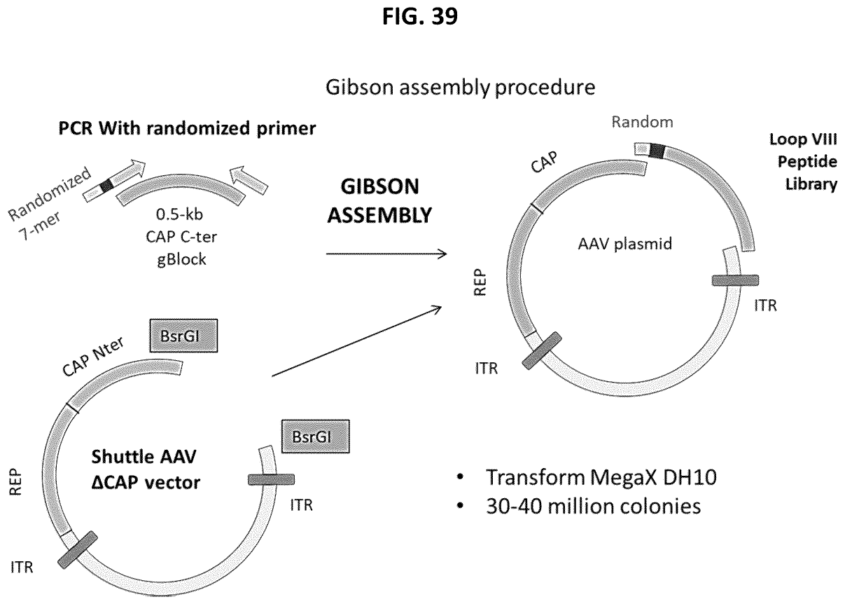

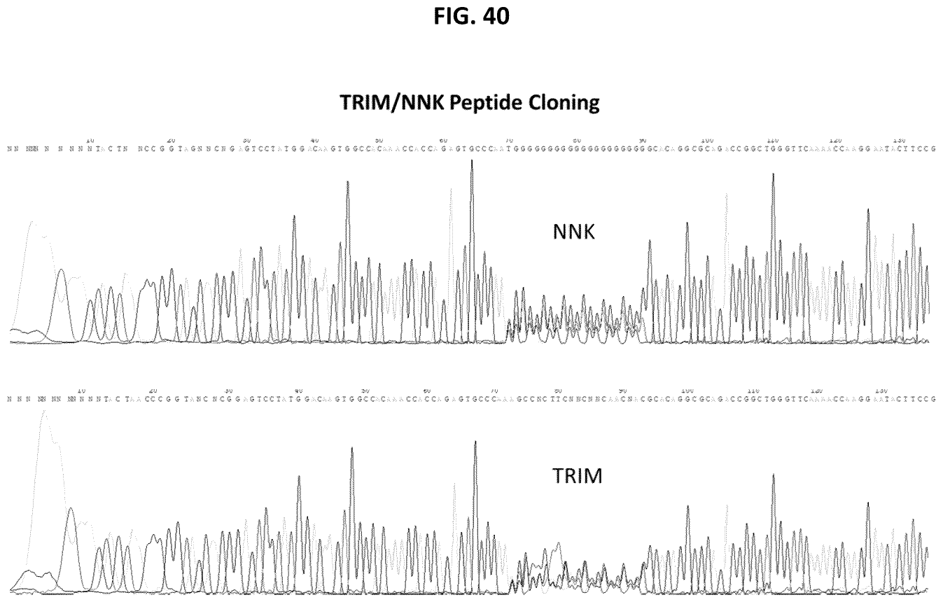

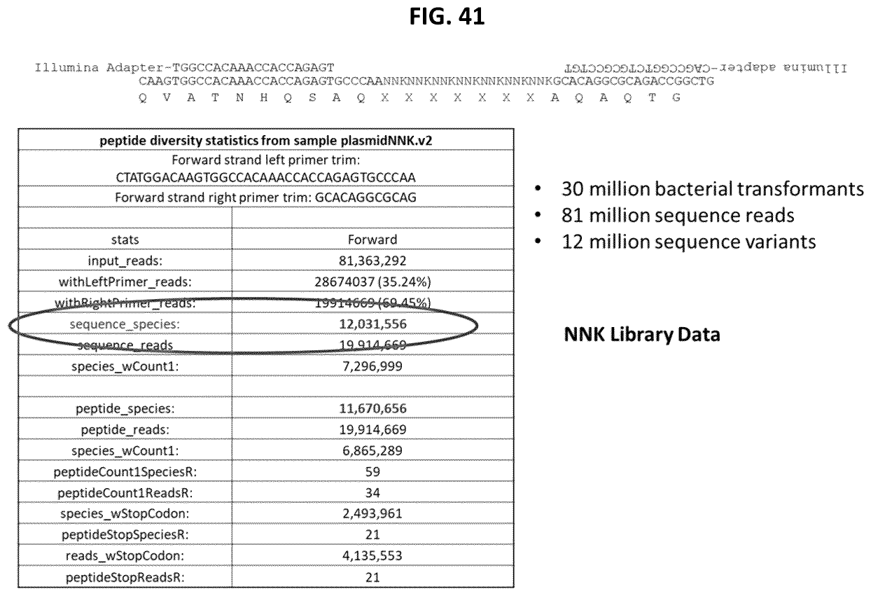

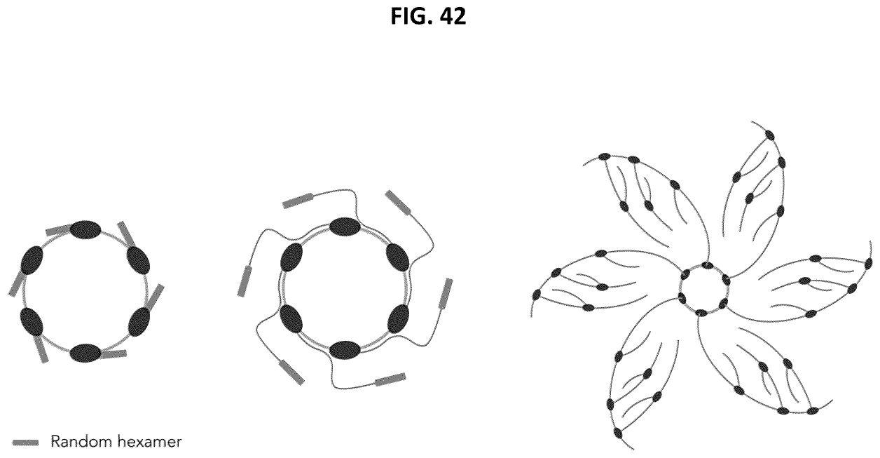

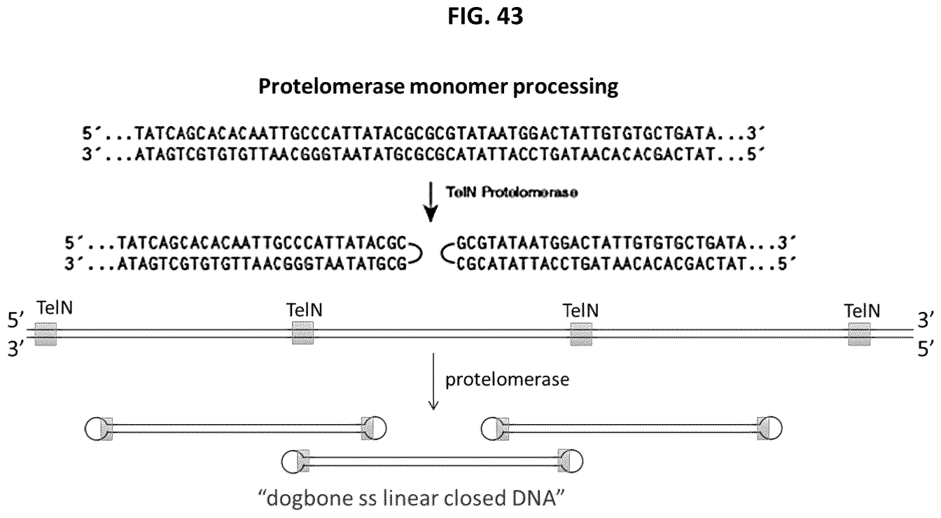

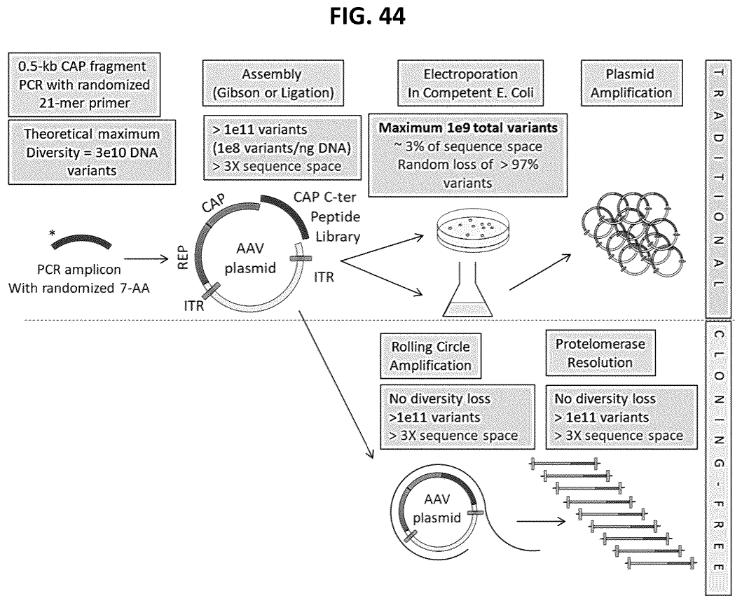

Several peptide display capsid libraries were generated by insertion of seven contiguous randomized amino acids into the surface-exposed hypervariable loop VIII region of AAV5, AAV6, or AAV-DJ8 capsids (FIG. 13 and FIG. 39) as well as AAV9 (FIG. 14). For AAV9 libraries, two extra libraries by modifying residues at positions −2, −1 and +1 of the insertion to match the flanking sequence of the highly neurotrophic PHP.eB vector (Chan et al., 2018). In order to facilitate the insertion of various loops and to prevent contamination by wild-type capsids, defective shuttle vectors were generated in which the C-terminal region of the capsid gene comprised between the loop VIII and the stop codon was deleted and replaced by a unique BsrGI restriction site (FIG. 15A, B). Degenerate primers containing randomized NNK (K=T or G) sequences able to encode all amino acids were synthesized by IDT and used to amplify the missing capsid fragment using gBlock (IDT) double-stranded linear DNA as templates (SEQ. ID NO 14, 15, 16, 17). Linear PCR templates were preferred to plasmids in order to completely prevent the possibility of plasmid carryover in the PCR reaction. Amplicons containing the random library sequence (500 ng) were inserted in the shuttle plasmid linearized by BsrGI (2 ug) using 100 ul of NEBuilder HiFi DNA assembly master mix (NEB) during 30 minutes at 50° C. Unassembled linear templates were eliminated by addition of 5 ul of T5 exonuclease to the reaction and digestion for 30 minutes at 37° C. The entire reaction was purified with DNA Clean and Concentrator-5 and quantified with a nanodrop to estimate the efficiency of assembly. This method routinely allows the recovery of 0.5-1 ug assembled material.

gBlock templates were engineered by introducing silent mutations to remove unique restriction sites, to allow selective elimination of wild-type virus contaminants from the libraries by restriction enzyme treatment. As an example, AAV9 gBlock was engineered to remove BamHI and AfeI sites present in the parental sequence (SEQ. ID NO 17).

### Example 5. Cloning Free Amplification

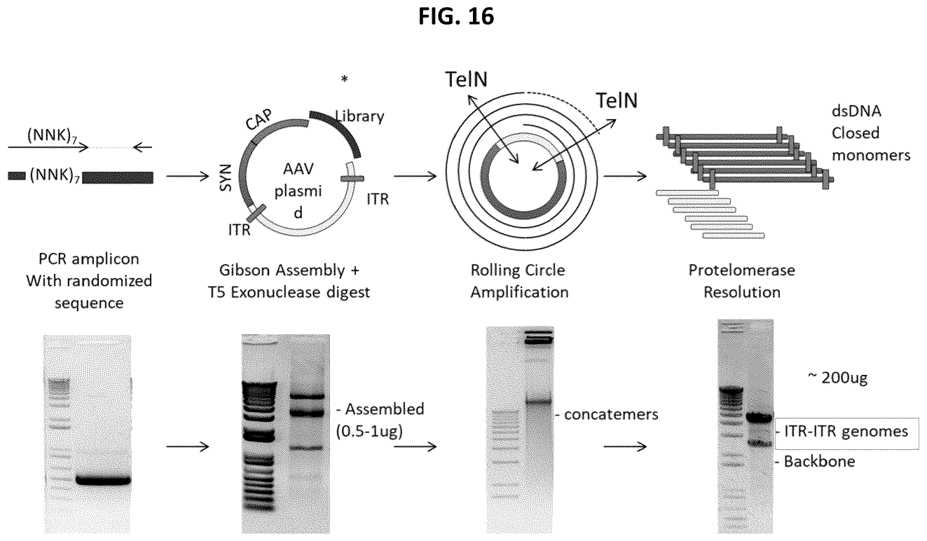

Transformation of assembled library DNA into competent bacteria represents a major bottleneck in library diversity, since even highly competent strains rarely exceed 1e7-1e8 colonies per transformation. By comparison, 100 nanograms of a 6-kilobase plasmid contain 1.5e10 DNA molecules. Therefore, bacterial transformation arbitrarily eliminates more than 99% of DNA species in a given pool. A cloning-free method was therefore created that allows >100-fold amplification of Gibson-assembled DNA while bypassing the bacterial transformation bottleneck (FIG. 16). A protocol based on rolling-circle amplification was optimized, which allows unbiased exponential amplification of circular DNA templates with an extremely low error rate (Hutchinson et al., 2005). One issue with rolling circle amplification is that it produces very large (˜70 kilobases on average) heavily branched concatemers that have to be cleaved into monomers for efficient cell transfection. This process can be accomplished by several methods, for example by using restriction enzymes to generate open-ended linear templates (Hutchinson et al., 2005, Huovinen, 2012), or CRE-Lox recombination to generates self-ligated circular templates (Huovinen et al., 2011). However, open-ended DNA is sensitive to degradation by cytoplasmic exonucleases, and the CRE recombination method showed relatively low efficiency (our unpublished observations). Therefore, an alternative monomer resolution method was chosen based on the use of TelN protelomerase (Rybchin et al., 1999), an enzyme that catalyzes the formation of closed-ended linear “dogbone” DNA monomers that are highly suitable for mammalian cell transfection (Heinrich et al., 2002).

To that end, the protelomerase recognition sequence TATCAGCACACAATTGCCCATTATACGC*GCGTATAATGGACTATTGTGTGCTGATA (SEQ ID NO: 176) was introduced outside both ITRs in all the BsrGI shuttle vectors used for capsid library insertion (the asterisk depicts the position were the two complementary strands get covalently linked to each other), in order to obtain the following plasmids: TelN-Syn9-BsrGI (SEQ ID NO 18), TelN-GFAP9-BsrGI (SEQ ID NO 19), TelN-Syn5-BsrGI (SEQ ID NO 20), TelN-GFAP5-BsrGI (SEQ ID NO 21), TelN-Syn6-BsrGI (SEQ ID NO 22), TeIN-GFAP6-BsrGI (SEQ ID NO 23), TelN-SynDJ8-BsrGI (SEQ ID NO 24), TelN-GFAPDJ8-BsrGI (SEQ ID NO 25). Several methods for rolling circle amplification were tested, and the best results (high yield and low non-specific amplification) were obtained with the TruePrime technology (Expedeon), which relies on primerless amplification (Picher et al., 2016).

Briefly, the entire column-purified assembly reaction was used in a 900-ul TruePrime reaction following the manufacturer's instructions and incubated overnight at 30° C. The following day, the rolling circle reaction product was incubated 10 minutes at 65° C. to inactivate the enzymes and was diluted 5-fold in 1× thermoPol buffer with 50 ul protelomerase (NEB) in a 4.5-ml reaction. After 1 hour at 30° C., the reaction was heat-treated for 10 minutes at 70° C. to inactivate the protelomerase, and a 4.5-ul aliquot was run on an agarose gel. The entire reaction was then purified on multiple (10-12) Qiagen QiaPrep 2.0 columns following manufacturer's instructions. The typical yield obtained with this method was 160-180 ug DNA, which indicates >100-fold amplification of the starting material (typically 0.5-1 ug) and provides enough DNA for transfection of 200 cell culture dishes (FIG. 16).

The composition of all libraries was tested by next-gen sequencing with an Illumina NextSeq sequencing platform to estimate the number of variants and the eventual contamination by wild-type viruses. Amplicons were generated by PCR with Q5 polymerase (NEB) using primers containing Illumina TruSeq adapters and index barcodes. Amplicons were obtained by low-cycle PCR amplification (15 cycles), ran on 3% agarose gels and purified using Zymo gel extraction reagents. Libraries were quantified using a nanodrop, pooled into equimolar mixes and re-quantified with a KAPA library quantification kit following manufacturer's instruction. Libraries were mixed with 20-40% of PhiX control library to increase sequence diversity.

All DNA libraries generated by rolling circle showed a high sequence diversity (typically >1e8 unique variants, beyond the limits of NextSeq sequencing). By comparison, plasmid libraries generated by bacterial transformation rarely exceeded 1-2e7 variants.

### Example 6. Prevention and/or Reduction of Contamination

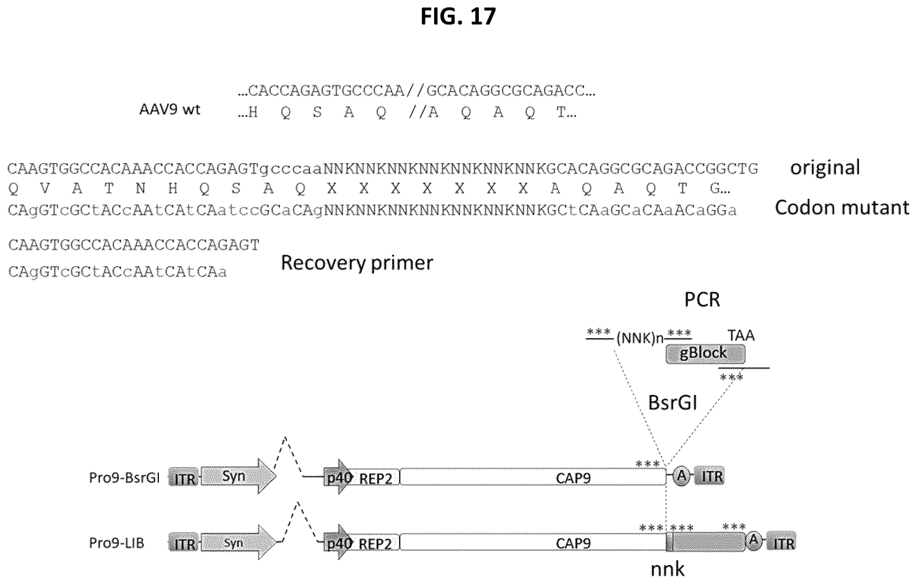

In another embodiment, a primer/vector system aimed at completely preventing contamination of AAV9 libraries by wild-type virus possibly recovered from environmental contamination or from naturally infected primate animal tissues was created. This was achieved by introducing a maximum number of silent mutations in the sequences surrounding the library insertion site, as well as the sequence immediately before the CAP stop codon, used for PCR amplification (FIG. 17). These libraries showed an extremely low number of wild-type AAV9 detection by NGS (<2 AAV9 reads per 5e7 total reads), suggesting that the alteration of codons surrounding the library amplification and cloning sites is a very efficient way to preserve libraries from environmental or experimental contaminations.

Libraries were produced as described previously by calcium phosphate transfection of HEK-293T cells, dual iodixanol gradient fractionation and membrane ultrafiltration using 100,000 Da MWCO Amicon-15 membranes (Millipore), quantified by real-time PCR and an aliquot was used for NGS amplicon generation and NextSeq sequencing. The diversity of viral libraries was significantly lower than that of DNA libraries (typically ˜1-2e7 unique variants) and showed a very strong counter-selection of variants containing stop codons (from 20% in DNA libraries to ˜1% in virus libraries), evincing a very high rate of cis-packaging, as observed in previous studies (Nonnenmacher et al., 2014).

### Example 7. In Vivo Selection of AAV9 Libraries for Mouse Brain Transduction

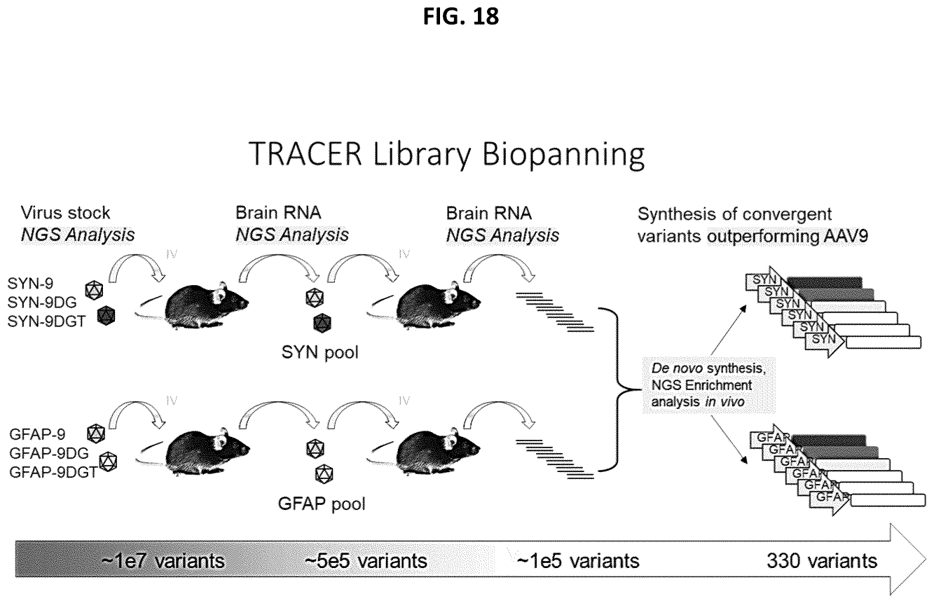

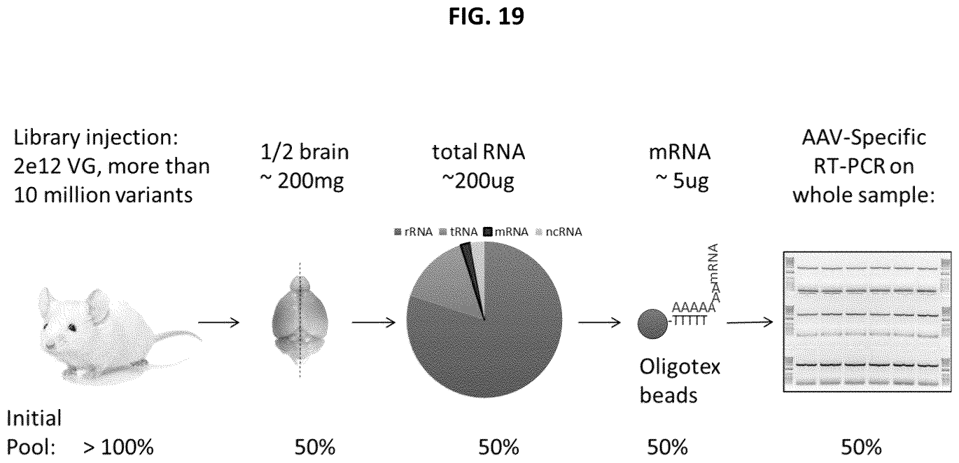

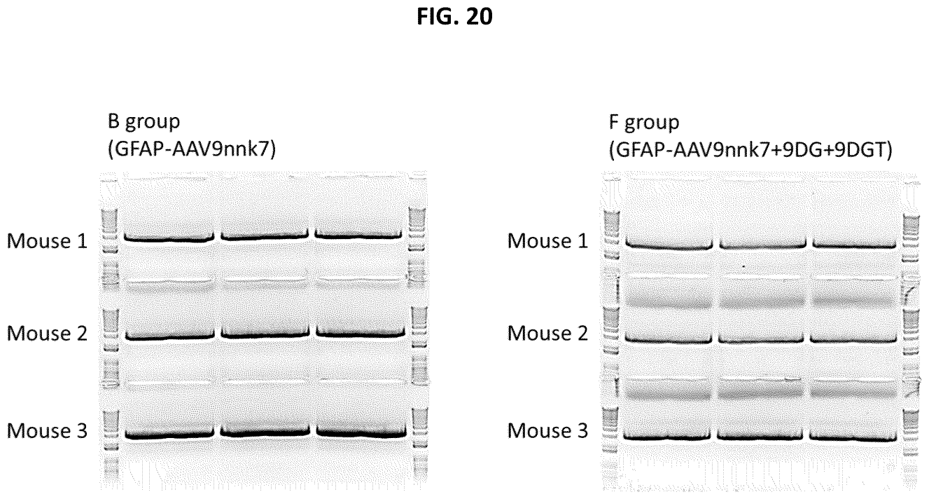

An RNA-driven library selection for increased brain transduction in a murine model was then developed. AAV9 libraries generated as described above were intravenously injected to male C57BL/6 mice at a dose 2e12 VG per mouse. Two groups of mice were injected with a single SYN-driven or GFAP-driven libraries derived from wild-type AAV9 flanking sequences, and two other groups received pooled libraries containing wild-type and PHP.eB-derived flanking sequences (FIG. 18). After one month, RNA was extracted from 200 mg of brain tissue corresponding to a whole hemisphere using RNeasy Universal Plus procedure (Qiagen). In order to minimize the possibility of RNA under sampling, the entire RNA preparation (˜200 ug) was subjected to mRNA enrichment using Oligotex beads (Qiagen) as recommended by the manufacturer. The entire preparation of enriched mRNA (˜5 ug, equivalent to 2% of total RNA) was then reverse transcribed in a 40-ul Superscript IV reaction (Life Technologies) using a library-specific primer with the following sequence: 5′-GAAACGAATTAAACGGTTTATTGATTAACAATCGATTA-3′ (SEQ ID NO: 415) (CAP stop codon is underlined) (FIG. 19). The entire pool of cDNA was then amplified 30 cycles with 55° C. annealing temperature and 2 minutes elongation in a 500-ul PCR reaction assembled with Q5 master mix, GloSpliceF6 forward primer and a CAP9-specific reverse primer: 5′-CGGTTTATTGATTAACAATCGATTACAGATTACGAGTCAGGTATC-3′ (SEQ ID NO: 416) (CAP stop codon is underlined). This method allowed recovery of abundant amplicons from all brain samples (FIG. 20).

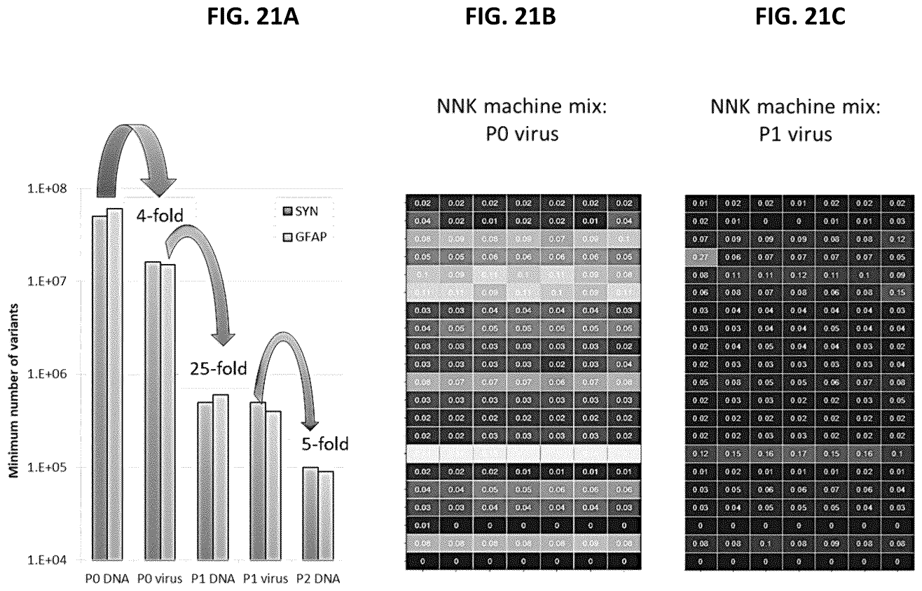

Full-length capsid amplicons were then used as templates for NGS library generation, as well as cloning into a P1 DNA library for the next round of biopanning, using the exact same assembly and cloning-free procedure. NGS analysis performed on PCR amplicons indicated that the library diversity dropped ˜25-fold (from 1e7 to 4e5) after the first round of biopanning for both Syn-driven and GFAP-driven libraries (FIG. 21). The number of 1^{st} pass variants (P1) recovered was too high to show any significant sequence convergence at this point, and there was very little overlap between the composition of pools recovered from individual animals. Therefore, a second round of selection was performed. After the second biopanning (P2), the total number of unique variants further dropped by 4-5-fold, down to <1e5 peptides. Importantly, some libraries recovered after the first round of biopanning showed significant counts of wild-type AAV9 and AAV-PHP.eB sequences, presumably from environmental contamination. These later became useful benchmarks in the second round of enrichment.

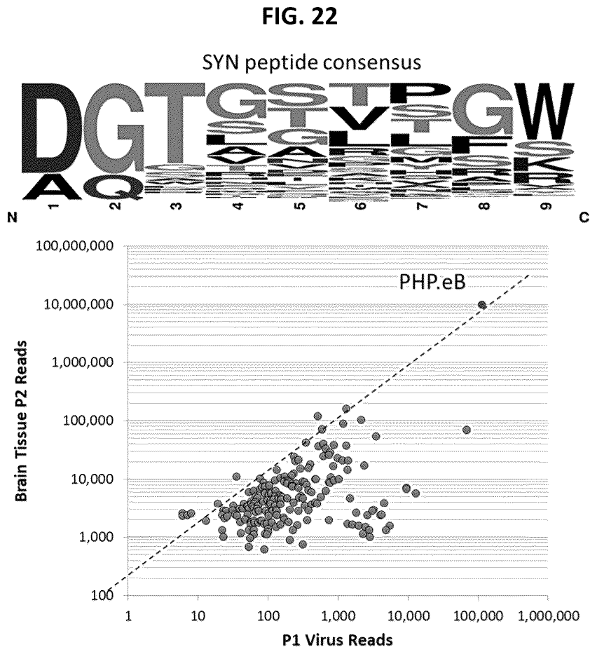

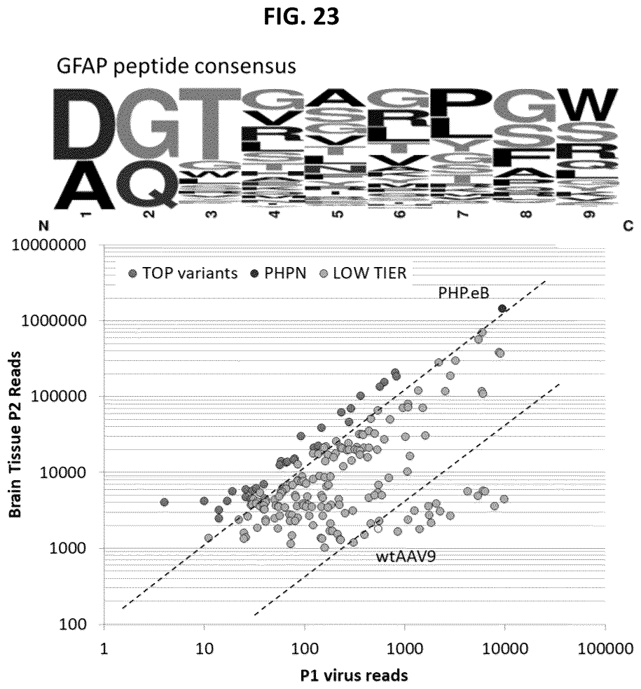

Following RNA recovery and PCR amplification, a systematic enrichment analysis by NGS was performed by calculating the ratio of P2/P1 reads and comparing it to AAV9 or PHP.eB P2/P1 ratio. As shown in FIG. 22, Table 4, FIG. 23 and Table 5, several capsids showed a higher enrichment ratio than the benchmark PHP.eB in both Syn-driven and GFAP-driven libraries, and sequence convergence was obvious, as represented by consensus sequence generation.

TABLE 4
Capsid analysis results

| Rank | | | SEQ | | | Brain/ |
|---|---|---|---|---|---|---|
| (enrichment | Ranking | | ID | Average | p1 | virus |
| factor) | (count) | Peptide | NO | of brain | AEvirus_S11 | stock |
| 1 | 136 | DGTLAVHFK | 417 | 2546.3 | 6 | 254.6 |
| 2 | 153 | DGTFAVPFK | 418 | 2321.7 | 6 | 232.2 |
| 3 | 155 | EGTLAVPFK | 419 | 2351.0 | 7 | 201.5 |
| 4 | 147 | DGTMAVPFK | 420 | 2547.0 | 8 | 191.0 |
| 5 | 32 | DGTGGTKGW | 107 | 11116.0 | 35 | 190.6 |
| 6 | 3 | AQWPTSYDA | 62 | 119359.7 | 512 | 139.9 |
| 7 | 99 | DGTLAVTFK | 421 | 3779.7 | 19 | 119.4 |
| 8 | 176 | DGTLAVPIK | 422 | 1882.0 | 13 | 86.9 |
| 9 | 36 | AQTTEKPWL | 83 | 10192.0 | 76 | 80.5 |
| 10 | 165 | DGTAIHLSS | 67 | 2885.0 | 23 | 75.3 |
| 11 | 13 | DGTLSQPFR | 65 | 42145.7 | 344 | 73.5 |
| 12 | 2 | DGTLAAPFK | 120 | 157129.3 | 1,300 | 72.5 |
| 13 | 8 | AQPEGSARW | 60 | 70884.0 | 594 | 71.6 |
| 14 | 48 | AQWPTAYDA | 256 | 5934.0 | 53 | 67.2 |
| 15 | 198 | DGTLQQPFR | 89 | 2793.3 | 25 | 67.0 |
| 16 | 104 | DGTLAVNFK | 346 | 3511.0 | 32 | 65.8 |
| 17 | 31 | DGTGNLSGW | 302 | 14521.3 | 133 | 65.5 |
| 18 | 158 | DGTLEVTFK | 423 | 2337.7 | 22 | 63.8 |
| 19 | 51 | DGTMDKPFR | 70 | 23962.3 | 234 | 61.4 |
| 20 | 80 | DGTGQVTGW | 68 | 6242.7 | 62 | 60.4 |
| 21 | 42 | AQFPTNYDS | 66 | 8640.0 | 86 | 60.3 |
| 22 | 127 | ERTLAVPFK | 424 | 2873.3 | 31 | 55.6 |
| 23 | 1 | DGTLAVPFK | 71 | 9885065.7 | 110,785 | 53.5 |
| 24 | 61 | DGTGTTMGW | 324 | 6753.0 | 76 | 53.3 |
| 25 | 69 | DGSQSTTGW | 136 | 7227.7 | 82 | 52.9 |
| 26 | 186 | DGTVSNPFR | 403 | 2074.3 | 24 | 51.9 |
| 27 | 160 | DGTLEVHFK | 348 | 2245.0 | 26 | 51.8 |
| 28 | 29 | DGTISQPFK | 105 | 20505.7 | 243 | 50.6 |
| 29 | 102 | AQGSWNPPA | 80 | 3746.0 | 45 | 49.9 |
| 30 | 59 | DGTHSTTGW | 145 | 7499.0 | 91 | 49.4 |
| 31 | 23 | DGTGSTTGW | 134 | 21582.0 | 272 | 47.6 |
| 32 | 142 | DGTGTTTGW | 130 | 3077.3 | 39 | 47.3 |
| 33 | 74 | DGTVTTTGW | 405 | 5088.7 | 66 | 46.3 |
| 34 | 35 | DGTTYVPPR | 75 | 9614.7 | 126 | 45.8 |
| 35 | 40 | DGTMDRPFK | 102 | 7868.3 | 104 | 45.4 |
| 36 | 4 | DGTGTTLGW | 323 | 88397.3 | 1,169 | 45.4 |
| 37 | 156 | DGTALMLSS | 280 | 2444.0 | 34 | 43.1 |
| 38 | 116 | DGTNTTHGW | 113 | 3065.0 | 43 | 42.8 |
| 39 | 98 | SGSLAVPFK | 425 | 4107.3 | 58 | 42.5 |
| 40 | 38 | DGTATTTGW | 285 | 10529.7 | 150 | 42.1 |
| 41 | 11 | DGTSYVPPR | 78 | 36293.3 | 526 | 41.4 |
| 42 | 89 | DGTGNTHGW | 72 | 3399.3 | 50 | 40.8 |
| 43 | 129 | DGTASVTGW | 283 | 4824.3 | 71 | 40.8 |
| 44 | 12 | AQWELSNGY | 246 | 40837.0 | 611 | 40.1 |
| 45 | 115 | DGTGNTSGW | 137 | 3405.0 | 51 | 40.1 |
| 46 | 67 | DGKGSTQGW | 272 | 5818.0 | 88 | 39.7 |
| 47 | 137 | DGTVIMLSS | 397 | 3781.0 | 58 | 39.1 |
| 48 | 119 | DGTGGVMGW | 297 | 2302.3 | 36 | 38.4 |
| 49 | 58 | DGGGTTTGW | 270 | 11174.3 | 175 | 38.3 |
| 50 | 71 | DGTSIHLSS | 378 | 5703.7 | 90 | 38.0 |

TABLE 5
Capsid analysis results

| Rank | | | SEQ | | | Brain/ |
|---|---|---|---|---|---|---|
| (enrichment | Ranking | | ID | Average | p1 | virus |
| factor) | (count) | Peptide | NO | of brain | AEvirus_S11 | stock |
| 1 | 106 | DGTGGTKGW | 107 | 3620.7 | 0 | NA |
| 2 | 264 | GGTRNTAPM | 426 | 831.0 | 0 | NA |
| 3 | 295 | AQGRMTDSQ | 199 | 716.0 | 0 | NA |
| 4 | 677 | DGNSYVPPR | 427 | 474.3 | 0 | NA |
| 5 | 700 | AQAGVSGQR | 428 | 456.0 | 0 | NA |
| 6 | 731 | AQAGNSNAV | 429 | 844.0 | 0 | NA |
| 7 | 181 | DGTGGLTGW | 294 | 4044.3 | 4 | 606.7 |
| 8 | 558 | AQWVYGQTV | 430 | 977.7 | 1 | 586.6 |
| 9 | 123 | DGTSFSPPK | 431 | 4227.3 | 10 | 253.6 |
| 10 | 35 | DGTIERPFR | 87 | 29872.0 | 92 | 194.8 |
| 11 | 105 | DGTTLVPPR | 116 | 5597.3 | 19 | 176.8 |
| 12 | 18 | DGTADKPFR | 63 | 103305.3 | 363 | 170.8 |
| 13 | 22 | DGTASYYDS | 61 | 61841.3 | 233 | 159.2 |
| 14 | 26 | AQTTDRPFL | 85 | 38893.7 | 147 | 158.7 |
| 15 | 8 | DGTQFSPPR | 108 | 206660.7 | 801 | 154.8 |
| 16 | 169 | DGTTTYGAR | 77 | 4237.3 | 17 | 149.6 |
| 17 | 11 | AQFVVGQQY | 95 | 152965.0 | 625 | 146.8 |
| 18 | 61 | DGTSYVPPR | 78 | 13968.0 | 58 | 144.5 |
| 19 | 16 | DGTAERPFR | 140 | 134132.7 | 565 | 142.4 |
| 20 | 21 | AQGENPGRW | 96 | 68919.7 | 292 | 141.6 |
| 21 | 157 | DGTSFTPPR | 88 | 3210.0 | 14 | 137.6 |
| 22 | 73 | AQTLARPFV | 98 | 5947.7 | 26 | 137.3 |
| 23 | 9 | DGTTWTPPR | 139 | 184936.7 | 825 | 134.5 |
| 24 | 721 | DGTATTMGW | 284 | 5562.3 | 25 | 133.5 |
| 25 | 129 | AQGTWNPPA | 82 | 12379.3 | 57 | 130.3 |
| 26 | 215 | DGTRLMLSS | 368 | 2505.0 | 12 | 125.3 |
| 27 | 60 | AQPLAVYGA | 217 | 13419.3 | 66 | 122.0 |
| 28 | 909 | AQGLDLGRW | 432 | 405.0 | 2 | 121.5 |
| 29 | 53 | DGTSFTPPK | 81 | 13673.3 | 68 | 120.6 |
| 30 | 412 | AQVMSGVGQ | 433 | 583.0 | 3 | 116.6 |
| 31 | 390 | AQKSVGSVY | 205 | 4415.7 | 23 | 115.2 |
| 32 | 70 | AQTREYLLG | 93 | 5752.7 | 30 | 115.1 |
| 33 | 43 | DGTNGLKGW | 76 | 15068.7 | 79 | 114.4 |
| 34 | 93 | AQYLAGYTV | 262 | 6223.3 | 33 | 113.2 |
| 35 | 54 | AQTGFAPPR | 161 | 14611.3 | 78 | 112.4 |
| 36 | 115 | DGTLNNPFR | 109 | 4719.7 | 26 | 108.9 |
| 37 | 968 | DGNGGLKGW | 167 | 3199.0 | 18 | 106.6 |
| 38 | 120 | AQSVAKPFL | 231 | 6929.7 | 39 | 106.6 |
| 39 | 544 | DGTHGLRGW | 434 | 528.0 | 3 | 105.6 |
| 40 | 159 | AQSVVRPFL | 233 | 2457.3 | 14 | 105.3 |
| 41 | 65 | DGTRNMYEG | 135 | 21086.3 | 124 | 102.0 |
| 42 | 556 | AQRWAADSS | 435 | 500.7 | 3 | 100.1 |
| 43 | 30 | AQGPTRPFL | 125 | 46225.3 | 279 | 99.4 |
| 44 | 64 | DGTVPYLSS | 401 | 22384.3 | 137 | 98.0 |
| 45 | 870 | AQTGASGAT | 436 | 473.7 | 3 | 94.7 |
| 46 | 341 | AQLVAGYSQ | 437 | 1240.0 | 8 | 93.0 |
| 47 | 375 | AQSGGVGQV | 228 | 768.3 | 5 | 92.2 |
| 48 | 145 | AQSLARLFP | 438 | 4435.3 | 29 | 91.8 |
| 49 | 1 | DGTLAVPFK | 71 | 1445517.0 | 9453 | 91.7 |
| 50 | 124 | DGTGNVTGW | 69 | 5424.3 | 36 | 90.4 |

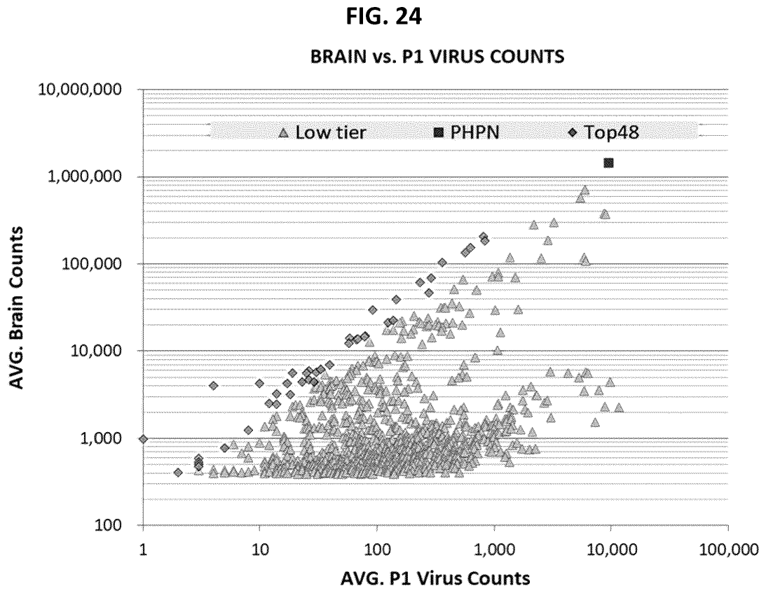

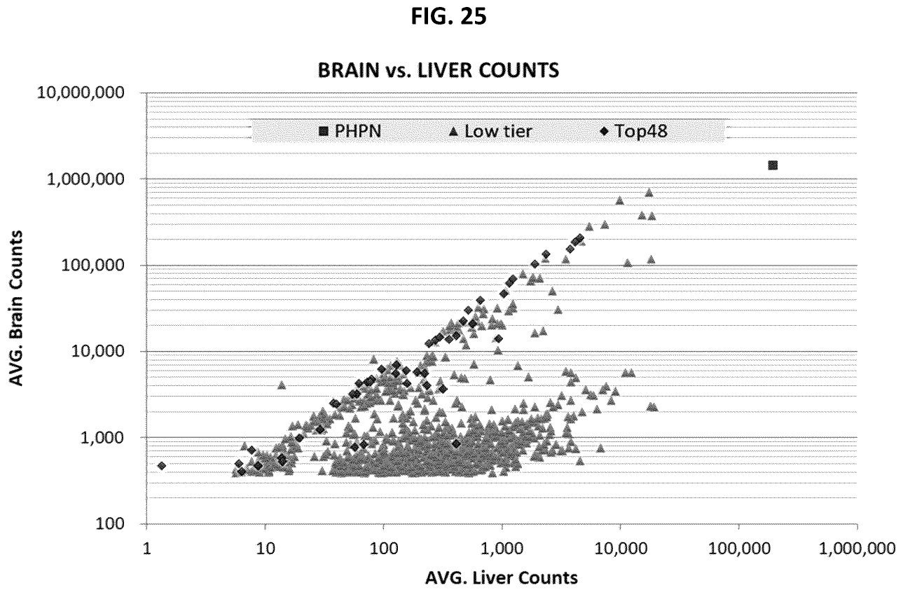

Importantly, there was also a strong sequence convergence between different animals, suggesting an efficient selection after only two passages. FIG. 24 and FIG. 25 provide an estimation of brain/liver specificity in GFAP-AAV9 peptide library candidates.

### Example 8. Multiplexing Selections

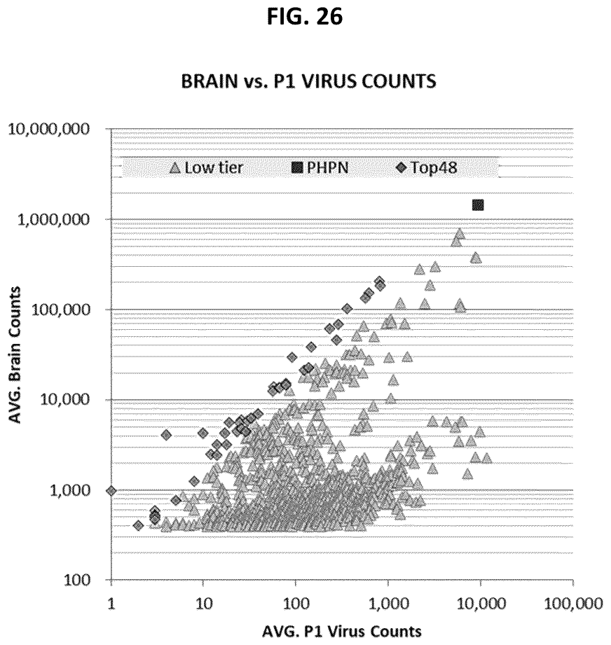

For the final multiplex in vivo screen by individual variant pooling in equimolar library, a subpopulation of variants with promising properties (such as, but not limited to, enrichment factor, liver detargeting, high counts in more than one mouse, etc.) may be selected as shown in FIG. 26 and then an equimolar pool of primers encoding all the 7-mers (microchip solid-phase synthesis, up to 3,800 primers per chip) can be synthesized. The limited diversity library may be produced including internal controls such as, but not limited to, PHP.N, PHP.B, wild-type AAV9 (wtAAV9) and/or any other serotype including those taught herein. The mice are injected and then the RNA enrichment is compared to internal controls in a similar manner to a barcoding study, which is known in the art and described herein.

### Example 9. Codon Optimization

Codon variants may be used to improve data strength when using synthesized libraries. A listing of NNK codons, NNM codons and the most favorable NNM codons in mammals for various amino acids is provided in Table 6. In Table 6, * means that no NNM codon was available and ** means “avoid homopolymeric stretches if possible.”

TABLE 6
Codon Variants

| | | | Most favorable |
|---|---|---|---|
| Amino | | | NNM codon |
| acid | NNK codon | NNM codons | in mammals |
| F | TTT | TTC | TTC |
| L | TTG, CTT, CTG | TTA, CTC, CTA | CTC |
| S | TCT, TCG, AGT | TCC, TCA, AGC | AGC |
| Y | TAT | TAC | TAC |
| C | TGT | TGC | TGC |
| W | TGG | | TGG* |
| P | CCT, CCG | CCC, CCA | CCA** |
| H | CAT | CAC | CAC |
| Q | CAG | CAA | CAA |
| R | CGT, CGG, AGG | CGC, CGA, AGA | AGA |
| I | ATT | ATC, ATA | ATC |
| M | ATG | | ATT* |
| T | ACT, ACG | ACC, ACA | ACC |
| N | AAT | AAC | AAC |
| K | AAG | AAA | AAA |
| V | GTT, GTG | GTC, GTA | GTC |
| A | GCT, GCG | GCC, GCA | GCC |
| D | GAT | GAC | GAC |
| E | GAG | GAA | GAA |
| G | GGT, GGG | GGC, GGA | GGC |
| stop | TAG | TAC, TAA | n/a |

*no NNM codon available
**avoid homopolymeric stretches if possible

In order to have a balanced library it is recommended to establish a list of potential candidates. Then, using Table 6, a pooled primer library containing every peptide variant with encoded by NNK codons (original from library) and non-NNK codons (maximum variation). If similar behavior is seen between the two variants of the same peptide, this would strengthen the analysis of that peptide. Additionally, it is recommended to choose the most favorable NNM codons (M-A or C).

### Example 10. Library Generation

The top-ranking 330 peptide variants from SYN-driven and GFAP-driven libraries that showed enhanced performance relative to the parental AAV9 were selected. A de novo library by pooled primer synthesis of all 330 peptide sequences plus AAV9, AAV-PHP.B and AAV-PHP.eB controls was generated (Table 7). In order to exclude potential artifacts due to the DNA sequence and to increase the robustness of the assay, each peptide variant was encoded by two different DNA sequences, one where all amino acids were encoded by NNK codons (identical to the original library) and another one where NNM codons were used whenever possible (M=C or A, Table 6).

TABLE 7
Peptide variants selected after 2 rounds
of RNA-driven mouse brain biopanning

| | SEQ | Nucleotide | SEQ | Nucleotide | SEQ |
|---|---|---|---|---|---|
| Peptide | ID | sequence | ID | sequence | ID |
| Sequence | NO: | (NNK codons) | NO: | (NNM codons) | NO: |
| AQ (AAV9) | | CAGAGTGCTCAG | 439 | CAGAGTGCCCAA | 772 |
| | | GCACAG | | GCACAG | |
| AQAGAGSER | 194 | CAGAGTGCCCAA | 440 | CAGAGTGCACAA | 773 |
| | | GCGGGTGCGGGG | | GCAGGAGCAGGA | |
| | | TCGGAGCGGGCA | | AGCGAAAGAGCA | |
| | | CAG | | CAG | |
| AQDQNPGRW | 195 | CAGAGTGCCCAA | 441 | CAGAGTGCACAA | 774 |
| | | GATCAGAATCCG | | GACCAAAACCCA | |
| | | GGGCGTTGGGCA | | GGAAGATGGGCA | |
| | | CAG | | CAG | |
| AQELTRPFL | 144 | CAGAGTGCCCAA | 442 | CAGAGTGCACAA | 775 |
| | | GAGTTGACGCGT | | GAACTCACAAGA | |
| | | CCGTTTTTGGCAC | | CCATTCCTCGCAC | |
| | | AG | | AG | |
| AQEVPGYRW | 196 | CAGAGTGCCCAA | 443 | CAGAGTGCACAA | 776 |
| | | GAGGTGCCTGGG | | GAAGTCCCAGGA | |
| | | TATAGGTGGGCA | | TACAGATGGGCA | |
| | | CAG | | CAG | |
| AQFPTNYDS | 66 | CAGAGTGCCCAA | 444 | CAGAGTGCACAA | 777 |
| | | TTTCCTACGAATT | | TTCCCAACAAACT | |
| | | ATGATTCTGCACA | | ACGACAGCGCAC | |
| | | G | | AG | |
| AQFVVGQQY | 95 | CAGAGTGCCCAA | 445 | CAGAGTGCACAA | 778 |
| | | TTTGTGGTTGGTC | | TTCGTCGTCGGAC | |
| | | AGCAGTATGCAC | | AACAATACGCAC | |
| | | AG | | AG | |
| AQGASPGRW | 149 | CAGAGTGCCCAA | 446 | CAGAGTGCACAA | 779 |
| | | GGGGCTAGTCCG | | GGAGCAAGCCCA | |
| | | GGGCGGTGGGCA | | GGAAGATGGGCA | |
| | | CAG | | CAG | |
| AQGENPGRW | 96 | CAGAGTGCCCAA | 447 | CAGAGTGCACAA | 780 |
| | | GGGGAGAATCCG | | GGAGAAAACCCA | |
| | | GGTAGGTGGGCA | | GGAAGATGGGCA | |
| | | CAG | | CAG | |
| AQGGNPGRW | 91 | CAGAGTGCCCAA | 448 | CAGAGTGCACAA | 781 |
| | | GGGGGGAATCCG | | GGAGGAAACCCA | |
| | | GGTCGGTGGGCA | | GGAAGATGGGCA | |
| | | CAG | | CAG | |
| AQGGSTGSN | 197 | CAGAGTGCCCAA | 449 | CAGAGTGCACAA | 782 |
| | | GGTGGTTCTACG | | GGAGGAAGCACA | |
| | | GGGTCGAATGCA | | GGAAGCAACGCA | |
| | | CAG | | CAG | |
| AQGPTRPFL | 125 | CAGAGTGCCCAA | 450 | CAGAGTGCACAA | 783 |
| | | GGGCCGACTAGG | | GGACCAACAAGA | |
| | | CCGTTTTTGGCAC | | CCATTCCTCGCAC | |
| | | AG | | AG | |
| AQGRDGWAA | 198 | CAGAGTGCCCAA | 451 | CAGAGTGCACAA | 784 |
| | | GGTCGGGATGGT | | GGAAGAGACGGA | |
| | | TGGGCGGCGGCA | | TGGGCAGCAGCA | |
| | | CAG | | CAG | |
| AQGRMTDSQ | 199 | CAGAGTGCCCAA | 452 | CAGAGTGCACAA | 785 |
| | | GGTCGTATGACT | | GGAAGAATGACA | |
| | | GATTCGCAGGCA | | GACAGCCAAGCA | |
| | | CAG | | CAG | |
| AQGSDVGRW | 128 | CAGAGTGCCCAA | 453 | CAGAGTGCACAA | 786 |
| | | GGTAGTGATGTG | | GGAAGCGACGTC | |
| | | GGGCGGTGGGCA | | GGAAGATGGGCA | |
| | | CAG | | CAG | |
| AQGSNPGRW | 103 | CAGAGTGCCCAA | 454 | CAGAGTGCACAA | 787 |
| | | GGTAGTAATCCG | | GGAAGCAACCCA | |
| | | GGGAGGTGGGCA | | GGAAGATGGGCA | |
| | | CAG | | CAG | |
| AQGSNSPQV | 200 | CAGAGTGCCCAA | 455 | CAGAGTGCACAA | 788 |
| | | GGGTCTAATTCGC | | GGAAGCAACAGC | |
| | | CTCAGGTGGCAC | | CCACAAGTCGCA | |
| | | AG | | CAG | |
| AQGSWNPPA | 80 | CAGAGTGCCCAA | 456 | CAGAGTGCACAA | 789 |
| | | GGTTCGTGGAAT | | GGAAGCTGGAAC | |
| | | CCGCCGGCGGCA | | CCACCAGCAGCA | |
| | | CAG | | CAG | |
| AQGTWNPPA | 82 | CAGAGTGCCCAA | 457 | CAGAGTGCACAA | 790 |
| | | GGTACTTGGAAT | | GGAACATGGAAC | |
| | | CCGCCGGCTGCA | | CCACCAGCAGCA | |
| | | CAG | | CAG | |
| AQGVFIPPK | 201 | CAGAGTGCCCAA | 458 | CAGAGTGCACAA | 791 |
| | | GGTGTTTTTATTC | | GGAGTCTTCATCC | |
| | | CGCCGAAGGCAC | | CACCAAAAGCAC | |
| | | AG | | AG | |
| AQHVNASQS | 202 | CAGAGTGCCCAA | 459 | CAGAGTGCACAA | 792 |
| | | CATGTGAATGCTT | | CACGTCAACGCA | |
| | | CTCAGTCTGCACA | | AGCCAAAGCGCA | |
| | | G | | CAG | |
| AQIKAGWAQ | 203 | CAGAGTGCCCAA | 460 | CAGAGTGCACAA | 793 |
| | | ATTAAGGCGGGG | | ATCAAAGCAGGA | |
| | | TGGGCGCAGGCA | | TGGGCACAAGCA | |
| | | CAG | | CAG | |
| AQIMSGYAQ | 204 | CAGAGTGCCCAA | 461 | CAGAGTGCACAA | 794 |
| | | ATTATGAGTGGG | | ATCATGAGCGGA | |
| | | TATGCTCAGGCA | | TACGCACAAGCA | |
| | | CAG | | CAG | |
| AQKSVGSVY | 205 | CAGAGTGCCCAA | 462 | CAGAGTGCACAA | 795 |
| | | AAGAGTGTGGGT | | AAAAGCGTCGGA | |
| | | AGTGTTTATGCAC | | AGCGTCTACGCA | |
| | | AG | | CAG | |
| AQLEHGFAQ | 206 | CAGAGTGCCCAA | 463 | CAGAGTGCACAA | 796 |
| | | CTTGAGCATGGG | | CTCGAACACGGA | |
| | | TTTGCTCAGGCAC | | TTCGCACAAGCA | |
| | | AG | | CAG | |
| AQLGGVLSA | 207 | CAGAGTGCCCAA | 464 | CAGAGTGCACAA | 797 |
| | | CTGGGTGGGGTG | | CTCGGAGGAGTC | |
| | | TTGAGTGCTGCAC | | CTCAGCGCAGCA | |
| | | AG | | CAG | |
| AQLGLSQGR | 208 | CAGAGTGCCCAA | 465 | CAGAGTGCACAA | 798 |
| | | CTGGGGCTTTCGC | | CTCGGACTCAGC | |
| | | AGGGGCGGGCAC | | CAAGGAAGAGCA | |
| | | AG | | CAG | |
| AQLGYGFAQ | 209 | CAGAGTGCCCAA | 466 | CAGAGTGCACAA | 799 |
| | | TTGGGGTATGGG | | CTCGGATACGGA | |
| | | TTTGCTCAGGCAC | | TTCGCACAAGCA | |
| | | AG | | CAG | |
| AQLKYGLAQ | 115 | CAGAGTGCCCAA | 467 | CAGAGTGCACAA | 800 |
| | | TTGAAGTATGGTC | | CTCAAATACGGA | |
| | | TTGCGCAGGCAC | | CTCGCACAAGCA | |
| | | AG | | CAG | |
| AQLRIGFAQ | 210 | CAGAGTGCCCAA | 468 | CAGAGTGCACAA | 801 |
| | | CTTCGGATTGGTT | | CTCAGAATCGGA | |
| | | TTGCTCAGGCAC | | TTCGCACAAGCA | |
| | | AG | | CAG | |
| AQLRMGYSQ | 211 | CAGAGTGCCCAA | 469 | CAGAGTGCACAA | 802 |
| | | TTGCGTATGGGTT | | CTCAGAATGGGA | |
| | | ATAGTCAGGCAC | | TACAGCCAAGCA | |
| | | AG | | CAG | |
| AQLRQGYAQ | 212 | CAGAGTGCCCAA | 470 | CAGAGTGCACAA | 803 |
| | | CTGAGGCAGGGG | | CTCAGACAAGGA | |
| | | TATGCTCAGGCA | | TACGCACAAGCA | |
| | | CAG | | CAG | |
| AQLRVGFAQ | 123 | CAGAGTGCCCAA | 471 | CAGAGTGCACAA | 804 |
| | | TTGCGTGTTGGTT | | CTCAGAGTCGGA | |
| | | TTGCGCAGGCAC | | TTCGCACAAGCA | |
| | | AG | | CAG | |
| AQLSCRSQM | 213 | CAGAGTGCCCAA | 472 | CAGAGTGCACAA | 805 |
| | | CTGTCGTGTCGGA | | CTCAGCTGCAGA | |
| | | GTCAGATGGCAC | | AGCCAAATGGCA | |
| | | AG | | CAG | |
| AQLTYSQSL | 214 | CAGAGTGCCCAA | 473 | CAGAGTGCACAA | 806 |
| | | TTGACGTATAGTC | | CTCACATACAGC | |
| | | AGTCGCTGGCAC | | CAAAGCCTCGCA | |
| | | AG | | CAG | |
| AQLYKGYSQ | 215 | CAGAGTGCCCAA | 474 | CAGAGTGCACAA | 807 |
| | | CTGTATAAGGGTT | | CTCTACAAAGGA | |
| | | ATAGTCAGGCAC | | TACAGCCAAGCA | |
| | | AG | | CAG | |
| AQMPQRPFL | 216 | CAGAGTGCCCAA | 475 | CAGAGTGCACAA | 808 |
| | | ATGCCTCAGCGG | | ATGCCACAAAGA | |
| | | CCGTTTTTGGCAC | | CCATTCCTCGCAC | |
| | | AG | | AG | |
| AQNGNPGRW | 84 | CAGAGTGCCCAA | 476 | CAGAGTGCACAA | 809 |
| | | AATGGTAATCCG | | AACGGAAACCCA | |
| | | GGGCGGTGGGCA | | GGAAGATGGGCA | |
| | | CAG | | CAG | |
| AQPEGSARW | 60 | CAGAGTGCCCAA | 477 | CAGAGTGCACAA | 810 |
| | | CCTGAGGGTAGT | | CCAGAAGGAAGC | |
| | | GCGAGGTGGGCA | | GCAAGATGGGCA | |
| | | CAG | | CAG | |
| AQPLAVYGA | 217 | CAGAGTGCCCAA | 478 | CAGAGTGCACAA | 811 |
| | | CCGTTGGCTGTTT | | CCACTCGCAGTCT | |
| | | ATGGGGCGGCAC | | ACGGAGCAGCAC | |
| | | AG | | AG | |
| AQPQSSSMS | 218 | CAGAGTGCCCAA | 479 | CAGAGTGCACAA | 812 |
| | | CCGCAGTCGTCGT | | CCACAAAGCAGC | |
| | | CGATGAGTGCAC | | AGCATGAGCGCA | |
| | | AG | | CAG | |
| AQPSVGGYW | 219 | CAGAGTGCCCAA | 480 | CAGAGTGCACAA | 813 |
| | | CCGAGTGTGGGT | | CCAAGCGTCGGA | |
| | | GGGTATTGGGCA | | GGATACTGGGCA | |
| | | CAG | | CAG | |
| AQQAVGQSW | 220 | CAGAGTGCCCAA | 481 | CAGAGTGCACAA | 814 |
| | | CAGGCTGTGGGT | | CAAGCAGTCGGA | |
| | | CAGTCTTGGGCA | | CAAAGCTGGGCA | |
| | | CAG | | CAG | |
| AQQRSLASG | 221 | CAGAGTGCCCAA | 482 | CAGAGTGCACAA | 815 |
| | | CAGCGTTCGCTG | | CAAAGAAGCCTC | |
| | | GCTTCGGGTGCA | | GCAAGCGGAGCA | |
| | | CAG | | CAG | |
| AQQVMNSQG | 222 | CAGAGTGCCCAA | 483 | CAGAGTGCACAA | 816 |
| | | CAGGTGATGAAT | | CAAGTCATGAAC | |
| | | AGTCAGGGGGCA | | AGCCAAGGAGCA | |
| | | CAG | | CAG | |
| AQRGVGLSQ | 223 | CAGAGTGCCCAA | 484 | CAGAGTGCACAA | 817 |
| | | CGTGGGGTTGGG | | AGAGGAGTCGGA | |
| | | TTGAGTCAGGCA | | CTCAGCCAAGCA | |
| | | CAG | | CAG | |
| AQRHDAEGS | 224 | CAGAGTGCCCAA | 485 | CAGAGTGCACAA | 818 |
| | | AGGCATGATGCG | | AGACACGACGCA | |
| | | GAGGGTAGTGCA | | GAAGGAAGCGCA | |
| | | CAG | | CAG | |
| AQRKGEPHY | 225 | CAGAGTGCCCAA | 486 | CAGAGTGCACAA | 819 |
| | | CGTAAGGGGGAG | | AGAAAAGGAGAA | |
| | | CCTCATTATGCAC | | CCACACTACGCA | |
| | | AG | | CAG | |
| AQRYTGDSS | 138 | CAGAGTGCCCAA | 487 | CAGAGTGCACAA | 820 |
| | | AGGTATACGGGG | | AGATACACAGGA | |
| | | GATTCTAGTGCAC | | GACAGCAGCGCA | |
| | | AG | | CAG | |
| AQSAMAAKG | 226 | CAGAGTGCCCAA | 488 | CAGAGTGCACAA | 821 |
| | | TCGGCGATGGCT | | AGCGCAATGGCA | |
| | | GCGAAGGGTGCA | | GCAAAAGGAGCA | |
| | | CAG | | CAG | |
| AQSGGLTGS | 227 | CAGAGTGCCCAA | 489 | CAGAGTGCACAA | 822 |
| | | TCTGGGGGTCTTA | | AGCGGAGGACTC | |
| | | CGGGGAGTGCAC | | ACAGGAAGCGCA | |
| | | AG | | CAG | |
| AQSGGVGQV | 228 | CAGAGTGCCCAA | 490 | CAGAGTGCACAA | 823 |
| | | TCGGGTGGGGTG | | AGCGGAGGAGTC | |
| | | GGGCAGGTGGCA | | GGACAAGTCGCA | |
| | | CAG | | CAG | |
| AQSLATPFR | 169 | CAGAGTGCCCAA | 491 | CAGAGTGCACAA | 824 |
| | | TCTCTGGCGACGC | | AGCCTCGCAACA | |
| | | CTTTTCGTGCACA | | CCATTCAGAGCA | |
| | | G | | CAG | |
| AQSMSRPFL | 229 | CAGAGTGCCCAA | 492 | CAGAGTGCACAA | 825 |
| | | AGTATGTCGCGTC | | AGCATGAGCAGA | |
| | | CGTTTCTGGCACA | | CCATTCCTCGCAC | |
| | | G | | AG | |
| AQSQLRPFL | 230 | CAGAGTGCCCAA | 493 | CAGAGTGCACAA | 826 |
| | | AGTCAGCTTAGG | | AGCCAACTCAGA | |
| | | CCGTTTCTTGCAC | | CCATTCCTCGCAC | |
| | | AG | | AG | |
| AQSVAKPFL | 231 | CAGAGTGCCCAA | 494 | CAGAGTGCACAA | 827 |
| | | TCTGTGGCTAAGC | | AGCGTCGCAAAA | |
| | | CTTTTTTGGCACA | | CCATTCCTCGCAC | |
| | | G | | AG | |
| AQSVSQPFR | 232 | CAGAGTGCCCAA | 495 | CAGAGTGCACAA | 828 |
| | | TCGGTTTCGCAGC | | AGCGTCAGCCAA | |
| | | CGTTTAGGGCAC | | CCATTCAGAGCA | |
| | | AG | | CAG | |
| AQSVVRPFL | 233 | CAGAGTGCCCAA | 496 | CAGAGTGCACAA | 829 |
| | | TCTGTGGTGCGTC | | AGCGTCGTCAGA | |
| | | CTTTTCTGGCACA | | CCATTCCTCGCAC | |
| | | G | | AG | |
| AQTALSSST | 234 | CAGAGTGCCCAA | 497 | CAGAGTGCACAA | 830 |
| | | ACTGCGCTTTCGT | | ACAGCACTCAGC | |
| | | CGTCGACGGCAC | | AGCAGCACAGCA | |
| | | AG | | CAG | |
| AQTEMGGRC | 235 | CAGAGTGCCCAA | 498 | CAGAGTGCACAA | 831 |
| | | ACGGAGATGGGT | | ACAGAAATGGGA | |
| | | GGGAGGTGTGCA | | GGAAGATGCGCA | |
| | | CAG | | CAG | |
| AQTGFAPPR | 161 | CAGAGTGCCCAA | 499 | CAGAGTGCACAA | 832 |
| | | ACGGGGTTTGCTC | | ACAGGATTCGCA | |
| | | CGCCGCGTGCAC | | CCACCAAGAGCA | |
| | | AG | | CAG | |
| AQTIRGYSS | 236 | CAGAGTGCCCAA | 500 | CAGAGTGCACAA | 833 |
| | | ACGATTCGGGGG | | ACAATCAGAGGA | |
| | | TATTCGTCTGCAC | | TACAGCAGCGCA | |
| | | AG | | CAG | |
| AQTISNYHT | 237 | CAGAGTGCCCAA | 501 | CAGAGTGCACAA | 834 |
| | | ACTATTTCTAATT | | ACAATCAGCAAC | |
| | | ATCATACGGCAC | | TACCACACAGCA | |
| | | AG | | CAG | |
| AQTLARPFV | 98 | CAGAGTGCCCAA | 502 | CAGAGTGCACAA | 835 |
| | | ACTTTGGCGCGTC | | ACACTCGCAAGA | |
| | | CGTTTGTGGCACA | | CCATTCGTCGCAC | |
| | | G | | AG | |
| AQTLAVPFK | 168 | CAGAGTGCCCAA | 503 | CAGAGTGCACAA | 836 |
| (PHP.B) | | ACTTTGGCGGTGC | | ACACTCGCAGTC | |
| | | CTTTTAAGGCACA | | CCATTCAAAGCA | |
| | | G | | CAG | |
| AQTPDRPWL | 238 | CAGAGTGCCCAA | 504 | CAGAGTGCACAA | 837 |
| | | ACTCCTGATCGTC | | ACACCAGACAGA | |
| | | CTTGGTTGGCACA | | CCATGGCTCGCA | |
| | | G | | CAG | |
| AQTRAGYAQ | 126 | CAGAGTGCCCAA | 505 | CAGAGTGCACAA | 838 |
| | | ACTCGGGCTGGG | | ACAAGAGCAGGA | |
| | | TATGCTCAGGCA | | TACGCACAAGCA | |
| | | CAG | | CAG | |
| AQTRAGYSQ | 141 | CAGAGTGCCCAA | 506 | CAGAGTGCACAA | 839 |
| | | ACTAGGGCGGGG | | ACAAGAGCAGGA | |
| | | TATTCTCAGGCAC | | TACAGCCAAGCA | |
| | | AG | | CAG | |
| AQTREYLLG | 93 | CAGAGTGCCCAA | 507 | CAGAGTGCACAA | 840 |
| | | ACGCGTGAGTAT | | ACAAGAGAATAC | |
| | | CTGCTGGGGGCA | | CTCCTCGGAGCA | |
| | | CAG | | CAG | |
| AQTSAKPFL | 163 | CAGAGTGCCCAA | 508 | CAGAGTGCACAA | 841 |
| | | ACTTCTGCGAAG | | ACAAGCGCAAAA | |
| | | CCGTTTCTTGCAC | | CCATTCCTCGCAC | |
| | | AG | | AG | |
| AQTSARPFL | 100 | CAGAGTGCCCAA | 509 | CAGAGTGCACAA | 842 |
| | | ACTTCTGCTAGGC | | ACAAGCGCAAGA | |
| | | CTTTTCTGGCACA | | CCATTCCTCGCAC | |
| | | G | | AG | |
| AQTTDRPFL | 85 | CAGAGTGCCCAA | 510 | CAGAGTGCACAA | 843 |
| | | ACTACTGATAGG | | ACAACAGACAGA | |
| | | CCTTTTTTGGCAC | | CCATTCCTCGCAC | |
| | | AG | | AG | |
| AQTTEKPWL | 83 | CAGAGTGCCCAA | 511 | CAGAGTGCACAA | 844 |
| | | ACGACTGAGAAG | | ACAACAGAAAAA | |
| | | CCGTGGCTGGCA | | CCATGGCTCGCA | |
| | | CAG | | CAG | |
| AQTVARPFY | 239 | CAGAGTGCCCAA | 512 | CAGAGTGCACAA | 845 |
| | | ACGGTTGCGCGG | | ACAGTCGCAAGA | |
| | | CCTTTTTATGCAC | | CCATTCTACGCAC | |
| | | AG | | AG | |
| AQTVATPFR | 240 | CAGAGTGCCCAA | 513 | CAGAGTGCACAA | 846 |
| | | ACTGTTGCTACGC | | ACAGTCGCAACA | |
| | | CGTTTAGGGCAC | | CCATTCAGAGCA | |
| | | AG | | CAG | |
| AQTVTQLFK | 241 | CAGAGTGCCCAA | 514 | CAGAGTGCACAA | 847 |
| | | ACGGTGACGCAG | | ACAGTCACACAA | |
| | | TTGTTTAAGGCAC | | CTCTTCAAAGCAC | |
| | | AG | | AG | |
| AQVHVGSVY | 165 | CAGAGTGCCCAA | 515 | CAGAGTGCACAA | 848 |
| | | GTTCATGTTGGGA | | GTCCACGTCGGA | |
| | | GTGTTTATGCACA | | AGCGTCTACGCA | |
| | | G | | CAG | |
| AQVLAGYNM | 242 | CAGAGTGCCCAA | 516 | CAGAGTGCACAA | 849 |
| | | GTTCTTGCTGGGT | | GTCCTCGCAGGA | |
| | | ATAATATGGCAC | | TACAACATGGCA | |
| | | AG | | CAG | |
| AQVSEARVR | 243 | CAGAGTGCCCAA | 517 | CAGAGTGCACAA | 850 |
| | | GTTTCTGAGGCG | | GTCAGCGAAGCA | |
| | | AGGGTTAGGGCA | | AGAGTCAGAGCA | |
| | | CAG | | CAG | |
| AQVVVGYSQ | 244 | CAGAGTGCCCAA | 518 | CAGAGTGCACAA | 851 |
| | | GTTGTGGTGGGTT | | GTCGTCGTCGGAT | |
| | | ATAGTCAGGCAC | | ACAGCCAAGCAC | |
| | | AG | | AG | |
| AQWAAGYNV | 245 | CAGAGTGCCCAA | 519 | CAGAGTGCACAA | 852 |
| | | TGGGCTGCTGGG | | TGGGCAGCAGGA | |
| | | TATAATGTGGCA | | TACAACGTCGCA | |
| | | CAG | | CAG | |
| AQWELSNGY | 246 | CAGAGTGCCCAA | 520 | CAGAGTGCACAA | 853 |
| | | TGGGAGCTGAGT | | TGGGAACTCAGC | |
| | | AATGGGTATGCA | | AACGGATACGCA | |
| | | CAG | | CAG | |
| AQWEVKGGY | 247 | CAGAGTGCCCAA | 521 | CAGAGTGCACAA | 854 |
| | | TGGGAGGTGAAG | | TGGGAAGTCAAA | |
| | | GGGGGTTATGCA | | GGAGGATACGCA | |
| | | CAG | | CAG | |
| AQWEVKRGY | 248 | CAGAGTGCCCAA | 522 | CAGAGTGCACAA | 855 |
| | | TGGGAGGTGAAG | | TGGGAAGTCAAA | |
| | | CGGGGGTATGCA | | AGAGGATACGCA | |
| | | CAG | | CAG | |
| AQWEVQSGF | 249 | CAGAGTGCCCAA | 523 | CAGAGTGCACAA | 856 |
| | | TGGGAGGTTCAG | | TGGGAAGTCCAA | |
| | | TCTGGGTTTGCAC | | AGCGGATTCGCA | |
| | | AG | | CAG | |
| AQWEVRGGY | 250 | CAGAGTGCCCAA | 524 | CAGAGTGCACAA | 857 |
| | | TGGGAGGTTCGT | | TGGGAAGTCAGA | |
| | | GGTGGTTATGCA | | GGAGGATACGCA | |
| | | CAG | | CAG | |
| AQWEVTSGW | 251 | CAGAGTGCCCAA | 525 | CAGAGTGCACAA | 858 |
| | | TGGGAGGTGACG | | TGGGAAGTCACA | |
| | | AGTGGTTGGGCA | | AGCGGATGGGCA | |
| | | CAG | | CAG | |
| AQWGAPSHG | 252 | CAGAGTGCCCAA | 526 | CAGAGTGCACAA | 859 |
| | | TGGGGGGCGCCG | | TGGGGAGCACCA | |
| | | AGTCATGGGGCA | | AGCCACGGAGCA | |
| | | CAG | | CAG | |
| AQWMELGSS | 253 | CAGAGTGCCCAA | 527 | CAGAGTGCACAA | 860 |
| | | TGGATGGAGCTT | | TGGATGGAACTC | |
| | | GGTAGTTCGGCA | | GGAAGCAGCGCA | |
| | | CAG | | CAG | |
| AQWMFGGSG | 254 | CAGAGTGCCCAA | 528 | CAGAGTGCACAA | 861 |
| | | TGGATGTTTGGG | | TGGATGTTCGGA | |
| | | GGTAGTGGGGCA | | GGAAGCGGAGCA | |
| | | CAG | | CAG | |
| AQWMLGGAQ | 255 | CAGAGTGCCCAA | 529 | CAGAGTGCACAA | 862 |
| | | TGGATGCTGGGG | | TGGATGCTCGGA | |
| | | GGGGCGCAGGCA | | GGAGCACAAGCA | |
| | | CAG | | CAG | |
| AQWPTAYDA | 256 | CAGAGTGCCCAA | 530 | CAGAGTGCACAA | 863 |
| | | TGGCCGACTGCTT | | TGGCCAACAGCA | |
| | | ATGATGCGGCAC | | TACGACGCAGCA | |
| | | AG | | CAG | |
| AQWPTSYDA | 62 | CAGAGTGCCCAA | 531 | CAGAGTGCACAA | 864 |
| | | TGGCCTACGAGTT | | TGGCCAACAAGC | |
| | | ATGATGCTGCAC | | TACGACGCAGCA | |
| | | AG | | CAG | |
| AQWQVQTGF | 257 | CAGAGTGCCCAA | 532 | CAGAGTGCACAA | 865 |
| | | TGGCAGGTTCAG | | TGGCAAGTCCAA | |
| | | ACGGGGTTTGCA | | ACAGGATTCGCA | |
| | | CAG | | CAG | |
| AQWSTEGGY | 258 | CAGAGTGCCCAA | 533 | CAGAGTGCACAA | 866 |
| | | TGGTCGACTGAG | | TGGAGCACAGAA | |
| | | GGTGGGTATGCA | | GGAGGATACGCA | |
| | | CAG | | CAG | |
| AQWTAAGGY | 259 | CAGAGTGCCCAA | 534 | CAGAGTGCACAA | 867 |
| | | TGGACTGCTGCG | | TGGACAGCAGCA | |
| | | GGTGGTTATGCA | | GGAGGATACGCA | |
| | | CAG | | CAG | |
| AQWTTESGY | 260 | CAGAGTGCCCAA | 535 | CAGAGTGCACAA | 868 |
| | | TGGACGACGGAG | | TGGACAACAGAA | |
| | | TCGGGTTATGCAC | | AGCGGATACGCA | |
| | | AG | | CAG | |
| AQWVYGSSH | 261 | CAGAGTGCCCAA | 536 | CAGAGTGCACAA | 869 |
| | | TGGGTTTATGGG | | TGGGTCTACGGA | |
| | | AGTTCGCATGCA | | AGCAGCCACGCA | |
| | | CAG | | CAG | |
| AQYLAGYTV | 262 | CAGAGTGCCCAA | 537 | CAGAGTGCACAA | 870 |
| | | TATTTGGCGGGGT | | TACCTCGCAGGA | |
| | | ATACGGTGGCAC | | TACACAGTCGCA | |
| | | AG | | CAG | |
| AQYLKGYSV | 152 | CAGAGTGCCCAA | 538 | CAGAGTGCACAA | 871 |
| | | TATCTGAAGGGG | | TACCTCAAAGGA | |
| | | TATTCTGTGGCAC | | TACAGCGTCGCA | |
| | | AG | | CAG | |
| AQYLSGYNT | 263 | CAGAGTGCCCAA | 539 | CAGAGTGCACAA | 872 |
| | | TATTTGTCGGGTT | | TACCTCAGCGGA | |
| | | ATAATACGGCAC | | TACAACACAGCA | |
| | | AG | | CAG | |
| DGAAATTGW | 264 | CAGAGTGATGGC | 540 | CAGAGTGACGGA | 873 |
| | | GCTGCGGCGACT | | GCAGCAGCAACA | |
| | | ACTGGGTGGGCA | | ACAGGATGGGCA | |
| | | CAG | | CAG | |
| DGAGGTSGW | 151 | CAGAGTGATGGC | 541 | CAGAGTGACGGA | 874 |
| | | GCGGGTGGGACG | | GCAGGAGGAACA | |
| | | AGTGGTTGGGCA | | AGCGGATGGGCA | |
| | | CAG | | CAG | |
| DGAGTTSGW | 265 | CAGAGTGATGGC | 542 | CAGAGTGACGGA | 875 |
| | | GCGGGTACTACTT | | GCAGGAACAACA | |
| | | CGGGTTGGGCAC | | AGCGGATGGGCA | |
| | | AG | | CAG | |
| DGAHGLSGW | 266 | CAGAGTGATGGC | 543 | CAGAGTGACGGA | 876 |
| | | GCTCATGGGCTGT | | GCACACGGACTC | |
| | | CGGGGTGGGCAC | | AGCGGATGGGCA | |
| | | AG | | CAG | |
| DGAHVGLSS | 267 | CAGAGTGATGGC | 544 | CAGAGTGACGGA | 877 |
| | | GCTCATGTTGGGC | | GCACACGTCGGA | |
| | | TGTCGTCGGCAC | | CTCAGCAGCGCA | |
| | | AG | | CAG | |
| DGARTVLQL | 268 | CAGAGTGATGGC | 545 | CAGAGTGACGGA | 878 |
| | | GCTCGGACGGTG | | GCAAGAACAGTC | |
| | | CTTCAGTTGGCAC | | CTCCAACTCGCAC | |
| | | AG | | AG | |
| DGEYQKPFR | 269 | CAGAGTGATGGC | 546 | CAGAGTGACGGA | 879 |
| | | GAGTATCAGAAG | | GAATACCAAAAA | |
| | | CCGTTTAGGGCA | | CCATTCAGAGCA | |
| | | CAG | | CAG | |
| DGGGTTTGW | 270 | CAGAGTGATGGC | 547 | CAGAGTGACGGA | 880 |
| | | GGTGGGACTACG | | GGAGGAACAACA | |
| | | ACGGGGTGGGCA | | ACAGGATGGGCA | |
| | | CAG | | CAG | |
| DGHATSMGW | 271 | CAGAGTGATGGC | 548 | CAGAGTGACGGA | 881 |
| | | CATGCGACGAGT | | CACGCAACAAGC | |
| | | ATGGGTTGGGCA | | ATGGGATGGGCA | |
| | | CAG | | CAG | |
| DGKGSTQGW | 272 | CAGAGTGATGGC | 549 | CAGAGTGACGGA | 882 |
| | | AAGGGTTCGACG | | AAAGGAAGCACA | |
| | | CAGGGGTGGGCA | | CAAGGATGGGCA | |
| | | CAG | | CAG | |
| DGKQYQLSS | 92 | CAGAGTGATGGC | 550 | CAGAGTGACGGA | 883 |
| | | AAGCAGTATCAG | | AAACAATACCAA | |
| | | CTGTCTTCGGCAC | | CTCAGCAGCGCA | |
| | | AG | | CAG | |
| DGNGGLKGW | 167 | CAGAGTGATGGC | 551 | CAGAGTGACGGA | 884 |
| | | AATGGTGGGTTG | | AACGGAGGACTC | |
| | | AAGGGGTGGGCA | | AAAGGATGGGCA | |
| | | CAG | | CAG | |
| DGQGGLSGW | 273 | CAGAGTGATGGC | 552 | CAGAGTGACGGA | 885 |
| | | CAGGGGGGTTTG | | CAAGGAGGACTC | |
| | | TCTGGGTGGGCA | | AGCGGATGGGCA | |
| | | CAG | | CAG | |
| DGQHFAPPR | 110 | CAGAGTGATGGC | 553 | CAGAGTGACGGA | 886 |
| | | CAGCATTTTGCTC | | CAACACTTCGCA | |
| | | CGCCGCGGGCAC | | CCACCAAGAGCA | |
| | | AG | | CAG | |
| DGRATKTLY | 274 | CAGAGTGATGGC | 554 | CAGAGTGACGGA | 887 |
| | | CGTGCGACTAAG | | AGAGCAACAAAA | |
| | | ACGCTTTATGCAC | | ACACTCTACGCA | |
| | | AG | | CAG | |
| DGRNALTGW | 275 | CAGAGTGATGGC | 555 | CAGAGTGACGGA | 888 |
| | | CGTAATGCGTTG | | AGAAACGCACTC | |
| | | ACGGGGTGGGCA | | ACAGGATGGGCA | |
| | | CAG | | CAG | |
| DGRRQVIQL | 276 | CAGAGTGATGGC | 556 | CAGAGTGACGGA | 889 |
| | | AGGAGGCAGGTG | | AGAAGACAAGTC | |
| | | ATTCAGCTGGCA | | ATCCAACTCGCA | |
| | | CAG | | CAG | |
| DGRVYGLSS | 277 | CAGAGTGATGGC | 557 | CAGAGTGACGGA | 890 |
| | | AGGGTTTATGGTC | | AGAGTCTACGGA | |
| | | TTTCGTCGGCACA | | CTCAGCAGCGCA | |
| | | G | | CAG | |
| DGSGRTTGW | 147 | CAGAGTGATGGC | 558 | CAGAGTGACGGA | 891 |
| | | AGTGGGCGTACG | | AGCGGAAGAACA | |
| | | ACGGGTTGGGCA | | ACAGGATGGGCA | |
| | | CAG | | CAG | |
| DGSGTTRGW | 114 | CAGAGTGATGGC | 559 | CAGAGTGACGGA | 892 |
| | | TCTGGTACGACG | | AGCGGAACAACA | |
| | | CGGGGTTGGGCA | | AGAGGATGGGCA | |
| | | CAG | | CAG | |
| DGSGTVSGW | 278 | CAGAGTGATGGC | 560 | CAGAGTGACGGA | 893 |
| | | TCGGGTACGGTT | | AGCGGAACAGTC | |
| | | AGTGGGTGGGCA | | AGCGGATGGGCA | |
| | | CAG | | CAG | |
| DGSPEKPFR | 160 | CAGAGTGATGGC | 561 | CAGAGTGACGGA | 894 |
| | | AGTCCGGAGAAG | | AGCCCAGAAAAA | |
| | | CCGTTTCGGGCAC | | CCATTCAGAGCA | |
| | | AG | | CAG | |
| DGSQSTTGW | 136 | CAGAGTGATGGC | 562 | CAGAGTGACGGA | 895 |
| | | AGTCAGTCTACTA | | AGCCAAAGCACA | |
| | | CGGGGTGGGCAC | | ACAGGATGGGCA | |
| | | AG | | CAG | |
| DGSSFYPPK | 127 | CAGAGTGATGGC | 563 | CAGAGTGACGGA | 896 |
| | | AGTAGTTTTTATC | | AGCAGCTTCTACC | |
| | | CTCCTAAGGCAC | | CACCAAAAGCAC | |
| | | AG | | AG | |
| DGSSSYYDA | 64 | CAGAGTGATGGC | 564 | CAGAGTGACGGA | 897 |
| | | AGTAGTTCTTATT | | AGCAGCAGCTAC | |
| | | ATGATGCGGCAC | | TACGACGCAGCA | |
| | | AG | | CAG | |
| DGSTERPFR | 99 | CAGAGTGATGGC | 565 | CAGAGTGACGGA | 898 |
| | | TCTACGGAGAGG | | AGCACAGAAAGA | |
| | | CCGTTTAGGGCA | | CCATTCAGAGCA | |
| | | CAG | | CAG | |
| DGTAARLSS | 132 | CAGAGTGATGGC | 566 | CAGAGTGACGGA | 899 |
| | | ACCGCGGCTCGG | | ACAGCAGCAAGA | |
| | | CTGTCGTCGGCAC | | CTCAGCAGCGCA | |
| | | AG | | CAG | |
| DGTADKPFR | 63 | CAGAGTGATGGC | 567 | CAGAGTGACGGA | 900 |
| | | ACCGCTGATAAG | | ACAGCAGACAAA | |
| | | CCGTTTCGGGCAC | | CCATTCAGAGCA | |
| | | AG | | CAG | |
| DGTADRPFR | 155 | CAGAGTGATGGC | 568 | CAGAGTGACGGA | 901 |
| | | ACGGCGGATCGT | | ACAGCAGACAGA | |
| | | CCTTTTCGGGCAC | | CCATTCAGAGCA | |
| | | AG | | CAG | |
| DGTAERPFR | 140 | CAGAGTGATGGC | 569 | CAGAGTGACGGA | 902 |
| | | ACCGCGGAGAGG | | ACAGCAGAAAGA | |
| | | CCTTTTAGGGCAC | | CCATTCAGAGCA | |
| | | AG | | CAG | |
| DGTAIHLSS | 67 | CAGAGTGATGGC | 570 | CAGAGTGACGGA | 903 |
| | | ACCGCGATTCATC | | ACAGCAATCCAC | |
| | | TTTCGTCTGCACA | | CTCAGCAGCGCA | |
| | | G | | CAG | |
| DGTAIYLSS | 279 | CAGAGTGATGGC | 571 | CAGAGTGACGGA | 904 |
| | | ACCGCGATTTATC | | ACAGCAATCTAC | |
| | | TGTCTTCTGCACA | | CTCAGCAGCGCA | |
| | | G | | CAG | |
| DGTALMLSS | 280 | CAGAGTGATGGC | 572 | CAGAGTGACGGA | 905 |
| | | ACCGCTCTTATGT | | ACAGCACTCATG | |
| | | TGTCGTCTGCACA | | CTCAGCAGCGCA | |
| | | G | | CAG | |
| DGTASISGW | 281 | CAGAGTGATGGC | 573 | CAGAGTGACGGA | 906 |
| | | ACCGCGAGTATT | | ACAGCAAGCATC | |
| | | AGTGGTTGGGCA | | AGCGGATGGGCA | |
| | | CAG | | CAG | |
| DGTASTSGW | 282 | CAGAGTGATGGC | 574 | CAGAGTGACGGA | 907 |
| | | ACCGCGTCGACG | | ACAGCAAGCACA | |
| | | AGTGGGTGGGCA | | AGCGGATGGGCA | |
| | | CAG | | CAG | |
| DGTASVTGW | 283 | CAGAGTGATGGC | 575 | CAGAGTGACGGA | 908 |
| | | ACCGCGTCGGTG | | ACAGCAAGCGTC | |
| | | ACGGGGTGGGCA | | ACAGGATGGGCA | |
| | | CAG | | CAG | |
| DGTASYYDS | 61 | CAGAGTGATGGC | 576 | CAGAGTGACGGA | 909 |
| | | ACCGCGAGTTATT | | ACAGCAAGCTAC | |
| | | ATGATTCTGCACA | | TACGACAGCGCA | |
| | | G | | CAG | |
| DGTATTMGW | 284 | CAGAGTGATGGC | 577 | CAGAGTGACGGA | 910 |
| | | ACCGCGACGACG | | ACAGCAACAACA | |
| | | ATGGGGTGGGCA | | ATGGGATGGGCA | |
| | | CAG | | CAG | |
| DGTATTTGW | 285 | CAGAGTGATGGC | 578 | CAGAGTGACGGA | 911 |
| | | ACCGCGACGACG | | ACAGCAACAACA | |
| | | ACGGGTTGGGCA | | ACAGGATGGGCA | |
| | | CAG | | CAG | |
| DGTAYRLSS | 286 | CAGAGTGATGGC | 579 | CAGAGTGACGGA | 912 |
| | | ACCGCGTATCGTT | | ACAGCATACAGA | |
| | | TGTCGTCTGCACA | | CTCAGCAGCGCA | |
| | | G | | CAG | |
| DGTDKMWSI | 287 | CAGAGTGATGGC | 580 | CAGAGTGACGGA | 913 |
| | | ACCGATAAGATG | | ACAGACAAAATG | |
| | | TGGAGTATTGCA | | TGGAGCATCGCA | |
| | | CAG | | CAG | |
| DGTGGIKGW | 131 | CAGAGTGATGGC | 581 | CAGAGTGACGGA | 914 |
| | | ACCGGTGGTATT | | ACAGGAGGAATC | |
| | | AAGGGGTGGGCA | | AAAGGATGGGCA | |
| | | CAG | | CAG | |
| DGTGGIMGW | 288 | CAGAGTGATGGC | 582 | CAGAGTGACGGA | 915 |
| | | ACCGGGGGGATT | | ACAGGAGGAATC | |
| | | ATGGGTTGGGCA | | ATGGGATGGGCA | |
| | | CAG | | CAG | |
| DGTGGISGW | 289 | CAGAGTGATGGC | 583 | CAGAGTGACGGA | 916 |
| | | ACCGGTGGGATT | | ACAGGAGGAATC | |
| | | TCGGGGTGGGCA | | AGCGGATGGGCA | |
| | | CAG | | CAG | |
| DGTGGLAGW | 290 | CAGAGTGATGGC | 584 | CAGAGTGACGGA | 917 |
| | | ACCGGGGGTCTT | | ACAGGAGGACTC | |
| | | GCTGGTTGGGCA | | GCAGGATGGGCA | |
| | | CAG | | CAG | |
| DGTGGLHGW | 291 | CAGAGTGATGGC | 585 | CAGAGTGACGGA | 918 |
| | | ACCGGGGGGTTG | | ACAGGAGGACTC | |
| | | CATGGTTGGGCA | | CACGGATGGGCA | |
| | | CAG | | CAG | |
| DGTGGLQGW | 292 | CAGAGTGATGGC | 586 | CAGAGTGACGGA | 919 |
| | | ACCGGGGGTTTG | | ACAGGAGGACTC | |
| | | CAGGGTTGGGCA | | CAAGGATGGGCA | |
| | | CAG | | CAG | |
| DGTGGLRGW | 154 | CAGAGTGATGGC | 587 | CAGAGTGACGGA | 920 |
| | | ACCGGGGGTTTG | | ACAGGAGGACTC | |
| | | CGTGGTTGGGCA | | AGAGGATGGGCA | |
| | | CAG | | CAG | |
| DGTGGLSGW | 293 | CAGAGTGATGGC | 588 | CAGAGTGACGGA | 921 |
| | | ACCGGTGGGTTG | | ACAGGAGGACTC | |
| | | TCGGGTTGGGCA | | AGCGGATGGGCA | |
| | | CAG | | CAG | |
| DGTGGLTGW | 294 | CAGAGTGATGGC | 589 | CAGAGTGACGGA | 922 |
| | | ACCGGGGGGTTG | | ACAGGAGGACTC | |
| | | ACGGGTTGGGCA | | ACAGGATGGGCA | |
| | | CAG | | CAG | |
| DGTGGTKGW | 107 | CAGAGTGATGGC | 590 | CAGAGTGACGGA | 923 |
| | | ACCGGTGGGACT | | ACAGGAGGAACA | |
| | | AAGGGTTGGGCA | | AAAGGATGGGCA | |
| | | CAG | | CAG | |
| DGTGGTSGW | 295 | CAGAGTGATGGC | 591 | CAGAGTGACGGA | 924 |
| | | ACCGGGGGGACG | | ACAGGAGGAACA | |
| | | AGTGGTTGGGCA | | AGCGGATGGGCA | |
| | | CAG | | CAG | |
| DGTGGVHGW | 296 | CAGAGTGATGGC | 592 | CAGAGTGACGGA | 925 |
| | | ACCGGTGGGGTG | | ACAGGAGGAGTC | |
| | | CATGGTTGGGCA | | CACGGATGGGCA | |
| | | CAG | | CAG | |
| DGTGGVMGW | 297 | CAGAGTGATGGC | 593 | CAGAGTGACGGA | 926 |
| | | ACCGGTGGTGTT | | ACAGGAGGAGTC | |
| | | ATGGGGTGGGCA | | ATGGGATGGGCA | |
| | | CAG | | CAG | |
| DGTGGVSGW | 298 | CAGAGTGATGGC | 594 | CAGAGTGACGGA | 927 |
| | | ACCGGGGGGGTG | | ACAGGAGGAGTC | |
| | | TCTGGTTGGGCAC | | AGCGGATGGGCA | |
| | | AG | | CAG | |
| DGTGGVTGW | 299 | CAGAGTGATGGC | 595 | CAGAGTGACGGA | 928 |
| | | ACCGGTGGTGTG | | ACAGGAGGAGTC | |
| | | ACGGGGTGGGCA | | ACAGGATGGGCA | |
| | | CAG | | CAG | |
| DGTGGVYGW | 300 | CAGAGTGATGGC | 596 | CAGAGTGACGGA | 929 |
| | | ACCGGTGGTGTG | | ACAGGAGGAGTC | |
| | | TATGGGTGGGCA | | TACGGATGGGCA | |
| | | CAG | | CAG | |
| DGTGNLQGW | 301 | CAGAGTGATGGC | 597 | CAGAGTGACGGA | 930 |
| | | ACCGGTAATTTGC | | ACAGGAAACCTC | |
| | | AGGGTTGGGCAC | | CAAGGATGGGCA | |
| | | AG | | CAG | |
| DGTGNLRGW | 133 | CAGAGTGATGGC | 598 | CAGAGTGACGGA | 931 |
| | | ACCGGGAATCTT | | ACAGGAAACCTC | |
| | | AGGGGGTGGGCA | | AGAGGATGGGCA | |
| | | CAG | | CAG | |
| DGTGNLSGW | 302 | CAGAGTGATGGC | 599 | CAGAGTGACGGA | 932 |
| | | ACCGGGAATTTG | | ACAGGAAACCTC | |
| | | AGTGGGTGGGCA | | AGCGGATGGGCA | |
| | | CAG | | CAG | |
| DGTGNTHGW | 72 | CAGAGTGATGGC | 600 | CAGAGTGACGGA | 933 |
| | | ACCGGGAATACT | | ACAGGAAACACA | |
| | | CATGGGTGGGCA | | CACGGATGGGCA | |
| | | CAG | | CAG | |
| DGTGNTRGW | 94 | CAGAGTGATGGC | 601 | CAGAGTGACGGA | 934 |
| | | ACCGGGAATACT | | ACAGGAAACACA | |
| | | CGGGGGTGGGCA | | AGAGGATGGGCA | |
| | | CAG | | CAG | |
| DGTGNTSGW | 137 | CAGAGTGATGGC | 602 | CAGAGTGACGGA | 935 |
| | | ACCGGTAATACT | | ACAGGAAACACA | |
| | | AGTGGTTGGGCA | | AGCGGATGGGCA | |
| | | CAG | | CAG | |
| DGTGNVSGW | 303 | CAGAGTGATGGC | 603 | CAGAGTGACGGA | 936 |
| | | ACCGGGAATGTG | | ACAGGAAACGTC | |
| | | TCGGGGTGGGCA | | AGCGGATGGGCA | |
| | | CAG | | CAG | |
| DGTGNVTGW | 69 | CAGAGTGATGGC | 604 | CAGAGTGACGGA | 937 |
| | | ACCGGTAATGTG | | ACAGGAAACGTC | |
| | | ACGGGGTGGGCA | | ACAGGATGGGCA | |
| | | CAG | | CAG | |
| DGTGQLVGW | 304 | CAGAGTGATGGC | 605 | CAGAGTGACGGA | 938 |
| | | ACCGGGCAGCTT | | ACAGGACAACTC | |
| | | GTGGGTTGGGCA | | GTCGGATGGGCA | |
| | | CAG | | CAG | |
| DGTGQTIGW | 305 | CAGAGTGATGGC | 606 | CAGAGTGACGGA | 939 |
| | | ACCGGTCAGACG | | ACAGGACAAACA | |
| | | ATTGGTTGGGCA | | ATCGGATGGGCA | |
| | | CAG | | CAG | |
| DGTGQVTGW | 68 | CAGAGTGATGGC | 607 | CAGAGTGACGGA | 940 |
| | | ACCGGGCAGGTG | | ACAGGACAAGTC | |
| | | ACTGGGTGGGCA | | ACAGGATGGGCA | |
| | | CAG | | CAG | |
| DGTGRLTGW | 159 | CAGAGTGATGGC | 608 | CAGAGTGACGGA | 941 |
| | | ACCGGTCGGTTG | | ACAGGAAGACTC | |
| | | ACGGGTTGGGCA | | ACAGGATGGGCA | |
| | | CAG | | CAG | |
| DGTGRTVGW | 117 | CAGAGTGATGGC | 609 | CAGAGTGACGGA | 942 |
| | | ACCGGTCGGACT | | ACAGGAAGAACA | |
| | | GTTGGGTGGGCA | | GTCGGATGGGCA | |
| | | CAG | | CAG | |
| DGTGSGMMT | 306 | CAGAGTGATGGC | 610 | CAGAGTGACGGA | 943 |
| | | ACCGGTTCGGGT | | ACAGGAAGCGGA | |
| | | ATGATGACGGCA | | ATGATGACAGCA | |
| | | CAG | | CAG | |
| DGTGSISGW | 307 | CAGAGTGATGGC | 611 | CAGAGTGACGGA | 944 |
| | | ACCGGGTCGATT | | ACAGGAAGCATC | |
| | | AGTGGGTGGGCA | | AGCGGATGGGCA | |
| | | CAG | | CAG | |
| DGTGSLAGW | 308 | CAGAGTGATGGC | 612 | CAGAGTGACGGA | 945 |
| | | ACCGGTTCTTTGG | | ACAGGAAGCCTC | |
| | | CGGGGTGGGCAC | | GCAGGATGGGCA | |
| | | AG | | CAG | |
| DGTGSLNGW | 309 | CAGAGTGATGGC | 613 | CAGAGTGACGGA | 946 |
| | | ACCGGGTCTTTGA | | ACAGGAAGCCTC | |
| | | ATGGGTGGGCAC | | AACGGATGGGCA | |
| | | AG | | CAG | |
| DGTGSLQGW | 310 | CAGAGTGATGGC | 614 | CAGAGTGACGGA | 947 |
| | | ACCGGGTCGCTG | | ACAGGAAGCCTC | |
| | | CAGGGTTGGGCA | | CAAGGATGGGCA | |
| | | CAG | | CAG | |
| DGTGSLSGW | 311 | CAGAGTGATGGC | 615 | CAGAGTGACGGA | 948 |
| | | ACCGGGAGTCTG | | ACAGGAAGCCTC | |
| | | TCGGGGTGGGCA | | AGCGGATGGGCA | |
| | | CAG | | CAG | |
| DGTGSLVGW | 312 | CAGAGTGATGGC | 616 | CAGAGTGACGGA | 949 |
| | | ACCGGGTCGTTG | | ACAGGAAGCCTC | |
| | | GTGGGTTGGGCA | | GTCGGATGGGCA | |
| | | CAG | | CAG | |
| DGTGSTHGW | 119 | CAGAGTGATGGC | 617 | CAGAGTGACGGA | 950 |
| | | ACCGGGAGTACG | | ACAGGAAGCACA | |
| | | CATGGGTGGGCA | | CACGGATGGGCA | |
| | | CAG | | CAG | |
| DGTGSTKGW | 313 | CAGAGTGATGGC | 618 | CAGAGTGACGGA | 951 |
| | | ACCGGGAGTACT | | ACAGGAAGCACA | |
| | | AAGGGGTGGGCA | | AAAGGATGGGCA | |
| | | CAG | | CAG | |
| DGTGSTMGW | 314 | CAGAGTGATGGC | 619 | CAGAGTGACGGA | 952 |
| | | ACCGGTTCTACTA | | ACAGGAAGCACA | |
| | | TGGGTTGGGCAC | | ATGGGATGGGCA | |
| | | AG | | CAG | |
| DGTGSTQGW | 315 | CAGAGTGATGGC | 620 | CAGAGTGACGGA | 953 |
| | | ACCGGTAGTACG | | ACAGGAAGCACA | |
| | | CAGGGTTGGGCA | | CAAGGATGGGCA | |
| | | CAG | | CAG | |
| DGTGSTSGW | 316 | CAGAGTGATGGC | 621 | CAGAGTGACGGA | 954 |
| | | ACCGGGAGTACT | | ACAGGAAGCACA | |
| | | TCGGGGTGGGCA | | AGCGGATGGGCA | |
| | | CAG | | CAG | |
| DGTGSTTGW | 134 | CAGAGTGATGGC | 622 | CAGAGTGACGGA | 955 |
| | | ACCGGGAGTACG | | ACAGGAAGCACA | |
| | | ACGGGGTGGGCA | | ACAGGATGGGCA | |
| | | CAG | | CAG | |
| DGTGSVMGW | 317 | CAGAGTGATGGC | 623 | CAGAGTGACGGA | 956 |
| | | ACCGGTTCGGTTA | | ACAGGAAGCGTC | |
| | | TGGGGTGGGCAC | | ATGGGATGGGCA | |
| | | AG | | CAG | |
| DGTGSVTGW | 318 | CAGAGTGATGGC | 624 | CAGAGTGACGGA | 957 |
| | | ACCGGGTCTGTG | | ACAGGAAGCGTC | |
| | | ACTGGGTGGGCA | | ACAGGATGGGCA | |
| | | CAG | | CAG | |
| DGTGTLAGW | 319 | CAGAGTGATGGC | 625 | CAGAGTGACGGA | 958 |
| | | ACCGGGACGCTT | | ACAGGAACACTC | |
| | | GCGGGGTGGGCA | | GCAGGATGGGCA | |
| | | CAG | | CAG | |
| DGTGTLHGW | 320 | CAGAGTGATGGC | 626 | CAGAGTGACGGA | 959 |
| | | ACCGGTACTTTGC | | ACAGGAACACTC | |
| | | ATGGTTGGGCAC | | CACGGATGGGCA | |
| | | AG | | CAG | |
| DGTGTLKGW | 321 | CAGAGTGATGGC | 627 | CAGAGTGACGGA | 960 |
| | | ACCGGTACTCTTA | | ACAGGAACACTC | |
| | | AGGGTTGGGCAC | | AAAGGATGGGCA | |
| | | AG | | CAG | |
| DGTGTLSGW | 322 | CAGAGTGATGGC | 628 | CAGAGTGACGGA | 961 |
| | | ACCGGGACTCTG | | ACAGGAACACTC | |
| | | TCGGGTTGGGCA | | AGCGGATGGGCA | |
| | | CAG | | CAG | |
| DGTGTTLGW | 323 | CAGAGTGATGGC | 629 | CAGAGTGACGGA | 962 |
| | | ACCGGGACTACG | | ACAGGAACAACA | |
| | | CTGGGGTGGGCA | | CTCGGATGGGCA | |
| | | CAG | | CAG | |
| DGTGTTMGW | 324 | CAGAGTGATGGC | 630 | CAGAGTGACGGA | 963 |
| | | ACCGGGACTACT | | ACAGGAACAACA | |
| | | ATGGGTTGGGCA | | ATGGGATGGGCA | |
| | | CAG | | CAG | |
| DGTGTTTGW | 130 | CAGAGTGATGGC | 631 | CAGAGTGACGGA | 964 |
| | | ACCGGGACTACT | | ACAGGAACAACA | |
| | | ACGGGGTGGGCA | | ACAGGATGGGCA | |
| | | CAG | | CAG | |
| DGTGTTVGW | 74 | CAGAGTGATGGC | 632 | CAGAGTGACGGA | 965 |
| | | ACCGGTACTACG | | ACAGGAACAACA | |
| | | GTGGGGTGGGCA | | GTCGGATGGGCA | |
| | | CAG | | CAG | |
| DGTGTTYGW | 325 | CAGAGTGATGGC | 633 | CAGAGTGACGGA | 966 |
| | | ACCGGGACGACG | | ACAGGAACAACA | |
| | | TATGGTTGGGCA | | TACGGATGGGCA | |
| | | CAG | | CAG | |
| DGTGTVHGW | 326 | CAGAGTGATGGC | 634 | CAGAGTGACGGA | 967 |
| | | ACCGGTACGGTT | | ACAGGAACAGTC | |
| | | CATGGTTGGGCA | | CACGGATGGGCA | |
| | | CAG | | CAG | |
| DGTGTVQGW | 327 | CAGAGTGATGGC | 635 | CAGAGTGACGGA | 968 |
| | | ACCGGGACTGTG | | ACAGGAACAGTC | |
| | | CAGGGGTGGGCA | | CAAGGATGGGCA | |
| | | CAG | | CAG | |
| DGTGTVSGW | 328 | CAGAGTGATGGC | 636 | CAGAGTGACGGA | 969 |
| | | ACCGGTACTGTTT | | ACAGGAACAGTC | |
| | | CTGGTTGGGCAC | | AGCGGATGGGCA | |
| | | AG | | CAG | |
| DGTGTVTGW | 329 | CAGAGTGATGGC | 637 | CAGAGTGACGGA | 970 |
| | | ACCGGTACTGTTA | | ACAGGAACAGTC | |
| | | CTGGGTGGGCAC | | ACAGGATGGGCA | |
| | | AG | | CAG | |
| DGTHARLSS | 330 | CAGAGTGATGGC | 638 | CAGAGTGACGGA | 971 |
| | | ACCCATGCGAGG | | ACACACGCAAGA | |
| | | TTGTCTTCGGCAC | | CTCAGCAGCGCA | |
| | | AG | | CAG | |
| DGTHAYMAS | 153 | CAGAGTGATGGC | 639 | CAGAGTGACGGA | 972 |
| | | ACCCATGCTTATA | | ACACACGCATAC | |
| | | TGGCGTCTGCAC | | ATGGCAAGCGCA | |
| | | AG | | CAG | |
| DGTHFAPPR | 112 | CAGAGTGATGGC | 640 | CAGAGTGACGGA | 973 |
| | | ACCCATTTTGCGC | | ACACACTTCGCA | |
| | | CGCCGCGTGCAC | | CCACCAAGAGCA | |
| | | AG | | CAG | |
| DGTHIHLSS | 162 | CAGAGTGATGGC | 641 | CAGAGTGACGGA | 974 |
| | | ACCCATATTCATC | | ACACACATCCAC | |
| | | TGAGTAGTGCAC | | CTCAGCAGCGCA | |
| | | AG | | CAG | |
| DGTHIRALS | 331 | CAGAGTGATGGC | 642 | CAGAGTGACGGA | 975 |
| | | ACCCATATTAGG | | ACACACATCAGA | |
| | | GCTCTGAGTGCA | | GCACTCAGCGCA | |
| | | CAG | | CAG | |
| DGTHIRLAS | 332 | CAGAGTGATGGC | 643 | CAGAGTGACGGA | 976 |
| | | ACCCATATTCGTT | | ACACACATCAGA | |
| | | TGGCGAGTGCAC | | CTCGCAAGCGCA | |
| | | AG | | CAG | |
| DGTHLQPFR | 333 | CAGAGTGATGGC | 644 | CAGAGTGACGGA | 977 |
| | | ACCCATCTGCAG | | ACACACCTCCAA | |
| | | CCGTTTAGGGCA | | CCATTCAGAGCA | |
| | | CAG | | CAG | |
| DGTHSFYDA | 334 | CAGAGTGATGGC | 645 | CAGAGTGACGGA | 978 |
| | | ACCCATAGTTTTT | | ACACACAGCTTCT | |
| | | ATGATGCGGCAC | | ACGACGCAGCAC | |
| | | AG | | AG | |
| DGTHSTTGW | 145 | CAGAGTGATGGC | 646 | CAGAGTGACGGA | 979 |
| | | ACCCATTCTACTA | | ACACACAGCACA | |
| | | CGGGTTGGGCAC | | ACAGGATGGGCA | |
| | | AG | | CAG | |
| DGTHTRTGW | 90 | CAGAGTGATGGC | 647 | CAGAGTGACGGA | 980 |
| | | ACCCATACGCGG | | ACACACACAAGA | |
| | | ACGGGTTGGGCA | | ACAGGATGGGCA | |
| | | CAG | | CAG | |
| DGTHVRALS | 335 | CAGAGTGATGGC | 648 | CAGAGTGACGGA | 981 |
| | | ACCCATGTTAGG | | ACACACGTCAGA | |
| | | GCGTTGTCGGCA | | GCACTCAGCGCA | |
| | | CAG | | CAG | |
| DGTHVYMAS | 336 | CAGAGTGATGGC | 649 | CAGAGTGACGGA | 982 |
| | | ACCCATGTTTATA | | ACACACGTCTAC | |
| | | TGGCTAGTGCAC | | ATGGCAAGCGCA | |
| | | AG | | CAG | |
| DGTHVYMSS | 337 | CAGAGTGATGGC | 650 | CAGAGTGACGGA | 983 |
| | | ACCCATGTGTATA | | ACACACGTCTAC | |
| | | TGTCTAGTGCACA | | ATGAGCAGCGCA | |
| | | G | | CAG | |
| DGTIALPFK | 338 | CAGAGTGATGGC | 651 | CAGAGTGACGGA | 984 |
| | | ACCATTGCGCTTC | | ACAATCGCACTC | |
| | | CGTTTAAGGCAC | | CCATTCAAAGCA | |
| | | AG | | CAG | |
| DGTIALPFR | 339 | CAGAGTGATGGC | 652 | CAGAGTGACGGA | 985 |
| | | ACCATTGCTTTGC | | ACAATCGCACTC | |
| | | CGTTTAGGGCAC | | CCATTCAGAGCA | |
| | | AG | | CAG | |
| DGTIATRYV | 340 | CAGAGTGATGGC | 653 | CAGAGTGACGGA | 986 |
| | | ACCATTGCGACG | | ACAATCGCAACA | |
| | | CGGTATGTGGCA | | AGATACGTCGCA | |
| | | CAG | | CAG | |
| DGTIERPFR | 87 | CAGAGTGATGGC | 654 | CAGAGTGACGGA | 987 |
| | | ACCATTGAGCGG | | ACAATCGAAAGA | |
| | | CCTTTTCGTGCAC | | CCATTCAGAGCA | |
| | | AG | | CAG | |
| DGTIGYAYV | 341 | CAGAGTGATGGC | 655 | CAGAGTGACGGA | 988 |
| | | ACCATTGGTTATG | | ACAATCGGATAC | |
| | | CGTATGTTGCACA | | GCATACGTCGCA | |
| | | G | | CAG | |
| DGTIQAPFK | 342 | CAGAGTGATGGC | 656 | CAGAGTGACGGA | 989 |
| | | ACCATTCAGGCTC | | ACAATCCAAGCA | |
| | | CGTTTAAGGCAC | | CCATTCAAAGCA | |
| | | AG | | CAG | |
| DGTIRLPFK | 343 | CAGAGTGATGGC | 657 | CAGAGTGACGGA | 990 |
| | | ACCATTCGTCTTC | | ACAATCAGACTC | |
| | | CTTTTAAGGCACA | | CCATTCAAAGCA | |
| | | G | | CAG | |
| DGTISKEVG | 344 | CAGAGTGATGGC | 658 | CAGAGTGACGGA | 991 |
| | | ACCATTTCTAAGG | | ACAATCAGCAAA | |
| | | AGGTGGGGGCAC | | GAAGTCGGAGCA | |
| | | AG | | CAG | |
| DGTISQPFK | 105 | CAGAGTGATGGC | 659 | CAGAGTGACGGA | 992 |
| | | ACCATTTCGCAGC | | ACAATCAGCCAA | |
| | | CTTTTAAGGCACA | | CCATTCAAAGCA | |
| | | G | | CAG | |
| DGTKIQLSS | 146 | CAGAGTGATGGC | 660 | CAGAGTGACGGA | 993 |
| | | ACCAAGATTCAG | | ACAAAAATCCAA | |
| | | CTGTCTAGTGCAC | | CTCAGCAGCGCA | |
| | | AG | | CAG | |
| DGTKIRLSS | 111 | CAGAGTGATGGC | 661 | CAGAGTGACGGA | 994 |
| | | ACCAAGATTCGG | | ACAAAAATCAGA | |
| | | TTGTCGTCTGCAC | | CTCAGCAGCGCA | |
| | | AG | | CAG | |
| DGTKLMLSS | 157 | CAGAGTGATGGC | 662 | CAGAGTGACGGA | 995 |
| | | ACCAAGCTGATG | | ACAAAACTCATG | |
| | | TTGAGTAGTGCA | | CTCAGCAGCGCA | |
| | | CAG | | CAG | |
| DGTKLRLSS | 118 | CAGAGTGATGGC | 663 | CAGAGTGACGGA | 996 |
| | | ACCAAGTTGAGG | | ACAAAACTCAGA | |
| | | CTTAGTTCTGCAC | | CTCAGCAGCGCA | |
| | | AG | | CAG | |
| DGTKMVLQL | 142 | CAGAGTGATGGC | 664 | CAGAGTGACGGA | 997 |
| | | ACCAAGATGGTG | | ACAAAAATGGTC | |
| | | TTGCAGCTGGCA | | CTCCAACTCGCAC | |
| | | CAG | | AG | |
| DGTKSLVQL | 345 | CAGAGTGATGGC | 665 | CAGAGTGACGGA | 998 |
| | | ACCAAGAGTCTT | | ACAAAAAGCCTC | |
| | | GTGCAGCTTGCA | | GTCCAACTCGCA | |
| | | CAG | | CAG | |
| DGTKVLVQL | 122 | CAGAGTGATGGC | 666 | CAGAGTGACGGA | 999 |
| | | ACCAAGGTGCTG | | ACAAAAGTCCTC | |
| | | GTGCAGTTGGCA | | GTCCAACTCGCA | |
| | | CAG | | CAG | |
| DGTLAAPFK | 120 | CAGAGTGATGGC | 667 | CAGAGTGACGGA | 1000 |
| | | ACCTTGGCTGCTC | | ACACTCGCAGCA | |
| | | CTTTTAAGGCACA | | CCATTCAAAGCA | |
| | | G | | CAG | |
| DGTLAVNFK | 346 | CAGAGTGATGGG | 668 | CAGAGTGACGGA | 1001 |
| | | ACTTTGGCGGTG | | ACACTCGCAGTC | |
| | | AATTTTAAGGCA | | AACTTCAAAGCA | |
| | | CAG | | CAG | |
| DGTLAVPFK | 71 | CAGAGTGATGGG | 669 | CAGAGTGACGGA | 1002 |
| (PHP.eB) | | ACTTTGGCGGTGC | | ACACTCGCAGTC | |
| | | CTTTTAAGGCACA | | CCATTCAAAGCA | |
| | | G | | CAG | |
| DGTLAYPFK | 347 | CAGAGTGATGGC | 670 | CAGAGTGACGGA | 1003 |
| | | ACCCTTGCGTATC | | ACACTCGCATAC | |
| | | CTTTTAAGGCACA | | CCATTCAAAGCA | |
| | | G | | CAG | |
| DGTLERPFR | 156 | CAGAGTGATGGC | 671 | CAGAGTGACGGA | 1004 |
| | | ACCCTGGAGAGG | | ACACTCGAAAGA | |
| | | CCGTTTCGGGCAC | | CCATTCAGAGCA | |
| | | AG | | CAG | |
| DGTLEVHFK | 348 | CAGAGTGATGGG | 672 | CAGAGTGACGGA | 1005 |
| | | ACTTTGGAGGTG | | ACACTCGAAGTC | |
| | | CATTTTAAGGCAC | | CACTTCAAAGCA | |
| | | AG | | CAG | |
| DGTLLRLSS | 121 | CAGAGTGATGGC | 673 | CAGAGTGACGGA | 1006 |
| | | ACCTTGCTGAGG | | ACACTCCTCAGA | |
| | | CTGAGTAGTGCA | | CTCAGCAGCGCA | |
| | | CAG | | CAG | |
| DGTLNNPFR | 109 | CAGAGTGATGGC | 674 | CAGAGTGACGGA | 1007 |
| | | ACCTTGAATAATC | | ACACTCAACAAC | |
| | | CGTTTAGGGCAC | | CCATTCAGAGCA | |
| | | AG | | CAG | |
| DGTLQQPFR | 89 | CAGAGTGATGGC | 675 | CAGAGTGACGGA | 1008 |
| | | ACCTTGCAGCAG | | ACACTCCAACAA | |
| | | CCGTTTCGGGCAC | | CCATTCAGAGCA | |
| | | AG | | CAG | |
| DGTLSQPFR | 65 | CAGAGTGATGGC | 676 | CAGAGTGACGGA | 1009 |
| | | ACCCTGTCTCAGC | | ACACTCAGCCAA | |
| | | CTTTTAGGGCACA | | CCATTCAGAGCA | |
| | | G | | CAG | |
| DGTLSRTLW | 349 | CAGAGTGATGGC | 677 | CAGAGTGACGGA | 1010 |
| | | ACCTTGTCGCGTA | | ACACTCAGCAGA | |
| | | CGCTTTGGGCAC | | ACACTCTGGGCA | |
| | | AG | | CAG | |
| DGTLSSPFR | 350 | CAGAGTGATGGC | 678 | CAGAGTGACGGA | 1011 |
| | | ACCCTGTCTAGTC | | ACACTCAGCAGC | |
| | | CGTTTAGGGCAC | | CCATTCAGAGCA | |
| | | AG | | CAG | |
| DGTLTVPFR | 351 | CAGAGTGATGGC | 679 | CAGAGTGACGGA | 1012 |
| | | ACCTTGACGGTTC | | ACACTCACAGTC | |
| | | CTTTTCGGGCACA | | CCATTCAGAGCA | |
| | | G | | CAG | |
| DGTLVAPFR | 352 | CAGAGTGATGGC | 680 | CAGAGTGACGGA | 1013 |
| | | ACCCTTGTTGCGC | | ACACTCGTCGCA | |
| | | CGTTTAGGGCAC | | CCATTCAGAGCA | |
| | | AG | | CAG | |
| DGTMDKPFR | 70 | CAGAGTGATGGC | 681 | CAGAGTGACGGA | 1014 |
| | | ACGATGGATAAG | | ACAATGGACAAA | |
| | | CCTTTTAGGGCAC | | CCATTCAGAGCA | |
| | | AG | | CAG | |
| DGTMDRPFK | 102 | CAGAGTGATGGC | 682 | CAGAGTGACGGA | 1015 |
| | | ACCATGGATAGG | | ACAATGGACAGA | |
| | | CCGTTTAAGGCA | | CCATTCAAAGCA | |
| | | CAG | | CAG | |
| DGTMLRLSS | 148 | CAGAGTGATGGC | 683 | CAGAGTGACGGA | 1016 |
| | | ACCATGTTGCGTC | | ACAATGCTCAGA | |
| | | TTAGTTCGGCACA | | CTCAGCAGCGCA | |
| | | G | | CAG | |
| DGTMQLTGW | 353 | CAGAGTGATGGC | 684 | CAGAGTGACGGA | 1017 |
| | | ACCATGCAGCTT | | ACAATGCAACTC | |
| | | ACGGGGTGGGCA | | ACAGGATGGGCA | |
| | | CAG | | CAG | |
| DGTNGLKGW | 76 | CAGAGTGATGGC | 685 | CAGAGTGACGGA | 1018 |
| | | ACCAATGGTCTG | | ACAAACGGACTC | |
| | | AAGGGGTGGGCA | | AAAGGATGGGCA | |
| | | CAG | | CAG | |
| DGTNSISGW | 354 | CAGAGTGATGGC | 686 | CAGAGTGACGGA | 1019 |
| | | ACCAATAGTATT | | ACAAACAGCATC | |
| | | AGTGGGTGGGCA | | AGCGGATGGGCA | |
| | | CAG | | CAG | |
| DGTNSLSGW | 355 | CAGAGTGATGGC | 687 | CAGAGTGACGGA | 1020 |
| | | ACCAATTCTCTGT | | ACAAACAGCCTC | |
| | | CGGGTTGGGCAC | | AGCGGATGGGCA | |
| | | AG | | CAG | |
| DGTNSTTGW | 143 | CAGAGTGATGGC | 688 | CAGAGTGACGGA | 1021 |
| | | ACCAATTCTACG | | ACAAACAGCACA | |
| | | ACGGGTTGGGCA | | ACAGGATGGGCA | |
| | | CAG | | CAG | |
| DGTNSVTGW | 356 | CAGAGTGATGGC | 689 | CAGAGTGACGGA | 1022 |
| | | ACCAATAGTGTT | | ACAAACAGCGTC | |
| | | ACGGGTTGGGCA | | ACAGGATGGGCA | |
| | | CAG | | CAG | |
| DGTNTINGW | 124 | CAGAGTGATGGC | 690 | CAGAGTGACGGA | 1023 |
| | | ACCAATACTATTA | | ACAAACACAATC | |
| | | ATGGGTGGGCAC | | AACGGATGGGCA | |
| | | AG | | CAG | |
| DGTNTLGGW | 357 | CAGAGTGATGGC | 691 | CAGAGTGACGGA | 1024 |
| | | ACCAATACGTTG | | ACAAACACACTC | |
| | | GGGGGGTGGGCA | | GGAGGATGGGCA | |
| | | CAG | | CAG | |
| DGTNTTHGW | 113 | CAGAGTGATGGC | 692 | CAGAGTGACGGA | 1025 |
| | | ACCAATACTACTC | | ACAAACACAACA | |
| | | ATGGGTGGGCAC | | CACGGATGGGCA | |
| | | AG | | CAG | |
| DGTNYRLSS | 358 | CAGAGTGATGGC | 693 | CAGAGTGACGGA | 1026 |
| | | ACCAATTATAGG | | ACAAACTACAGA | |
| | | CTGTCGAGTGCA | | CTCAGCAGCGCA | |
| | | CAG | | CAG | |
| DGTQALSGW | 359 | CAGAGTGATGGC | 694 | CAGAGTGACGGA | 1027 |
| | | ACCCAGGCGCTG | | ACACAAGCACTC | |
| | | TCGGGGTGGGCA | | AGCGGATGGGCA | |
| | | CAG | | CAG | |
| DGTQFRLSS | 129 | CAGAGTGATGGC | 695 | CAGAGTGACGGA | 1028 |
| | | ACCCAGTTTAGGT | | ACACAATTCAGA | |
| | | TGTCTTCGGCACA | | CTCAGCAGCGCA | |
| | | G | | CAG | |
| DGTQFSPPR | 108 | CAGAGTGATGGC | 696 | CAGAGTGACGGA | 1029 |
| | | ACCCAGTTTAGTC | | ACACAATTCAGC | |
| | | CTCCGCGTGCAC | | CCACCAAGAGCA | |
| | | AG | | CAG | |
| DGTQGLKGW | 158 | CAGAGTGATGGC | 697 | CAGAGTGACGGA | 1030 |
| | | ACCCAGGGGCTG | | ACACAAGGACTC | |
| | | AAGGGGTGGGCA | | AAAGGATGGGCA | |
| | | CAG | | CAG | |
| DGTQTTSGW | 360 | CAGAGTGATGGC | 698 | CAGAGTGACGGA | 1031 |
| | | ACCCAGACTACG | | ACACAAACAACA | |
| | | AGTGGGTGGGCA | | AGCGGATGGGCA | |
| | | CAG | | CAG | |
| DGTRALTGW | 361 | CAGAGTGATGGC | 699 | CAGAGTGACGGA | 1032 |
| | | ACCAGGGCTCTT | | ACAAGAGCACTC | |
| | | ACGGGTTGGGCA | | ACAGGATGGGCA | |
| | | CAG | | CAG | |
| DGTRFSLSS | 362 | CAGAGTGATGGC | 700 | CAGAGTGACGGA | 1033 |
| | | ACCCGGTTTTCGC | | ACAAGATTCAGC | |
| | | TTTCGAGTGCACA | | CTCAGCAGCGCA | |
| | | G | | CAG | |
| DGTRGLSGW | 363 | CAGAGTGATGGC | 701 | CAGAGTGACGGA | 1034 |
| | | ACCAGGGGGTTG | | ACAAGAGGACTC | |
| | | TCGGGGTGGGCA | | AGCGGATGGGCA | |
| | | CAG | | CAG | |
| DGTRIGLSS | 364 | CAGAGTGATGGC | 702 | CAGAGTGACGGA | 1035 |
| | | ACCAGGATTGGG | | ACAAGAATCGGA | |
| | | CTGAGTAGTGCA | | CTCAGCAGCGCA | |
| | | CAG | | CAG | |
| DGTRLHLAS | 365 | CAGAGTGATGGC | 703 | CAGAGTGACGGA | 1036 |
| | | ACCAGGCTTCATC | | ACAAGACTCCAC | |
| | | TGGCGAGTGCAC | | CTCGCAAGCGCA | |
| | | AG | | CAG | |
| DGTRLHLSS | 366 | CAGAGTGATGGC | 704 | CAGAGTGACGGA | 1037 |
| | | ACCAGGCTTCATC | | ACAAGACTCCAC | |
| | | TGTCGTCGGCAC | | CTCAGCAGCGCA | |
| | | AG | | CAG | |
| DGTRLLLSS | 367 | CAGAGTGATGGC | 705 | CAGAGTGACGGA | 1038 |
| | | ACCCGTTTGCTGC | | ACAAGACTCCTC | |
| | | TGTCGAGTGCAC | | CTCAGCAGCGCA | |
| | | AG | | CAG | |
| DGTRLMLSS | 368 | CAGAGTGATGGC | 706 | CAGAGTGACGGA | 1039 |
| | | ACCCGTTTGATGC | | ACAAGACTCATG | |
| | | TTTCTAGTGCACA | | CTCAGCAGCGCA | |
| | | G | | CAG | |
| DGTRLNLSS | 369 | CAGAGTGATGGC | 707 | CAGAGTGACGGA | 1040 |
| | | ACCCGTTTGAATC | | ACAAGACTCAAC | |
| | | TTAGTTCGGCACA | | CTCAGCAGCGCA | |
| | | G | | CAG | |
| DGTRMVVQL | 370 | CAGAGTGATGGC | 708 | CAGAGTGACGGA | 1041 |
| | | ACCCGGATGGTT | | ACAAGAATGGTC | |
| | | GTTCAGCTTGCAC | | GTCCAACTCGCA | |
| | | AG | | CAG | |
| DGTRNMYEG | 135 | CAGAGTGATGGC | 709 | CAGAGTGACGGA | 1042 |
| | | ACCCGTAATATGT | | ACAAGAAACATG | |
| | | ATGAGGGGGCAC | | TACGAAGGAGCA | |
| | | AG | | CAG | |
| DGTRSITGW | 371 | CAGAGTGATGGC | 710 | CAGAGTGACGGA | 1043 |
| | | ACCAGGAGTATT | | ACAAGAAGCATC | |
| | | ACGGGGTGGGCA | | ACAGGATGGGCA | |
| | | CAG | | CAG | |
| DGTRSLHGW | 372 | CAGAGTGATGGC | 711 | CAGAGTGACGGA | 1044 |
| | | ACCAGGAGTTTG | | ACAAGAAGCCTC | |
| | | CATGGGTGGGCA | | CACGGATGGGCA | |
| | | CAG | | CAG | |
| DGTRSTTGW | 373 | CAGAGTGATGGC | 712 | CAGAGTGACGGA | 1045 |
| | | ACCCGGAGTACT | | ACAAGAAGCACA | |
| | | ACGGGTTGGGCA | | ACAGGATGGGCA | |
| | | CAG | | CAG | |
| DGTRTTTGW | 106 | CAGAGTGATGGC | 713 | CAGAGTGACGGA | 1046 |
| | | ACCCGTACTACG | | ACAAGAACAACA | |
| | | ACGGGTTGGGCA | | ACAGGATGGGCA | |
| | | CAG | | CAG | |
| DGTRTVTGW | 374 | CAGAGTGATGGC | 714 | CAGAGTGACGGA | 1047 |
| | | ACCCGGACGGTG | | ACAAGAACAGTC | |
| | | ACTGGTTGGGCA | | ACAGGATGGGCA | |
| | | CAG | | CAG | |
| DGTRTVVQL | 375 | CAGAGTGATGGC | 715 | CAGAGTGACGGA | 1048 |
| | | ACCCGTACTGTG | | ACAAGAACAGTC | |
| | | GTGCAGTTGGCA | | GTCCAACTCGCA | |
| | | CAG | | CAG | |
| DGTRVHLSS | 376 | CAGAGTGATGGC | 716 | CAGAGTGACGGA | 1049 |
| | | ACCCGGGTGCAT | | ACAAGAGTCCAC | |
| | | CTTTCTAGTGCAC | | CTCAGCAGCGCA | |
| | | AG | | CAG | |
| DGTSFPYAR | 86 | CAGAGTGATGGC | 717 | CAGAGTGACGGA | 1050 |
| | | ACCTCGTTTCCGT | | ACAAGCTTCCCAT | |
| | | ATGCTCGGGCAC | | ACGCAAGAGCAC | |
| | | AG | | AG | |
| DGTSFTPPK | 81 | CAGAGTGATGGC | 718 | CAGAGTGACGGA | 1051 |
| | | ACCTCGTTTACGC | | ACAAGCTTCACA | |
| | | CGCCTAAGGCAC | | CCACCAAAAGCA | |
| | | AG | | CAG | |
| DGTSFTPPR | 88 | CAGAGTGATGGC | 719 | CAGAGTGACGGA | 1052 |
| | | ACCTCGTTTACTC | | ACAAGCTTCACA | |
| | | CGCCGCGGGCAC | | CCACCAAGAGCA | |
| | | AG | | CAG | |
| DGTSGLHGW | 377 | CAGAGTGATGGC | 720 | CAGAGTGACGGA | 1053 |
| | | ACCTCTGGGTTGC | | ACAAGCGGACTC | |
| | | ATGGGTGGGCAC | | CACGGATGGGCA | |
| | | AG | | CAG | |
| DGTSGLKGW | 101 | CAGAGTGATGGC | 721 | CAGAGTGACGGA | 1054 |
| | | ACCAGTGGGCTT | | ACAAGCGGACTC | |
| | | AAGGGGTGGGCA | | AAAGGATGGGCA | |
| | | CAG | | CAG | |
| DGTSIHLSS | 378 | CAGAGTGATGGC | 722 | CAGAGTGACGGA | 1055 |
| | | ACCTCGATTCATT | | ACAAGCATCCAC | |
| | | TGAGTAGTGCAC | | CTCAGCAGCGCA | |
| | | AG | | CAG | |
| DGTSIMLSS | 379 | CAGAGTGATGGC | 723 | CAGAGTGACGGA | 1056 |
| | | ACCTCGATTATGT | | ACAAGCATCATG | |
| | | TGAGTTCTGCACA | | CTCAGCAGCGCA | |
| | | G | | CAG | |
| DGTSLRLSS | 166 | CAGAGTGATGGC | 724 | CAGAGTGACGGA | 1057 |
| | | ACCTCTTTGCGGC | | ACAAGCCTCAGA | |
| | | TTTCTTCTGCACA | | CTCAGCAGCGCA | |
| | | G | | CAG | |
| DGTSNYGAR | 380 | CAGAGTGATGGC | 725 | CAGAGTGACGGA | 1058 |
| | | ACCTCTAATTATG | | ACAAGCAACTAC | |
| | | GGGCGCGGGCAC | | GGAGCAAGAGCA | |
| | | AG | | CAG | |
| DGTSSYYDA | 381 | CAGAGTGATGGC | 726 | CAGAGTGACGGA | 1059 |
| | | ACCAGTTCGTATT | | ACAAGCAGCTAC | |
| | | ATGATGCGGCAC | | TACGACGCAGCA | |
| | | AG | | CAG | |
| DGTSSYYDS | 59 | CAGAGTGATGGC | 727 | CAGAGTGACGGA | 1060 |
| | | ACCTCGAGTTATT | | ACAAGCAGCTAC | |
| | | ATGATTCTGCACA | | TACGACAGCGCA | |
| | | G | | CAG | |
| DGTSTISGW | 382 | CAGAGTGATGGC | 728 | CAGAGTGACGGA | 1061 |
| | | ACCTCTACGATTT | | ACAAGCACAATC | |
| | | CTGGTTGGGCAC | | AGCGGATGGGCA | |
| | | AG | | CAG | |
| DGTSTITGW | 383 | CAGAGTGATGGC | 729 | CAGAGTGACGGA | 1062 |
| | | ACCAGTACTATTA | | ACAAGCACAATC | |
| | | CGGGTTGGGCAC | | ACAGGATGGGCA | |
| | | AG | | CAG | |
| DGTSTLHGW | 384 | CAGAGTGATGGC | 730 | CAGAGTGACGGA | 1063 |
| | | ACCTCGACGTTGC | | ACAAGCACACTC | |
| | | ATGGGTGGGCAC | | CACGGATGGGCA | |
| | | AG | | CAG | |
| DGTSTLRGW | 385 | CAGAGTGATGGC | 731 | CAGAGTGACGGA | 1064 |
| | | ACCTCTACTCTGC | | ACAAGCACACTC | |
| | | GTGGGTGGGCAC | | AGAGGATGGGCA | |
| | | AG | | CAG | |
| DGTSTLSGW | 386 | CAGAGTGATGGC | 732 | CAGAGTGACGGA | 1065 |
| | | ACCTCGACGCTGT | | ACAAGCACACTC | |
| | | CGGGGTGGGCAC | | AGCGGATGGGCA | |
| | | AG | | CAG | |
| DGTSYVPPK | 97 | CAGAGTGATGGC | 733 | CAGAGTGACGGA | 1066 |
| | | ACCTCTTATGTGC | | ACAAGCTACGTC | |
| | | CGCCGAAGGCAC | | CCACCAAAAGCA | |
| | | AG | | CAG | |
| DGTSYVPPR | 78 | CAGAGTGATGGC | 734 | CAGAGTGACGGA | 1067 |
| | | ACCAGTTATGTGC | | ACAAGCTACGTC | |
| | | CGCCTCGGGCAC | | CCACCAAGAGCA | |
| | | AG | | CAG | |
| DGTTATYYK | 387 | CAGAGTGATGGC | 735 | CAGAGTGACGGA | 1068 |
| | | ACCACGGCGACT | | ACAACAGCAACA | |
| | | TATTATAAGGCA | | TACTACAAAGCA | |
| | | CAG | | CAG | |
| DGTTFTPPR | 79 | CAGAGTGATGGC | 736 | CAGAGTGACGGA | 1069 |
| | | ACCACTTTTACTC | | ACAACATTCACA | |
| | | CTCCTCGGGCAC | | CCACCAAGAGCA | |
| | | AG | | CAG | |
| DGTTLAPFR | 388 | CAGAGTGATGGC | 737 | CAGAGTGACGGA | 1070 |
| | | ACCACTCTGGCTC | | ACAACACTCGCA | |
| | | CTTTTAGGGCACA | | CCATTCAGAGCA | |
| | | G | | CAG | |
| DGTTLVPPR | 116 | CAGAGTGATGGC | 738 | CAGAGTGACGGA | 1071 |
| | | ACCACTTTGGTTC | | ACAACACTCGTC | |
| | | CGCCGCGTGCAC | | CCACCAAGAGCA | |
| | | AG | | CAG | |
| DGTTSKTLW | 389 | CAGAGTGATGGC | 739 | CAGAGTGACGGA | 1072 |
| | | ACCACGAGTAAG | | ACAACAAGCAAA | |
| | | ACGCTTTGGGCA | | ACACTCTGGGCA | |
| | | CAG | | CAG | |
| DGTTSRTLW | 390 | CAGAGTGATGGC | 740 | CAGAGTGACGGA | 1073 |
| | | ACCACTTCTAGG | | ACAACAAGCAGA | |
| | | ACTTTGTGGGCAC | | ACACTCTGGGCA | |
| | | AG | | CAG | |
| DGTTTRSLY | 391 | CAGAGTGATGGC | 741 | CAGAGTGACGGA | 1074 |
| | | ACCACGACTCGT | | ACAACAACAAGA | |
| | | AGTTTGTATGCAC | | AGCCTCTACGCA | |
| | | AG | | CAG | |
| DGTTTTTGW | 392 | CAGAGTGATGGC | 742 | CAGAGTGACGGA | 1075 |
| | | ACCACTACGACT | | ACAACAACAACA | |
| | | ACGGGTTGGGCA | | ACAGGATGGGCA | |
| | | CAG | | CAG | |
| DGTTTYGAR | 77 | CAGAGTGATGGC | 743 | CAGAGTGACGGA | 1076 |
| | | ACCACTACGTAT | | ACAACAACATAC | |
| | | GGGGCTCGTGCA | | GGAGCAAGAGCA | |
| | | CAG | | CAG | |
| DGTTWTPPR | 139 | CAGAGTGATGGC | 744 | CAGAGTGACGGA | 1077 |
| | | ACCACTTGGACG | | ACAACATGGACA | |
| | | CCGCCGCGTGCA | | CCACCAAGAGCA | |
| | | CAG | | CAG | |
| DGTTYMLSS | 393 | CAGAGTGATGGC | 745 | CAGAGTGACGGA | 1078 |
| | | ACCACGTATATG | | ACAACATACATG | |
| | | CTTAGTAGTGCAC | | CTCAGCAGCGCA | |
| | | AG | | CAG | |
| DGTTYVPPR | 75 | CAGAGTGATGGC | 746 | CAGAGTGACGGA | 1079 |
| | | ACCACGTATGTTC | | ACAACATACGTC | |
| | | CTCCGCGGGCAC | | CCACCAAGAGCA | |
| | | AG | | CAG | |
| DGTVANPFR | 394 | CAGAGTGATGGC | 747 | CAGAGTGACGGA | 1080 |
| | | ACCGTGGCGAAT | | ACAGTCGCAAAC | |
| | | CCTTTTCGGGCAC | | CCATTCAGAGCA | |
| | | AG | | CAG | |
| DGTVDRPFK | 395 | CAGAGTGATGGC | 748 | CAGAGTGACGGA | 1081 |
| | | ACCGTGGATCGG | | ACAGTCGACAGA | |
| | | CCTTTTAAGGCAC | | CCATTCAAAGCA | |
| | | AG | | CAG | |
| DGTVIHLSS | 73 | CAGAGTGATGGC | 749 | CAGAGTGACGGA | 1082 |
| | | ACCGTTATTCATC | | ACAGTCATCCAC | |
| | | TGAGTAGTGCAC | | CTCAGCAGCGCA | |
| | | AG | | CAG | |
| DGTVILLSS | 396 | CAGAGTGATGGC | 750 | CAGAGTGACGGA | 1083 |
| | | ACCGTTATTCTGT | | ACAGTCATCCTCC | |
| | | TGTCGAGTGCAC | | TCAGCAGCGCAC | |
| | | AG | | AG | |
| DGTVIMLSS | 397 | CAGAGTGATGGC | 751 | CAGAGTGACGGA | 1084 |
| | | ACCGTGATTATGC | | ACAGTCATCATG | |
| | | TGTCGAGTGCAC | | CTCAGCAGCGCA | |
| | | AG | | CAG | |
| DGTVLHLSS | 398 | CAGAGTGATGGC | 752 | CAGAGTGACGGA | 1085 |
| | | ACCGTGCTTCATT | | ACAGTCCTCCACC | |
| | | TGTCGTCTGCACA | | TCAGCAGCGCAC | |
| | | G | | AG | |
| DGTVLMLSS | 399 | CAGAGTGATGGC | 753 | CAGAGTGACGGA | 1086 |
| | | ACCGTTTTGATGC | | ACAGTCCTCATGC | |
| | | TGAGTAGTGCAC | | TCAGCAGCGCAC | |
| | | AG | | AG | |
| DGTVLVPFR | 150 | CAGAGTGATGGC | 754 | CAGAGTGACGGA | 1087 |
| | | ACCGTGTTGGTGC | | ACAGTCCTCGTCC | |
| | | CGTTTAGGGCAC | | CATTCAGAGCAC | |
| | | AG | | AG | |
| DGTVPYLAS | 400 | CAGAGTGATGGC | 755 | CAGAGTGACGGA | 1088 |
| | | ACCGTTCCGTATC | | ACAGTCCCATAC | |
| | | TTGCTTCTGCACA | | CTCGCAAGCGCA | |
| | | G | | CAG | |
| DGTVPYLSS | 401 | CAGAGTGATGGC | 756 | CAGAGTGACGGA | 1089 |
| | | ACCGTGCCGTATT | | ACAGTCCCATAC | |
| | | TGTCTTCGGCACA | | CTCAGCAGCGCA | |
| | | G | | CAG | |
| DGTVRVPFR | 164 | CAGAGTGATGGC | 757 | CAGAGTGACGGA | 1090 |
| | | ACCGTTCGTGTGC | | ACAGTCAGAGTC | |
| | | CGTTTAGGGCAC | | CCATTCAGAGCA | |
| | | AG | | CAG | |
| DGTVSMPFK | 402 | CAGAGTGATGGC | 758 | CAGAGTGACGGA | 1091 |
| | | ACCGTGTCGATG | | ACAGTCAGCATG | |
| | | CCGTTTAAGGCA | | CCATTCAAAGCA | |
| | | CAG | | CAG | |
| DGTVSNPFR | 403 | CAGAGTGATGGC | 759 | CAGAGTGACGGA | 1092 |
| | | ACCGTGTCTAATC | | ACAGTCAGCAAC | |
| | | CGTTTAGGGCAC | | CCATTCAGAGCA | |
| | | AG | | CAG | |
| DGTVSTRWV | 404 | CAGAGTGATGGC | 760 | CAGAGTGACGGA | 1093 |
| | | ACCGTTTCTACGC | | ACAGTCAGCACA | |
| | | GTTGGGTGGCAC | | AGATGGGTCGCA | |
| | | AG | | CAG | |
| DGTVTTTGW | 405 | CAGAGTGATGGC | 761 | CAGAGTGACGGA | 1094 |
| | | ACCGTGACGACG | | ACAGTCACAACA | |
| | | ACTGGGTGGGCA | | ACAGGATGGGCA | |
| | | CAG | | CAG | |
| DGTVTVTGW | 406 | CAGAGTGATGGC | 762 | CAGAGTGACGGA | 1095 |
| | | ACCGTGACGGTT | | ACAGTCACAGTC | |
| | | ACGGGGTGGGCA | | ACAGGATGGGCA | |
| | | CAG | | CAG | |
| DGTVWVPPR | 407 | CAGAGTGATGGC | 763 | CAGAGTGACGGA | 1096 |
| | | ACCGTTTGGGTGC | | ACAGTCTGGGTC | |
| | | CTCCTAGGGCAC | | CCACCAAGAGCA | |
| | | AG | | CAG | |
| DGTVYRLSS | 408 | CAGAGTGATGGC | 764 | CAGAGTGACGGA | 1097 |
| | | ACCGTTTATAGGT | | ACAGTCTACAGA | |
| | | TGTCGAGTGCAC | | CTCAGCAGCGCA | |
| | | AG | | CAG | |
| DGTYARLSS | 409 | CAGAGTGATGGC | 765 | CAGAGTGACGGA | 1098 |
| | | ACCTATGCGCGTT | | ACATACGCAAGA | |
| | | TGTCTTCTGCACA | | CTCAGCAGCGCA | |
| | | G | | CAG | |
| DGTYGNKLW | 410 | CAGAGTGATGGC | 766 | CAGAGTGACGGA | 1099 |
| | | ACCTATGGTAAT | | ACATACGGAAAC | |
| | | AAGTTGTGGGCA | | AAACTCTGGGCA | |
| | | CAG | | CAG | |
| DGTYIHLSS | 411 | CAGAGTGATGGC | 767 | CAGAGTGACGGA | 1100 |
| | | ACCTATATTCATC | | ACATACATCCAC | |
| | | TGTCTTCGGCACA | | CTCAGCAGCGCA | |
| | | G | | CAG | |
| DGTYSTSGW | 412 | CAGAGTGATGGC | 768 | CAGAGTGACGGA | 1101 |
| | | ACCTATTCGACG | | ACATACAGCACA | |
| | | AGTGGGTGGGCA | | AGCGGATGGGCA | |
| | | CAG | | CAG | |
| DGVHPGLSS | 104 | CAGAGTGATGGC | 769 | CAGAGTGACGGA | 1102 |
| | | GTGCATCCTGGG | | GTCCACCCAGGA | |
| | | CTTTCGAGTGCAC | | CTCAGCAGCGCA | |
| | | AG | | CAG | |
| DGVVALLAS | 413 | CAGAGTGATGGC | 770 | CAGAGTGACGGA | 1103 |
| | | GTGGTTGCGTTGC | | GTCGTCGCACTCC | |
| | | TTGCTAGTGCACA | | TCGCAAGCGCAC | |
| | | G | | AG | |
| DGYVGVGSL | 414 | CAGAGTGATGGC | 771 | CAGAGTGACGGA | 1104 |
| | | TATGTGGGTGTTG | | TACGTCGGAGTC | |
| | | GTAGTTTGGCAC | | GGAAGCCTCGCA | |
| | | AG | | CAG | |

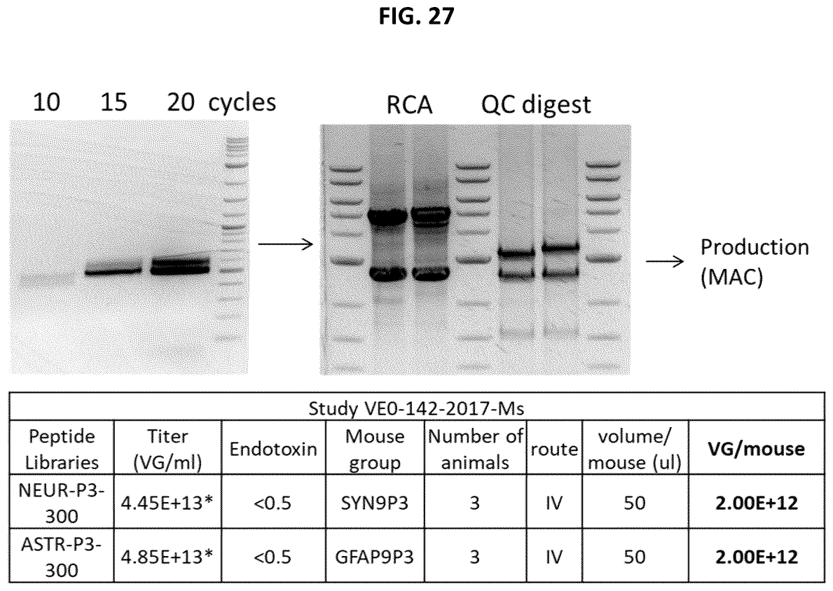

Primer pools were produced by Twist biosciences using solid-phase synthesis and were used to generate a balanced library of 666 nucleotide variants by PCR amplification of CAP C-terminus and Gibson assembly as described in FIG. 27. 666 primers were provided a 1 fmole each, resulting in 0.6 pmole (regular PCR requires ˜25 pmole of primer). Primerless amplification on capsid gBlock template was performed over 10 cycles. Forward and reverse primers were added, followed by an additional 10, 15 or 20 PCR cycles. Constructs were then cloned into AAV9 backbone plasmids by Gibson/RCA (like regular libraries).

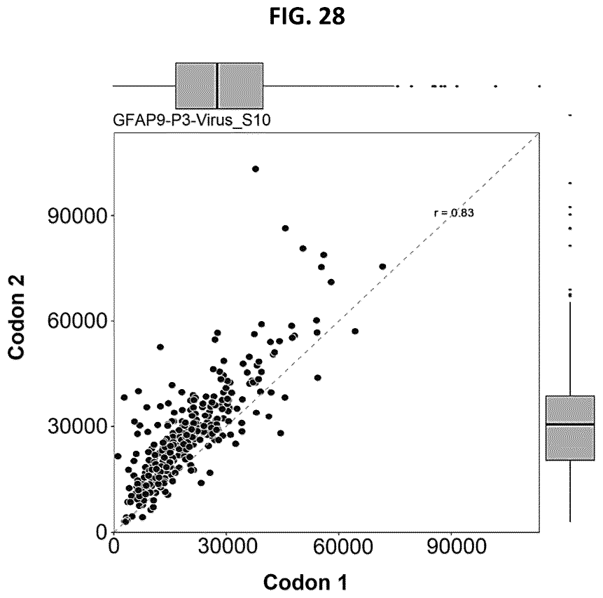

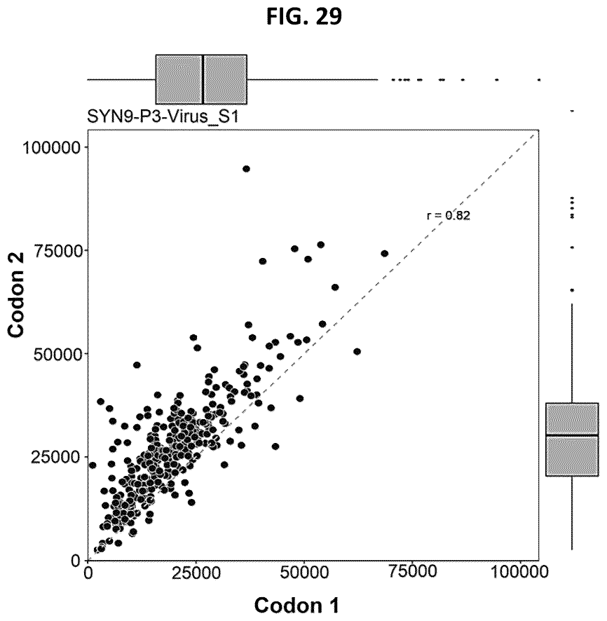

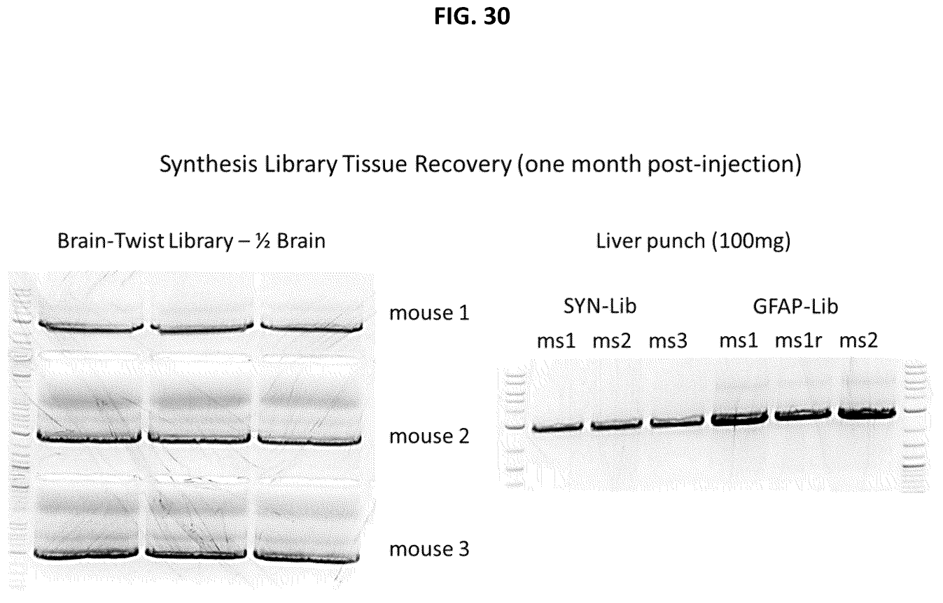

NGS analysis of SYN- and GFAP-driven AAV libraries produced with the pooled DNA showed a good correlation between the codon variants of each peptide, suggesting that the DNA sequence itself had little influence on virus production (FIG. 28 and FIG. 29). The pooled synthetic library was injected intravenously to C57BL/6 mice (5e11 VG per mouse, N=9), BALB/C mice (5e11 VG per mouse, N=6) and to rats (5e12 VG per rat, N=6), and after one month in-life RNA was extracted from the brain and spinal cord, and DNA was extracted from liver and heart tissue samples for biodistribution analysis (FIG. 30). Because the Synapsin and GFAP promoters are not fully active in non-CNS tissue, DNA was analyzed instead of RNA in peripheral organs. The initial focus was on the C57BL/6 mouse analysis because this is the mouse strain in which library evolution was performed.

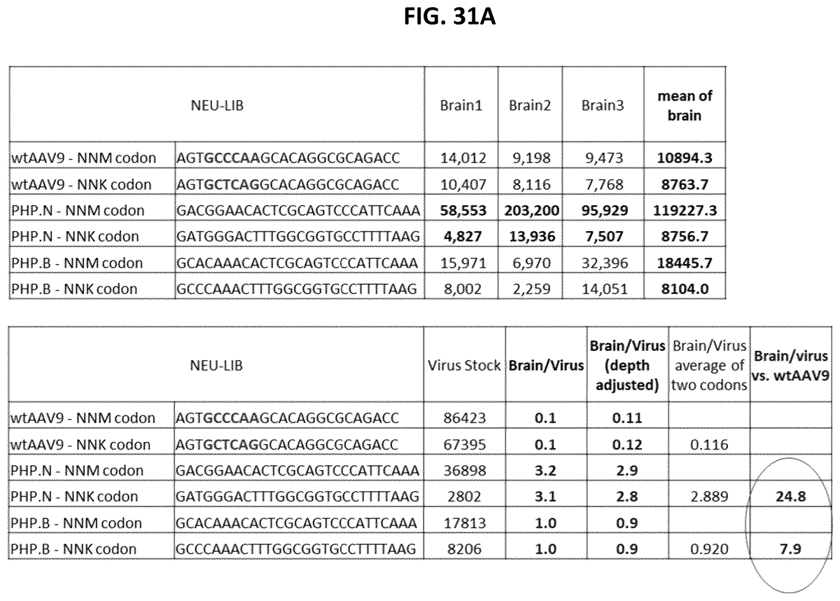

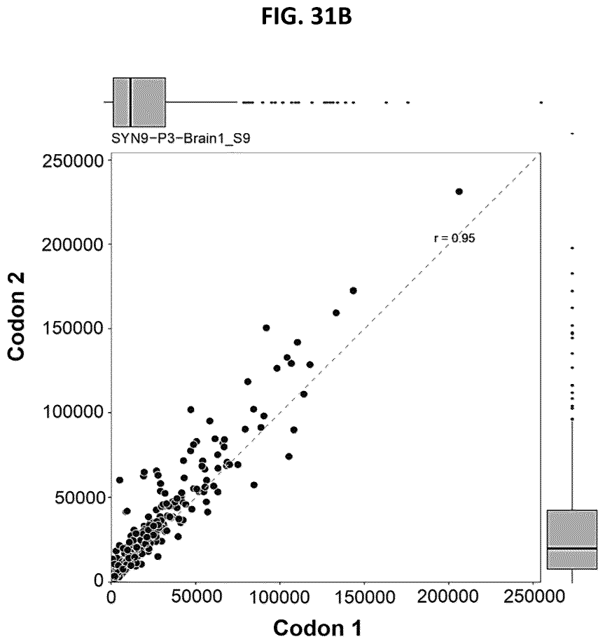

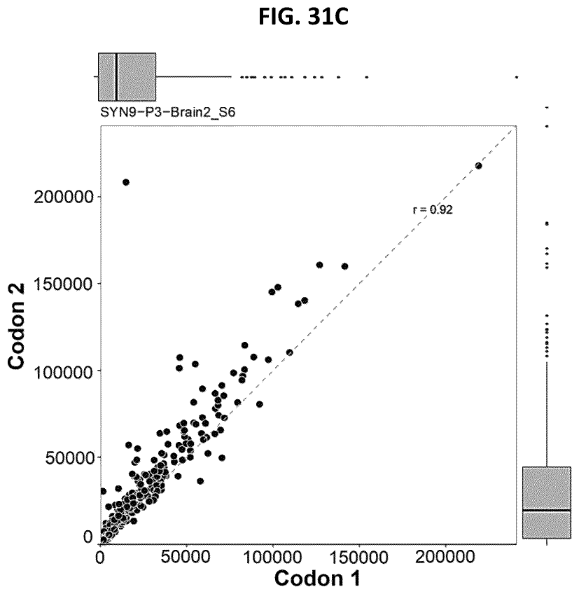

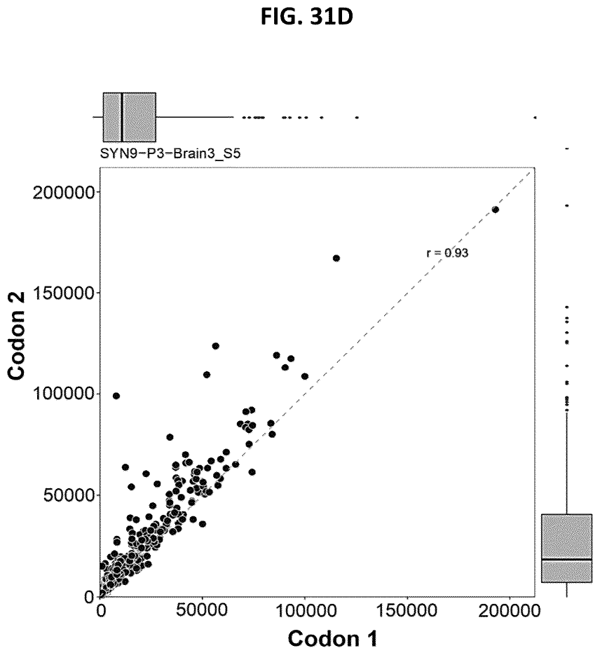

The enrichment score of each capsid was determined by NGS analysis and defined as the ratio of reads per million (RPM) in the target tissue versus RPM in the inoculum. An example of analysis performed on the control capsids is shown in FIG. 31A. As expected from the published data, the PHP.B and PHP.eB (aka, PHP.N) capsids allowed significantly higher RNA expression in neurons compared to the AAV9 parental capsid (8-fold and 25-fold, respectively). There was a very high correlation between the codon variants of each peptide species in each animal (r=0.92, 0.93 and 0.95), confirming the robustness of the NGS assay (FIG. 31B-FIG. 31D).

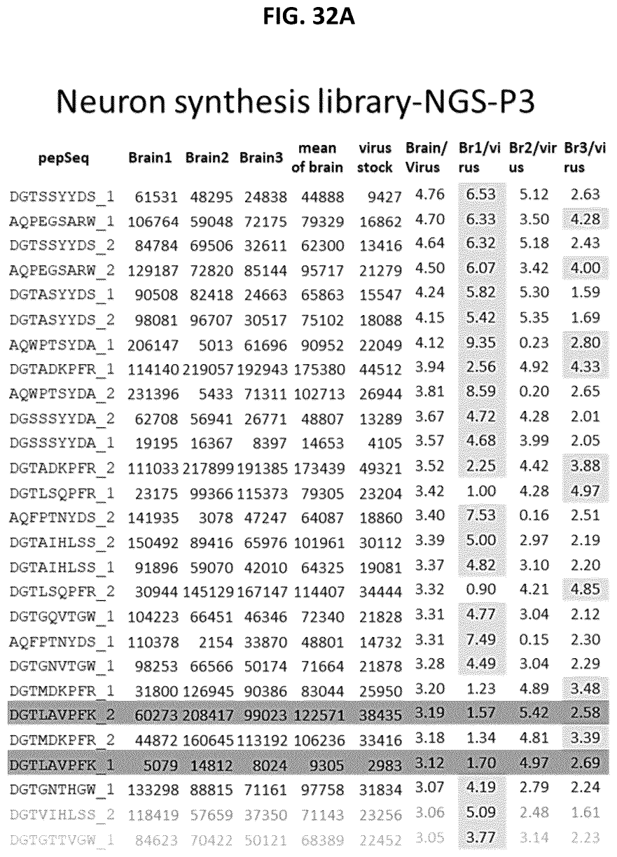

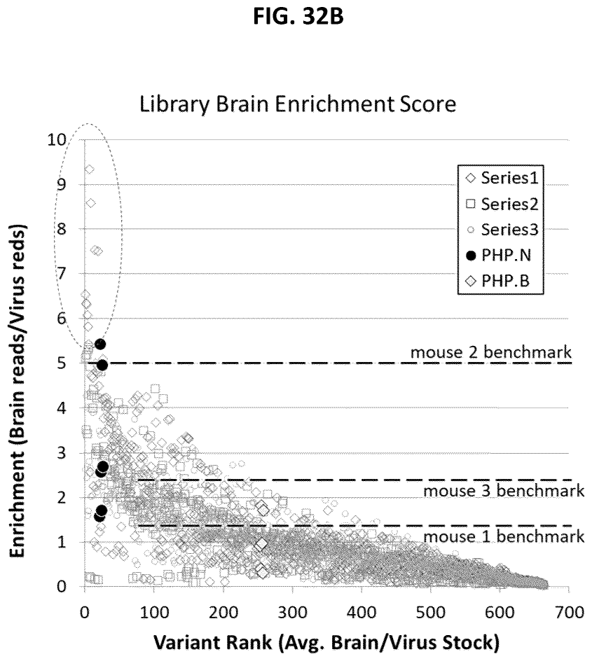

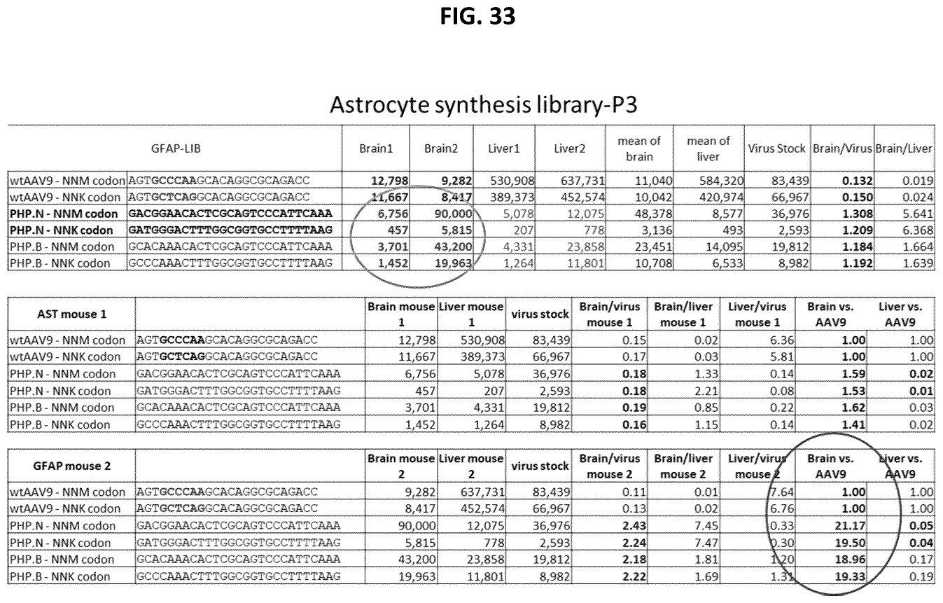

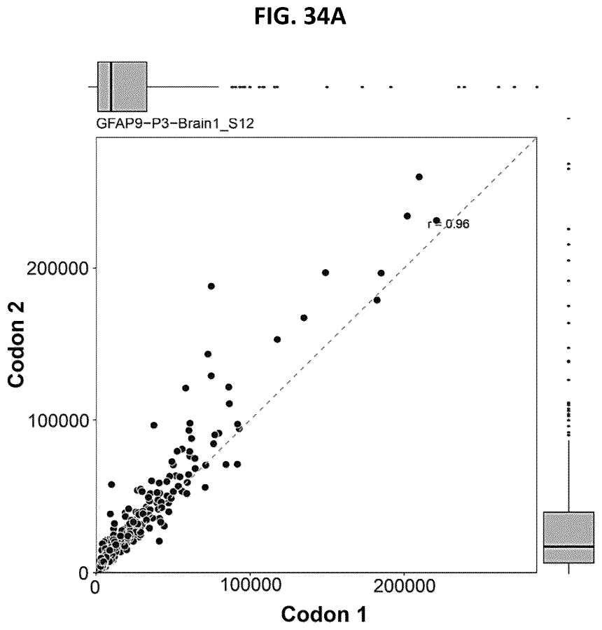

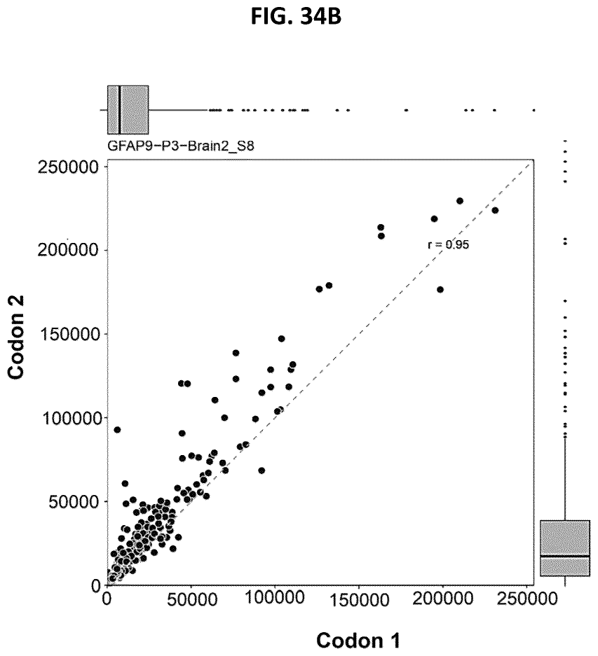

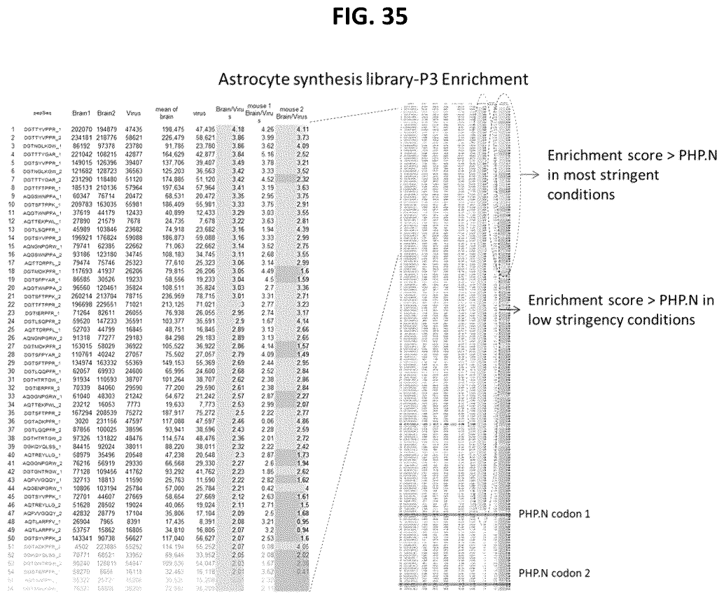

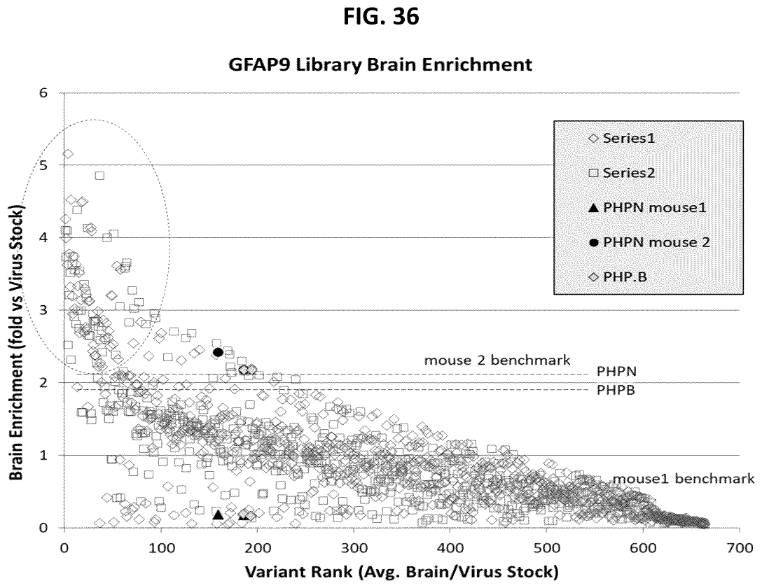

An example of enrichment analysis is presented in FIG. 32A-FIG. 36. The 333 capsid variants are ranked by average brain enrichment score from all animals, and the individual enrichment values are indicated by a color scale. As indicated by the position of the reference capsids, a group of novel variants showed a higher enrichment score than the PHP.eB benchmark capsid in both neurons (Syn-driven) and astrocytes (GFAP-driven). Interestingly, many variants showed a different enrichment score in neurons vs. astrocytes, as indicated by the medium level of correlation between Syn- and GFAP-driven RNA. This suggests that certain capsids display an enhanced tropism for neurons, and others for astrocytes (FIG. 33).

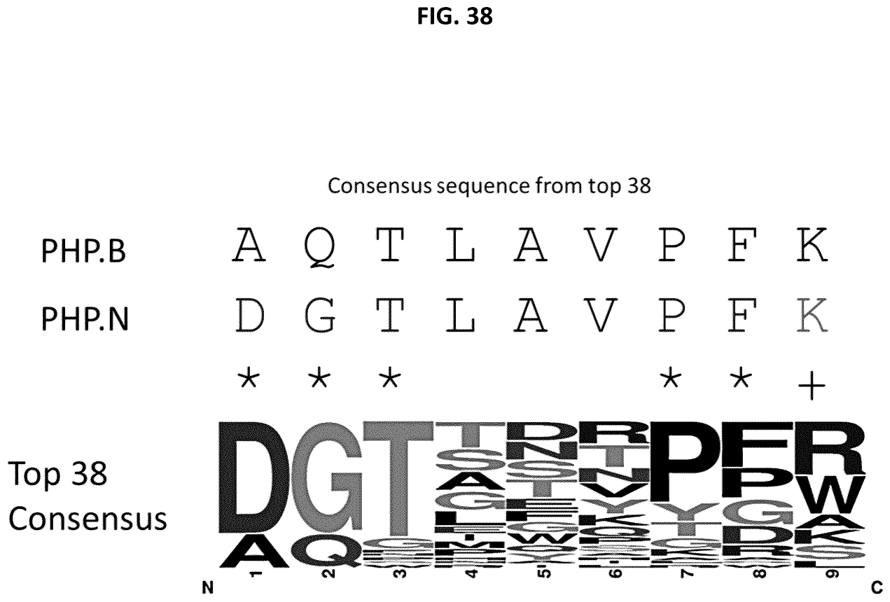

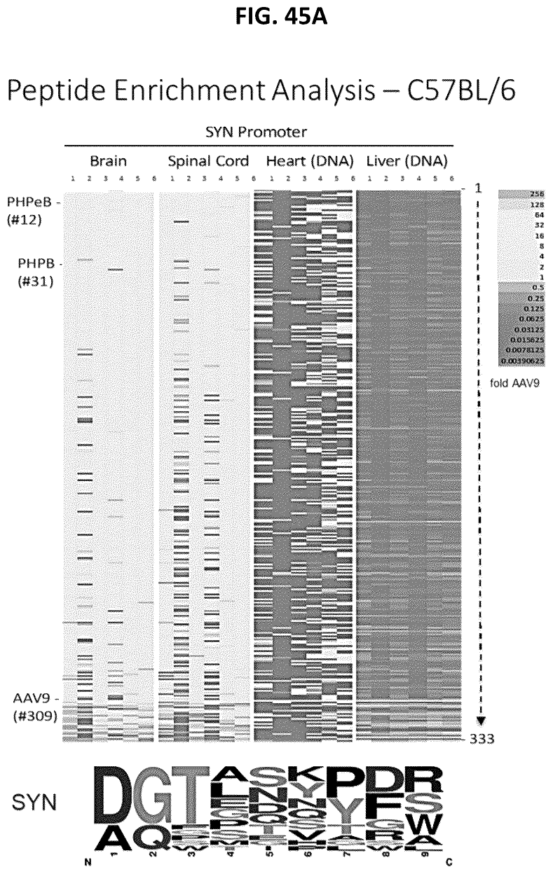

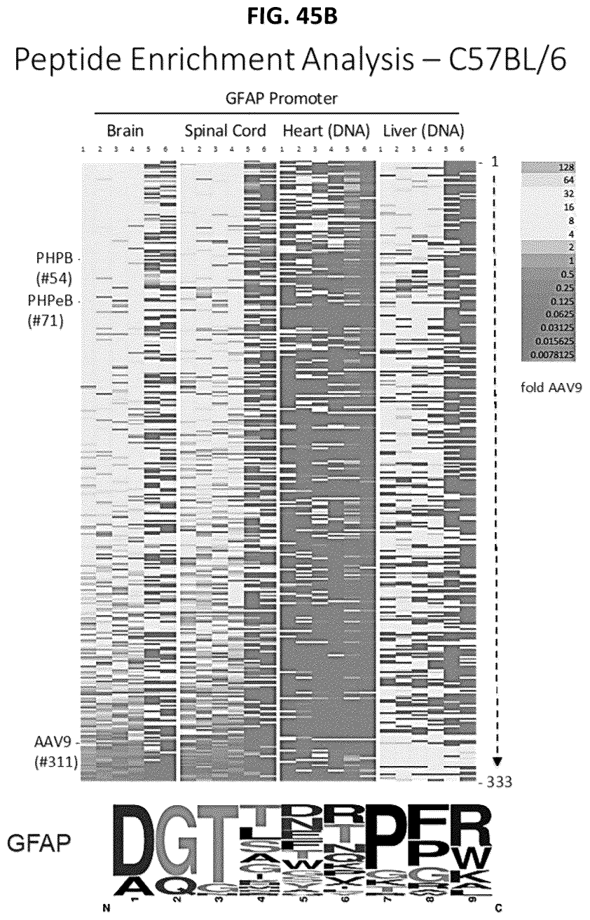

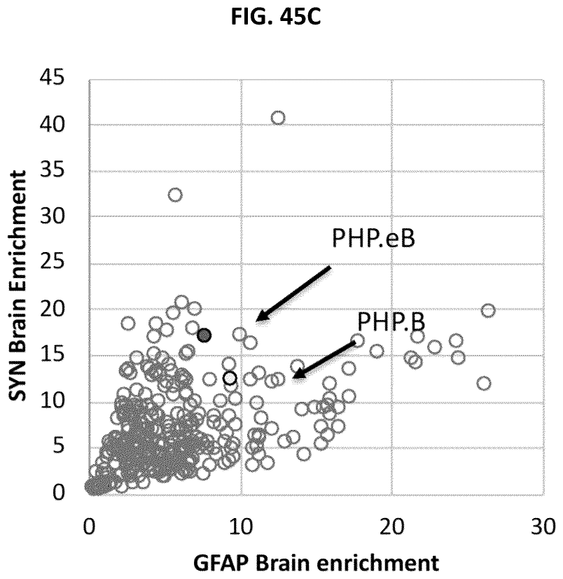

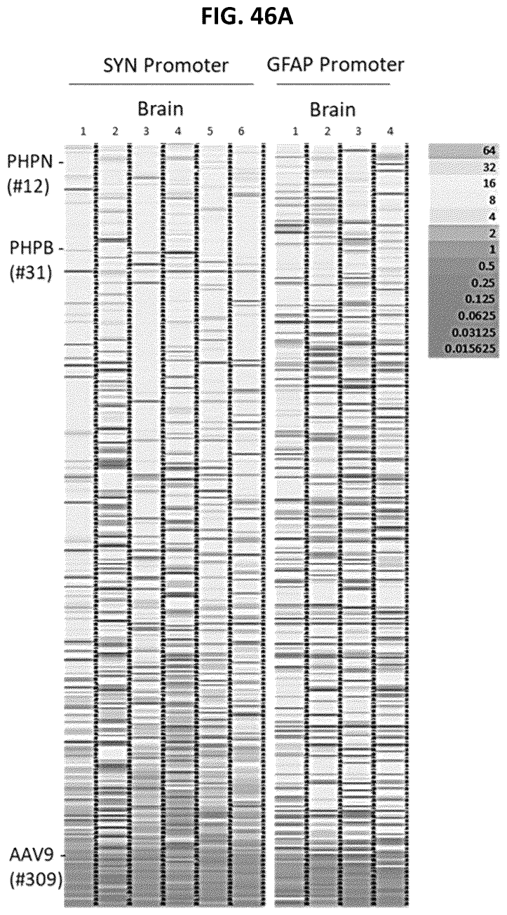

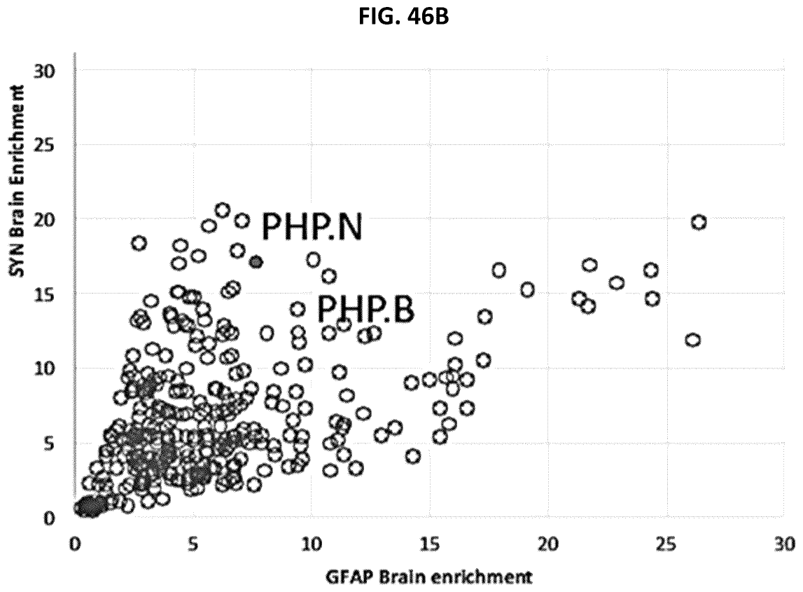

A group of 38 capsids showed potentially interesting properties based on their tropism for neurons, astrocytes or both (Table 8A and Table 8B) (FIG. 38) and showed a strong consensus peptide sequence similarity, different between neuron- and astrocyte-targeting variants (FIG. 45A-FIG. 45C and FIG. 46A-FIG. 46B).

TABLE 8A
TOP 38 candidates from C57BL/6 screen #1 (N = 3)

| | | | SEQ ID | SYN | GFAP |
|---|---|---|---|---|---|
| Groups | variant | peptide | NO: | ranking | ranking |
| A | 9p32 | DGTAIHLSS | 67 | 15, 16 | 113, 133 |
| B | 9p35 | DGTSSYYDS | 59 | 1, 3 | 565, 581 |
| | 9p36 | DGSSSYYDA | 64 | 10, 11 | 591, 594 |
| | 9p37 | DGTASYYDS | 61 | 5, 6 | 553, 560 |
| C | 9p26 | DGTTTYGAR | 77 | 225, 262 | 49, 56 |
| D | 9p2 | AQNGNPGRW | 84 | 156, 160 | 38, 44 |
| | 9p13 | AQGENPGRW | 96 | 77, 87 | 7, 13 |
| | 9p30 | AQPEGSARW | 60 | 2, 4 | 154, 160 |
| E | 9p1 | AQGSWNPPA | 80 | 348, 361 | 8, 15 |
| | 9p14 | AQGTWNPPA | 82 | 448, 467 | 14, 17 |
| F | 9p29 | AQFPTNYDS | 66 | 14, 19 | 490, 537 |
| | 9p31 | AQWPTSYDA | 62 | 7, 9 | 290, 304 |
| G | 9p3 | AQTTEKPWL | 83 | 53, 72 | 35, 70 |
| | 9p15 | AQTTDRPFL | 85 | 206, 219 | 26, 43 |
| H | 9p10 | DGTRTTTGW | 106 | 161, 220 | 10, 22 |
| | 9p18 | DGTGGIKGW | 131 | 346, 388 | 41, 68 |
| | 9p19 | DGTGNTRGW | 94 | 322, 340 | 45, 54 |
| | 9p20 | DGTHTRTGW | 90 | 380, 427 | 31, 39 |
| | 9p23 | DGTNGLKGW | 76 | 132, 153 | 5, 16 |
| | 9p33 | DGTGQVTGW | 68 | 18, 33 | 172, 213 |
| | 9p38 | DGTGNVTGW | 69 | 20, 31 | 117, 137 |
| I | 9p11 | DGTTFTPPR | 79 | 183, 199 | 11, 19 |
| | 9p12 | DGTTYVPPR | 75 | 146, 154 | 4, 9 |
| | 9p24 | DGTSFTPPK | 81 | 210, 243 | 29, 40 |
| | 9p25 | DGTSFTPPR | 88 | 250, 273 | 28, 37 |
| | 9p27 | DGTTWTPPR | 139 | 567, 570 | 46, 59 |
| | 9p28 | DGTSYVPPR | 78 | 162, 179 | 20, 25 |
| J | 9p4 | DGTADRPFR | 155 | 109, 118 | 48, 57 |
| | 9p9 | DGTMDRPFK | 102 | 102, 113 | 23 ,34 |
| | 9p16 | DGTADKPFR | 63 | 8, 12 | 1, 6 |
| | 9p17 | DGTAERPFR | 140 | 106, 138 | 42, 50 |
| | 9p21 | DGTIERPFR | 87 | 186, 235 | 21, 33 |
| | 9p34 | DGTMDKPFR | 70 | 21, 23 | 107, 112 |
| K | 9p5 | DGTISQPFK | 105 | 184, 193 | 12, 18 |
| | 9p6 | DGTLAAPFK | 120 | 110, 112 | 27, 30 |
| | 9p7 | DGTLQQPFR | 89 | 46, 57 | 32, 47 |
| | 9p8 | DGTLSQPFR | 65 | 13, 17 | 2, 3 |
| | 9p22 | DGTLNNPFR | 109 | 30, 41 | 24, 36 |
| Ref. | PHPN | DGTLAVPFK | 71 | 22, 24 | 51, 60 |
| | PHPB | AQTLAVPFK | 168 | 253, 261 | 61, 62 |
| | wtAAV9 | AQ | | 630, 631 | 611, 620 |

TABLE 8B
Variant 9 mer and encoding sequences

| | | SEQ | NNK | SEQ | NNM | SEQ |
|---|---|---|---|---|---|---|
| | 9 mer | ID | nucleotide | ID | nucleotide | ID |
| variant | peptide | NO: | sequences | NO: | sequences | NO: |
| 9p1 | AQGSWNPPA | 80 | GCCCAAGGTT | 1105 | GCACAAGGAAG | 1143 |
| | | | CGTGGAATCC | | CTGGAACCCACC | |
| | | | GCCGGCG | | AGCA | |
| 9p2 | AQNGNPGRW | 84 | GCCCAAAATG | 1106 | GCACAAAACGG | 1144 |
| | | | GTAATCCGGG | | AAACCCAGGAA | |
| | | | GCGGTGG | | GATGG | |
| 9p3 | AQTIEKPWL | 83 | GCCCAAACGA | 1107 | GCACAAACAAC | 1145 |
| | | | CTGAGAAGCC | | AGAAAAACCAT | |
| | | | GTGGCTG | | GGCTC | |
| 9p4 | DGTADRPFR | 155 | GATGGCACGG | 1108 | GACGGAACAGC | 1146 |
| | | | CGGATCGTCCT | | AGACAGACCATT | |
| | | | TTTCGG | | CAGA | |
| 9p5 | DGTISQPFK | 105 | GATGGCACCA | 1109 | GACGGAACAAT | 1147 |
| | | | TTTCGCAGCCT | | CAGCCAACCATT | |
| | | | TTTAAG | | CAAA | |
| 9p6 | DGTLAAPFK | 120 | GATGGCACCTT | 1110 | GACGGAACACTC | 1148 |
| | | | GGCTGCTCCTT | | GCAGCACCATTC | |
| | | | TTAAG | | AAA | |
| 9p7 | DGTLQQPFR | 89 | GATGGCACCTT | 1111 | GACGGAACACTC | 1149 |
| | | | GCAGCAGCCG | | CAACAACCATTC | |
| | | | TTTCGG | | AGA | |
| 9p8 | DGTLSQPFR | 65 | GATGGCACCC | 1112 | GACGGAACACTC | 1150 |
| | | | TGTCTCAGCCT | | AGCCAACCATTC | |
| | | | TTTAGG | | AGA | |
| 9p9 | DGTMDRPFK | 102 | GATGGCACCA | 1113 | GACGGAACAAT | 1151 |
| | | | TGGATAGGCC | | GGACAGACCATT | |
| | | | GTTTAAG | | CAAA | |
| 9p10 | DGTRTTTGW | 106 | GATGGCACCC | 1114 | GACGGAACAAG | 1152 |
| | | | GTACTACGAC | | AACAACAACAG | |
| | | | GGGTTGG | | GATGG | |
| 9p11 | DGTTFTPPR | 79 | GATGGCACCA | 1115 | GACGGAACAAC | 1153 |
| | | | CTTTTACTCCT | | ATTCACACCACC | |
| | | | CCTCGG | | AAGA | |
| 9p12 | DGTTYVPPR | 75 | GATGGCACCA | 1116 | GACGGAACAAC | 1154 |
| | | | CGTATGTTCCT | | ATACGTCCCACC | |
| | | | CCGCGG | | AAGA | |
| 9p13 | AQGENPGRW | 96 | GCCCAAGGGG | 1117 | GCACAAGGAGA | 1155 |
| | | | AGAATCCGGG | | AAACCCAGGAA | |
| | | | TAGGTGG | | GATGG | |
| 9p14 | AQGTWNPPA | 82 | GCCCAAGGTA | 1118 | GCACAAGGAAC | 1156 |
| | | | CTTGGAATCCG | | ATGGAACCCACC | |
| | | | CCGGCT | | AGCA | |
| 9p15 | AQTTDRPFL | 85 | GCCCAAACTA | 1119 | GCACAAACAAC | 1157 |
| | | | CTGATAGGCCT | | AGACAGACCATT | |
| | | | TTTTTG | | CCTC | |
| 9p16 | DGTADKPFR | 63 | GATGGCACCG | 1120 | GACGGAACAGC | 1158 |
| | | | CTGATAAGCC | | AGACAAACCATT | |
| | | | GTTTCGG | | CAGA | |
| 9p17 | DGTAERPFR | 140 | GATGGCACCG | 1121 | GACGGAACAGC | 1159 |
| | | | CGGAGAGGCC | | AGAAAGACCATT | |
| | | | TTTTAGG | | CAGA | |
| 9p18 | DGTGGIKGW | 131 | GATGGCACCG | 1122 | GACGGAACAGG | 1160 |
| | | | GTGGTATTAA | | AGGAATCAAAG | |
| | | | GGGGTGG | | GATGG | |
| 9p19 | DGTGNTRGW | 94 | GATGGCACCG | 1123 | GACGGAACAGG | 1161 |
| | | | GGAATACTCG | | AAACACAAGAG | |
| | | | GGGGTGG | | GATGG | |
| 9p20 | DGTHTRTGW | 90 | GATGGCACCC | 1124 | GACGGAACACA | 1162 |
| | | | ATACGCGGAC | | CACAAGAACAG | |
| | | | GGGTTGG | | GATGG | |
| 9p21 | DGTIERPFR | 87 | GATGGCACCA | 1125 | GACGGAACAAT | 1163 |
| | | | TTGAGCGGCCT | | CGAAAGACCATT | |
| | | | TTTCGT | | CAGA | |
| 9p22 | DGTLNNPFR | 109 | GATGGCACCTT | 1126 | GACGGAACACTC | 1164 |
| | | | GAATAATCCG | | AACAACCCATTC | |
| | | | TTTAGG | | AGA | |
| 9p23 | DGTNGLKGW | 76 | GATGGCACCA | 1127 | GACGGAACAAA | 1165 |
| | | | ATGGTCTGAA | | CGGACTCAAAG | |
| | | | GGGGTGG | | GATGG | |
| 9p24 | DGTSFTPPK | 81 | GATGGCACCT | 1128 | GACGGAACAAG | 1166 |
| | | | CGTTTACGCCG | | CTTCACACCACC | |
| | | | CCTAAG | | AAAA | |
| 9p25 | DGTSFTPPR | 88 | GATGGCACCT | 1129 | GACGGAACAAG | 1167 |
| | | | CGTTTACTCCG | | CTTCACACCACC | |
| | | | CCGCGG | | AAGA | |
| 9p26 | DGTTTYGAR | 77 | GATGGCACCA | 1130 | GACGGAACAAC | 1168 |
| | | | CTACGTATGG | | AACATACGGAG | |
| | | | GGCTCGT | | CAAGA | |
| 9p27 | DGTTWTPPR | 139 | GATGGCACCA | 1131 | GACGGAACAAC | 1169 |
| | | | CTTGGACGCC | | ATGGACACCACC | |
| | | | GCCGCGT | | AAGA | |
| 9p28 | DGTSYVPPR | 78 | GATGGCACCA | 1132 | GACGGAACAAG | 1170 |
| | | | GTTATGTTCCT | | CTACGTCCCACC | |
| | | | CCGAGG | | AAGA | |
| 9p29 | AQFPTNYDS | 66 | GCCCAATTTCC | 1133 | GCACAATTCCCA | 1171 |
| | | | TACGAATTATG | | ACAAACTACGAC | |
| | | | ATTCT | | AGC | |
| 9p30 | AQPEGSARW | 60 | GCCCAACCTG | 1134 | GCACAACCAGA | 1172 |
| | | | AGGGTAGTGC | | AGGAAGCGCAA | |
| | | | GAGGTGG | | GATGG | |
| 9p31 | AQWPTSYDA | 62 | GCCCAATGGC | 1135 | GCACAATGGCCA | 1173 |
| | | | CTACGAGTTAT | | ACAAGCTACGAC | |
| | | | GATGCT | | GCA | |
| 9p32 | DGTAIHLSS | 67 | GATGGCACCG | 1136 | GACGGAACAGC | 1174 |
| | | | CGATTCATCTT | | AATCCACCTCAG | |
| | | | TCGTCT | | CAGC | |
| 9p33 | DGTGQVTGW | 68 | GATGGCACCG | 1137 | GACGGAACAGG | 1175 |
| | | | GGCAGGTGAC | | ACAAGTCACAG | |
| | | | TGGGTGG | | GATGG | |
| 9p34 | DGTMDKPFR | 70 | GATGGCACGA | 1138 | GACGGAACAAT | 1176 |
| | | | TGGATAAGCC | | GGACAAACCATT | |
| | | | TTTTAGG | | CAGA | |
| 9p35 | DGTSSYYDS | 59 | GATGGCACCT | 1139 | GACGGAACAAG | 1177 |
| | | | CGAGTTATTAT | | CAGCTACTACGA | |
| | | | GATTCT | | CAGC | |
| 9p36 | DGSSSYYDA | 64 | GATGGCAGTA | 1140 | GACGGAAGCAG | 1178 |
| | | | GTTCTTATTAT | | CAGCTACTACGA | |
| | | | GATGCG | | CGCA | |
| 9p37 | DGTASYYDS | 61 | GATGGCACCG | 1141 | GACGGAACAGC | 1179 |
| | | | CGAGTTATTAT | | AAGCTACTACGA | |
| | | | GATTCT | | CAGC | |
| 9p38 | DGTGNVTGW | 69 | GATGGCACCG | 1142 | GACGGAACAGG | 1180 |
| | | | GTAATGTGAC | | AAACGTCACAG | |
| | | | GGGGTGG | | GATGG | |
| AAV9 | AQ | | AGTGCTCAGG | 54 | AGTGCCCAAGCA | 53 |
| | | | CACAGGCGCA | | CAGGCGCAGAC | |
| | | | GACC | | C | |
| PHPN | DGTLAVPFK | 71 | GATGGGACTTT | 56 | GACGGAACACTC | 55 |
| | | | GGCGGTGCCTT | | GCAGTCCCATTC | |
| | | | TTAAG | | AAA | |
| PHPB | AQTLAVPFK | 168 | GCCCAAACTTT | 58 | GCACAAACACTC | 57 |
| | | | GGCGGTGCCTT | | GCAGTCCCATTC | |
| | | | TTAAG | | AAA | |

### Example 11. Phylogenetic Grouping

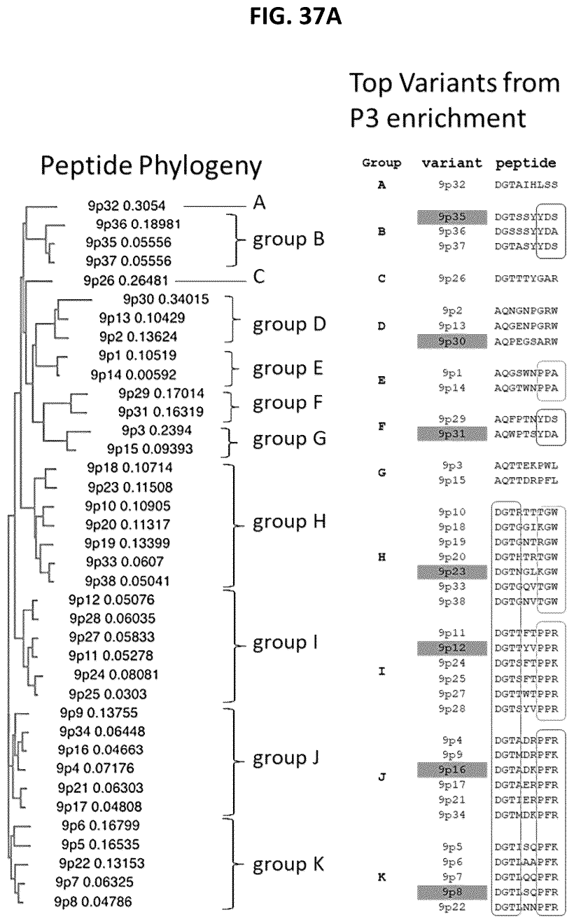

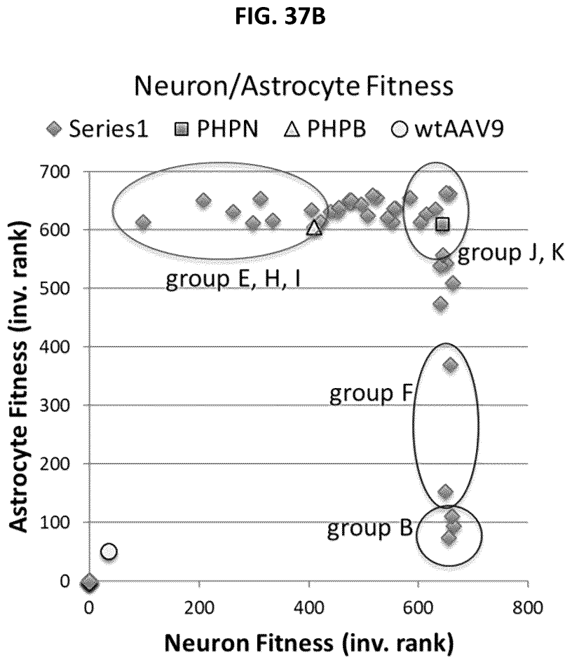

Phylogenetic grouping of peptide sequences showed an evident correlation between sequence homology clusters and capsid phenotypes (FIG. 37). For example, 9-mer variants with the sequence DGTxxxPFK/R (SEQ ID NO: 1181) presented a similar behavior as PHP.eB capsid (high transduction of both neurons and astrocytes), whereas variants harboring the sequence DGTxxxYDS/A (SEQ ID NO: 1182) showed a preference for neuron transduction. By contrast, peptides with the consensus DGTxxxxGW (SEQ ID NO: 1183) or CGTxxxPPR/K (SEQ ID NO: 1184) presented a higher tropism for astrocytes.

### Example 12. Capsid Testing

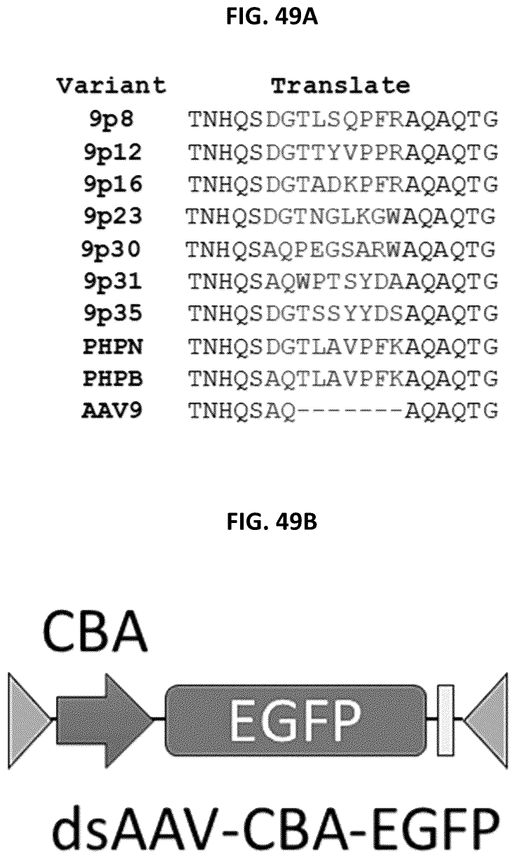

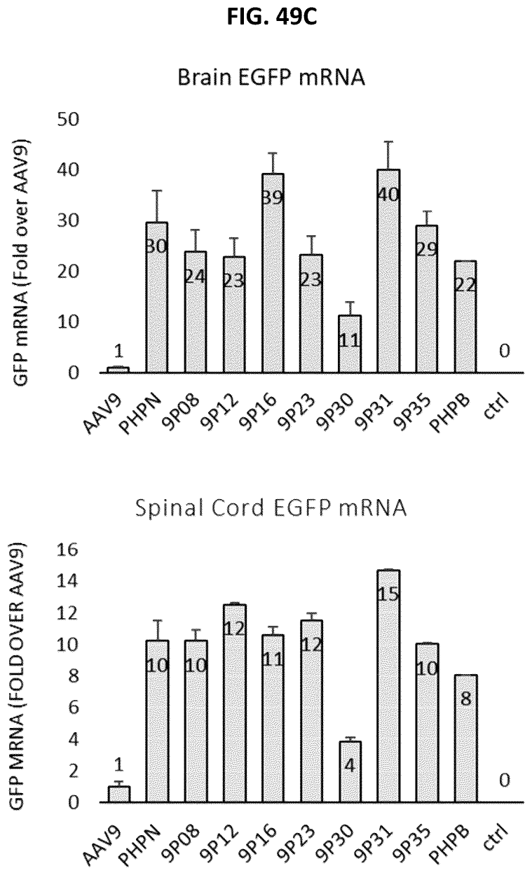

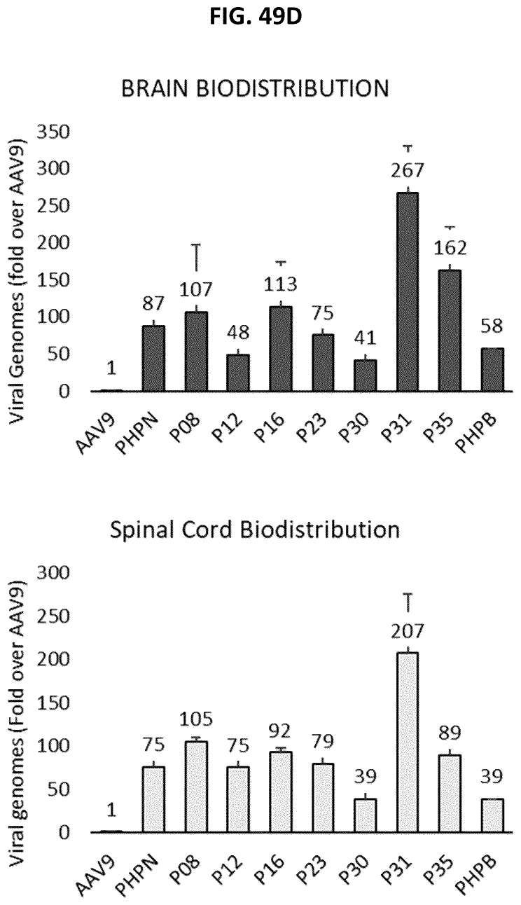

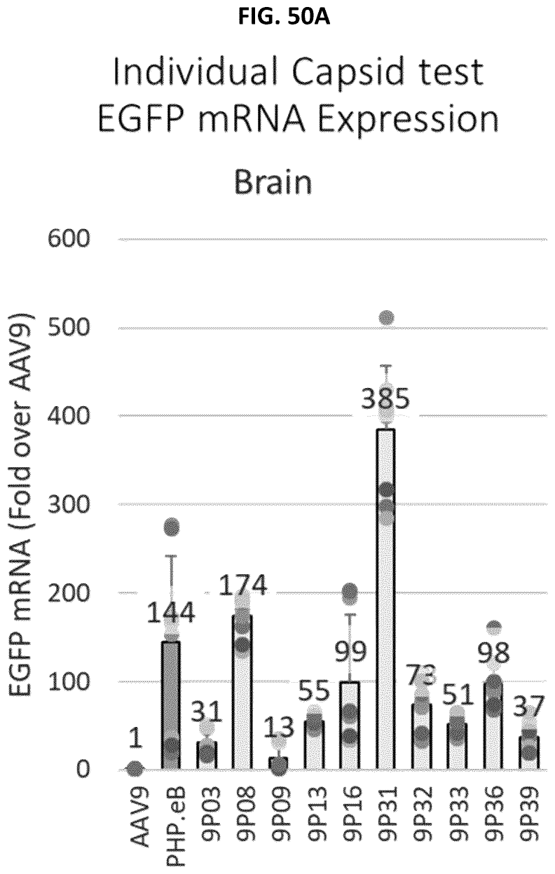

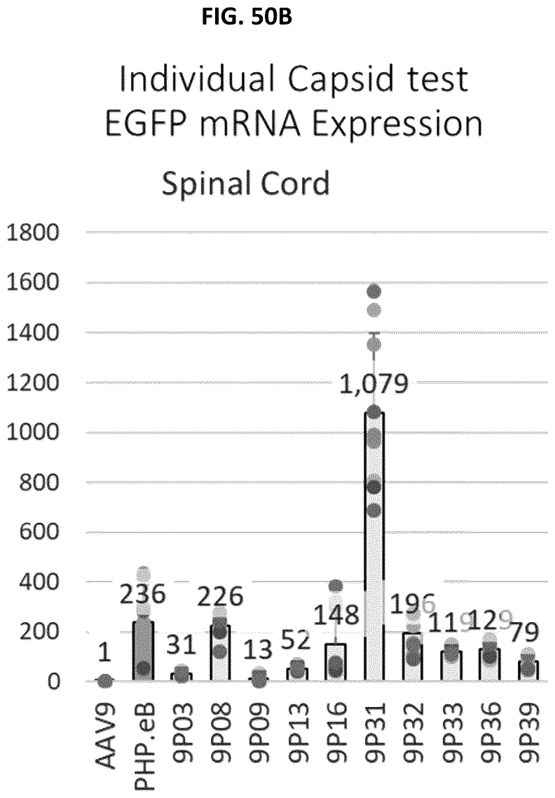

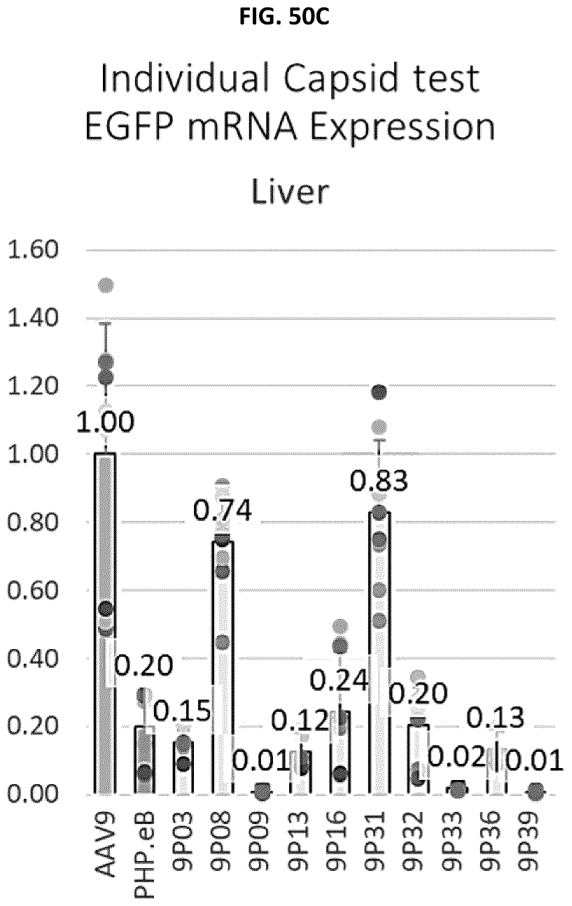

Capsid variants representative of distinct sequence clusters (highlighted in FIG. 37B) were chosen for individual transduction analysis in C57BL/6 mice. Each capsid was produced as a recombinant AAV packaging a self-complementary EGFP transgene driven by the ubiquitous promoter (FIG. 49A, B). Mouse groups (N=3) were injected intravenously with 6e10 VG and transduction efficiency was assessed after 1 month by quantifying EGFP mRNA in the brain, spinal cord, and liver tissue. EGFP mRNA expression was normalized using mouse TBP as a housekeeping gene, and DNA biodistribution was normalized to the single-copy mouse TfR gene (FIG. 50A-FIG. 50C). Reverse transcription was performed with the Quantitect kit and included a DNA removal treatment. All capsid variants showed a significant improvement in brain and spinal cord mRNA expression by comparison to the parent AAV9 capsid, and 3 out of 7 variants (9P16, 9P31 and 9P35) showed similar or higher transduction than the PHP.eB benchmark capsid (FIG. 49C, Table 10). The viral DNA biodistribution showed a very strong tropism of 9P31 and 9P35 for the brain and spinal cord, but all the variants showed a 40- to 260-fold increase of biodistribution compared to AAV9 (FIG. 49D, Table 10).

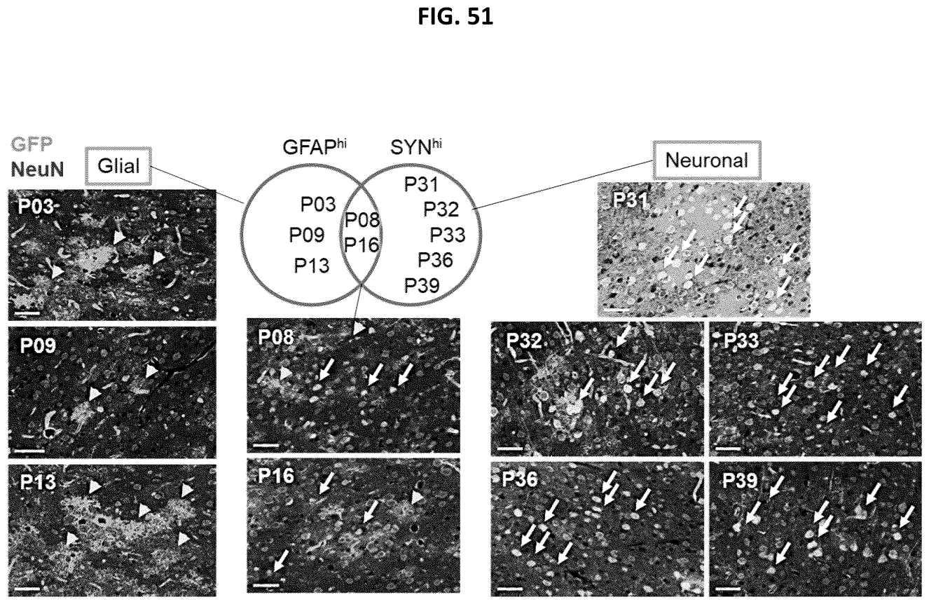

Expected cellular tropism was tested using an NGS screen by labeling the neuronal NeuN marker (FIG. 51). Within the cortex, the top capsids in the GFAP screen showed mostly GFP expression in NeuN-negative cells with glial morphology. Conversely, top capsids in the SYN screen showed a very high transduction of NeuN-positive cells, and the dual-specificity capsids 9P08 and 9P16—ranking high in both assays-showed mixed cell preference with multiple NeuN+ cells and glial cells.

Cellular tropism was also tested using mouse brain microvascular EC (mBMVEC) binding relative to AAV9. Results are shown in Table 9.

TABLE 9
mBMVEC binding results

| | | | BINDING TO |
|---|---|---|---|
| | | SEQUENCE | mBMVEC (fold |
| PEPTIDE | SEQUENCE | ID | over AAV9) |
| AAV9 | AQ | | 1 |
| PHP.eB | DGTLAVPFK | 71 | 153 |
| 9P03 | AQTTEKPWL | 83 | 170 |
| 9P08 | DGTLSQPFR | 65 | 349 |
| 9P09 | DGTMDRPFK | 102 | 222 |
| 9P13 | AQGENPGRW | 96 | 2.5 |
| 9P16 | DGTADKPFR | 63 | 176 |
| 9P31 | AQWPTSYDA | 62 | 2 |
| 9P32 | DGTAIHLSS | 67 | 16 |
| 9P33 | DGTGQVTGW | 68 | 5 |
| 9P36 | DGSSSYYDA | 64 | 0 |
| 9P39 | DGTGSTTGW | 134 | 2 |

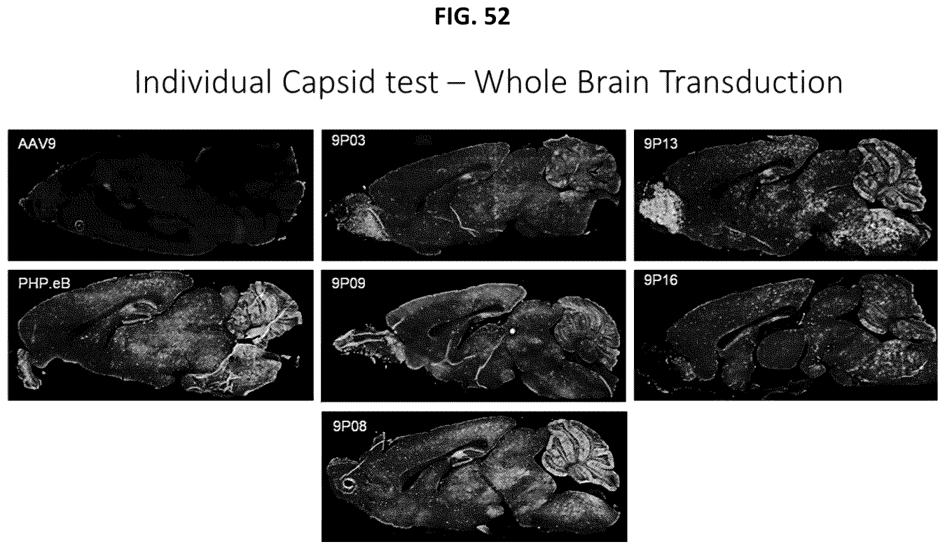

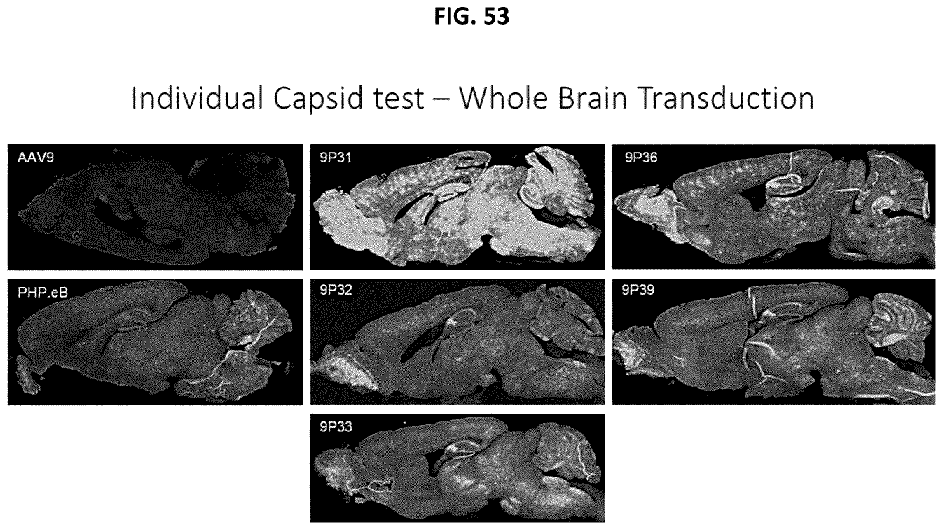

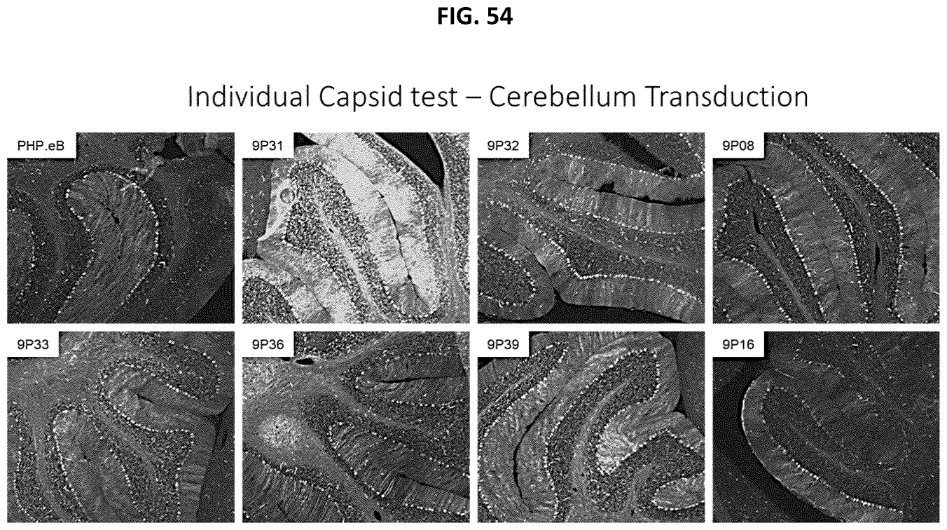

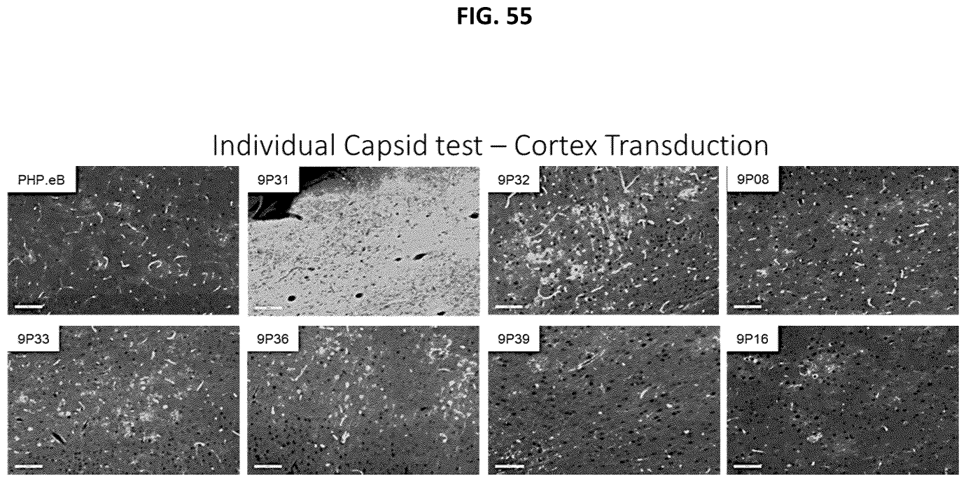

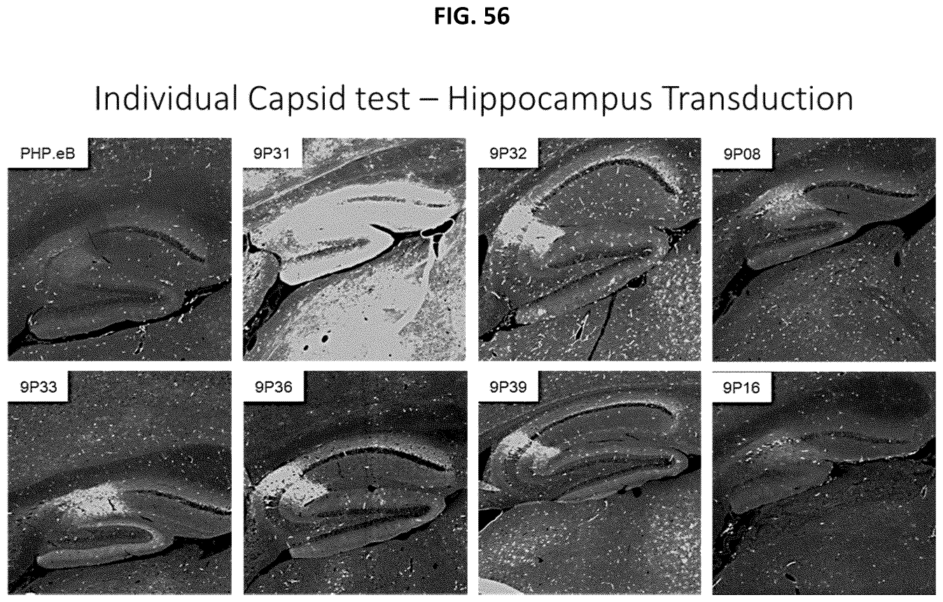

Fluorescent EGFP expression in tissues of whole brain, cerebellum, cortex, and hippocampus revealed transduction patterns across a spectrum and demonstrate the identification of tissue-specific capsids (FIG. 52-FIG. 56).

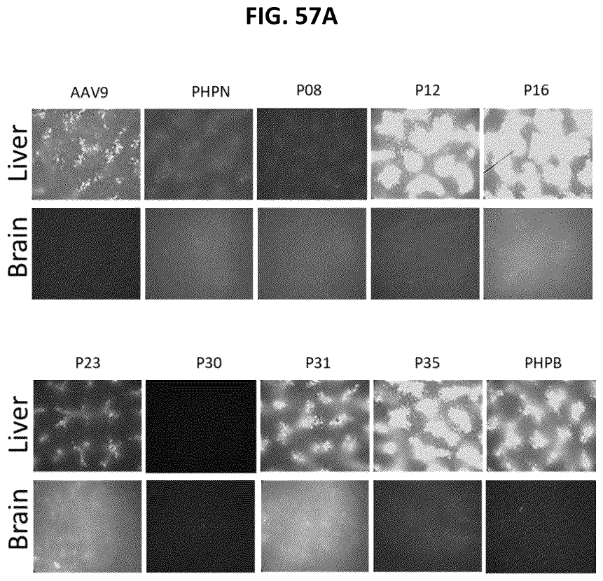

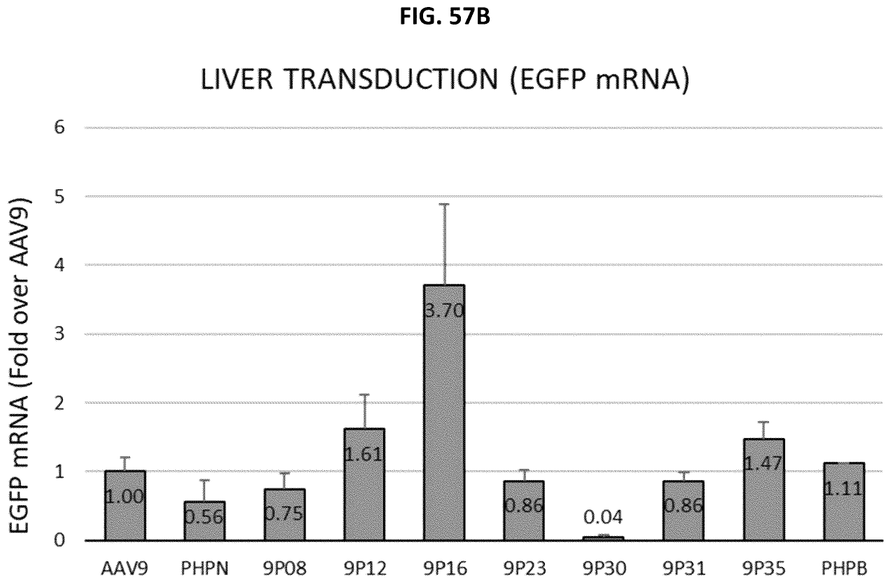

The liver transduction, measured by mRNA expression and by whole tissue GFP expression, showed that several variants outperformed AAV9, which was unexpected in light of the NGS results. Some variants, such as 9P08 or 9P23, showed a relative liver detargeting by comparison with AAV9 (FIG. 57A-FIG. 57B).

TABLE 10
Brain and Spinal cord tropism

| | BRAIN EGFP mRNA* | | | | | | |
|---|---|---|---|---|---|---|---|
| | EGFP/TBP | EGFP/TBP | EGFP/TBP | group | group | Mean Fold | Fold |
| CAPSID | m1 | m2 | m3 | mean | SD | over AAV9 | SDEV |
| AAV9 | 0.11 | 0.1 | 0.15 | 0.12 | 0.03 | 1 | 0.21 |
| PHPN | 2.94 | 4.44 | 3.42 | 3.6 | 0.77 | 30 | 6.38 |
| 9P08 | 2.46 | 3.47 | 2.73 | 2.89 | 0.53 | 24 | 4.38 |
| 9P12 | 3.07 | 2.27 | 2.98 | 2.77 | 0.44 | 23 | 3.65 |
| 9P16 | 4.31 | 4.75 | 5.28 | 4.78 | 0.49 | 39 | 4.06 |
| 9P23 | 3.28 | 2.37 | 2.79 | 2.81 | 0.46 | 23 | 3.79 |
| 9P30 | 1.06 | 1.7 | 1.32 | 1.36 | 0.32 | 11 | 2.66 |
| 9P31 | 4.87 | 5.53 | 4.2 | 4.87 | 0.66 | 40 | 5.54 |
| 9P35 | 3.9 | 3.24 | 3.45 | 3.53 | 0.33 | 29 | 2.78 |
| PHPB*** | 2.68 | 2.68 | 2.68 | 2.68 | 0 | 22 | 0 |
| ctrl | 0 | 0 | 0 | 0 | 0 | 0 | 0 |

| | SPINAL CORD EGFP mRNA* | | | | | | |
|---|---|---|---|---|---|---|---|
| | EGFP/TBP | EGFP/TBP | EGFP/TBP | group | group | Mean Fold | Fold |
| CAPSID | m1 | m2 | m3 | mean | SD | over AAV9 | SD |
| AAV9 | 0.84 | 0.29 | 0.3 | 0.48 | 0.31 | 1 | 0.66 |
| PHPN | 3.36 | 5.8 | 5.4 | 4.86 | 1.31 | 10.22 | 2.75 |
| 9P08 | 4.3 | 5.62 | 4.65 | 4.86 | 0.68 | 10.22 | 1.43 |
| 9P12 | 6.09 | 5.94 | 5.78 | 5.94 | 0.16 | 12.49 | 0.33 |
| 9P16 | 4.42 | 5.31 | 5.37 | 5.04 | 0.53 | 10.6 | 1.12 |
| 9P23 | 5.41 | 5.95 | 5.04 | 5.47 | 0.46 | 11.5 | 0.96 |
| 9P30 | 1.53 | 1.83 | 2.11 | 1.82 | 0.29 | 3.84 | 0.61 |
| 9P31 | 6.92 | 7.06 | 6.94 | 6.98 | 0.08 | 14.68 | 0.16 |
| 9P35 | 4.68 | 4.81 | 4.79 | 4.76 | 0.07 | 10.02 | 0.15 |
| PHPB | 3.84 | 3.84 | 3.84 | 3.84 | 0 | 8.09 | 0 |
| ctrl | 0 | 0 | 0 | 0 | 0 | 0 | 0 |

| | BRAIN EGFP DNA** (VG/Cell) | | | | | | |
|---|---|---|---|---|---|---|---|
| | EGFP/TERT | EGFP/TERT | EGFP/TERT | Group | Group | Mean Fold | Fold |
| CAPSID | m1 | m2 | m3 | mean | SD | over AAV9 | SDEV |
| AAV9 | 0.03 | 0.04 | 0.01 | 0.03 | 0.01 | 1 | 0 |
| PHPN | 2.07 | 2.79 | 1.94 | 2.27 | 0.46 | 87 | 18 |
| P08 | 1.25 | 1.62 | 5.47 | 2.78 | 2.34 | 107 | 90 |
| P12 | 1.43 | 0.94 | 1.41 | 1.26 | 0.27 | 48 | 10 |
| P16 | 4.13 | 1.15 | 3.56 | 2.95 | 1.58 | 113 | 60 |
| P23 | 1.34 | 2.68 | 1.87 | 1.96 | 0.68 | 75 | 26 |
| P30 | 0.59 | 1.42 | 1.21 | 1.08 | 0.43 | 41 | 17 |
| P31 | 6.47 | 5.6 | 8.81 | 6.96 | 1.66 | 267 | 64 |
| P35 | 4.62 | 5.55 | 2.52 | 4.23 | 1.55 | 162 | 59 |
| PHPB | 1.5 | 1.5 | 1.5 | 1.5 | 0 | 58 | 0 |
| ctrl | 0 | 0 | 0 | 0 | 0 | 0 | 0 |
| | SPINAL CORD EGFP DNA** (VG/Cell) | | | | | | |
| | EGFP/TERT | EGFP/TERT | EGFP/TERT | Group | Group | Mean Fold | Fold |
| CAPSID | m 1 | m 2 | m 3 | AVG | SD | over AAV9 | SDEV |
| AAV9 | 0.03 | 0.04 | 0.04 | 0.03 | 0.007 | 1 | 0.2 |
| PHPN | 1.75 | 2.96 | 3.14 | 2.62 | 0.752 | 75 | 21.7 |
| P08 | 3.81 | 3.47 | 3.66 | 3.65 | 0.174 | 105 | 5 |
| P12 | 1.62 | 3.31 | 2.87 | 2.6 | 0.873 | 75 | 25.2 |
| P16 | 3.3 | 3.34 | 2.96 | 3.2 | 0.211 | 92 | 6.1 |
| P23 | 2.63 | 2.47 | 3.1 | 2.73 | 0.322 | 79 | 9.3 |
| P30 | 0.8 | 1.8 | 1.43 | 1.34 | 0.507 | 39 | 14.6 |
| P31 | 9.88 | 6.19 | 5.47 | 7.18 | 2.366 | 207 | 68.2 |
| P35 | 2.95 | 3.92 | 2.41 | 3.1 | 0.765 | 89 | 22 |
| PHPB | 1.34 | 1.34 | 1.34 | 1.34 | 0 | 39 | 0 |
| ctrl | 0 | 0 | 0 | 0 | 0 | 0 | 0 |

*EGFP mRNA expression was normalized to TBP as a housekeeping marker
**GFP DNA was normalized to single-copy TfR DNA
***N = 1

### Example 13. Multi-Rodent Testing (Cross Species)

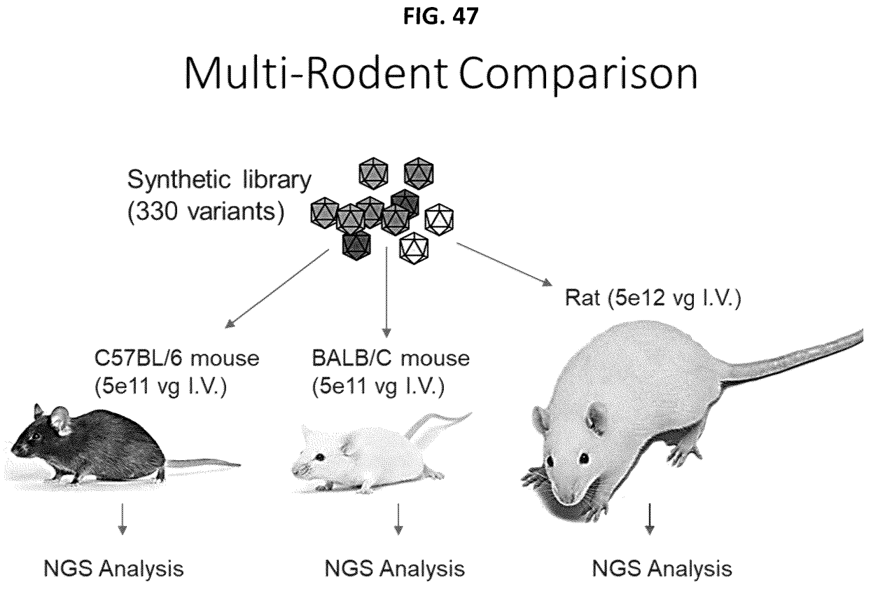

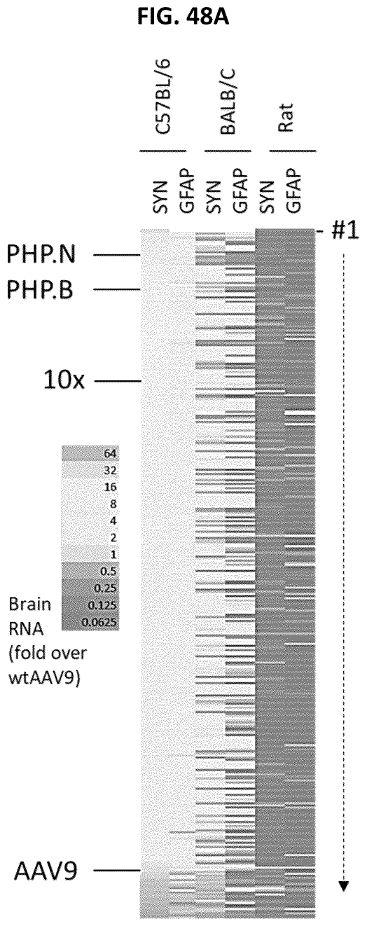

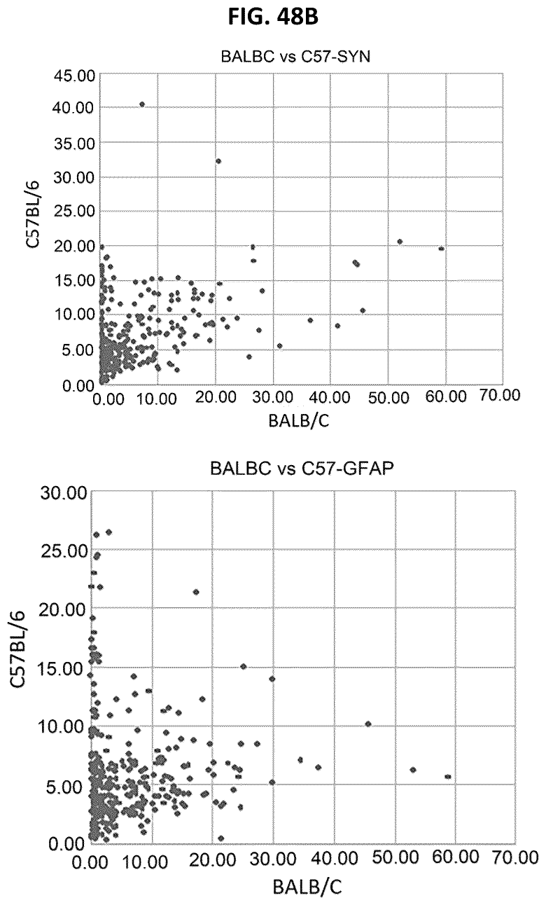

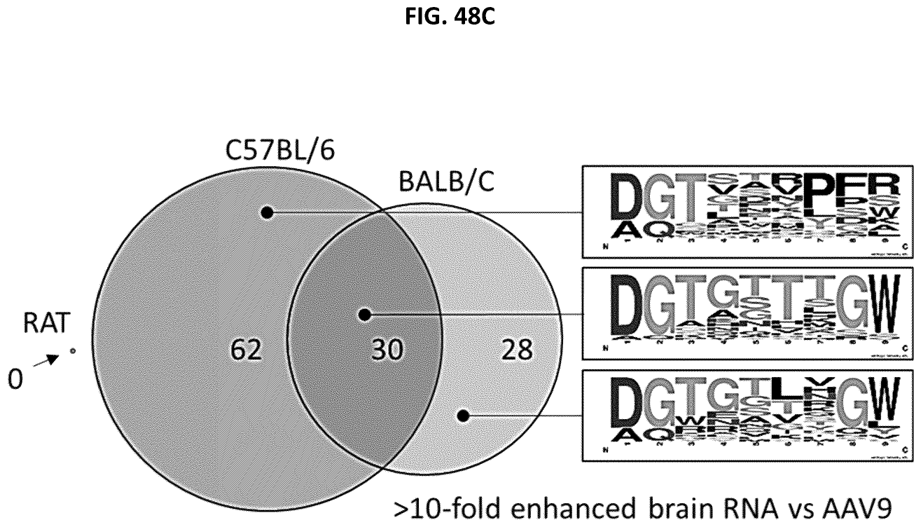

The efficacy of the 333 capsid variants to transduce CNS was tested in other rodent strains or species (FIG. 47). Side-by-side comparison of neuron and astrocyte transduction in C57BL/6 mice, BALB/C mice and rats showed major differences in the enrichment scores of multiple variants between the two mouse strains, and even more pronounced differences between mice and rats (FIG. 48A-FIG. 48C). Strikingly, the most efficient capsid for rat brain transduction was the parental AAV9, which suggests that directed evolution “bottlenecks” capsid variants that are highly performant in one given species, as opposed to the versatility of wild-type AAV capsids.

Correlation analysis showed that some capsids shared high CNS transduction between C57BL/6 and BALB/C mice, whereas others were restricted to only one strain (FIG. 48B).

Interestingly, the PHP.B and PHP.eB capsid showed poor brain transduction in BALB/C mice, in line with a recent publication (Hordeaux et al., 2018). When focusing on the capsids that showed >10-fold increase in brain transduction, 62 variants were improved only in C57BL/6 mice, 28 variants were improved only in BALB/C mice and 30 variants showed improved brain transduction in both strains (Table 11). Consensus sequence analysis showed a “C57BL/6 signature” closely resembling the PHP.eB peptide (DGTxxxPFR (SEQ ID NO: 1185)) whereas the BALB/C signature showed a different consensus (DGTxxxxGW (SEQ ID NO: 1183)), suggesting the use of a different cellular receptor (FIG. 48C).

TABLE 11
TOP 30 candidates from C57BL/6 and BALB/C mouse screen
SYNAPSIN PROMOTER

| C57BL/6 | | | BALB/C | | |
|---|---|---|---|---|---|
| REPLICATE 1 (N = 3) | | REPLICATE 2 (N = 6) | | REPLICATE 1 (N = 6) | |
| | Brain | | Brain | | Brain |
| | Enrichment | | Enrichment | | Enrichment |
| 9-mer peptide | Factor (fold | 9-mer peptide | Factor (fold | 9-mer peptide | Factor (fold |
| insert | over AAV9) | insert | over AAV9) | insert | over AAV9) |
| DGTSSYYDS | 36.40 | AQWPTSYDA | 39.97 | DGTGSTTGW | 57.05 |
| (SEQ ID NO: | | (SEQ ID NO: | | (SEQ ID NO: | |
| 59) | | 62) | | 134) | |
| AQPEGSARW | 35.95 | AQPEGSARW | 31.83 | DGTGQVTGW | 49.87 |
| (SEQ ID NO: | | (SEQ ID NO: | | (SEQ ID NO: | |
| 60) | | 60) | | 68) | |
| DGTASYYDS | 32.34 | DGTGQVTGW | 20.35 | DGTGSTHGW | 43.08 |
| (SEQ ID NO: | | (SEQ ID NO: | | (SEQ ID NO: | |
| 61) | | 68) | | 119) | |
| AQWPTSYDA | 30.81 | DGTAIHLSS | 19.55 | DGTGSTQGW | 38.31 |
| (SEQ ID NO: | | (SEQ ID NO: | | (SEQ ID NO: | |
| 62) | | 67) | | 315) | |
| DGTADKPFR | 29.30 | DGTMDRPFK | 19.48 | DGTGTTTGW | 37.29 |
| (SEQ ID NO: | | (SEQ ID NO: | | (SEQ ID NO: | |
| 63) | | 102) | | 130) | |
| DGSSSYYDA | 28.05 | DGTGSTTGW | 19.20 | AQWAAGYNV | 34.57 |
| (SEQ ID NO: | | (SEQ ID NO: | | (SEQ ID NO: | |
| 64) | | 134) | | 245) | |
| DGTLSQPFR | 26.73 | DGSSSYYDA | 18.08 | DGTGGTKGW | 33.59 |
| (SEQ ID NO: | | (SEQ ID NO: | | (SEQ ID NO: | |
| 65) | | 64) | | 107) | |
| DGTAIHLSS | 26.23 | DGTSSYYDA | 17.93 | DGTGSTKGW | 29.64 |
| (SEQ ID NO: | | (SEQ ID NO: | | (SEQ ID NO: | |
| 67) | | 381) | | 313) | |
| AQFPTNYDS | 26.07 | DGSQSTTGW | 17.59 | DGSQSTTGW | 25.19 |
| (SEQ ID NO: | | (SEQ ID NO: | | (SEQ ID NO: | |
| 66) | | 136) | | 136) | |
| DGTMDKPFR | 25.05 | DGTGSTQGW | 17.24 | AQWEVKGGY | 23.44 |
| (SEQ ID NO: | | (SEQ ID NO: | | (SEQ ID NO: | |
| 70) | | 315) | | 247) | |
| DGTLAVPFK | 24.62 | DGTGTTTGW | 17.00 | DGTAIHLSS | 22.81 |
| (SEQ ID NO: | | (SEQ ID NO: | | (SEQ ID NO: | |
| 71) | | 130) | | 67) | |
| DGTGNVTGW | 24.05 | DGTLAVPFK | 16.84 | DGGGTTTGW | 22.62 |
| (SEQ ID NO: | | (SEQ ID NO: | | (SEQ ID NO: | |
| 69) | | 71) | | 270) | |
| DGTGQVTGW | 23.83 | DGTASYYDS | 16.68 | DGTGGLTGW | 22.42 |
| (SEQ ID NO: | | (SEQ ID NO: | | (SEQ ID NO: | |
| 68) | | 61) | | 294) | |
| DGTHIHLSS | 22.93 | DGTMDKPFR | 16.68 | DGTNTINGW | 20.76 |
| (SEQ ID NO: | | (SEQ ID NO: | | (SEQ ID NO: | |
| 162) | | 70) | | 124) | |
| DGTGNTHGW | 22.63 | DGTVANPFR | 16.32 | DGAGGTSGW | 19.55 |
| (SEQ ID NO: | | (SEQ ID NO: | | (SEQ ID NO: | |
| 72) | | 394) | | 151) | |
| DGTVIHLSS | 22.62 | DGTLNNPFR | 16.24 | DGTNTTHGW | 18.99 |
| (SEQ ID NO: | | (SEQ ID NO: | | (SEQ ID NO: | |
| 73) | | 109) | | 113) | |
| DGTLNNPFR | 22.33 | DGTLAAPFK | 15.96 | DGTGTVQGW | 18.84 |
| (SEQ ID NO: | | (SEQ ID NO: | | (SEQ ID NO: | |
| 109) | | 120) | | 327) | |
| DGTGNTSGW | 22.10 | DGTLSQPFR | 15.43 | DGTGQTIGW | 18.55 |
| (SEQ ID NO: | | (SEQ ID NO: | | (SEQ ID NO: | |
| 137) | | 65) | | 305) | |
| DGTGTTVGW | 21.72 | DGTHIHLSS | 15.11 | AQWELSNGY | 18.13 |
| (SEQ ID NO: | | (SEQ ID NO: | | (SEQ ID NO: | |
| 74) | | 162) | | 246) | |
| DGTSSYYDA | 20.94 | AQTTEKPWL | 15.00 | DGTGSLNGW | 17.93 |
| (SEQ ID NO: | | (SEQ ID NO: | | (SEQ ID NO: | |
| 381) | | 83) | | 309) | |
| DGAGTTSGW | 20.42 | DGTGNVTGW | 14.90 | DGTGTTLGW | 17.48 |
| (SEQ ID NO: | | (SEQ ID NO: | | (SEQ ID NO: | |
| 265) | | 69) | | 323) | |
| DGGGTTTGW | 20.27 | DGTGGVTGW | 14.89 | AQPEGSARW | 17.11 |
| (SEQ ID NO: | | (SEQ ID NO: | | (SEQ ID NO: | |
| 270) | | 299) | | 60) | |
| DGTLQQPFR | 19.88 | DGTSSYYDS | 14.80 | DGTGSTMGW | 16.91 |
| (SEQ ID NO: | | (SEQ ID NO: | | (SEQ ID NO: | |
| 89) | | 59) | | 314) | |
| DGTGQTIGW | 19.52 | DGTGNTSGW | 14.48 | DGTGNTHGW | 16.47 |
| (SEQ ID NO: | | (SEQ ID NO: | | (SEQ ID NO: | |
| 305) | | 137) | | 72) | |
| DGTVTTTGW | 19.49 | AQWPTAYDA | 14.48 | DGSGTTRGW | 15.83 |
| (SEQ ID NO: | | (SEQ ID NO: | | (SEQ ID NO: | |
| 405) | | 256) | | 114) | |
| DGTSIHLSS | 19.45 | AQGENPGRW | 14.41 | DGTNSTTGW | 15.48 |
| (SEQ ID NO: | | (SEQ ID NO: | | (SEQ ID NO: | |
| 378) | | 96) | | 143) | |
| DGTGSTTGW | 19.45 | DGTADKPFR | 14.32 | DGRNALTGW | 15.13 |
| (SEQ ID NO: | | (SEQ ID NO: | | (SEQ ID NO: | |
| 134) | | 63) | | 275) | |
| DGTGGVTGW | 19.44 | DGTGQTIGW | 14.27 | DGAAATTGW | 15.02 |
| (SEQ ID NO: | | (SEQ ID NO: | | (SEQ ID NO: | |
| 299) | | 305) | | 264) | |
| DGTVANPFR | 19.42 | DGTISQPFK | 13.84 | DGTATTMGW | 14.54 |
| (SEQ ID NO: | | (SEQ ID NO: | | (SEQ ID NO: | |
| 394) | | 105) | | 284) | |
| DGTGTTTGW | 19.16 | DGTKLMLSS | 13.71 | AQRYTGDSS | 14.35 |
| (SEQ ID NO: | | (SEQ ID NO: | | (SEQ ID NO: | |
| 130) | | 157) | | 138) | |
| DGAGGTSGW | 18.99 | AQTLAVPFK | 13.69 | DGAGTTSGW | 14.29 |
| (SEQ ID NO: | | (SEQ ID NO: | | (SEQ ID NO: | |
| 151) | | 168) | | 265) | |

GFAP PROMOTER

| C57BL/6 | | | BALB/C | | |
|---|---|---|---|---|---|
| REPLICATE 1 (N = 2) | | REPLICATE 2 (N = 6) | | REPLICATE 1 (N = 6) | |
| | Brain | | Brain | | Brain |
| | Enrichment | | Enrichment | | Enrichment |
| 9-mer peptide | Factor (fold | 9-mer peptide | Factor (fold | 9-mer peptide | Factor (fold |
| insert | over AAV9) | insert | over AAV9) | insert | over AAV9) |
| DGTADKPFR | 37.60 | DGTMDRPFK | 24.89 | DGTGSTTGW | 21.03 |
| (SEQ ID NO: | | (SEQ ID NO: | | (SEQ ID NO: | |
| 63) | | 102) | | 134) | |
| DGTLSQPFR | 35.97 | DGTAERPFR | 24.66 | DGTGQVTGW | 19.24 |
| (SEQ ID NO: | | (SEQ ID NO: | | (SEQ ID NO: | |
| 65) | | 140) | | 68) | |
| DGTTYVPPR | 33.09 | DGTADKPFR | 23.03 | DGTGTTTGW | 15.56 |
| (SEQ ID NO: | | (SEQ ID NO: | | (SEQ ID NO: | |
| 75) | | 63) | | 130) | |
| DGTNGLKGW | 32.14 | DGTLNNPFR | 22.91 | DGTGSTHGW | 14.45 |
| (SEQ ID NO: | | (SEQ ID NO: | | (SEQ ID NO: | |
| 76) | | 109) | | 119) | |
| AQGENPGRW | 31.99 | DGTLSQPFR | 21.60 | DGTAIHLSS | 11.74 |
| (SEQ ID NO: | | (SEQ ID NO: | | (SEQ ID NO: | |
| 96) | | 65) | | 67) | |
| AQGSWNPPA | 30.78 | DGTMDKPFR | 20.52 | DGTGSTQGW | 11.40 |
| (SEQ ID NO: | | (SEQ ID NO: | | (SEQ ID NO: | |
| 80) | | 70) | | 315) | |
| AQGTWNPPA | 29.19 | DGTISQPFK | 20.47 | DGTGGLTGW | 8.87 |
| (SEQ ID NO: | | (SEQ ID NO: | | (SEQ ID NO: | |
| 82) | | 105) | | 294) | |
| DGTISQPFK | 29.01 | AQGENPGRW | 20.09 | AQNGNPGRW | 8.82 |
| (SEQ ID NO: | | (SEQ ID NO: | | (SEQ ID NO: | |
| 105) | | 96) | | 84) | |
| DGTTFTPPR | 28.94 | AQTTEKPWL | 18.04 | DGTGGIKGW | 8.62 |
| (SEQ ID NO: | | (SEQ ID NO: | | (SEQ ID NO: | |
| 79) | | 83) | | 131) | |
| DGTRTTTGW | 28.59 | DGTVANPFR | 16.87 | DGRNALTGW | 8.39 |
| (SEQ ID NO: | | (SEQ ID NO: | | (SEQ ID NO: | |
| 106) | | 394) | | 275) | |
| DGTSYVPPR | 26.17 | DGTTYVPPR | 16.31 | DGTGSTKGW | 8.38 |
| (SEQ ID NO: | | (SEQ ID NO: | | (SEQ ID NO: | |
| 78) | | 75) | | 313) | |
| DGTIERPFR | 25.37 | AQTTDRPFL | 16.27 | AQRYTGDSS | 8.13 |
| (SEQ ID NO: | | (SEQ ID NO: | | (SEQ ID NO: | |
| 87) | | 85) | | 138) | |
| DGTMDRPFK | 24.85 | DGTTTYGAR | 15.62 | DGTGGTKGW | 8.06 |
| (SEQ ID NO: | | (SEQ ID NO: | | (SEQ ID NO: | |
| 102) | | 77) | | 107) | |
| DGTLAAPFK | 24.67 | DGTADRPFR | 15.60 | DGTATTTGW | 8.04 |
| (SEQ ID NO: | | (SEQ ID NO: | | (SEQ ID NO: | |
| 120) | | 155) | | 285) | |
| DGTLNNPFR | 24.62 | DGTIERPFR | 15.11 | DGTKMVLQL | 7.87 |
| (SEQ ID NO: | | (SEQ ID NO: | | (SEQ ID NO: | |
| 109) | | 87) | | 142) | |
| DGTSFTPPR | 24.14 | AQGSWNPPA | 15.11 | DGTGSLNGW | 7.71 |
| (SEQ ID NO: | | (SEQ ID NO: | | (SEQ ID NO: | |
| 88) | | 80) | | 309) | |
| AQTTDRPFL | 23.85 | AQGTWNPPA | 15.03 | DGTNTINGW | 7.59 |
| (SEQ ID NO: | | (SEQ ID NO: | | (SEQ ID NO: | |
| 85) | | 82) | | 124) | |
| DGTSFTPPK | 23.75 | DGSTERPFR | 15.01 | AQWELSNGY | 7.57 |
| (SEQ ID NO: | | (SEQ ID NO: | | (SEQ ID NO: | |
| 81) | | 99) | | 246) | |
| DGTHTRTGW | 23.54 | AQSVAKPFL | 14.90 | DGTNGLKGW | 7.50 |
| (SEQ ID NO: | | (SEQ ID NO: | | (SEQ ID NO: | |
| 90) | | 231) | | 76) | |
| DGTLQQPFR | 22.94 | DGTVDRPFK | 14.74 | DGTNTTHGW | 7.25 |
| (SEQ ID NO: | | (SEQ ID NO: | | (SEQ ID NO: | |
| 89) | | 395) | | 113) | |
| AQNGNPGRW | 22.80 | DGTTFTPPR | 14.56 | DGTRMVVQL | 7.25 |
| (SEQ ID NO: | | (SEQ ID NO: | | (SEQ ID NO: | |
| 84) | | 79) | | 370) | |
| DGTAERPFR | 21.65 | AQTLARPFV | 14.51 | DGTNSTTGW | 6.41 |
| (SEQ ID NO: | | (SEQ ID NO: | | (SEQ ID NO: | |
| 140) | | 98) | | 143) | |
| DGTGNTRGW | 21.12 | DGTGGTKGW | 14.13 | DGSQSTTGW | 6.29 |
| (SEQ ID NO: | | (SEQ ID NO: | | (SEQ ID NO: | |
| 94) | | 107) | | 136) | |
| AQTTEKPWL | 20.58 | AQGPTRPFL | 13.47 | AQPEGSARW | 6.23 |
| (SEQ ID NO: | | (SEQ ID NO: | | (SEQ ID NO: | |
| 83) | | 125) | | 60) | |
| DGTADRPFR | 20.49 | DGTRTTTGW | 13.39 | DGTGQTIGW | 6.16 |
| (SEQ ID NO: | | (SEQ ID NO: | | (SEQ ID NO: | |
| 155) | | 106) | | 305) | |
| DGTTWTPPR | 20.44 | AQNGNPGRW | 13.09 | DGTGGVTGW | 6.07 |
| (SEQ ID NO: | | (SEQ ID NO: | | (SEQ ID NO: | |
| 139) | | 84) | | 299) | |
| DGTTTYGAR | 20.43 | DGTVSNPFR | 12.77 | DGTVTTTGW | 6.04 |
| (SEQ ID NO: | | (SEQ ID NO: | | (SEQ ID NO: | |
| 77) | | 403) | | 405) | |
| DGTGGIKGW | 20.20 | AQGGNPGRW | 12.21 | DGKGSTQGW | 5.97 |
| (SEQ ID NO: | | (SEQ ID NO: | | (SEQ ID NO: | |
| 131) | | 91) | | 272) | |
| DGTLAVPFK | 19.43 | AQWPTSYDA | 11.93 | AQGENPGRW | 5.88 |
| (SEQ ID NO: | | (SEQ ID NO: | | (SEQ ID NO: | |
| 71) | | 62) | | 96) | |
| DGKQYQLSS | 18.74 | DGTLQQPFR | 11.92 | DGNGGLKGW | 5.82 |
| (SEQ ID NO: | | (SEQ ID NO: | | (SEQ ID NO: | |
| 92) | | 89) | | 167) | |
| DGSPEKPFR | 18.73 | DGTNGLKGW | 11.53 | DGTGTVHGW | 5.82 |
| (SEQ ID NO: | | (SEQ ID NO: | | (SEQ ID NO: | |
| 160) | | 76) | | 326) | |

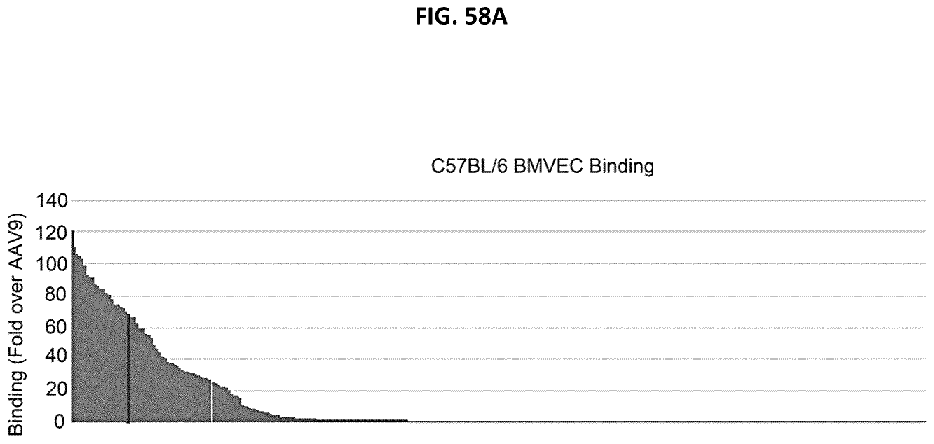

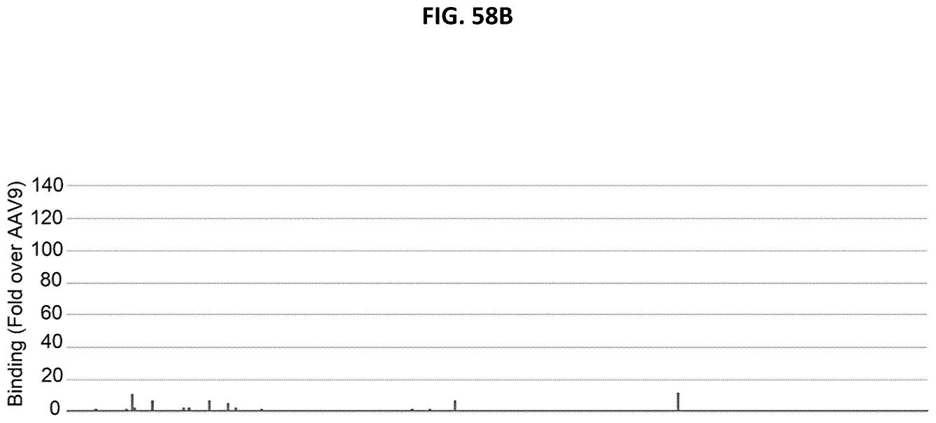

The efficacy of the 333 capsid variants to transduce CNS was also compared for C57BL/6 mice BMVEC and Human BMVEC (FIG. 58A and FIG. 58B).

### Example 14. Engineering of a NGS-Driven Selection System for Full-Length Capsid Variants

A barcode system was engineered to allow enrichment studies with full capsid length modifications. While the TRACER platform described here was initially developed for the use of peptide display libraries, where the randomized peptide sequence itself can be used for Illumina NGS analysis due to its short size, the Illumina sequencing technology does not typically allow sequencing of more than 300 contiguous bases, and therefore our platform cannot be used for NGS analysis of full-length capsid variants, such as those generated by DNA shuffling technology or error-prone PCR.

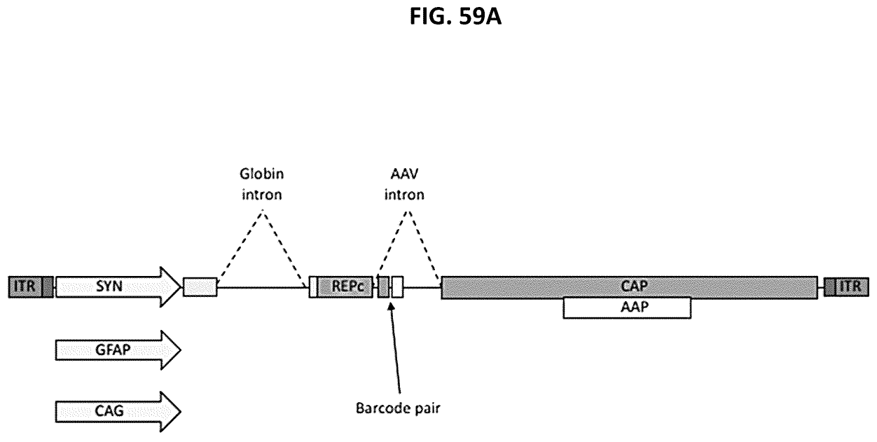

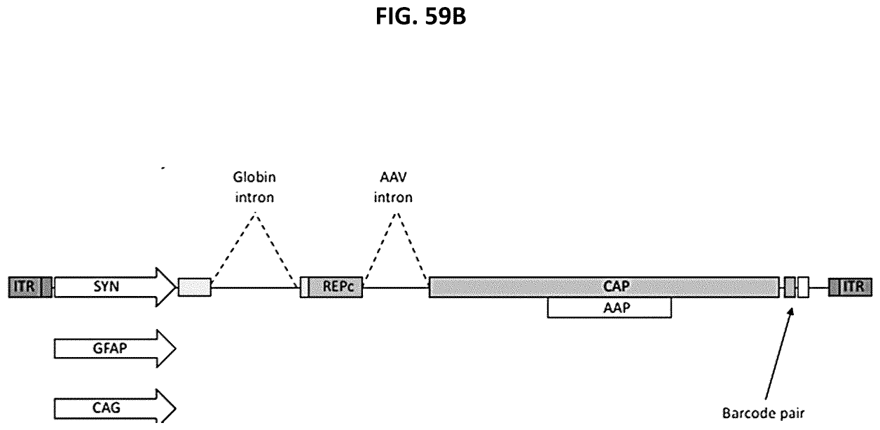

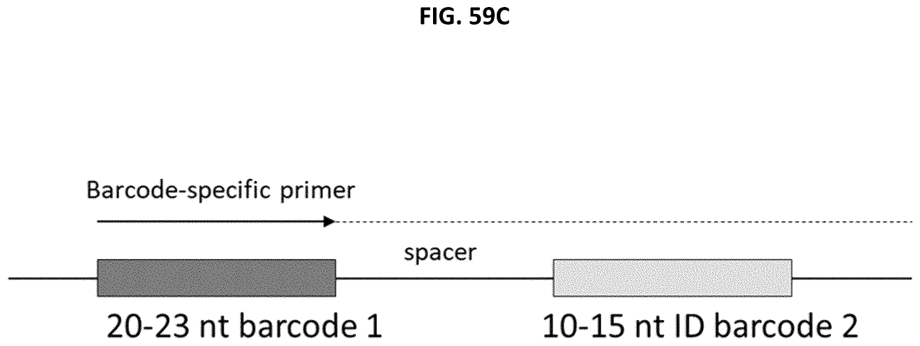

An alternative RNA-driven platform for full-length capsid libraries in which a unique molecular identified (UMI) is placed outside the capsid gene and can be used for NGS enrichment analysis was designed (FIG. 59A-FIG. 59C). Once the variants with desired properties are identified by UMI enrichment analysis from animal tissue, the UMI sequence must allow highly specific recovery of the full-length capsid from the starting material with a minimal error rate. The system should have one or more of the following properties to be effective: 1) the UMI should be transcribed under control of a cell type-specific promoter, 2) the UMI should not interfere with capsid expression or splicing during virus production, 3) the UMI should be short enough for Illumina NGS sequencing (typically less than 60 nt for standard single-end 75 nt sequencing), and 4) the UMI should allow sequence-specific recovery of full-length capsids of interest from the starting DNA/virus library with a minimal error rate.

To address these properties: 1) the UMI was placed in the transcribed region of capsid library (i.e., anywhere between the transcription start site and the polyadenylation signal), 2) the UMI was placed either in various locations of the AAV intron (which mostly unspliced in the absence of helper functions) or between the capsid stop codon and the polyadenylation signal, 3) the UMI cassette was composed of two randomized 21-nt sequences separated by a 15-nt spacer, for a total length of 57 nt, which allows 18 extra nucleotides for primer annealing, and 4) the UMI randomized sequences were formed of NSW triplets (N=A, T, G, C; S=G, C; W=A, T) to prevent large variations in annealing temperature with amplification primers, avoid homopolymeric stretches and prevent the formation of a premature polyA signal (AATAAA).

Importantly, the UMI cassette contained two random sequences in tandem. The first sequence (outermost) is used to design a matching capsid recovery primer, and the second sequence (innermost) to confirm the identity of the capsid amplicon after cloning. This method should allow to eliminate all clones containing non-specific amplification products. In an alternative embodiment, the innermost sequence can also be used to design a nested PCR primer in order to increase the specificity of amplification (FIG. 59A-FIG. 59C).

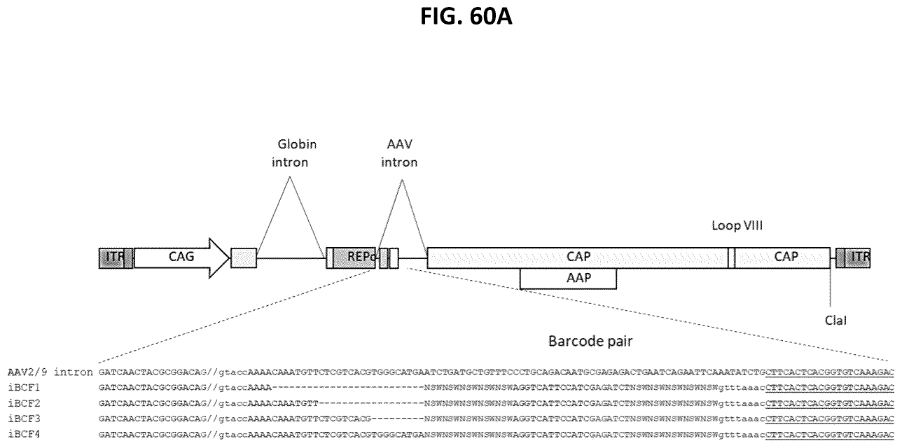

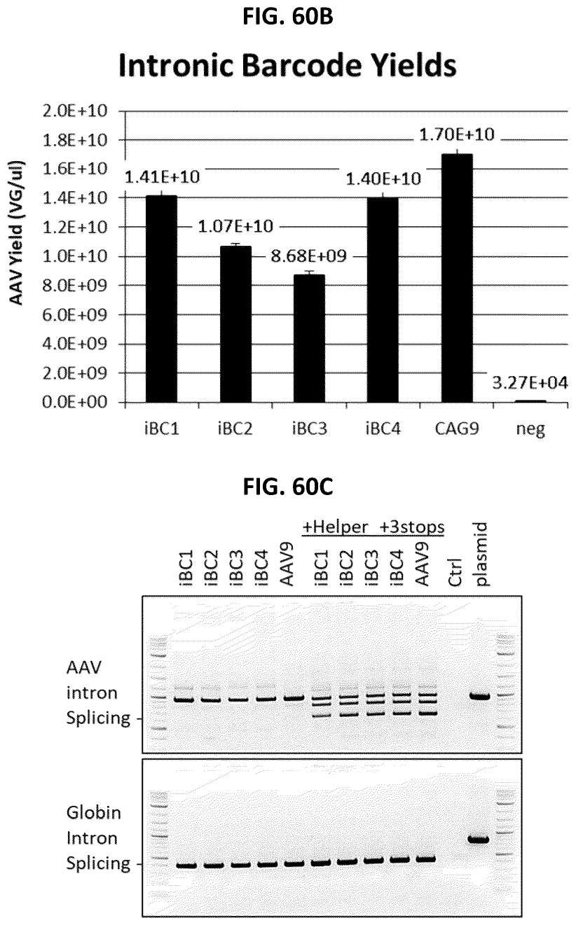

Several insertion sites of the tandem barcode to test the impact on virus viability and titers were explored. A series of constructs were engineered with the barcode inserted in the AAV intron of the CAG9 plasmid (FIG. 60A). Since AAV intron is spliced during virus production, the presence of the barcode should have only a minimal impact on the yields. Conversely, the AAV splicing is very ineffective in the absence of helper functions (Mouw et al., 2000), therefore the barcode sequence will be preserved in the RNA recovered from animal tissue. All intronic barcode constructs were tested for their ability to produce high titer AAV progeny by cotransfecting them with pHelper and pREP3stop plasmids. All constructs allowed high titer AAV production going from 50% to 80% of non-barcoded CAG9 virus (FIG. 60B).

RNA splicing analysis from transfected cells showed that the rate of AAV intron splicing was slightly different between constructs and was more efficient when the intronic barcode was inserted after a conserved intervening sequence downstream of the splice donor (FIG. 58C, upper panel).

Globin intron splicing was 100% effective in all tested conditions (FIG. 60C, lower panel). As expected, AAV intron splicing was almost undetectable in the absence of helper functions.

An alternative platform was tested where the tandem barcode was placed between the capsid stop codon and the polyadenylation signal (FIG. 59B). Titers produced by the 3′-barcoded constructs were identical to the non-barcoded CAG9 construct.

Overall, external barcoding of full-length capsid allows highly efficient AAV production, and the novel tandem barcode platform allows NGS-driven sequence-specific recovery from library preparations with high confidence.

TABLE 12
Sequences

| DESCRIPTION | |
|---|---|
| SEQ ID NO: | NUCLEIC ACID SEQUENCE |
| PREP2 SEQ ID | CGCAGGGTCTCCATTTTGAAGCGGGAGGTTTGAACGCGCAGCCGCCATGCCGGGGTTTTA |
| NO: 4 | CGAGATTGTGATTAAGGTCCCCAGCGACCTTGACGAGCATCTGCCCGGCATTTCTGACAG |
| | CTTTGTGAACTGGGTGGCCGAGAAGGAATGGGAGTTGCCGCCAGATTCTGACATGGATCT |
| | GAATCTGATTGAGCAGGCACCCCTGACCGTGGCCGAGAAGCTGCAGCGCGACTTTCTGAC |
| | GGAATGGCGCCGTGTGAGTAAGGCCCCGGAGGCTCTTTTCTTTGTGCAATTTGAGAAGGG |
| | AGAGAGCTACTTCCACATGCACGTGCTCGTGGAAACCACCGGGGTGAAATCCATGGTTTT |
| | GGGACGTTTCCTGAGTCAGATTCGCGAAAAACTGATTCAGAGAATTTACCGCGGGATCGA |
| | GCCGACTTTGCCAAACTGGTTCGCGGTCACAAAGACCAGAAATGGCGCCGGAGGCGGGA |
| | ACAAGGTGGTGGATGAGTGCTACATCCCCAATTACTTGCTCCCCAAAACCCAGCCTGAGC |
| | TCCAGTGGGCGTGGACTAATATGGAACAGTATTTAAGCGCCTGTTTGAATCTCACGGAGC |
| | GTAAACGGTTGGTGGCGCAGCATCTGACGCACGTGTCGCAGACGCAGGAGCAGAACAAA |
| | GAGAATCAGAATCCCAATTCTGATGCGCCGGTGATCAGATCAAAAACTTCAGCCAGGTAC |
| | ATGGAGCTGGTCGGGTGGCTCGTGGACAAGGGGATTACCTCGGAGAAGCAGTGGATCCA |
| | GGAGGACCAGGCCTCATACATCTCCTTCAATGCGGCCTCCAACTCGCGGTCCCAAATCAA |
| | GGCTGCCTTGGACAATGCGGGAAAGATTATGAGCCTGACTAAAACCGCCCCCGACTACCT |
| | GGTGGGCCAGCAGCCCGTGGAGGACATTTCCAGCAATCGGATTTATAAAATTTTGGAACT |
| | AAACGGGTACGATCCCCAATATGCGGCTTCCGTCTTTCTGGGATGGGCCACGAAAAAGTT |
| | CGGCAAGAGGAACACCATCTGGCTGTTTGGGCCTGCAACTACCGGGAAGACCAACATCG |
| | CGGAGGCCATAGCCCACACTGTGCCCTTCTACGGGTGCGTAAACTGGACCAATGAGAACT |
| | TTCCCTTCAACGACTGTGTCGACAAGATGGTGATCTGGTGGGAGGAGGGGAAGATGACC |
| | GCCAAGGTCGTGGAGTCGGCCAAAGCCATTCTCGGAGGAAGCAAGGTGCGCGTGGACCA |
| | GAAATGCAAGTCCTCGGCCCAGATAGACCCGACTCCCGTGATCGTCACCTCCAACACCAA |
| | CATGTGCGCCGTGATTGACGGGAACTCAACGACCTTCGAACACCAGCAGCCGTTGCAAGA |
| | CCGGATGTTCAAATTTGAACTCACCCGCCGTCTGGATCATGACTTTGGGAAGGTCACCAA |
| | GCAGGAAGTCAAAGACTTTTTCCGGTGGGCAAAGGATCACGTGGTTGAGGTGGAGCATG |
| | AATTCTACGTCAAAAAGGGTGGAGCCAAGAAAAGACCCGCCCCCAGTGACGCAGATATA |
| | AGTGAGCCCAAACGGGTGCGCGAGTCAGTTGCGCAGCCATCGACGTCAGACGCGGAAGC |
| | TTCGATCAACTACGCAGACAGGTACCAAAACAAATGTTCTCGTCACGTGGGCATGAATCT |
| | GATGCTGTTTCCCTGCAGACAATGCGAGAGAATGAATCAGAATTCAAATATCTGCTTCAC |
| | TCACGGACAGAAAGACTGTTTAGAGTGCTTTCCCGTGTCAGAATCTCAACCCGTTTCTGTC |
| | GTCAAAAAGGCGTATCAGAAACTGTGCTACATTCATCATATCATGGGAAAGGTGCCAGAC |
| | GCTTGCACTGCCTGCGATCTGGTCAATGTGGATTTGGATGACTGCATCTTTGAACAATAA |
| | ATGATTTAAATCAGGTATGGCTGCCGATGGTTATCTTCCAGATTGGCTCGAGGACACTCTC |
| | TCTGAAGGAATAAGACAGTGGTGGAAGCTCAAACCTGGCCCACCACCACCAAAGCCCGC |
| | AGAGCGGCATAAGGACGACAGCAGGGGTCTTGTGCTTCCTGGGTACAAGTACCTCGGAC |
| | CCTTCAACGGACTCGACAAGGGAGAGCCGGTCAACGAGGCAGACGCCGCGGCCCTCGAG |
| | CACGACAAAGCCTACGACCGGCAGCTCGACAGCGGAGACAACCCGTACCTCAAGTACAA |
| | CCACGCCGACGCGGAGTTTCAGGAGCGCCTTAAAGAAGATACGTCTTTTGGGGGCAACCT |
| | CGGACGAGCAGTCTTCCAGGCGAAAAAGAGGGTTCTTGAACCTCTGGGCCTGGTCCACCA |
| | TACCTTCGATTATCCGATTTGCTTGTTAATCAATAAACCGTTTAATTCGTTTCAGTTGAACT |
| | TTGGTCTCTGCGTATTTCTTTCTTATCTAGTTTCCATGCTCTAGAGCGGCCGCCACCGCGGT |
| | GGAGCTCCAGCTTTTGT |
| CMV9-BSTEII | TTGGCCACTCCCTCTCTGCGCGCTCGCTCGCTCACTGAGGCCGGGCGACCAAAGGTCGCC |
| SEQ ID NO: 5 | CGACGCCCGGGCTTTGCCCGGGCGGCCTCAGTGAGCGAGCGAGCGCGCAGAGAGGGAGT |
| | GGCCAACTCCATCACTAGGGGTTCCTGGAGGGGTGGAGTCGTGACGATATCGTTTAAACC |
| | GCGTCGTTACATAACTTACGGTAAATGGCCCGCCTGGCTGACCGCCCAACGACCCCCGCC |
| | CATTGACGTCAATAATGACGTATGTTCCCATAGTAACGCCAATAGGGACTTTCCATTGAC |
| | GTCAATGGGTGGAGTATTTACGGTAAACTGCCCACTTGGCAGTACATCAAGTGTATCATA |
| | TGCCAAGTACGCCCCCTATTGACGTCAATGACGGTAAATGGCCCGCCTGGCATTATGCCC |
| | AGTACATGACCTTATGGGACTTTCCTACTTGGCAGTACATCTACGTATTAGTCATCGCTAT |
| | TACCATGGTGATGCGGTTTTGGCAGTACATCAATGGGCGTGGATAGCGGTTTGACTCACG |
| | GGGATTTCCAAGTCTCCACCCCATTGACGTCAATGGGAGTTTGTTTTGGCACCAAAATCA |
| | ACGGGACTTTCCAAAATGTCGTAACAACTCCGCCCCATTGACGCAAATGGGCGGTAGGCG |
| | TGTACGGTGGGAGGTCTATATAAGCAGAGCTCGGGAGCGGTCACCAAGCAGGAAGTCAA |
| | AGACTTTTTCCGGTGGGCAAAGGATCACGTGGTTGAGGTGGAGCATGAATTCTACGTCAA |
| | AAAGGGTGGAGCCAAGAAAAGACCCGCCCCCAGTGACGCAGATATAAGTGAGCCCAAAC |
| | GGGTGCGCGAGTCAGTTGCGCAGCCATCGACGTCAGACGCGGAAGCTTCGATCAACTAC |
| | GCGGACAGGTACCAAAACAAATGTTCTCGTCACGTGGGCATGAATCTGATGCTGTTTCCC |
| | TGCAGACAATGCGAGAGACTGAATCAGAATTCAAATATCTGCTTCACTCACGGTGTCAAA |
| | GACTGTTTAGAGTGCTTTCCCGTGTCAGAATCTCAACCCGTTTCTGTCGTCAAAAAGGCGT |
| | ATCAGAAACTGTGCTACATTCATCACATCATGGGAAAGGTGCCAGACGCTTGCACTGCTT |
| | GCGACCTGGTCAATGTGGACTTGGATGACTGTGTTTCTGAACAATAAATGACTTAAACCA |
| | GGTATGGCTGCCGATGGTTATCTTCCAGATTGGCTCGAGGACAACCTTAGTGAAGGAATT |
| | CGCGAGTGGTGGGCTTTGAAACCTGGAGCCCCTCAACCCAAGGCAAATCAACAACATCA |
| | AGACAACGCTCGAGGTCTTGTGCTTCCGGGTTACAAATACCTTGGACCCGGCAACGGACT |
| | CGACAAGGGGGAGCCGGTCAACGCAGCAGACGCGGCGGCCCTCGAGCACGACAAGGCCT |
| | ACGACCAGCAGCTCAAGGCCGGAGACAACCCGTACCTCAAGTACAACCACGCCGACGCC |
| | GAGTTCCAGGAGCGGCTCAAAGAAGATACGTCTTTTGGGGGCAACCTCGGGCGAGCAGT |
| | CTTCCAGGCCAAAAAGAGGCTTCTTGAACCTCTTGGTCTGGTTGAGGAAGCGGCTAAGAC |
| | GGCTCCTGGAAAGAAGAGGCCTGTAGAGCAGTCTCCTCAGGAACCGGACTCCTCCGCGG |
| | GTATTGGCAAATCGGGTGCACAGCCCGCTAAAAAGAGACTCAATTTCGGTCAGACTGGCG |
| | ACACAGAGTCAGTCCCAGACCCTCAACCAATCGGAGAACCTCCCGCAGCCCCCTCAGGTG |
| | TGGGATCTCTTACAATGGCTTCAGGTGGTGGCGCACCAGTGGCAGACAATAACGAAGGTG |
| | CCGATGGAGTGGGTAGTTCCTCGGGAAATTGGCATTGCGATTCCCAATGGCTGGGGGACA |
| | GAGTCATCACCACCAGCACCCGAACCTGGGCCCTGCCCACCTACAACAATCACCTCTACA |
| | AGCAAATCTCCAACAGCACATCTGGAGGATCTTCAAATGACAACGCCTACTTCGGCTACA |
| | GCACCCCCTGGGGGTATTTTGACTTCAACAGATTCCACTGCCACTTCTCACCACGTGACTG |
| | GCAGCGACTCATCAACAACAACTGGGGATTCCGGCCTAAGCGACTCAACTTCAAGCTCTT |
| | CAACATTCAGGTCAAAGAGGTTACGGACAACAATGGAGTCAAGACCATCGCCAATAACC |
| | TTACCAGCACGGTCCAGGTCTTCACGGACTCAGACTATCAGCTCCCGTACGTGCTCGGGT |
| | CGGCTCACGAGGGCTGCCTCCCGCCGTTCCCAGCGGACGTTTTCATGATTCCTCAGTACG |
| | GGTATCTGACGCTTAATGATGGAAGCCAGGCCGTGGGTCGTTCGTCCTTTTACTGCCTGG |
| | AATATTTCCCGTCGCAAATGCTAAGAACGGGTAACAACTTCCAGTTCAGCTACGAGTTTG |
| | AGAACGTACCTTTCCATAGCAGCTACGCTCACAGCCAAAGCCTGGACCGACTAATGAATC |
| | CACTCATCGACCAATACTTGTACTATCTCTCAAAGACTATTAACGGTTCTGGACAGAATC |
| | AACAAACGCTAAAATTCAGTGTGGCCGGACCCAGCAACATGGCTGTCCAGGGAAGAAAC |
| | TACATACCTGGACCCAGCTACCGACAACAACGTGTCTCAACCACTGTGACTCAAAACAAC |
| | AACAGCGAATTTGCTTGGCCTGGAGCTTCTTCTTGGGCTCTCAATGGACGTAATAGCTTGA |
| | TGAATCCTGGACCTGCTATGGCCAGCCACAAAGAAGGAGAGGACCGTTTCTTTCCTTTGT |
| | CTGGATCTTTAATTTTTGGCAAACAAGGAACTGGAAGAGACAACGTGGATGCGGACAAA |
| | GTCATGATAACCAACGAAGAAGAAATTAAAACTACTAACCCGGTAGCAACGGAGTCCTA |
| | TGGACAAGTGGCCACAAACCACCAGAGTGCCCAAGCACAGGCGCAGACCGGCTGGGTTC |
| | AAAACCAAGGAATACTTCCGGGTATGGTTTGGCAGGACAGAGATGTGTACCTGCAAGGA |
| | CCCATTTGGGCCAAAATTCCTCACACGGACGGCAACTTTCACCCTTCTCCGCTGATGGGA |
| | GGGTTTGGAATGAAGCACCCGCCTCCTCAGATCCTCATCAAAAACACACCTGTACCTGCG |
| | GATCCTCCAACGGCCTTCAACAAGGACAAGCTGAACTCTTTCATCACCCAGTATTCTACT |
| | GGCCAAGTCAGCGTGGAGATCGAGTGGGAGCTGCAGAAGGAAAACAGCAAGCGCTGGA |
| | ACCCGGAGATCCAGTACACTTCCAACTATTACAAGTCTAATAATGTTGAATTTGCTGTTAA |
| | TACTGAAGGTGTATATAGTGAACCCCGCCCCATTGGCACCAGATACCTGACTCGTAATCT |
| | GTAATCGATTGTTAATCAATAAACCGTTTAATTCGTTTCAGTTGAACTTTGGTCTCTGCGT |
| | ATTTCTTTCTTATCTAGTTTCCATGGCTACGTAGATAAGTAGCATGGCGGGTTAATCATTA |
| | ACTACAAGGAACCCCTAGTGATGGAGTTGGCCACTCCCTCTCTGCGCGCTCGCTCGCTCA |
| | CTGAGGCCGGGCGACCAAAGGTCGCCCGACGCCCGGGCTTTGCCCGGGCGGCCTCAGTG |
| | AGCGAGCGAGCGCGCAGAGAGGGAGTGGCCAA |
| PREP-AAP | GTCGACGGTATCGGGGGAGCTCGCAGGGTCTCCATTTTGAAGCGGGAGGTTTGAACGCGCAGC |
| SEQ ID NO: 6 | CGCCATGCCGGGGTTTTACGAGATTGTGATTAAGGTCCCCAGCGACCTTGACGAGCATCTGCCC |
| | GGCATTTCTGACAGCTTTGTGAACTGGGTGGCCGAGAAGGAATGGGAGTTGCCGCCAGATTCT |
| | GACATGGATCTGAATCTGATTGAGCAGGCACCCCTGACCGTGGCCGAGAAGCTGCAGCGCGAC |
| | TTTCTGACGGAATGGCGCCGTGTGAGTAAGGCCCCGGAGGCTCTTTTCTTTGTGCAATTTGAGA |
| | AGGGAGAGAGCTACTTCCACATGCACGTGCTCGTGGAAACCACCGGGGTGAAATCCATGGTTT |
| | TGGGACGTTTCCTGAGTCAGATTCGCGAAAAACTGATTCAGAGAATTTACCGCGGGATCGAGC |
| | CGACTTTGCCAAACTGGTTCGCGGTCACAAAGACCAGAAATGGCGCCGGAGGCGGGAACAAG |
| | GTGGTGGATGAGTGCTACATCCCCAATTACTTGCTCCCCAAAACCCAGCCTGAGCTCCAGTGGG |
| | CGTGGACTAATATGGAACAGTATTTAAGCGCCTGTTTGAATCTCACGGAGCGTAAACGGTTGGT |
| | GGCGCAGCATCTGACGCACGTGTCGCAGACGCAGGAGCAGAACAAAGAGAATCAGAATCCCA |
| | ATTCTGATGCGCCGGTGATCAGATCAAAAACTTCAGCCAGGTACATGGAGCTGGTCGGGTGGC |
| | TCGTGGACAAGGGGATTACCTCGGAGAAGCAGTGGATCCAGGAGGACCAGGCCTCATACATCT |
| | CCTTCAATGCGGCCTCCAACTCGCGGTCCCAAATCAAGGCTGCCTTGGACAATGCGGGAAAGA |
| | TTATGAGCCTGACTAAAACCGCCCCCGACTACCTGGTGGGCCAGCAGCCCGTGGAGGACATTT |
| | CCAGCAATCGGATTTATAAAATTTTGGAACTAAACGGGTACGATCCCCAATATGCGGCTTCCGT |
| | CTTTCTGGGATGGGCCACGAAAAAGTTCGGCAAGAGGAACACCATCTGGCTGTTTGGGCCTGC |
| | AACTACCGGGAAGACCAACATCGCGGAGGCCATAGCCCACACTGTGCCCTTCTACGGGTGCGT |
| | AAACTGGACCAATGAGAACTTTCCCTTCAACGACTGTGTCGACAAGATGGTGATCTGGTGGGA |
| | GGAGGGGAAGATGACCGCCAAGGTCGTGGAGTCGGCCAAAGCCATTCTCGGAGGAAGCAAGG |
| | TGCGCGTGGACCAGAAATGCAAGTCCTCGGCCCAGATAGACCCGACTCCCGTGATCGTCACCT |
| | CCAACACCAACATGTGCGCCGTGATTGACGGGAACTCAACGACCTTCGAACACCAGCAGCCGT |
| | TGCAAGACCGGATGTTCAAATTTGAACTCACCCGCCGTCTGGATCATGACTTTGGGAAGGTCAC |
| | CAAGCAGGAAGTCAAAGACTTTTTCCGGTGGGCAAAGGATCACGTGGTTGAGGTGGAGCATGA |
| | ATTCTACGTCAAAAAGGGTGGAGCCAAGAAAAGACCCGCCCCCAGTGACGCAGATATAAGTGA |
| | GCCCAAACGGGTGCGCGAGTCAGTTGCGCAGCCATCGACGTCAGACGCGGAAGCTTCGATCAA |
| | CTACGCGGACAGGTACCAAAACAAATGTTCTCGTCACGTGGGCATGAATCTGATGCTGTTTCCC |
| | TGCAGACAATGCGAGAGACTGAATCAGAATTCAAATATCTGCTTCACTCACGGTGTCAAAGAC |
| | TGTTTAGAGTGCTTTCCCGTGTCAGAATCTCAACCCGTTTCTGTCGTCAAAAAGGCGTATCAGA |
| | AACTGTGCTACATTCATCACATCATGGGAAAGGTGCCAGACGCTTGCACTGCTTGCGACCTGGT |
| | CAATGTGGACTTGGATGACTGTGTTTCTGAACAATAAATGACTTAAACCAGGTATGGCTGCCGA |
| | TGGTTATCTTCCAGATTGGCTCGAGGACAACCTTAGTGAAGGAATTCGCGAGTGGTGGGCTTTG |
| | AAACCTGGAGCCCCTCAACCCAAGGCAAATCAACAACATCAAGACAACGCTCGAGGTCTTGTG |
| | CTTCCGGGTTACAAATACCTTGGACCCGGCAACGGACTCGACAAGGGGGAGCCGGTCAACGCA |
| | GCAGACGCGGCGGCCCTCGAGCACGACAAGGCCTACGACCAGCAGCTCAAGGCCGGAGACAA |
| | CCCGTACCTCAAGTACAACCACGCCGACGCCGAGTTCCAGGAGCGGCTCAAAGAAGATACGTC |
| | TTTTGGGGGCAACCTCGGGCGAGCAGTCTTCCAGGCCAAAAAGAGGCTTCTTGAACCTCTTGGT |
| | CTGGTTGAGGAAGCGGCTAAGACGGCTCCTGGAAAGAAGAGGCCTGTAGAGCAGTCTCCTCAG |
| | GAACCGGACTCCTCCGCGGGTATTGGCAAATCGGGTGCACAGCCCGCTAAAAAGAGACTCAAT |
| | TTCGGTCAGACTGGCGACACAGAGTCAGTCCCAGACCCTCAACCAATCGGAGAACCTCCCGCA |
| | GCCCCCTCAGGTGTGGGATCTCTTACAATGGCTTCAGGTGGTGGCGCACCAGTGGCAGACAAT |
| | AACGAAGGTGCCGATGGAGTGGGTAGTTCCTCGGGAAATTGGCATTGCGATTCCCAATGGCTG |
| | GGGGACAGAGTCATCACCACCAGCACCCGAACCTGGGCCCTGCCCACCTACAACAATCACCTC |
| | TACAAGCAAATCTCCAACAGCACATCTGGAGGATCTTCAAATGACAACGCCTACTTCGGCTAC |
| | AGCACCCCCTGGGGGTATTTTGACTTCAACAGATTCCACTGCCACTTCTCACCACGTGACTGGC |
| | AGCGACTCATCAACAACAACTGGGGATTCCGGCCTAAGCGACTCAACTTCAAGCTCTTCAACAT |
| | TCAGGTCAAAGAGGTTACGGACAACAATGGAGTCAAGACCATCGCCAATAACCTTACCAGCAC |
| | GGTCCAGGTCTTCACGGACTCAGACTATCAGCTCCCGTACGTGCTCGGGTCGGCTCACGAGGGC |
| | TGCCTCCCGCCGTTCCCAGCGGACGTTTTCATGATTCCTCAGTACGGGTATCTGACGCTTAATG |
| | ATGGAAGCCAGGCCGTGGGTCGTTCGTCCTTTTACTGCCTGGAATATTTCCCGTCGCAAATGCT |
| | AAGAACGGGTAACAACTTCCAGTTCAGCTACGAGTTTGAGAACGTACCTTTCCATAGCAGCTAC |
| | GCTCACAGCCAAAGCCTGGACCGACTAATGAATCCACTCATCGACCAATACTTGTACTATCTCT |
| | CAAAGACTATTAACGGTTCTGGACAGAATCAACAAACGCTAAAATTCAGTGTGGCCGGACCCA |
| | GCAACATGGCTGTCCAGGGAAGAAACTACATACCTGGACCCAGCTACCGACAACAACGTGTCT |
| | CAACCACTGTGACTCAAAACAACAACAGCGAATTTGCTTGGCCTGGAGCTTCTTCTTGGGCTCT |
| | CAATGGACGTAATAGCTTGATGAATCCTGGACCTGCTATGGCCAAGTCAGCGTGGAGATCGAG |
| | TGGGAGCTGCAGAAGGAAAACAGCAAGCGCTGGAACCCGGAGATCCAGTACACTTCCAACTAT |
| | TACAAGTCTAATAATGTTGAATTTGCTGTTAATACTGAAGGTGTATATAGTGAACCCCGCCCCA |
| | TTGGCACCAGATACCTGACTCGTAATCTGTAATTGCTTGTTAATCAATAAACCGTTTAATTCGTT |
| | TCAGTTGAACTTTGGTCTC |
| PREP3STOP | GTCGACGGTATCGGGGGAGCTCGCAGGGTCTCCATTTTGAAGCGGGAGGTTTGAACGCGCAGC |
| SEQ ID NO: 7 | CGCCATGCCGGGGTTTTACGAGATTGTGATTAAGGTCCCCAGCGACCTTGACGAGCATCTGCCC |
| | GGCATTTCTGACAGCTTTGTGAACTGGGTGGCCGAGAAGGAATGGGAGTTGCCGCCAGATTCT |
| | GACATGGATCTGAATCTGATTGAGCAGGCACCCCTGACCGTGGCCGAGAAGCTGCAGCGCGAC |
| | TTTCTGACGGAATGGCGCCGTGTGAGTAAGGCCCCGGAGGCTCTTTTCTTTGTGCAATTTGAGA |
| | AGGGAGAGAGCTACTTCCACATGCACGTGCTCGTGGAAACCACCGGGGTGAAATCCATGGTTT |
| | TGGGACGTTTCCTGAGTCAGATTCGCGAAAAACTGATTCAGAGAATTTACCGCGGGATCGAGC |
| | CGACTTTGCCAAACTGGTTCGCGGTCACAAAGACCAGAAATGGCGCCGGAGGCGGGAACAAG |
| | GTGGTGGATGAGTGCTACATCCCCAATTACTTGCTCCCCAAAACCCAGCCTGAGCTCCAGTGGG |
| | CGTGGACTAATATGGAACAGTATTTAAGCGCCTGTTTGAATCTCACGGAGCGTAAACGGTTGGT |
| | GGCGCAGCATCTGACGCACGTGTCGCAGACGCAGGAGCAGAACAAAGAGAATCAGAATCCCA |
| | ATTCTGATGCGCCGGTGATCAGATCAAAAACTTCAGCCAGGTACATGGAGCTGGTCGGGTGGC |
| | TCGTGGACAAGGGGATTACCTCGGAGAAGCAGTGGATCCAGGAGGACCAGGCCTCATACATCT |
| | CCTTCAATGCGGCCTCCAACTCGCGGTCCCAAATCAAGGCTGCCTTGGACAATGCGGGAAAGA |
| | TTATGAGCCTGACTAAAACCGCCCCCGACTACCTGGTGGGCCAGCAGCCCGTGGAGGACATTT |
| | CCAGCAATCGGATTTATAAAATTTTGGAACTAAACGGGTACGATCCCCAATATGCGGCTTCCGT |
| | CTTTCTGGGATGGGCCACGAAAAAGTTCGGCAAGAGGAACACCATCTGGCTGTTTGGGCCTGC |
| | AACTACCGGGAAGACCAACATCGCGGAGGCCATAGCCCACACTGTGCCCTTCTACGGGTGCGT |
| | AAACTGGACCAATGAGAACTTTCCCTTCAACGACTGTGTCGACAAGATGGTGATCTGGTGGGA |
| | GGAGGGGAAGATGACCGCCAAGGTCGTGGAGTCGGCCAAAGCCATTCTCGGAGGAAGCAAGG |
| | TGCGCGTGGACCAGAAATGCAAGTCCTCGGCCCAGATAGACCCGACTCCCGTGATCGTCACCT |
| | CCAACACCAACATGTGCGCCGTGATTGACGGGAACTCAACGACCTTCGAACACCAGCAGCCGT |
| | TGCAAGACCGGATGTTCAAATTTGAACTCACCCGCCGTCTGGATCATGACTTTGGGAAGGTCAC |
| | CAAGCAGGAAGTCAAAGACTTTTTCCGGTGGGCAAAGGATCACGTGGTTGAGGTGGAGCATGA |
| | ATTCTACGTCAAAAAGGGTGGAGCCAAGAAAAGACCCGCCCCCAGTGACGCAGATATAAGTGA |
| | GCCCAAACGGGTGCGCGAGTCAGTTGCGCAGCCATCGACGTCAGACGCGGAAGCTTCGATCAA |
| | CTACGCGGACAGGTACCAAAACAAATGTTCTCGTCACGTGGGCATGAATCTGATGCTGTTTCCC |
| | TGCAGACAATGCGAGAGACTGAATCAGAATTCAAATATCTGCTTCACTCACGGTGTCAAAGAC |
| | TGTTTAGAGTGCTTTCCCGTGTCAGAATCTCAACCCGTTTCTGTCGTCAAAAAGGCGTATCAGA |
| | AACTGTGCTACATTCATCACATCATGGGAAAGGTGCCAGACGCTTGCACTGCTTGCGACCTGGT |
| | CAATGTGGACTTGGATGACTGTGTTTCTGAACAATAAATGACTTAAACCAGGTATGGCTGCCGA |
| | TGGTTAGCTTCCAGATTGGCTCGAGGACAACCTTAGTGAAGGAATTCGCGAGTGGTGGGCTTTG |
| | AAACCTGGAGCCCCTCAACCCAAGGCAAATCAACAACATCAAGACAACGCTCGAGGTCTTGTG |
| | CTTCCGGGTTACAAATACCTTGGACCCGGCAACGGACTCGACAAGGGGGAGCCGGTCAACGCA |
| | GCAGACGCGGCGGCCCTCGAGCACGACAAGGCCTACGACCAGCAGCTCAAGGCCGGAGACAA |
| | CCCGTACCTCAAGTACAACCACGCCGACGCCGAGTTCCAGGAGCGGCTCAAAGAAGATACGTC |
| | TTTTGGGGGCAACCTCGGGCGAGCAGTCTTCCAGGCCAAAAAGAGGCTTCTTGAACCTCTTGGT |
| | CTGGTTGAGGAAGCGGCTAAGACGGCTCCTGGAAAGTAGAGGCCTGTAGAGCAGTCTCCTCAG |
| | GAACCGGACTCCTCCGCGGGTATTGGCAAATCGGGTGCACAGCCCGCTAAAAAGAGACTCAAT |
| | TTCGGTCAGACTGGCGACACAGAGTCAGTCCCAGACCCTCAACCAATCGGAGAACCTCCCGCA |
| | GCCCCCTCAGGTGTGGGATCTCTTACAATGGCTTCAGGTGGTGGCGCACCAGTGGCAGACAAT |
| | AACTAAGGTGCCGATGGAGTGGGTAGTTCCTCGGGAAATTGGCATTGCGATTCCCAATGGCTG |
| | GGGGACAGAGTCATCACCACCAGCACCCGAACCTGGGCCCTGCCCACCTACAACAATCACCTC |
| | TACAAGCAAATCTCCAACAGCACATCTGGAGGATCTTCAAATGACAACGCCTACTTCGGCTAC |
| | AGCACCCCCTGGGGGTATTTTGACTTCAACAGATTCCACTGCCACTTCTCACCACGTGACTGGC |
| | AGCGACTCATCAACAACAACTGGGGATTCCGGCCTAAGCGACTCAACTTCAAGCTCTTCAACAT |
| | TCAGGTCAAAGAGGTTACGGACAACAATGGAGTCAAGACCATCGCCAATAACCTTACCAGCAC |
| | GGTCCAGGTCTTCACGGACTCAGACTATCAGCTCCCGTACGTGCTCGGGTCGGCTCACGAGGGC |
| | TGCCTCCCGCCGTTCCCAGCGGACGTTTTCATGATTCCTCAGTACGGGTATCTGACGCTTAATG |
| | ATGGAAGCCAGGCCGTGGGTCGTTCGTCCTTTTACTGCCTGGAATATTTCCCGTCGCAAATGCT |
| | AAGAACGGGTAACAACTTCCAGTTCAGCTACGAGTTTGAGAACGTACCTTTCCATAGCAGCTAC |
| | GCTCACAGCCAAAGCCTGGACCGACTAATGAATCCACTCATCGACCAATACTTGTACTATCTCT |
| | CAAAGACTATTAACGGTTCTGGACAGAATCAACAAACGCTAAAATTCAGTGTGGCCGGACCCA |
| | GCAACATGGCTGTCCAGGGAAGAAACTACATACCTGGACCCAGCTACCGACAACAACGTGTCT |
| | CAACCACTGTGACTCAAAACAACAACAGCGAATTTGCTTGGCCTGGAGCTTCTTCTTGGGCTCT |
| | CAATGGACGTAATAGCTTGATGAATCCTGGACCTGCTATGGCCAAGTCAGCGTGGAGATCGAG |
| | TGGGAGCTGCAGAAGGAAAACAGCAAGCGCTGGAACCCGGAGATCCAGTACACTTCCAACTAT |
| | TACAAGTCTAATAATGTTGAATTTGCTGTTAATACTGAAGGTGTATATAGTGAACCCCGCCCCA |
| | TTGGCACCAGATACCTGACTCGTAATCTGTAATTGCTTGTTAATCAATAAACCGTTTAATTCGTT |
| | TCAGTTGAACTTTGGTCTC |
| SYN-CAP9 | TTGGCCACTCCCTCTCTGCGCGCTCGCTCGCTCACTGAGGCCGGGCGACCAAAGGTCGCCCGAC |
| SEQ ID NO: 8 | GCCCGGGCTTTGCCCGGGCGGCCTCAGTGAGCGAGCGAGCGCGCAGAGAGGGAGTGGCCAACT |
| | CCATCACTAGGGGTTCCTGGAGGGGTGGAGTCGTGACGATATCTAGTATCTGCAGAGGGCCCT |
| | GCGTATGAGTGCAAGTGGGTTTTAGGACCAGGATGAGGCGGGGTGGGGGTGCCTACCTGACGA |
| | CCGACCCCGACCCACTGGACAAGCACCCAACCCCCATTCCCCAAATTGCGCATCCCCTATCAGA |
| | GAGGGGGAGGGGAAACAGGATGCGGCGAGGCGCGTGCGCACTGCCAGCTTCAGCACCGCGGA |
| | CAGTGCCTTCGCCCCCGCCTGGCGGCGCGCGCCACCGCCGCCTCAGCACTGAAGGCGCGCTGA |
| | CGTCACTCGCCGGTCCCCCGCAAACTCCCCTTCCCGGCCACCTTGGTCGCGTCCGCGCCGCCGC |
| | CGGCCCAGCCGGACCGCACCACGCGAGGCGCGAGATAGGGGGGCACGGGCGCGACCATCTGC |
| | GCTGCGGCGCCGGCGACTCAGCGCTGCCTCAGTCTGCGGTGGGCAGCGGAGGAGTCGTGTCGT |
| | GCCTGAGAGCGCAGCTGTGCTCCTGGGCACCGCGCAGTCCGCCCCCGCGGCTCCTGGCCAGAC |
| | CACCCCTAGGACCCCCTGCCCCAAGTCGCAGCCGGTCACCAAGCAGGAAGTCAAAGACTTTTT |
| | CCGGTGGGCAAAGGATCACGTGGTTGAGGTGGAGCATGAATTCTACGTCAAAAAGGGTGGAGC |
| | CAAGAAAAGACCCGCCCCCAGTGACGCAGATATAAGTGAGCCCAAACGGGTGCGCGAGTCAG |
| | TTGCGCAGCCATCGACGTCAGACGCGGAAGCTTCGATCAACTACGCGGACAGGTACCAAAACA |
| | AATGTTCTCGTCACGTGGGCATGAATCTGATGCTGTTTCCCTGCAGACAATGCGAGAGACTGAA |
| | TCAGAATTCAAATATCTGCTTCACTCACGGTGTCAAAGACTGTTTAGAGTGCTTTCCCGTGTCA |
| | GAATCTCAACCCGTTTCTGTCGTCAAAAAGGCGTATCAGAAACTGTGCTACATTCATCACATCA |
| | TGGGAAAGGTGCCAGACGCTTGCACTGCTTGCGACCTGGTCAATGTGGACTTGGATGACTGTGT |
| | TTCTGAACAATAAATGACTTAAACCAGGTATGGCTGCCGATGGTTATCTTCCAGATTGGCTCGA |
| | GGACAACCTTAGTGAAGGAATTCGCGAGTGGTGGGCTTTGAAACCTGGAGCCCCTCAACCCAA |
| | GGCAAATCAACAACATCAAGACAACGCTCGAGGTCTTGTGCTTCCGGGTTACAAATACCTTGG |
| | ACCCGGCAACGGACTCGACAAGGGGGAGCCGGTCAACGCAGCAGACGCGGCGGCCCTCGAGC |
| | ACGACAAGGCCTACGACCAGCAGCTCAAGGCCGGAGACAACCCGTACCTCAAGTACAACCACG |
| | CCGACGCCGAGTTCCAGGAGCGGCTCAAAGAAGATACGTCTTTTGGGGGCAACCTCGGGCGAG |
| | CAGTCTTCCAGGCCAAAAAGAGGCTTCTTGAACCTCTTGGTCTGGTTGAGGAAGCGGCTAAGA |
| | CGGCTCCTGGAAAGAAGAGGCCTGTAGAGCAGTCTCCTCAGGAACCGGACTCCTCCGCGGGTA |
| | TTGGCAAATCGGGTGCACAGCCCGCTAAAAAGAGACTCAATTTCGGTCAGACTGGCGACACAG |
| | AGTCAGTCCCAGACCCTCAACCAATCGGAGAACCTCCCGCAGCCCCCTCAGGTGTGGGATCTCT |
| | TACAATGGCTTCAGGTGGTGGCGCACCAGTGGCAGACAATAACGAAGGTGCCGATGGAGTGGG |
| | TAGTTCCTCGGGAAATTGGCATTGCGATTCCCAATGGCTGGGGGACAGAGTCATCACCACCAG |
| | CACCCGAACCTGGGCCCTGCCCACCTACAACAATCACCTCTACAAGCAAATCTCCAACAGCAC |
| | ATCTGGAGGATCTTCAAATGACAACGCCTACTTCGGCTACAGCACCCCCTGGGGGTATTTTGAC |
| | TTCAACAGATTCCACTGCCACTTCTCACCACGTGACTGGCAGCGACTCATCAACAACAACTGGG |
| | GATTCCGGCCTAAGCGACTCAACTTCAAGCTCTTCAACATTCAGGTCAAAGAGGTTACGGACA |
| | ACAATGGAGTCAAGACCATCGCCAATAACCTTACCAGCACGGTCCAGGTCTTCACGGACTCAG |
| | ACTATCAGCTCCCGTACGTGCTCGGGTCGGCTCACGAGGGCTGCCTCCCGCCGTTCCCAGCGGA |
| | CGTTTTCATGATTCCTCAGTACGGGTATCTGACGCTTAATGATGGAAGCCAGGCCGTGGGTCGT |
| | TCGTCCTTTTACTGCCTGGAATATTTCCCGTCGCAAATGCTAAGAACGGGTAACAACTTCCAGT |
| | TCAGCTACGAGTTTGAGAACGTACCTTTCCATAGCAGCTACGCTCACAGCCAAAGCCTGGACCG |
| | ACTAATGAATCCACTCATCGACCAATACTTGTACTATCTCTCAAAGACTATTAACGGTTCTGGA |
| | CAGAATCAACAAACGCTAAAATTCAGTGTGGCCGGACCCAGCAACATGGCTGTCCAGGGAAGA |
| | AACTACATACCTGGACCCAGCTACCGACAACAACGTGTCTCAACCACTGTGACTCAAAACAAC |
| | AACAGCGAATTTGCTTGGCCTGGAGCTTCTTCTTGGGCTCTCAATGGACGTAATAGCTTGATGA |
| | ATCCTGGACCTGCTATGGCCAGCCACAAAGAAGGAGAGGACCGTTTCTTTCCTTTGTCTGGATC |
| | TTTAATTTTTGGCAAACAAGGAACTGGAAGAGACAACGTGGATGCGGACAAAGTCATGATAAC |
| | CAACGAAGAAGAAATTAAAACTACTAACCCGGTAGCAACGGAGTCCTATGGACAAGTGGCCAC |
| | AAACCACCAGAGTGCCCAAGCACAGGCGCAGACCGGCTGGGTTCAAAACCAAGGAATACTTCC |
| | GGGTATGGTTTGGCAGGACAGAGATGTGTACCTGCAAGGACCCATTTGGGCCAAAATTCCTCA |
| | CACGGACGGCAACTTTCACCCTTCTCCGCTGATGGGAGGGTTTGGAATGAAGCACCCGCCTCCT |
| | CAGATCCTCATCAAAAACACACCTGTACCTGCGGATCCTCCAACGGCCTTCAACAAGGACAAG |
| | CTGAACTCTTTCATCACCCAGTATTCTACTGGCCAAGTCAGCGTGGAGATCGAGTGGGAGCTGC |
| | AGAAGGAAAACAGCAAGCGCTGGAACCCGGAGATCCAGTACACTTCCAACTATTACAAGTCTA |
| | ATAATGTTGAATTTGCTGTTAATACTGAAGGTGTATATAGTGAACCCCGCCCCATTGGCACCAG |
| | ATACCTGACTCGTAATCTGTAATCGATTGTTAATCAATAAACCGTTTAATTCGTTTCAGTTGAAC |
| | TTTGGTCTCTGCGTATTTCTTTCTTATCTAGTTTCCATGGCTACGTAGATAAGTAGCATGGCGGG |
| | TTAATCATTAACTACAAGGAACCCCTAGTGATGGAGTTGGCCACTCCCTCTCTGCGCGCTCGCT |
| | CGCTCACTGAGGCCGGGCGACCAAAGGTCGCCCGACGCCCGGGCTTTGCCCGGGCGGCCTCAG |
| | TGAGCGAGCGAGCGCGCAGAGAGGGAGTGGCCAA |
| GFAP-CAP9 | TTGGCCACTCCCTCTCTGCGCGCTCGCTCGCTCACTGAGGCCGGGCGACCAAAGGTCGCCCGAC |
| SEQ ID NO: 9 | GCCCGGGCTTTGCCCGGGCGGCCTCAGTGAGCGAGCGAGCGCGCAGAGAGGGAGTGGCCAACT |
| | CCATCACTAGGGGTTCCTGGAGGGGTGGAGTCGTGACGATATCGATCTAACATATCCTGGTGTG |
| | GAGTAGCGGACGCTGCTATGACAGAGGCTCGGGGGCCTGAGCTGGCTCTGTGAGCTGGGGAGG |
| | AGGCAGACAGCCAGGCCTTGTCTGCAAGCAGACCTGGCAGCATTGGGCTGGCCGCCCCCCAGG |
| | GCCTCCTCTTCATGCCCAGTGAATGACTCACCTTGGCACAGACACAATGTTCGGGGTGGGCACA |
| | GTGCCTGCTTCCCGCCGCACCCCAGCCCCCCTCAAATGCCTTCCGAGAAGCCCATTGAGCAGGG |
| | GGCTTGCATTGCACCCCAGCCTGACAGCCTGGCATCTTGGGATAAAAGCAGCACAGCCCCCTA |
| | GGGGCTGCCCTTGCTGTGTGGCGCCACCGGCGGTGGAGAACAAGGCTCTATTCAGCCTGTGCCC |
| | AGGAAAGGGGATCAGGGGATGCCCAGGCATGGACAGTGGGTGGCAGGGGGGGAGAGGAGGG |
| | CTGTCTGCTTCCCAGAAGTCCAAGGACACAAATGGGTGAGGGGAGAGCTCTCCCCATAGCTGG |
| | GCTGCGGCCCAACCCCACCCCCTCAGGCTATGCCAGGGGGTGTTGCCAGGGGCACCCGGGCAT |
| | CGCCAGTCTAGCCCACTCCTTCATAAAGCCCTCGCATCCCAGGAGCGAGCAGAGCCAGAGCAG |
| | GTTGGAGAGGAGACGCATCACCTCCGCTGCTCGCGGGGATCCTCTAGGGTCACCAAGCAGGAA |
| | GTCAAAGACTTTTTCCGGTGGGCAAAGGATCACGTGGTTGAGGTGGAGCATGAATTCTACGTC |
| | AAAAAGGGTGGAGCCAAGAAAAGACCCGCCCCCAGTGACGCAGATATAAGTGAGCCCAAACG |
| | GGTGCGCGAGTCAGTTGCGCAGCCATCGACGTCAGACGCGGAAGCTTCGATCAACTACGCGGA |
| | CAGGTACCAAAACAAATGTTCTCGTCACGTGGGCATGAATCTGATGCTGTTTCCCTGCAGACAA |
| | TGCGAGAGACTGAATCAGAATTCAAATATCTGCTTCACTCACGGTGTCAAAGACTGTTTAGAGT |
| | GCTTTCCCGTGTCAGAATCTCAACCCGTTTCTGTCGTCAAAAAGGCGTATCAGAAACTGTGCTA |
| | CATTCATCACATCATGGGAAAGGTGCCAGACGCTTGCACTGCTTGCGACCTGGTCAATGTGGAC |
| | TTGGATGACTGTGTTTCTGAACAATAAATGACTTAAACCAGGTATGGCTGCCGATGGTTATCTT |
| | CCAGATTGGCTCGAGGACAACCTTAGTGAAGGAATTCGCGAGTGGTGGGCTTTGAAACCTGGA |
| | GCCCCTCAACCCAAGGCAAATCAACAACATCAAGACAACGCTCGAGGTCTTGTGCTTCCGGGT |
| | TACAAATACCTTGGACCCGGCAACGGACTCGACAAGGGGGAGCCGGTCAACGCAGCAGACGC |
| | GGCGGCCCTCGAGCACGACAAGGCCTACGACCAGCAGCTCAAGGCCGGAGACAACCCGTACCT |
| | CAAGTACAACCACGCCGACGCCGAGTTCCAGGAGCGGCTCAAAGAAGATACGTCTTTTGGGGG |
| | CAACCTCGGGCGAGCAGTCTTCCAGGCCAAAAAGAGGCTTCTTGAACCTCTTGGTCTGGTTGAG |
| | GAAGCGGCTAAGACGGCTCCTGGAAAGAAGAGGCCTGTAGAGCAGTCTCCTCAGGAACCGGA |
| | CTCCTCCGCGGGTATTGGCAAATCGGGTGCACAGCCCGCTAAAAAGAGACTCAATTTCGGTCA |
| | GACTGGCGACACAGAGTCAGTCCCAGACCCTCAACCAATCGGAGAACCTCCCGCAGCCCCCTC |
| | AGGTGTGGGATCTCTTACAATGGCTTCAGGTGGTGGCGCACCAGTGGCAGACAATAACGAAGG |
| | TGCCGATGGAGTGGGTAGTTCCTCGGGAAATTGGCATTGCGATTCCCAATGGCTGGGGGACAG |
| | AGTCATCACCACCAGCACCCGAACCTGGGCCCTGCCCACCTACAACAATCACCTCTACAAGCA |
| | AATCTCCAACAGCACATCTGGAGGATCTTCAAATGACAACGCCTACTTCGGCTACAGCACCCCC |
| | TGGGGGTATTTTGACTTCAACAGATTCCACTGCCACTTCTCACCACGTGACTGGCAGCGACTCA |
| | TCAACAACAACTGGGGATTCCGGCCTAAGCGACTCAACTTCAAGCTCTTCAACATTCAGGTCAA |
| | AGAGGTTACGGACAACAATGGAGTCAAGACCATCGCCAATAACCTTACCAGCACGGTCCAGGT |
| | CTTCACGGACTCAGACTATCAGCTCCCGTACGTGCTCGGGTCGGCTCACGAGGGCTGCCTCCCG |
| | CCGTTCCCAGCGGACGTTTTCATGATTCCTCAGTACGGGTATCTGACGCTTAATGATGGAAGCC |
| | AGGCCGTGGGTCGTTCGTCCTTTTACTGCCTGGAATATTTCCCGTCGCAAATGCTAAGAACGGG |
| | TAACAACTTCCAGTTCAGCTACGAGTTTGAGAACGTACCTTTCCATAGCAGCTACGCTCACAGC |
| | CAAAGCCTGGACCGACTAATGAATCCACTCATCGACCAATACTTGTACTATCTCTCAAAGACTA |
| | TTAACGGTTCTGGACAGAATCAACAAACGCTAAAATTCAGTGTGGCCGGACCCAGCAACATGG |
| | CTGTCCAGGGAAGAAACTACATACCTGGACCCAGCTACCGACAACAACGTGTCTCAACCACTG |
| | TGACTCAAAACAACAACAGCGAATTTGCTTGGCCTGGAGCTTCTTCTTGGGCTCTCAATGGACG |
| | TAATAGCTTGATGAATCCTGGACCTGCTATGGCCAGCCACAAAGAAGGAGAGGACCGTTTCTTT |
| | CCTTTGTCTGGATCTTTAATTTTTGGCAAACAAGGAACTGGAAGAGACAACGTGGATGCGGAC |
| | AAAGTCATGATAACCAACGAAGAAGAAATTAAAACTACTAACCCGGTAGCAACGGAGTCCTAT |
| | GGACAAGTGGCCACAAACCACCAGAGTGCCCAAGCACAGGCGCAGACCGGCTGGGTTCAAAA |
| | CCAAGGAATACTTCCGGGTATGGTTTGGCAGGACAGAGATGTGTACCTGCAAGGACCCATTTG |
| | GGCCAAAATTCCTCACACGGACGGCAACTTTCACCCTTCTCCGCTGATGGGAGGGTTTGGAATG |
| | AAGCACCCGCCTCCTCAGATCCTCATCAAAAACACACCTGTACCTGCGGATCCTCCAACGGCCT |
| | TCAACAAGGACAAGCTGAACTCTTTCATCACCCAGTATTCTACTGGCCAAGTCAGCGTGGAGAT |
| | CGAGTGGGAGCTGCAGAAGGAAAACAGCAAGCGCTGGAACCCGGAGATCCAGTACACTTCCA |
| | ACTATTACAAGTCTAATAATGTTGAATTTGCTGTTAATACTGAAGGTGTATATAGTGAACCCCG |
| | CCCCATTGGCACCAGATACCTGACTCGTAATCTGTAATCGATTGTTAATCAATAAACCGTTTAA |
| | TTCGTTTCAGTTGAACTTTGGTCTCTGCGTATTTCTTTCTTATCTAGTTTCCATGGCTACGTAGAT |
| | AAGTAGCATGGCGGGTTAATCATTAACTACAAGGAACCCCTAGTGATGGAGTTGGCCACTCCC |
| | TCTCTGCGCGCTCGCTCGCTCACTGAGGCCGGGCGACCAAAGGTCGCCCGACGCCCGGGCTTTG |
| | CCCGGGCGGCCTCAGTGAGCGAGCGAGCGCGCAGAGAGGGAGTGGCCAA |
| CAG-CAP9 | TTGGCCACTCCCTCTCTGCGCGCTCGCTCGCTCACTGAGGCCGGGCGACCAAAGGTCGCC |
| SEQ ID NO: 10 | CGACGCCCGGGCTTTGCCCGGGCGGCCTCAGTGAGCGAGCGAGCGCGCAGAGAGGGAGT |
| | GGCCAACTCCATCACTAGGGGTTCCTGGAGGGGTGGAGTCGTGACGATATCCATGCGTCG |
| | ACATAACGCGTCGACATTGATTATTGACTAGTTATTAATAGTAATCAATTACGGGGTCATT |
| | AGTTCATAGCCCATATATGGAGTTCCGCGTTACATAACTTACGGTAAATGGCCCGCCTGG |
| | CTGACCGCCCAACGACCCCCGCCCATTGACGTCAATAATGACGTATGTTCCCATAGTAAC |
| | GCCAATAGGGACTTTCCATTGACGTCAATGGGTGGAGTATTTACGGTAAACTGCCCACTT |
| | GGCAGTACATCAAGTGTATCATATGCCAAGTACGCCCCCTATTGACGTCAATGACGGTAA |
| | ATGGCCCGCCTGGCATTATGCCCAGTACATGACCTTATGGGACTTTCCTACTTGGCAGTAC |
| | ATCTACGTATTAGTCATCGCTATTACCATGTCGAGGCCACGTTCTGCTTCACTCTCCCCAT |
| | CTCCCCCCCCTCCCCACCCCCAATTTTGTATTTATTTATTTTTTAATTATTTTGTGCAGCGA |
| | TGGGGGCGGGGGGGGGGGGCGCGCGCCAGGCGGGGCGGGGCGGGGCGAGGGGCGGGGC |
| | GGGGCGAGGCGGAGAGGTGCGGCGGCAGCCAATCAGAGCGGCGCGCTCCGAAAGTTTCC |
| | TTTTATGGCGAGGCGGCGGCGGCGGCGGCCCTATAAAAAGCGAAGCGCGCGGCGGGCGG |
| | GAGCAAGCTTCGTTTAGTGAACCGTCAGATCGCCTGGAGACGCCATCCACGCTGTTTTGA |
| | CCTCCATAGAAGACACCGGGACCGATCCAGCCTCCGCGGATTCGAATCCCGGCCGGGAA |
| | CGGTGCATTGGAACGCGGATTCCCCGTGCCAAGAGTGACGTAAGTACCGCCTATAGAGTC |
| | TATAGGCCCACAAAAAATGCTTTCTTCTTTTAATATACTTTTTTGTTTATCTTATTTCTAAT |
| | ACTTTCCCTAATCTCTTTCTTTCAGGGCAATAATGATACAATGTATCATGCCTCTTTGCAC |
| | CATTCTAAAGAATAACAGTGATAATTTCTGGGTTAAGGCAATAGCAATATTTCTGCATAT |
| | AAATATTTCTGCATATAAATTGTAACTGATGTAAGAGGTTTCATATTGCTAATAGCAGCTA |
| | CAATCCAGCTACCATTCTGCTTTTATTTTATGGTTGGGATAAGGCTGGATTATTCTGAGTC |
| | CAAGCTAGGCCCTTTTGCTAATCATGTTCATACCTCTTATCTTCCTCCCACAGCTCCTGGG |
| | CAACGTGCTGGTCTGTGTGCTGGCCCATCACTTTGGCAAAGAATTGGGATTCGAACCGGT |
| | CACCAAGCAGGAAGTCAAAGACTTTTTCCGGTGGGCAAAGGATCACGTGGTTGAGGTGG |
| | AGCATGAATTCTACGTCAAAAAGGGTGGAGCCAAGAAAAGACCCGCCCCCAGTGACGCA |
| | GATATAAGTGAGCCCAAACGGGTGCGCGAGTCAGTTGCGCAGCCATCGACGTCAGACGC |
| | GGAAGCTTCGATCAACTACGCGGACAGGTACCAAAACAAATGTTCTCGTCACGTGGGCAT |
| | GAATCTGATGCTGTTTCCCTGCAGACAATGCGAGAGACTGAATCAGAATTCAAATATCTG |
| | CTTCACTCACGGTGTCAAAGACTGTTTAGAGTGCTTTCCCGTGTCAGAATCTCAACCCGTT |
| | TCTGTCGTCAAAAAGGCGTATCAGAAACTGTGCTACATTCATCACATCATGGGAAAGGTG |
| | CCAGACGCTTGCACTGCTTGCGACCTGGTCAATGTGGACTTGGATGACTGTGTTTCTGAAC |
| | AATAAATGACTTAAACCAGGTATGGCTGCCGATGGTTATCTTCCAGATTGGCTCGAGGAC |
| | AACCTTAGTGAAGGAATTCGCGAGTGGTGGGCTTTGAAACCTGGAGCCCCTCAACCCAAG |
| | GCAAATCAACAACATCAAGACAACGCTCGAGGTCTTGTGCTTCCGGGTTACAAATACCTT |
| | GGACCCGGCAACGGACTCGACAAGGGGGAGCCGGTCAACGCAGCAGACGCGGCGGCCCT |
| | CGAGCACGACAAGGCCTACGACCAGCAGCTCAAGGCCGGAGACAACCCGTACCTCAAGT |
| | ACAACCACGCCGACGCCGAGTTCCAGGAGCGGCTCAAAGAAGATACGTCTTTTGGGGGC |
| | AACCTCGGGCGAGCAGTCTTCCAGGCCAAAAAGAGGCTTCTTGAACCTCTTGGTCTGGTT |
| | GAGGAAGCGGCTAAGACGGCTCCTGGAAAGAAGAGGCCTGTAGAGCAGTCTCCTCAGGA |
| | ACCGGACTCCTCCGCGGGTATTGGCAAATCGGGTGCACAGCCCGCTAAAAAGAGACTCA |
| | ATTTCGGTCAGACTGGCGACACAGAGTCAGTCCCAGACCCTCAACCAATCGGAGAACCTC |
| | CCGCAGCCCCCTCAGGTGTGGGATCTCTTACAATGGCTTCAGGTGGTGGCGCACCAGTGG |
| | CAGACAATAACGAAGGTGCCGATGGAGTGGGTAGTTCCTCGGGAAATTGGCATTGCGATT |
| | CCCAATGGCTGGGGGACAGAGTCATCACCACCAGCACCCGAACCTGGGCCCTGCCCACCT |
| | ACAACAATCACCTCTACAAGCAAATCTCCAACAGCACATCTGGAGGATCTTCAAATGACA |
| | ACGCCTACTTCGGCTACAGCACCCCCTGGGGGTATTTTGACTTCAACAGATTCCACTGCCA |
| | CTTCTCACCACGTGACTGGCAGCGACTCATCAACAACAACTGGGGATTCCGGCCTAAGCG |
| | ACTCAACTTCAAGCTCTTCAACATTCAGGTCAAAGAGGTTACGGACAACAATGGAGTCAA |
| | GACCATCGCCAATAACCTTACCAGCACGGTCCAGGTCTTCACGGACTCAGACTATCAGCT |
| | CCCGTACGTGCTCGGGTCGGCTCACGAGGGCTGCCTCCCGCCGTTCCCAGCGGACGTTTT |
| | CATGATTCCTCAGTACGGGTATCTGACGCTTAATGATGGAAGCCAGGCCGTGGGTCGTTC |
| | GTCCTTTTACTGCCTGGAATATTTCCCGTCGCAAATGCTAAGAACGGGTAACAACTTCCA |
| | GTTCAGCTACGAGTTTGAGAACGTACCTTTCCATAGCAGCTACGCTCACAGCCAAAGCCT |
| | GGACCGACTAATGAATCCACTCATCGACCAATACTTGTACTATCTCTCAAAGACTATTAA |
| | CGGTTCTGGACAGAATCAACAAACGCTAAAATTCAGTGTGGCCGGACCCAGCAACATGG |
| | CTGTCCAGGGAAGAAACTACATACCTGGACCCAGCTACCGACAACAACGTGTCTCAACCA |
| | CTGTGACTCAAAACAACAACAGCGAATTTGCTTGGCCTGGAGCTTCTTCTTGGGCTCTCA |
| | ATGGACGTAATAGCTTGATGAATCCTGGACCTGCTATGGCCAGCCACAAAGAAGGAGAG |
| | GACCGTTTCTTTCCTTTGTCTGGATCTTTAATTTTTGGCAAACAAGGAACTGGAAGAGACA |
| | ACGTGGATGCGGACAAAGTCATGATAACCAACGAAGAAGAAATTAAAACTACTAACCCG |
| | GTAGCAACGGAGTCCTATGGACAAGTGGCCACAAACCACCAGAGTGCCCAAGCACAGGC |
| | GCAGACCGGCTGGGTTCAAAACCAAGGAATACTTCCGGGTATGGTTTGGCAGGACAGAG |
| | ATGTGTACCTGCAAGGACCCATTTGGGCCAAAATTCCTCACACGGACGGCAACTTTCACC |
| | CTTCTCCGCTGATGGGAGGGTTTGGAATGAAGCACCCGCCTCCTCAGATCCTCATCAAAA |
| | ACACACCTGTACCTGCGGATCCTCCAACGGCCTTCAACAAGGACAAGCTGAACTCTTTCA |
| | TCACCCAGTATTCTACTGGCCAAGTCAGCGTGGAGATCGAGTGGGAGCTGCAGAAGGAA |
| | AACAGCAAGCGCTGGAACCCGGAGATCCAGTACACTTCCAACTATTACAAGTCTAATAAT |
| | GTTGAATTTGCTGTTAATACTGAAGGTGTATATAGTGAACCCCGCCCCATTGGCACCAGA |
| | TACCTGACTCGTAATCTGTAATCGATTGTTAATCAATAAACCGTTTAATTCGTTTCAGTTG |
| | AACTTTGGTCTCTGCGTATTTCTTTCTTATCTAGTTTCCATGGCTACGTAGATAAGTAGCAT |
| | GGCGGGTTAATCATTAACTACAAGGAACCCCTAGTGATGGAGTTGGCCACTCCCTCTCTG |
| | CGCGCTCGCTCGCTCACTGAGGCCGGGCGACCAAAGGTCGCCCGACGCCCGGGCTTTGCC |
| | CGGGCGGCCTCAGTGAGCGAGCGAGCGCGCAGAGAGGGAGTGGCCAA |
| SYNG-CAP9 | TTGGCCACTCCCTCTCTGCGCGCTCGCTCGCTCACTGAGGCCGGGCGACCAAAGGTCGCC |
| SEQ ID NO: 11 | CGACGCCCGGGCTTTGCCCGGGCGGCCTCAGTGAGCGAGCGAGCGCGCAGAGAGGGAGT |
| | GGCCAACTCCATCACTAGGGGTTCCTGGAGGGGTGGAGTCGTGACGATATCCATGCGTCG |
| | ACATAACGCGTGATCTAACATATCCTGGTGTGGAGTAGCGGACGCTGCTATGACAGAGGC |
| | TCGGGGGCCTGAGCTGGCTCTGTGAGCTGGGGAGGAGGCAGACAGCCAGGCCTTGTCTG |
| | CAAGCAGACCTGGCAGCATTGGGCTGGCCGCCCCCCAGGGCCTCCTCTTCATGCCCAGTG |
| | AATGACTCACCTTGGCACAGACACAATGTTCGGGGTGGGCACAGTGCCTGCTTCCCGCCG |
| | CACCCCAGCCCCCCTCAAATGCCTTCCGAGAAGCCCATTGAGCAGGGGGCTTGCATTGCA |
| | CCCCAGCCTGACAGCCTGGCATCTTGGGATAAAAGCAGCACAGCCCCCTAGGGGCTGCCC |
| | TTGCTGTGTGGCGCCACCGGCGGTGGAGAACAAGGCTCTATTCAGCCTGTGCCCAGGAAA |
| | GGGGATCAGGGGATGCCCAGGCATGGACAGTGGGTGGCAGGGGGGGAGAGGAGGGCTG |
| | TCTGCTTCCCAGAAGTCCAAGGACACAAATGGGTGAGGGGAGAGCTCTCCCCATAGCTGG |
| | GCTGCGGCCCAACCCCACCCCCTCAGGCTATGCCAGGGGGTGTTGCCAGGGGCACCCGGG |
| | CATCGCCAGTCTAGCCCACTCCTTCATAAAGCCCTCGCATCCCAGGAGCGAGCAGAGCCA |
| | GAGCAGGTTGGAGAGGAGACGCATCACCTCCGCTGCTCGCGGGGATCCTCTAGAAGCTTC |
| | GTTTAGTGAACCGTCAGATCGCCTGGAGACGCCATCCACGCTGTTTTGACCTCCATAGAA |
| | GACACCGGGACCGATCCAGCCTCCGCGGATTCGAATCCCGGCCGGGAACGGTGCATTGG |
| | AACGCGGATTCCCCGTGCCAAGAGTGACGTAAGTACCGCCTATAGAGTCTATAGGCCCAC |
| | AAAAAATGCTTTCTTCTTTTAATATACTTTTTTGTTTATCTTATTTCTAATACTTTCCCTAAT |
| | CTCTTTCTTTCAGGGCAATAATGATACAATGTATCATGCCTCTTTGCACCATTCTAAAGAA |
| | TAACAGTGATAATTTCTGGGTTAAGGCAATAGCAATATTTCTGCATATAAATATTTCTGCA |
| | TATAAATTGTAACTGATGTAAGAGGTTTCATATTGCTAATAGCAGCTACAATCCAGCTAC |
| | CATTCTGCTTTTATTTTATGGTTGGGATAAGGCTGGATTATTCTGAGTCCAAGCTAGGCCC |
| | TTTTGCTAATCATGTTCATACCTCTTATCTTCCTCCCACAGCTCCTGGGCAACGTGCTGGTC |
| | TGTGTGCTGGCCCATCACTTTGGCAAAGAATTGGGATTCGAACCGGTCGCCACCGGTCAC |
| | CAAGCAGGAAGTCAAAGACTTTTTCCGGTGGGCAAAGGATCACGTGGTTGAGGTGGAGC |
| | ATGAATTCTACGTCAAAAAGGGTGGAGCCAAGAAAAGACCCGCCCCCAGTGACGCAGAT |
| | ATAAGTGAGCCCAAACGGGTGCGCGAGTCAGTTGCGCAGCCATCGACGTCAGACGCGGA |
| | AGCTTCGATCAACTACGCGGACAGGTACCAAAACAAATGTTCTCGTCACGTGGGCATGAA |
| | TCTGATGCTGTTTCCCTGCAGACAATGCGAGAGACTGAATCAGAATTCAAATATCTGCTT |
| | CACTCACGGTGTCAAAGACTGTTTAGAGTGCTTTCCCGTGTCAGAATCTCAACCCGTTTCT |
| | GTCGTCAAAAAGGCGTATCAGAAACTGTGCTACATTCATCACATCATGGGAAAGGTGCCA |
| | GACGCTTGCACTGCTTGCGACCTGGTCAATGTGGACTTGGATGACTGTGTTTCTGAACAAT |
| | AAATGACTTAAACCAGGTATGGCTGCCGATGGTTATCTTCCAGATTGGCTCGAGGACAAC |
| | CTTAGTGAAGGAATTCGCGAGTGGTGGGCTTTGAAACCTGGAGCCCCTCAACCCAAGGCA |
| | AATCAACAACATCAAGACAACGCTCGAGGTCTTGTGCTTCCGGGTTACAAATACCTTGGA |
| | CCCGGCAACGGACTCGACAAGGGGGAGCCGGTCAACGCAGCAGACGCGGCGGCCCTCGA |
| | GCACGACAAGGCCTACGACCAGCAGCTCAAGGCCGGAGACAACCCGTACCTCAAGTACA |
| | ACCACGCCGACGCCGAGTTCCAGGAGCGGCTCAAAGAAGATACGTCTTTTGGGGGCAAC |
| | CTCGGGCGAGCAGTCTTCCAGGCCAAAAAGAGGCTTCTTGAACCTCTTGGTCTGGTTGAG |
| | GAAGCGGCTAAGACGGCTCCTGGAAAGAAGAGGCCTGTAGAGCAGTCTCCTCAGGAACC |
| | GGACTCCTCCGCGGGTATTGGCAAATCGGGTGCACAGCCCGCTAAAAAGAGACTCAATTT |
| | CGGTCAGACTGGCGACACAGAGTCAGTCCCAGACCCTCAACCAATCGGAGAACCTCCCG |
| | CAGCCCCCTCAGGTGTGGGATCTCTTACAATGGCTTCAGGTGGTGGCGCACCAGTGGCAG |
| | ACAATAACGAAGGTGCCGATGGAGTGGGTAGTTCCTCGGGAAATTGGCATTGCGATTCCC |
| | AATGGCTGGGGGACAGAGTCATCACCACCAGCACCCGAACCTGGGCCCTGCCCACCTAC |
| | AACAATCACCTCTACAAGCAAATCTCCAACAGCACATCTGGAGGATCTTCAAATGACAAC |
| | GCCTACTTCGGCTACAGCACCCCCTGGGGGTATTTTGACTTCAACAGATTCCACTGCCACT |
| | TCTCACCACGTGACTGGCAGCGACTCATCAACAACAACTGGGGATTCCGGCCTAAGCGAC |
| | TCAACTTCAAGCTCTTCAACATTCAGGTCAAAGAGGTTACGGACAACAATGGAGTCAAGA |
| | CCATCGCCAATAACCTTACCAGCACGGTCCAGGTCTTCACGGACTCAGACTATCAGCTCC |
| | CGTACGTGCTCGGGTCGGCTCACGAGGGCTGCCTCCCGCCGTTCCCAGCGGACGTTTTCA |
| | TGATTCCTCAGTACGGGTATCTGACGCTTAATGATGGAAGCCAGGCCGTGGGTCGTTCGT |
| | CCTTTTACTGCCTGGAATATTTCCCGTCGCAAATGCTAAGAACGGGTAACAACTTCCAGTT |
| | CAGCTACGAGTTTGAGAACGTACCTTTCCATAGCAGCTACGCTCACAGCCAAAGCCTGGA |
| | CCGACTAATGAATCCACTCATCGACCAATACTTGTACTATCTCTCAAAGACTATTAACGGT |
| | TCTGGACAGAATCAACAAACGCTAAAATTCAGTGTGGCCGGACCCAGCAACATGGCTGTC |
| | CAGGGAAGAAACTACATACCTGGACCCAGCTACCGACAACAACGTGTCTCAACCACTGT |
| | GACTCAAAACAACAACAGCGAATTTGCTTGGCCTGGAGCTTCTTCTTGGGCTCTCAATGG |
| | ACGTAATAGCTTGATGAATCCTGGACCTGCTATGGCCAGCCACAAAGAAGGAGAGGACC |
| | GTTTCTTTCCTTTGTCTGGATCTTTAATTTTTGGCAAACAAGGAACTGGAAGAGACAACGT |
| | GGATGCGGACAAAGTCATGATAACCAACGAAGAAGAAATTAAAACTACTAACCCGGTAG |
| | CAACGGAGTCCTATGGACAAGTGGCCACAAACCACCAGAGTGCCCAAGCACAGGCGCAG |
| | ACCGGCTGGGTTCAAAACCAAGGAATACTTCCGGGTATGGTTTGGCAGGACAGAGATGT |
| | GTACCTGCAAGGACCCATTTGGGCCAAAATTCCTCACACGGACGGCAACTTTCACCCTTC |
| | TCCGCTGATGGGAGGGTTTGGAATGAAGCACCCGCCTCCTCAGATCCTCATCAAAAACAC |
| | ACCTGTACCTGCGGATCCTCCAACGGCCTTCAACAAGGACAAGCTGAACTCTTTCATCAC |
| | CCAGTATTCTACTGGCCAAGTCAGCGTGGAGATCGAGTGGGAGCTGCAGAAGGAAAACA |
| | GCAAGCGCTGGAACCCGGAGATCCAGTACACTTCCAACTATTACAAGTCTAATAATGTTG |
| | AATTTGCTGTTAATACTGAAGGTGTATATAGTGAACCCCGCCCCATTGGCACCAGATACC |
| | TGACTCGTAATCTGTAATCGATTGTTAATCAATAAACCGTTTAATTCGTTTCAGTTGAACT |
| | TTGGTCTCTGCGTATTTCTTTCTTATCTAGTTTCCATGGCTACGTAGATAAGTAGCATGGC |
| | GGGTTAATCATTAACTACAAGGAACCCCTAGTGATGGAGTTGGCCACTCCCTCTCTGCGC |
| | GCTCGCTCGCTCACTGAGGCCGGGCGACCAAAGGTCGCCCGACGCCCGGGCTTTGCCCGG |
| | GCGGCCTCAGTGAGCGAGCGAGCGCGCAGAGAGGGAGTGGCCAA |
| GFAPG-CAP9 | TTGGCCACTCCCTCTCTGCGCGCTCGCTCGCTCACTGAGGCCGGGCGACCAAAGGTCGCC |
| SEQ ID NO: 12 | CGACGCCCGGGCTTTGCCCGGGCGGCCTCAGTGAGCGAGCGAGCGCGCAGAGAGGGAGT |
| | GGCCAACTCCATCACTAGGGGTTCCTGGAGGGGTGGAGTCGTGACGATATCCATGCGTCG |
| | ACATAACGCGTTAGTATCTGCAGAGGGCCCTGCGTATGAGTGCAAGTGGGTTTTAGGACC |
| | AGGATGAGGCGGGGTGGGGGTGCCTACCTGACGACCGACCCCGACCCACTGGACAAGCA |
| | CCCAACCCCCATTCCCCAAATTGCGCATCCCCTATCAGAGAGGGGGAGGGGAAACAGGA |
| | TGCGGCGAGGCGCGTGCGCACTGCCAGCTTCAGCACCGCGGACAGTGCCTTCGCCCCCGC |
| | CTGGCGGCGCGCGCCACCGCCGCCTCAGCACTGAAGGCGCGCTGACGTCACTCGCCGGTC |
| | CCCCGCAAACTCCCCTTCCCGGCCACCTTGGTCGCGTCCGCGCCGCCGCCGGCCCAGCCG |
| | GACCGCACCACGCGAGGCGCGAGATAGGGGGGCACGGGCGCGACCATCTGCGCTGCGGC |
| | GCCGGCGACTCAGCGCTGCCTCAGTCTGCGGTGGGCAGCGGAGGAGTCGTGTCGTGCCTG |
| | AGAGCGCAGCTGTGCTCCTGGGCACCGCGCAGTCCGCCCCCGCGGCTCCTGGCCAGACCA |
| | CCCCTAGGACCCCCTGCCCCAAGTCGCAGCCAAGCTTCGTTTAGTGAACCGTCAGATCGC |
| | CTGGAGACGCCATCCACGCTGTTTTGACCTCCATAGAAGACACCGGGACCGATCCAGCCT |
| | CCGCGGATTCGAATCCCGGCCGGGAACGGTGCATTGGAACGCGGATTCCCCGTGCCAAG |
| | AGTGACGTAAGTACCGCCTATAGAGTCTATAGGCCCACAAAAAATGCTTTCTTCTTTTAAT |
| | ATACTTTTTTGTTTATCTTATTTCTAATACTTTCCCTAATCTCTTTCTTTCAGGGCAATAATG |
| | ATACAATGTATCATGCCTCTTTGCACCATTCTAAAGAATAACAGTGATAATTTCTGGGTTA |
| | AGGCAATAGCAATATTTCTGCATATAAATATTTCTGCATATAAATTGTAACTGATGTAAG |
| | AGGTTTCATATTGCTAATAGCAGCTACAATCCAGCTACCATTCTGCTTTTATTTTATGGTT |
| | GGGATAAGGCTGGATTATTCTGAGTCCAAGCTAGGCCCTTTTGCTAATCATGTTCATACCT |
| | CTTATCTTCCTCCCACAGCTCCTGGGCAACGTGCTGGTCTGTGTGCTGGCCCATCACTTTG |
| | GCAAAGAATTGGGATTCGAACCGGTCGCCACCGGTCACCAAGCAGGAAGTCAAAGACTT |
| | TTTCCGGTGGGCAAAGGATCACGTGGTTGAGGTGGAGCATGAATTCTACGTCAAAAAGG |
| | GTGGAGCCAAGAAAAGACCCGCCCCCAGTGACGCAGATATAAGTGAGCCCAAACGGGTG |
| | CGCGAGTCAGTTGCGCAGCCATCGACGTCAGACGCGGAAGCTTCGATCAACTACGCGGA |
| | CAGGTACCAAAACAAATGTTCTCGTCACGTGGGCATGAATCTGATGCTGTTTCCCTGCAG |
| | ACAATGCGAGAGACTGAATCAGAATTCAAATATCTGCTTCACTCACGGTGTCAAAGACTG |
| | TTTAGAGTGCTTTCCCGTGTCAGAATCTCAACCCGTTTCTGTCGTCAAAAAGGCGTATCAG |
| | AAACTGTGCTACATTCATCACATCATGGGAAAGGTGCCAGACGCTTGCACTGCTTGCGAC |
| | CTGGTCAATGTGGACTTGGATGACTGTGTTTCTGAACAATAAATGACTTAAACCAGGTAT |
| | GGCTGCCGATGGTTATCTTCCAGATTGGCTCGAGGACAACCTTAGTGAAGGAATTCGCGA |
| | GTGGTGGGCTTTGAAACCTGGAGCCCCTCAACCCAAGGCAAATCAACAACATCAAGACA |
| | ACGCTCGAGGTCTTGTGCTTCCGGGTTACAAATACCTTGGACCCGGCAACGGACTCGACA |
| | AGGGGGAGCCGGTCAACGCAGCAGACGCGGCGGCCCTCGAGCACGACAAGGCCTACGAC |
| | CAGCAGCTCAAGGCCGGAGACAACCCGTACCTCAAGTACAACCACGCCGACGCCGAGTT |
| | CCAGGAGCGGCTCAAAGAAGATACGTCTTTTGGGGGCAACCTCGGGCGAGCAGTCTTCCA |
| | GGCCAAAAAGAGGCTTCTTGAACCTCTTGGTCTGGTTGAGGAAGCGGCTAAGACGGCTCC |
| | TGGAAAGAAGAGGCCTGTAGAGCAGTCTCCTCAGGAACCGGACTCCTCCGCGGGTATTG |
| | GCAAATCGGGTGCACAGCCCGCTAAAAAGAGACTCAATTTCGGTCAGACTGGCGACACA |
| | GAGTCAGTCCCAGACCCTCAACCAATCGGAGAACCTCCCGCAGCCCCCTCAGGTGTGGGA |
| | TCTCTTACAATGGCTTCAGGTGGTGGCGCACCAGTGGCAGACAATAACGAAGGTGCCGAT |
| | GGAGTGGGTAGTTCCTCGGGAAATTGGCATTGCGATTCCCAATGGCTGGGGGACAGAGTC |
| | ATCACCACCAGCACCCGAACCTGGGCCCTGCCCACCTACAACAATCACCTCTACAAGCAA |
| | ATCTCCAACAGCACATCTGGAGGATCTTCAAATGACAACGCCTACTTCGGCTACAGCACC |
| | CCCTGGGGGTATTTTGACTTCAACAGATTCCACTGCCACTTCTCACCACGTGACTGGCAGC |
| | GACTCATCAACAACAACTGGGGATTCCGGCCTAAGCGACTCAACTTCAAGCTCTTCAACA |
| | TTCAGGTCAAAGAGGTTACGGACAACAATGGAGTCAAGACCATCGCCAATAACCTTACC |
| | AGCACGGTCCAGGTCTTCACGGACTCAGACTATCAGCTCCCGTACGTGCTCGGGTCGGCT |
| | CACGAGGGCTGCCTCCCGCCGTTCCCAGCGGACGTTTTCATGATTCCTCAGTACGGGTATC |
| | TGACGCTTAATGATGGAAGCCAGGCCGTGGGTCGTTCGTCCTTTTACTGCCTGGAATATTT |
| | CCCGTCGCAAATGCTAAGAACGGGTAACAACTTCCAGTTCAGCTACGAGTTTGAGAACGT |
| | ACCTTTCCATAGCAGCTACGCTCACAGCCAAAGCCTGGACCGACTAATGAATCCACTCAT |
| | CGACCAATACTTGTACTATCTCTCAAAGACTATTAACGGTTCTGGACAGAATCAACAAAC |
| | GCTAAAATTCAGTGTGGCCGGACCCAGCAACATGGCTGTCCAGGGAAGAAACTACATAC |
| | CTGGACCCAGCTACCGACAACAACGTGTCTCAACCACTGTGACTCAAAACAACAACAGC |
| | GAATTTGCTTGGCCTGGAGCTTCTTCTTGGGCTCTCAATGGACGTAATAGCTTGATGAATC |
| | CTGGACCTGCTATGGCCAGCCACAAAGAAGGAGAGGACCGTTTCTTTCCTTTGTCTGGAT |
| | CTTTAATTTTTGGCAAACAAGGAACTGGAAGAGACAACGTGGATGCGGACAAAGTCATG |
| | ATAACCAACGAAGAAGAAATTAAAACTACTAACCCGGTAGCAACGGAGTCCTATGGACA |
| | AGTGGCCACAAACCACCAGAGTGCCCAAGCACAGGCGCAGACCGGCTGGGTTCAAAACC |
| | AAGGAATACTTCCGGGTATGGTTTGGCAGGACAGAGATGTGTACCTGCAAGGACCCATTT |
| | GGGCCAAAATTCCTCACACGGACGGCAACTTTCACCCTTCTCCGCTGATGGGAGGGTTTG |
| | GAATGAAGCACCCGCCTCCTCAGATCCTCATCAAAAACACACCTGTACCTGCGGATCCTC |
| | CAACGGCCTTCAACAAGGACAAGCTGAACTCTTTCATCACCCAGTATTCTACTGGCCAAG |
| | TCAGCGTGGAGATCGAGTGGGAGCTGCAGAAGGAAAACAGCAAGCGCTGGAACCCGGA |
| | GATCCAGTACACTTCCAACTATTACAAGTCTAATAATGTTGAATTTGCTGTTAATACTGAA |
| | GGTGTATATAGTGAACCCCGCCCCATTGGCACCAGATACCTGACTCGTAATCTGTAATCG |
| | ATTGTTAATCAATAAACCGTTTAATTCGTTTCAGTTGAACTTTGGTCTCTGCGTATTTCTTT |
| | CTTATCTAGTTTCCATGGCTACGTAGATAAGTAGCATGGCGGGTTAATCATTAACTACAA |
| | GGAACCCCTAGTGATGGAGTTGGCCACTCCCTCTCTGCGCGCTCGCTCGCTCACTGAGGC |
| | CGGGCGACCAAAGGTCGCCCGACGCCCGGGCTTTGCCCGGGCGGCCTCAGTGAGCGAGC |
| | GAGCGCGCAGAGAGGGAGTGGCCAA |
| GLOSPLICEF6 | GTGCCAAGAGTGACCTCCTG |
| SEQ ID NO: 13 | |
| CAP5L8 | ACTGCCCCCGCGACCGGCACGTACAACCTCCAGGAAATCGTGCCCGGCAGCGTGTGGATG |
| GBLOCK SEQ | GAGAGGGACGTGTACCTCCAAGGACCCATCTGGGCCAAGATCCCAGAGACGGGGGCGCA |
| ID NO: 14 | CTTTCACCCCTCTCCGGCTATGGGCGGATTCGGACTCAAACACCCACCGCCCATGATGCTC |
| | ATCAAGAACACGCCTGTGCCCGGAAATATCACCAGCTTCTCGGACGTGCCCGTCAGCAGC |
| | TTCATCACCCAGTACAGCACCGGGCAGGTCACCGTGGAGATGGAGTGGGAGCTCAAGAA |
| | GGAAAACTCCAAGAGGTGGAACCCAGAGATCCAGTACACAAACAACTACAACGACCCCC |
| | AGTTTGTGGACTTTGCCCCGGACAGCACCGGGGAATACAGAACCACCAGACCTATCGGA |
| | ACCCGATACCTTACCCGACCCCTTTAA |
| CAP6L8 | ACCGGAGATGTGCATGTTATGGGAGCCTTACCTGGAATGGTGTGGCAAGACAGGGACGT |
| GBLOCK SEQ | CTACCTGCAGGGTCCTATTTGGGCCAAAATTCCTCACACGGATGGACACTTTCACCCATCT |
| ID NO: 15 | CCTCTCATGGGCGGCTTTGGACTTAAGCACCCGCCTCCTCAGATCCTCATCAAAAACACG |
| | CCTGTTCCTGCGAATCCTCCGGCAGAGTTTTCGGCTACAAAGTTTGCTTCATTCATCACCC |
| | AGTATTCCACAGGACAAGTGAGCGTGGAGATTGAATGGGAGCTGCAGAAAGAAAACAGC |
| | AAACGCTGGAATCCCGAAGTGCAATATACATCTAACTATGCAAAATCTGCCAACGTTGAT |
| | TTCACTGTGGACAACAATGGACTTTATACTGAGCCTCGCCCCATTGGCACCCGTTACCTCA |
| | CCCGTCCCCTGTAATCGAT |
| CAPDJ8L8 | ACACAAGCAGCTACCGCAGATGTCAACACACAAGGCGTTCTTCCAGGCATGGTCTGGCAG |
| GBLOCK SEQ | GACAGAGATGTGTACCTTCAGGGGCCCATCTGGGCAAAGATTCCACACACGGACGGACA |
| ID NO: 16 | TTTTCACCCCTCTCCCCTCATGGGTGGATTCGGACTTAAACACCCTCCGCCTCAGATCCTG |
| | ATCAAGAACACGCCTGTACCTGCGGACCCTCCGACCACCTTCAACCAGTCAAAGCTGAAC |
| | TCTTTCATCACCCAGTATTCTACTGGCCAAGTCAGCGTGGAGATCGAGTGGGAGCTGCAG |
| | AAGGAAAACAGCAAGCGCTGGAACCCCGAGATCCAGTACACCTCCAACTACTACAAATC |
| | TACAAGTGTGGACTTTGCTGTTAATACAGAAGGCGTGTACTCTGAACCCCGCCCCATTGG |
| | CACCCGTTACCTCACCCGTAATCTGTAA |
| CAP9L8M | GCACAGGCGCAGACCGGCTGGGTTCAAAACCAAGGAATACTTCCGGGTATGGTTTGGCA |
| GBLOCK SEQ | GGACAGAGATGTGTACCTGCAAGGACCCATTTGGGCCAAAATTCCTCACACGGACGGCA |
| ID NO: 17 | ACTTTCACCCTTCTCCGCTGATGGGAGGGTTTGGAATGAAGCACCCGCCTCCTCAGATCCT |
| | CATCAAAAACACACCTGTACCTGCCGATCCTCCAACGGCCTTCAACAAGGACAAGCTGAA |
| | CTCTTTCATCACCCAGTATTCTACTGGCCAAGTCAGCGTGGAGATCGAGTGGGAGCTGCA |
| | GAAGGAAAACAGCAAGCGGTGGAACCCGGAGATCCAGTACACTTCCAACTATTACAAGT |
| | CTAATAATGTTGAATTTGCTGTTAATACTGAAGGTGTATATAGTGAACCCCGCCCCATTGG |
| | CACCAGATACCTGACTCGTAATCTGTAA |
| TELN-SYNG9- | TATCAGCACACAATAGTCCATTATACGCGCGTATAATGGGCAATTGTGTGCTGATACAGC |
| BSRGI SEQ ID | TGGCACGACAGGTTTCCCGACTGGAAAGCGGGCAGTGAGCGCAACGCAATTAATGTGAG |
| NO: 18 | TTAGCTCACTCATTAGGCACCCCAGGCTTTACACTTTATGCTTCCGGCTCGTATGTTGTGT |
| | GGAATTGTGAGCGGATAACAATTTCACACAGGAAACAGCTATGACCATGATTACGCCAG |
| | ATTTAATTAAGGCCTTAATTAGGCTAGCTTGGCCACTCCCTCTCTGCGCGCTCGCTCGCTC |
| | ACTGAGGCCGGGCGACCAAAGGTCGCCCGACGCCCGGGCTTTGCCCGGGCGGCCTCAGT |
| | GAGCGAGCGAGCGCGCAGAGAGGGAGTGGCCAACTCCATCACTAGGGGTTCCTGGAGGG |
| | GTGGAGTCGTGACGATATCCATGCGTCGACATAACGCGTTAGTATCTGCAGAGGGCCCTG |
| | CGTATGAGTGCAAGTGGGTTTTAGGACCAGGATGAGGCGGGGTGGGGGTGCCTACCTGA |
| | CGACCGACCCCGACCCACTGGACAAGCACCCAACCCCCATTCCCCAAATTGCGCATCCCC |
| | TATCAGAGAGGGGGAGGGGAAACAGGATGCGGCGAGGCGCGTGCGCACTGCCAGCTTCA |
| | GCACCGCGGACAGTGCCTTCGCCCCCGCCTGGCGGCGCGCGCCACCGCCGCCTCAGCACT |
| | GAAGGCGCGCTGACGTCACTCGCCGGTCCCCCGCAAACTCCCCTTCCCGGCCACCTTGGT |
| | CGCGTCCGCGCCGCCGCCGGCCCAGCCGGACCGCACCACGCGAGGCGCGAGATAGGGGG |
| | GCACGGGCGCGACCATCTGCGCTGCGGCGCCGGCGACTCAGCGCTGCCTCAGTCTGCGGT |
| | GGGCAGCGGAGGAGTCGTGTCGTGCCTGAGAGCGCAGCTGTGCTCCTGGGCACCGCGCA |
| | GTCCGCCCCCGCGGCTCCTGGCCAGACCACCCCTAGGACCCCCTGCCCCAAGTCGCAGCC |
| | AAGCTTCGTTTAGTGAACCGTCAGATCGCCTGGAGACGCCATCCACGCTGTTTTGACCTCC |
| | ATAGAAGACACCGGGACCGATCCAGCCTCCGCGGATTCGAATCCCGGCCGGGAACGGTG |
| | CATTGGAACGCGGATTCCCCGTGCCAAGAGTGACGTAAGTACCGCCTATAGAGTCTATAG |
| | GCCCACAAAAAATGCTTTCTTCTTTTAATATACTTTTTTGTTTATCTTATTTCTAATACTTTC |
| | CCTAATCTCTTTCTTTCAGGGCAATAATGATACAATGTATCATGCCTCTTTGCACCATTCT |
| | AAAGAATAACAGTGATAATTTCTGGGTTAAGGCAATAGCAATATTTCTGCATATAAATAT |
| | TTCTGCATATAAATTGTAACTGATGTAAGAGGTTTCATATTGCTAATAGCAGCTACAATCC |
| | AGCTACCATTCTGCTTTTATTTTATGGTTGGGATAAGGCTGGATTATTCTGAGTCCAAGCT |
| | AGGCCCTTTTGCTAATCATGTTCATACCTCTTATCTTCCTCCCACAGCTCCTGGGCAACGT |
| | GCTGGTCTGTGTGCTGGCCCATCACTTTGGCAAAGAATTGGGATTCGAACCGGTCGCCAC |
| | CGGTCACCAAGCAGGAAGTCAAAGACTTTTTCCGGTGGGCAAAGGATCACGTGGTTGAG |
| | GTGGAGCATGAATTCTACGTCAAAAAGGGTGGAGCCAAGAAAAGACCCGCCCCCAGTGA |
| | CGCAGATATAAGTGAGCCCAAACGGGTGCGCGAGTCAGTTGCGCAGCCATCGACGTCAG |
| | ACGCGGAAGCTTCGATCAACTACGCGGACAGGTACCAAAACAAATGTTCTCGTCACGTGG |
| | GCATGAATCTGATGCTGTTTCCCTGCAGACAATGCGAGAGACTGAATCAGAATTCAAATA |
| | TCTGCTTCACTCACGGTGTCAAAGACTGTTTAGAGTGCTTTCCCGTGTCAGAATCTCAACC |
| | CGTTTCTGTCGTCAAAAAGGCGTATCAGAAACTGTGCTACATTCATCACATCATGGGAAA |
| | GGTGCCAGACGCTTGCACTGCTTGCGACCTGGTCAATGTGGACTTGGATGACTGTGTTTCT |
| | GAACAATAAATGACTTAAACCAGGTATGGCTGCCGATGGTTATCTTCCAGATTGGCTCGA |
| | GGACAACCTTAGTGAAGGAATTCGCGAGTGGTGGGCTTTGAAACCTGGAGCCCCTCAACC |
| | CAAGGCAAATCAACAACATCAAGACAACGCTCGAGGTCTTGTGCTTCCGGGTTACAAATA |
| | CCTTGGACCCGGCAACGGACTCGACAAGGGGGAGCCGGTCAACGCAGCAGACGCGGCGG |
| | CCCTCGAGCACGACAAGGCCTACGACCAGCAGCTCAAGGCCGGAGACAACCCGTACCTC |
| | AAGTACAACCACGCCGACGCCGAGTTCCAGGAGCGGCTCAAAGAAGATACGTCTTTTGG |
| | GGGCAACCTCGGGCGAGCAGTCTTCCAGGCCAAAAAGAGGCTTCTTGAACCTCTTGGTCT |
| | GGTTGAGGAAGCGGCTAAGACGGCTCCTGGAAAGAAGAGGCCTGTAGAGCAGTCTCCTC |
| | AGGAACCGGACTCCTCCGCGGGTATTGGCAAATCGGGTGCACAGCCCGCTAAAAAGAGA |
| | CTCAATTTCGGTCAGACTGGCGACACAGAGTCAGTCCCAGACCCTCAACCAATCGGAGAA |
| | CCTCCCGCAGCCCCCTCAGGTGTGGGATCTCTTACAATGGCTTCAGGTGGTGGCGCACCA |
| | GTGGCAGACAATAACGAAGGTGCCGATGGAGTGGGTAGTTCCTCGGGAAATTGGCATTG |
| | CGATTCCCAATGGCTGGGGGACAGAGTCATCACCACCAGCACCCGAACCTGGGCCCTGCC |
| | CACCTACAACAATCACCTCTACAAGCAAATCTCCAACAGCACATCTGGAGGATCTTCAAA |
| | TGACAACGCCTACTTCGGCTACAGCACCCCCTGGGGGTATTTTGACTTCAACAGATTCCA |
| | CTGCCACTTCTCACCACGTGACTGGCAGCGACTCATCAACAACAACTGGGGATTCCGGCC |
| | TAAGCGACTCAACTTCAAGCTCTTCAACATTCAGGTCAAAGAGGTTACGGACAACAATGG |
| | AGTCAAGACCATCGCCAATAACCTTACCAGCACGGTCCAGGTCTTCACGGACTCAGACTA |
| | TCAGCTCCCGTACGTGCTCGGGTCGGCTCACGAGGGCTGCCTCCCGCCGTTCCCAGCGGA |
| | CGTTTTCATGATTCCTCAGTACGGGTATCTGACGCTTAATGATGGAAGCCAGGCCGTGGG |
| | TCGTTCGTCCTTTTACTGCCTGGAATATTTCCCGTCGCAAATGCTAAGAACGGGTAACAAC |
| | TTCCAGTTCAGCTACGAGTTTGAGAACGTACCTTTCCATAGCAGCTACGCTCACAGCCAA |
| | AGCCTGGACCGACTAATGAATCCACTCATCGACCAATACTTGTACTATCTCTCAAAGACT |
| | ATTAACGGTTCTGGACAGAATCAACAAACGCTAAAATTCAGTGTGGCCGGACCCAGCAA |
| | CATGGCTGTCCAGGGAAGAAACTACATACCTGGACCCAGCTACCGACAACAACGTGTCTC |
| | AACCACTGTGACTCAAAACAACAACAGCGAATTTGCTTGGCCTGGAGCTTCTTCTTGGGC |
| | TCTCAATGGACGTAATAGCTTGATGAATCCTGGACCTGCTATGGCCAGCCACAAAGAAGG |
| | AGAGGACCGTTTCTTTCCTTTGTCTGGATCTTTAATTTTTGGCAAACAAGGAACTGGAAGA |
| | GACAACGTGGATGCGGACAAAGTCATGATAACCAACGAAGAAGAAATTAAAACTACTAA |
| | CCCGGTAGCAACGGAGTCCTATGGACAAGTGGCCACAAACCACCAGAGTGTACATCGAT |
| | TGTTAATCAATAAACCGTTTAATTCGTTTCAGTTGAACTTTGGTCTCTGCGTATTTCTTTCT |
| | TATCTAGTTTCCATGGCTACGTAGATAAGTAGCATGGCGGGTTAATCATTAACTACAAGG |
| | AACCCCTAGTGATGGAGTTGGCCACTCCCTCTCTGCGCGCTCGCTCGCTCACTGAGGCCG |
| | GGCGACCAAAGGTCGCCCGACGCCCGGGCTTTGCCCGGGCGGCCTCAGTGAGCGAGCGA |
| | GCGCGCAGAGAGGGAGTGGCCAAGCATGCAATTAACTGGCCGTCGTTTTACAACGTCGTG |
| | ACTGGGAAAACCCTGGCGTTACCCAACTTAATCGCCTTGCAGCACATCCCCCTTTCGCCA |
| | GCTGTATCAGCACACAATTGCCCATTATACGCGCGTATAATGGACTATTGTGTGCTGATA |
| TELN- | TATCAGCACACAATAGTCCATTATACGCGCGTATAATGGGCAATTGTGTGCTGATACAGC |
| GFAPG9- | TGGCACGACAGGTTTCCCGACTGGAAAGCGGGCAGTGAGCGCAACGCAATTAATGTGAG |
| BSRGI SEQ ID | TTAGCTCACTCATTAGGCACCCCAGGCTTTACACTTTATGCTTCCGGCTCGTATGTTGTGT |
| NO: 19 | GGAATTGTGAGCGGATAACAATTTCACACAGGAAACAGCTATGACCATGATTACGCCAG |
| | ATTTAATTAAGGCCTTAATTAGGCTAGCTTGGCCACTCCCTCTCTGCGCGCTCGCTCGCTC |
| | ACTGAGGCCGGGCGACCAAAGGTCGCCCGACGCCCGGGCTTTGCCCGGGCGGCCTCAGT |
| | GAGCGAGCGAGCGCGCAGAGAGGGAGTGGCCAACTCCATCACTAGGGGTTCCTGGAGGG |
| | GTGGAGTCGTGACGATATCCATGCGTCGACATAACGCGTGATCTAACATATCCTGGTGTG |
| | GAGTAGCGGACGCTGCTATGACAGAGGCTCGGGGGCCTGAGCTGGCTCTGTGAGCTGGG |
| | GAGGAGGCAGACAGCCAGGCCTTGTCTGCAAGCAGACCTGGCAGCATTGGGCTGGCCGC |
| | CCCCCAGGGCCTCCTCTTCATGCCCAGTGAATGACTCACCTTGGCACAGACACAATGTTC |
| | GGGGTGGGCACAGTGCCTGCTTCCCGCCGCACCCCAGCCCCCCTCAAATGCCTTCCGAGA |
| | AGCCCATTGAGCAGGGGGCTTGCATTGCACCCCAGCCTGACAGCCTGGCATCTTGGGATA |
| | AAAGCAGCACAGCCCCCTAGGGGCTGCCCTTGCTGTGTGGCGCCACCGGCGGTGGAGAA |
| | CAAGGCTCTATTCAGCCTGTGCCCAGGAAAGGGGATCAGGGGATGCCCAGGCATGGACA |
| | GTGGGTGGCAGGGGGGGAGAGGAGGGCTGTCTGCTTCCCAGAAGTCCAAGGACACAAAT |
| | GGGTGAGGGGAGAGCTCTCCCCATAGCTGGGCTGCGGCCCAACCCCACCCCCTCAGGCTA |
| | TGCCAGGGGGTGTTGCCAGGGGCACCCGGGCATCGCCAGTCTAGCCCACTCCTTCATAAA |
| | GCCCTCGCATCCCAGGAGCGAGCAGAGCCAGAGCAGGTTGGAGAGGAGACGCATCACCT |
| | CCGCTGCTCGCGGGGATCCTCTAGAAGCTTCGTTTAGTGAACCGTCAGATCGCCTGGAGA |
| | CGCCATCCACGCTGTTTTGACCTCCATAGAAGACACCGGGACCGATCCAGCCTCCGCGGA |
| | TTCGAATCCCGGCCGGGAACGGTGCATTGGAACGCGGATTCCCCGTGCCAAGAGTGACGT |
| | AAGTACCGCCTATAGAGTCTATAGGCCCACAAAAAATGCTTTCTTCTTTTAATATACTTTT |
| | TTGTTTATCTTATTTCTAATACTTTCCCTAATCTCTTTCTTTCAGGGCAATAATGATACAAT |
| | GTATCATGCCTCTTTGCACCATTCTAAAGAATAACAGTGATAATTTCTGGGTTAAGGCAAT |
| | AGCAATATTTCTGCATATAAATATTTCTGCATATAAATTGTAACTGATGTAAGAGGTTTCA |
| | TATTGCTAATAGCAGCTACAATCCAGCTACCATTCTGCTTTTATTTTATGGTTGGGATAAG |
| | GCTGGATTATTCTGAGTCCAAGCTAGGCCCTTTTGCTAATCATGTTCATACCTCTTATCTTC |
| | CTCCCACAGCTCCTGGGCAACGTGCTGGTCTGTGTGCTGGCCCATCACTTTGGCAAAGAA |
| | TTGGGATTCGAACCGGTCGCCACCGGTCACCAAGCAGGAAGTCAAAGACTTTTTCCGGTG |
| | GGCAAAGGATCACGTGGTTGAGGTGGAGCATGAATTCTACGTCAAAAAGGGTGGAGCCA |
| | AGAAAAGACCCGCCCCCAGTGACGCAGATATAAGTGAGCCCAAACGGGTGCGCGAGTCA |
| | GTTGCGCAGCCATCGACGTCAGACGCGGAAGCTTCGATCAACTACGCGGACAGGTACCA |
| | AAACAAATGTTCTCGTCACGTGGGCATGAATCTGATGCTGTTTCCCTGCAGACAATGCGA |
| | GAGACTGAATCAGAATTCAAATATCTGCTTCACTCACGGTGTCAAAGACTGTTTAGAGTG |
| | CTTTCCCGTGTCAGAATCTCAACCCGTTTCTGTCGTCAAAAAGGCGTATCAGAAACTGTGC |
| | TACATTCATCACATCATGGGAAAGGTGCCAGACGCTTGCACTGCTTGCGACCTGGTCAAT |
| | GTGGACTTGGATGACTGTGTTTCTGAACAATAAATGACTTAAACCAGGTATGGCTGCCGA |
| | TGGTTATCTTCCAGATTGGCTCGAGGACAACCTTAGTGAAGGAATTCGCGAGTGGTGGGC |
| | TTTGAAACCTGGAGCCCCTCAACCCAAGGCAAATCAACAACATCAAGACAACGCTCGAG |
| | GTCTTGTGCTTCCGGGTTACAAATACCTTGGACCCGGCAACGGACTCGACAAGGGGGAGC |
| | CGGTCAACGCAGCAGACGCGGCGGCCCTCGAGCACGACAAGGCCTACGACCAGCAGCTC |
| | AAGGCCGGAGACAACCCGTACCTCAAGTACAACCACGCCGACGCCGAGTTCCAGGAGCG |
| | GCTCAAAGAAGATACGTCTTTTGGGGGCAACCTCGGGCGAGCAGTCTTCCAGGCCAAAA |
| | AGAGGCTTCTTGAACCTCTTGGTCTGGTTGAGGAAGCGGCTAAGACGGCTCCTGGAAAGA |
| | AGAGGCCTGTAGAGCAGTCTCCTCAGGAACCGGACTCCTCCGCGGGTATTGGCAAATCGG |
| | GTGCACAGCCCGCTAAAAAGAGACTCAATTTCGGTCAGACTGGCGACACAGAGTCAGTC |
| | CCAGACCCTCAACCAATCGGAGAACCTCCCGCAGCCCCCTCAGGTGTGGGATCTCTTACA |
| | ATGGCTTCAGGTGGTGGCGCACCAGTGGCAGACAATAACGAAGGTGCCGATGGAGTGGG |
| | TAGTTCCTCGGGAAATTGGCATTGCGATTCCCAATGGCTGGGGGACAGAGTCATCACCAC |
| | CAGCACCCGAACCTGGGCCCTGCCCACCTACAACAATCACCTCTACAAGCAAATCTCCAA |
| | CAGCACATCTGGAGGATCTTCAAATGACAACGCCTACTTCGGCTACAGCACCCCCTGGGG |
| | GTATTTTGACTTCAACAGATTCCACTGCCACTTCTCACCACGTGACTGGCAGCGACTCATC |
| | AACAACAACTGGGGATTCCGGCCTAAGCGACTCAACTTCAAGCTCTTCAACATTCAGGTC |
| | AAAGAGGTTACGGACAACAATGGAGTCAAGACCATCGCCAATAACCTTACCAGCACGGT |
| | CCAGGTCTTCACGGACTCAGACTATCAGCTCCCGTACGTGCTCGGGTCGGCTCACGAGGG |
| | CTGCCTCCCGCCGTTCCCAGCGGACGTTTTCATGATTCCTCAGTACGGGTATCTGACGCTT |
| | AATGATGGAAGCCAGGCCGTGGGTCGTTCGTCCTTTTACTGCCTGGAATATTTCCCGTCGC |
| | AAATGCTAAGAACGGGTAACAACTTCCAGTTCAGCTACGAGTTTGAGAACGTACCTTTCC |
| | ATAGCAGCTACGCTCACAGCCAAAGCCTGGACCGACTAATGAATCCACTCATCGACCAAT |
| | ACTTGTACTATCTCTCAAAGACTATTAACGGTTCTGGACAGAATCAACAAACGCTAAAAT |
| | TCAGTGTGGCCGGACCCAGCAACATGGCTGTCCAGGGAAGAAACTACATACCTGGACCC |
| | AGCTACCGACAACAACGTGTCTCAACCACTGTGACTCAAAACAACAACAGCGAATTTGCT |
| | TGGCCTGGAGCTTCTTCTTGGGCTCTCAATGGACGTAATAGCTTGATGAATCCTGGACCTG |
| | CTATGGCCAGCCACAAAGAAGGAGAGGACCGTTTCTTTCCTTTGTCTGGATCTTTAATTTT |
| | TGGCAAACAAGGAACTGGAAGAGACAACGTGGATGCGGACAAAGTCATGATAACCAACG |
| | AAGAAGAAATTAAAACTACTAACCCGGTAGCAACGGAGTCCTATGGACAAGTGGCCACA |
| | AACCACCAGAGTGTACATCGATTGTTAATCAATAAACCGTTTAATTCGTTTCAGTTGAACT |
| | TTGGTCTCTGCGTATTTCTTTCTTATCTAGTTTCCATGGCTACGTAGATAAGTAGCATGGC |
| | GGGTTAATCATTAACTACAAGGAACCCCTAGTGATGGAGTTGGCCACTCCCTCTCTGCGC |
| | GCTCGCTCGCTCACTGAGGCCGGGCGACCAAAGGTCGCCCGACGCCCGGGCTTTGCCCGG |
| | GCGGCCTCAGTGAGCGAGCGAGCGCGCAGAGAGGGAGTGGCCAAGCATGCAATTAACTG |
| | GCCGTCGTTTTACAACGTCGTGACTGGGAAAACCCTGGCGTTACCCAACTTAATCGCCTT |
| | GCAGCACATCCCCCTTTCGCCAGCTGTATCAGCACACAATTGCCCATTATACGCGCGTAT |
| | AATGGACTATTGTGTGCTGATA |
| TELN-SYNG5- | TATCAGCACACAATAGTCCATTATACGCGCGTATAATGGGCAATTGTGTGCTGATACAGC |
| BSRGI SEQ ID | TGGCACGACAGGTTTCCCGACTGGAAAGCGGGCAGTGAGCGCAACGCAATTAATGTGAG |
| NO: 20 | TTAGCTCACTCATTAGGCACCCCAGGCTTTACACTTTATGCTTCCGGCTCGTATGTTGTGT |
| | GGAATTGTGAGCGGATAACAATTTCACACAGGAAACAGCTATGACCATGATTACGCCAG |
| | ATTTAATTAAGGCCTTAATTAGGCTAGCTTGGCCACTCCCTCTCTGCGCGCTCGCTCGCTC |
| | ACTGAGGCCGGGCGACCAAAGGTCGCCCGACGCCCGGGCTTTGCCCGGGCGGCCTCAGT |
| | GAGCGAGCGAGCGCGCAGAGAGGGAGTGGCCAACTCCATCACTAGGGGTTCCTGGAGGG |
| | GTGGAGTCGTGACGATATCCATGCGTCGACATAACGCGTTAGTATCTGCAGAGGGCCCTG |
| | CGTATGAGTGCAAGTGGGTTTTAGGACCAGGATGAGGCGGGGTGGGGGTGCCTACCTGA |
| | CGACCGACCCCGACCCACTGGACAAGCACCCAACCCCCATTCCCCAAATTGCGCATCCCC |
| | TATCAGAGAGGGGGAGGGGAAACAGGATGCGGCGAGGCGCGTGCGCACTGCCAGCTTCA |
| | GCACCGCGGACAGTGCCTTCGCCCCCGCCTGGCGGCGCGCGCCACCGCCGCCTCAGCACT |
| | GAAGGCGCGCTGACGTCACTCGCCGGTCCCCCGCAAACTCCCCTTCCCGGCCACCTTGGT |
| | CGCGTCCGCGCCGCCGCCGGCCCAGCCGGACCGCACCACGCGAGGCGCGAGATAGGGGG |
| | GCACGGGCGCGACCATCTGCGCTGCGGCGCCGGCGACTCAGCGCTGCCTCAGTCTGCGGT |
| | GGGCAGCGGAGGAGTCGTGTCGTGCCTGAGAGCGCAGCTGTGCTCCTGGGCACCGCGCA |
| | GTCCGCCCCCGCGGCTCCTGGCCAGACCACCCCTAGGACCCCCTGCCCCAAGTCGCAGCC |
| | AAGCTTCGTTTAGTGAACCGTCAGATCGCCTGGAGACGCCATCCACGCTGTTTTGACCTCC |
| | ATAGAAGACACCGGGACCGATCCAGCCTCCGCGGATTCGAATCCCGGCCGGGAACGGTG |
| | CATTGGAACGCGGATTCCCCGTGCCAAGAGTGACGTAAGTACCGCCTATAGAGTCTATAG |
| | GCCCACAAAAAATGCTTTCTTCTTTTAATATACTTTTTTGTTTATCTTATTTCTAATACTTTC |
| | CCTAATCTCTTTCTTTCAGGGCAATAATGATACAATGTATCATGCCTCTTTGCACCATTCT |
| | AAAGAATAACAGTGATAATTTCTGGGTTAAGGCAATAGCAATATTTCTGCATATAAATAT |
| | TTCTGCATATAAATTGTAACTGATGTAAGAGGTTTCATATTGCTAATAGCAGCTACAATCC |
| | AGCTACCATTCTGCTTTTATTTTATGGTTGGGATAAGGCTGGATTATTCTGAGTCCAAGCT |
| | AGGCCCTTTTGCTAATCATGTTCATACCTCTTATCTTCCTCCCACAGCTCCTGGGCAACGT |
| | GCTGGTCTGTGTGCTGGCCCATCACTTTGGCAAAGAATTGGGATTCGAACCGGTCGCCAC |
| | CGGTCACAAGCAGGAAGTCAAAGACTTTTTCCGGTGGGCAAAGGATCACGTGGTTGAGG |
| | TGGAGCATGAATTCTACGTCAAAAAGGGTGGAGCCAAGAAAAGACCCGCCCCCAGTGAC |
| | GCAGATATAAGTGAGCCCAAACGGGTGCGCGAGTCAGTTGCGCAGCCATCGACGTCAGA |
| | CGCGGAAGCTTCGATCAACTACGCGGACAGGTACCAAAACAAATGTTCTCGTCACGTGGG |
| | CATGAATCTGATGCTGTTTCCCTGCAGACAATGCGAGAGAATGAATCAGAATTCAAATAT |
| | CTGCTTCACTCACGGACAGAAAGACTGTTTAGAGTGCTTTCCCGTGTCAGAATCTCAACC |
| | CGTTTCTGTCGTCAAAAAGGCGTATCAGAAACTGTGCTACATTCATCATATCATGGGAAA |
| | GGTGCCAGACGCTTGCACTGCCTGCGATCTGGTCAATGTGGATTTGGATGACTGCATCTTT |
| | GAACAATAAATGATTTAAATCAGGTATGTCTTTTGTTGATCACCCTCCAGATTGGTTGGAA |
| | GAAGTTGGTGAAGGTCTTCGCGAGTTTTTGGGCCTTGAAGCGGGCCCACCGAAACCAAAA |
| | CCCAATCAGCAGCATCAAGATCAAGCCCGTGGTCTTGTGCTGCCTGGTTATAACTATCTC |
| | GGACCCGGAAACGGTCTCGATCGAGGAGAGCCTGTCAACAGGGCAGACGAGGTCGCGCG |
| | AGAGCACGACATCTCGTACAACGAGCAGCTTGAGGCGGGAGACAACCCCTACCTCAAGT |
| | ACAACCACGCGGACGCCGAGTTTCAGGAGAAGCTCGCCGACGACACATCCTTCGGGGGA |
| | AACCTCGGAAAGGCAGTCTTTCAGGCCAAGAAAAGGGTTCTCGAACCTTTTGGCCTGGTT |
| | GAAGAGGGTGCTAAGACGGCCCCTACCGGAAAGCGGATAGACGACCACTTTCCAAAAAG |
| | AAAGAAGGCCCGGACCGAAGAGGACTCCAAGCCTTCCACCTCGTCAGACGCCGAAGCTG |
| | GACCCAGCGGATCCCAGCAGCTGCAAATCCCAGCCCAACCAGCCTCAAGTTTGGGAGCTG |
| | ATACAATGTCTGCGGGAGGTGGCGGCCCATTGGGCGACAATAACCAAGGTGCCGATGGA |
| | GTGGGCAATGCCTCGGGAGATTGGCATTGCGATTCCACGTGGATGGGGGACAGAGTCGTC |
| | ACCAAGTCCACCCGAACCTGGGTGCTGCCCAGCTACAACAACCACCAGTACCGAGAGAT |
| | CAAAAGCGGCTCCGTCGACGGAAGCAACGCCAACGCCTACTTTGGATACAGCACCCCCTG |
| | GGGGTACTTTGACTTTAACCGCTTCCACAGCCACTGGAGCCCCCGAGACTGGCAAAGACT |
| | CATCAACAACTACTGGGGCTTCAGACCCCGGTCCCTCAGAGTCAAAATCTTCAACATTCA |
| | AGTCAAAGAGGTCACGGTGCAGGACTCCACCACCACCATCGCCAACAACCTCACCTCCAC |
| | CGTCCAAGTGTTTACGGACGACGACTACCAGCTGCCCTACGTCGTCGGCAACGGGACCGA |
| | GGGATGCCTGCCGGCCTTCCCTCCGCAGGTCTTTACGCTGCCGCAGTACGGTTACGCGAC |
| | GCTGAACCGCGACAACACAGAAAATCCCACCGAGAGGAGCAGCTTCTTCTGCCTAGAGT |
| | ACTTTCCCAGCAAGATGCTGAGAACGGGCAACAACTTTGAGTTTACCTACAACTTTGAGG |
| | AGGTGCCCTTCCACTCCAGCTTCGCTCCCAGTCAGAACCTCTTCAAGCTGGCCAACCCGCT |
| | GGTGGACCAGTACTTGTACCGCTTCGTGAGCACAAATAACACTGGCGGAGTCCAGTTCAA |
| | CAAGAACCTGGCCGGGAGATACGCCAACACCTACAAAAACTGGTTCCCGGGGCCCATGG |
| | GCCGAACCCAGGGCTGGAACCTGGGCTCCGGGGTCAACCGCGCCAGTGTCAGCGCCTTCG |
| | CCACGACCAATAGGATGGAGCTCGAGGGCGCGAGTTACCAGGTGCCCCCGCAGCCGAAC |
| | GGCATGACCAACAACCTCCAGGGCAGCAACACCTATGCCCTGGAGAACACTATGATCTTC |
| | AACAGCCAGCCGGCGAACCCGGGCACCACCGCCACGTACCTCGAGGGCAACATGCTCAT |
| | CACCAGCGAGAGCGAGACGCAGCCGGTGAACCGCGTGGCGTACAACGTCGGCGGGCAGA |
| | TGGCCACCAACAACCAGAGCTCTGTACATCGATTGTTAATCAATAAACCGTTTAATTCGTT |
| | TCAGTTGAACTTTGGTCTCTGCGTATTTCTTTCTTATCTAGTTTCCATGGCTACGTAGATAA |
| | GTAGCATGGCGGGTTAATCATTAACTACAAGGAACCCCTAGTGATGGAGTTGGCCACTCC |
| | CTCTCTGCGCGCTCGCTCGCTCACTGAGGCCGGGCGACCAAAGGTCGCCCGACGCCCGGG |
| | CTTTGCCCGGGCGGCCTCAGTGAGCGAGCGAGCGCGCAGAGAGGGAGTGGCCAAGCATG |
| | CAATTAACTGGCCGTCGTTTTACAACGTCGTGACTGGGAAAACCCTGGCGTTACCCAACT |
| | TAATCGCCTTGCAGCACATCCCCCTTTCGCCAGCTGTATCAGCACACAATTGCCCATTATA |
| | CGCGCGTATAATGGACTATTGTGTGCTGATA |
| TELN- | TATCAGCACACAATAGTCCATTATACGCGCGTATAATGGGCAATTGTGTGCTGATACAGC |
| GFAPG5- | TGGCACGACAGGTTTCCCGACTGGAAAGCGGGCAGTGAGCGCAACGCAATTAATGTGAG |
| BSRGI SEQ ID | TTAGCTCACTCATTAGGCACCCCAGGCTTTACACTTTATGCTTCCGGCTCGTATGTTGTGT |
| NO: 21 | GGAATTGTGAGCGGATAACAATTTCACACAGGAAACAGCTATGACCATGATTACGCCAG |
| | ATTTAATTAAGGCCTTAATTAGGCTAGCTTGGCCACTCCCTCTCTGCGCGCTCGCTCGCTC |
| | ACTGAGGCCGGGCGACCAAAGGTCGCCCGACGCCCGGGCTTTGCCCGGGCGGCCTCAGT |
| | GAGCGAGCGAGCGCGCAGAGAGGGAGTGGCCAACTCCATCACTAGGGGTTCCTGGAGGG |
| | GTGGAGTCGTGACGATATCCATGCGTCGACATAACGCGTGATCTAACATATCCTGGTGTG |
| | GAGTAGCGGACGCTGCTATGACAGAGGCTCGGGGGCCTGAGCTGGCTCTGTGAGCTGGG |
| | GAGGAGGCAGACAGCCAGGCCTTGTCTGCAAGCAGACCTGGCAGCATTGGGCTGGCCGC |
| | CCCCCAGGGCCTCCTCTTCATGCCCAGTGAATGACTCACCTTGGCACAGACACAATGTTC |
| | GGGGTGGGCACAGTGCCTGCTTCCCGCCGCACCCCAGCCCCCCTCAAATGCCTTCCGAGA |
| | AGCCCATTGAGCAGGGGGCTTGCATTGCACCCCAGCCTGACAGCCTGGCATCTTGGGATA |
| | AAAGCAGCACAGCCCCCTAGGGGCTGCCCTTGCTGTGTGGCGCCACCGGCGGTGGAGAA |
| | CAAGGCTCTATTCAGCCTGTGCCCAGGAAAGGGGATCAGGGGATGCCCAGGCATGGACA |
| | GTGGGTGGCAGGGGGGGAGAGGAGGGCTGTCTGCTTCCCAGAAGTCCAAGGACACAAAT |
| | GGGTGAGGGGAGAGCTCTCCCCATAGCTGGGCTGCGGCCCAACCCCACCCCCTCAGGCTA |
| | TGCCAGGGGGTGTTGCCAGGGGCACCCGGGCATCGCCAGTCTAGCCCACTCCTTCATAAA |
| | GCCCTCGCATCCCAGGAGCGAGCAGAGCCAGAGCAGGTTGGAGAGGAGACGCATCACCT |
| | CCGCTGCTCGCGGGGATCCTCTAGAAGCTTCGTTTAGTGAACCGTCAGATCGCCTGGAGA |
| | CGCCATCCACGCTGTTTTGACCTCCATAGAAGACACCGGGACCGATCCAGCCTCCGCGGA |
| | TTCGAATCCCGGCCGGGAACGGTGCATTGGAACGCGGATTCCCCGTGCCAAGAGTGACGT |
| | AAGTACCGCCTATAGAGTCTATAGGCCCACAAAAAATGCTTTCTTCTTTTAATATACTTTT |
| | TTGTTTATCTTATTTCTAATACTTTCCCTAATCTCTTTCTTTCAGGGCAATAATGATACAAT |
| | GTATCATGCCTCTTTGCACCATTCTAAAGAATAACAGTGATAATTTCTGGGTTAAGGCAAT |
| | AGCAATATTTCTGCATATAAATATTTCTGCATATAAATTGTAACTGATGTAAGAGGTTTCA |
| | TATTGCTAATAGCAGCTACAATCCAGCTACCATTCTGCTTTTATTTTATGGTTGGGATAAG |
| | GCTGGATTATTCTGAGTCCAAGCTAGGCCCTTTTGCTAATCATGTTCATACCTCTTATCTTC |
| | CTCCCACAGCTCCTGGGCAACGTGCTGGTCTGTGTGCTGGCCCATCACTTTGGCAAAGAA |
| | TTGGGATTCGAACCGGTCGCCACCGGTCACCAAGCAGGAAGTCAAAGACTTTTTCCGGTG |
| | GGCAAAGGATCACGTGGTTGAGGTGGAGCATGAATTCTACGTCAAAAAGGGTGGAGCCA |
| | AGAAAAGACCCGCCCCCAGTGACGCAGATATAAGTGAGCCCAAACGGGTGCGCGAGTCA |
| | GTTGCGCAGCCATCGACGTCAGACGCGGAAGCTTCGATCAACTACGCGGACAGGTACCA |
| | AAACAAATGTTCTCGTCACGTGGGCATGAATCTGATGCTGTTTCCCTGCAGACAATGCGA |
| | GAGAATGAATCAGAATTCAAATATCTGCTTCACTCACGGACAGAAAGACTGTTTAGAGTG |
| | CTTTCCCGTGTCAGAATCTCAACCCGTTTCTGTCGTCAAAAAGGCGTATCAGAAACTGTGC |
| | TACATTCATCATATCATGGGAAAGGTGCCAGACGCTTGCACTGCCTGCGATCTGGTCAAT |
| | GTGGATTTGGATGACTGCATCTTTGAACAATAAATGATTTAAATCAGGTATGTCTTTTGTT |
| | GATCACCCTCCAGATTGGTTGGAAGAAGTTGGTGAAGGTCTTCGCGAGTTTTTGGGCCTT |
| | GAAGCGGGCCCACCGAAACCAAAACCCAATCAGCAGCATCAAGATCAAGCCCGTGGTCT |
| | TGTGCTGCCTGGTTATAACTATCTCGGACCCGGAAACGGTCTCGATCGAGGAGAGCCTGT |
| | CAACAGGGCAGACGAGGTCGCGCGAGAGCACGACATCTCGTACAACGAGCAGCTTGAGG |
| | CGGGAGACAACCCCTACCTCAAGTACAACCACGCGGACGCCGAGTTTCAGGAGAAGCTC |
| | GCCGACGACACATCCTTCGGGGGAAACCTCGGAAAGGCAGTCTTTCAGGCCAAGAAAAG |
| | GGTTCTCGAACCTTTTGGCCTGGTTGAAGAGGGTGCTAAGACGGCCCCTACCGGAAAGCG |
| | GATAGACGACCACTTTCCAAAAAGAAAGAAGGCCCGGACCGAAGAGGACTCCAAGCCTT |
| | CCACCTCGTCAGACGCCGAAGCTGGACCCAGCGGATCCCAGCAGCTGCAAATCCCAGCCC |
| | AACCAGCCTCAAGTTTGGGAGCTGATACAATGTCTGCGGGAGGTGGCGGCCCATTGGGCG |
| | ACAATAACCAAGGTGCCGATGGAGTGGGCAATGCCTCGGGAGATTGGCATTGCGATTCC |
| | ACGTGGATGGGGGACAGAGTCGTCACCAAGTCCACCCGAACCTGGGTGCTGCCCAGCTA |
| | CAACAACCACCAGTACCGAGAGATCAAAAGCGGCTCCGTCGACGGAAGCAACGCCAACG |
| | CCTACTTTGGATACAGCACCCCCTGGGGGTACTTTGACTTTAACCGCTTCCACAGCCACTG |
| | GAGCCCCCGAGACTGGCAAAGACTCATCAACAACTACTGGGGCTTCAGACCCCGGTCCCT |
| | CAGAGTCAAAATCTTCAACATTCAAGTCAAAGAGGTCACGGTGCAGGACTCCACCACCAC |
| | CATCGCCAACAACCTCACCTCCACCGTCCAAGTGTTTACGGACGACGACTACCAGCTGCC |
| | CTACGTCGTCGGCAACGGGACCGAGGGATGCCTGCCGGCCTTCCCTCCGCAGGTCTTTAC |
| | GCTGCCGCAGTACGGTTACGCGACGCTGAACCGCGACAACACAGAAAATCCCACCGAGA |
| | GGAGCAGCTTCTTCTGCCTAGAGTACTTTCCCAGCAAGATGCTGAGAACGGGCAACAACT |
| | TTGAGTTTACCTACAACTTTGAGGAGGTGCCCTTCCACTCCAGCTTCGCTCCCAGTCAGAA |
| | CCTCTTCAAGCTGGCCAACCCGCTGGTGGACCAGTACTTGTACCGCTTCGTGAGCACAAA |
| | TAACACTGGCGGAGTCCAGTTCAACAAGAACCTGGCCGGGAGATACGCCAACACCTACA |
| | AAAACTGGTTCCCGGGGCCCATGGGCCGAACCCAGGGCTGGAACCTGGGCTCCGGGGTC |
| | AACCGCGCCAGTGTCAGCGCCTTCGCCACGACCAATAGGATGGAGCTCGAGGGCGCGAG |
| | TTACCAGGTGCCCCCGCAGCCGAACGGCATGACCAACAACCTCCAGGGCAGCAACACCT |
| | ATGCCCTGGAGAACACTATGATCTTCAACAGCCAGCCGGCGAACCCGGGCACCACCGCC |
| | ACGTACCTCGAGGGCAACATGCTCATCACCAGCGAGAGCGAGACGCAGCCGGTGAACCG |
| | CGTGGCGTACAACGTCGGCGGGCAGATGGCCACCAACAACCAGAGCTCTGTACATCGATT |
| | GTTAATCAATAAACCGTTTAATTCGTTTCAGTTGAACTTTGGTCTCTGCGTATTTCTTTCTT |
| | ATCTAGTTTCCATGGCTACGTAGATAAGTAGCATGGCGGGTTAATCATTAACTACAAGGA |
| | ACCCCTAGTGATGGAGTTGGCCACTCCCTCTCTGCGCGCTCGCTCGCTCACTGAGGCCGG |
| | GCGACCAAAGGTCGCCCGACGCCCGGGCTTTGCCCGGGCGGCCTCAGTGAGCGAGCGAG |
| | CGCGCAGAGAGGGAGTGGCCAAGCATGCAATTAACTGGCCGTCGTTTTACAACGTCGTGA |
| | CTGGGAAAACCCTGGCGTTACCCAACTTAATCGCCTTGCAGCACATCCCCCTTTCGCCAG |
| | CTGTATCAGCACACAATTGCCCATTATACGCGCGTATAATGGACTATTGTGTGCTGATA |
| TELN-SYNG6- | TATCAGCACACAATAGTCCATTATACGCGCGTATAATGGGCAATTGTGTGCTGATACAGC |
| BSRGI SEQ ID | TGGCACGACAGGTTTCCCGACTGGAAAGCGGGCAGTGAGCGCAACGCAATTAATGTGAG |
| NO: 22 | TTAGCTCACTCATTAGGCACCCCAGGCTTTACACTTTATGCTTCCGGCTCGTATGTTGTGT |
| | GGAATTGTGAGCGGATAACAATTTCACACAGGAAACAGCTATGACCATGATTACGCCAG |
| | ATTTAATTAAGGCCTTAATTAGGCTAGCTTGGCCACTCCCTCTCTGCGCGCTCGCTCGCTC |
| | ACTGAGGCCGGGCGACCAAAGGTCGCCCGACGCCCGGGCTTTGCCCGGGCGGCCTCAGT |
| | GAGCGAGCGAGCGCGCAGAGAGGGAGTGGCCAACTCCATCACTAGGGGTTCCTGGAGGG |
| | GTGGAGTCGTGACGATATCCATGCGTCGACATAACGCGTTAGTATCTGCAGAGGGCCCTG |
| | CGTATGAGTGCAAGTGGGTTTTAGGACCAGGATGAGGCGGGGTGGGGGTGCCTACCTGA |
| | CGACCGACCCCGACCCACTGGACAAGCACCCAACCCCCATTCCCCAAATTGCGCATCCCC |
| | TATCAGAGAGGGGGAGGGGAAACAGGATGCGGCGAGGCGCGTGCGCACTGCCAGCTTCA |
| | GCACCGCGGACAGTGCCTTCGCCCCCGCCTGGCGGCGCGCGCCACCGCCGCCTCAGCACT |
| | GAAGGCGCGCTGACGTCACTCGCCGGTCCCCCGCAAACTCCCCTTCCCGGCCACCTTGGT |
| | CGCGTCCGCGCCGCCGCCGGCCCAGCCGGACCGCACCACGCGAGGCGCGAGATAGGGGG |
| | GCACGGGCGCGACCATCTGCGCTGCGGCGCCGGCGACTCAGCGCTGCCTCAGTCTGCGGT |
| | GGGCAGCGGAGGAGTCGTGTCGTGCCTGAGAGCGCAGCTGTGCTCCTGGGCACCGCGCA |
| | GTCCGCCCCCGCGGCTCCTGGCCAGACCACCCCTAGGACCCCCTGCCCCAAGTCGCAGCC |
| | AAGCTTCGTTTAGTGAACCGTCAGATCGCCTGGAGACGCCATCCACGCTGTTTTGACCTCC |
| | ATAGAAGACACCGGGACCGATCCAGCCTCCGCGGATTCGAATCCCGGCCGGGAACGGTG |
| | CATTGGAACGCGGATTCCCCGTGCCAAGAGTGACGTAAGTACCGCCTATAGAGTCTATAG |
| | GCCCACAAAAAATGCTTTCTTCTTTTAATATACTTTTTTGTTTATCTTATTTCTAATACTTTC |
| | CCTAATCTCTTTCTTTCAGGGCAATAATGATACAATGTATCATGCCTCTTTGCACCATTCT |
| | AAAGAATAACAGTGATAATTTCTGGGTTAAGGCAATAGCAATATTTCTGCATATAAATAT |
| | TTCTGCATATAAATTGTAACTGATGTAAGAGGTTTCATATTGCTAATAGCAGCTACAATCC |
| | AGCTACCATTCTGCTTTTATTTTATGGTTGGGATAAGGCTGGATTATTCTGAGTCCAAGCT |
| | AGGCCCTTTTGCTAATCATGTTCATACCTCTTATCTTCCTCCCACAGCTCCTGGGCAACGT |
| | GCTGGTCTGTGTGCTGGCCCATCACTTTGGCAAAGAATTGGGATTCGAACCGGTCGCCAC |
| | CGGTCACAAGCAGGAAGTCAAAGACTTTTTCCGGTGGGCAAAGGATCACGTGGTTGAGG |
| | TGGAGCATGAATTCTACGTCAAAAAGGGTGGAGCCAAGAAAAGACCCGCCCCCAGTGAC |
| | GCAGATATAAGTGAGCCCAAACGGGTGCGCGAGTCAGTTGCGCAGCCATCGACGTCAGA |
| | CGCGGAAGCTTCGATCAACTACGCGGACAGGTACCAAAACAAATGTTCTCGTCACGTGGG |
| | CATGAATCTGATGCTGTTTCCCTGCAGACAATGCGAGAGAATGAATCAGAATTCAAATAT |
| | CTGCTTCACTCACGGACAGAAAGACTGTTTAGAGTGCTTTCCCGTGTCAGAATCTCAACC |
| | CGTTTCTGTCGTCAAAAAGGCGTATCAGAAACTGTGCTACATTCATCATATCATGGGAAA |
| | GGTGCCAGACGCTTGCACTGCCTGCGATCTGGTCAATGTGGATTTGGATGACTGCATCTTT |
| | GAACAATAAATGATTTAAATCAGGTATGGCTGCCGATGGTTATCTTCCAGATTGGCTCGA |
| | GGACAACCTCTCTGAGGGCATTCGCGAGTGGTGGGACTTGAAACCTGGAGCCCCGAAAC |
| | CCAAAGCCAACCAGCAAAAGCAGGACGACGGCCGGGGTCTGGTGCTTCCTGGCTACAAG |
| | TACCTCGGACCCTTCAACGGACTCGACAAGGGGGAGCCCGTCAACGCGGCGGATGCAGC |
| | GGCCCTCGAGCACGACAAGGCCTACGACCAGCAGCTCAAAGCGGGTGACAATCCGTACC |
| | TGCGGTATAACCACGCCGACGCCGAGTTTCAGGAGCGTCTGCAAGAAGATACGTCTTTTG |
| | GGGGCAACCTCGGGCGAGCAGTCTTCCAGGCCAAGAAGAGGGTTCTCGAACCTTTTGGTC |
| | TGGTTGAGGAAGGTGCTAAGACGGCTCCTGGAAAGAAACGTCCGGTAGAGCAGTCGCCA |
| | CAAGAGCCAGACTCCTCCTCGGGCATTGGCAAGACAGGCCAGCAGCCCGCTAAAAAGAG |
| | ACTCAATTTTGGTCAGACTGGCGACTCAGAGTCAGTCCCCGACCCACAACCTCTCGGAGA |
| | ACCTCCAGCAACCCCCGCTGCTGTGGGACCTACTACAATGGCTTCAGGCGGTGGCGCACC |
| | AATGGCAGACAATAACGAAGGCGCCGACGGAGTGGGTAATGCCTCAGGAAATTGGCATT |
| | GCGATTCCACATGGCTGGGCGACAGAGTCATCACCACCAGCACCCGAACATGGGCCTTGC |
| | CCACCTATAACAACCACCTCTACAAGCAAATCTCCAGTGCTTCAACGGGGGCCAGCAACG |
| | ACAACCACTACTTCGGCTACAGCACCCCCTGGGGGTATTTTGATTTCAACAGATTCCACTG |
| | CCATTTCTCACCACGTGACTGGCAGCGACTCATCAACAACAATTGGGGATTCCGGCCCAA |
| | GAGACTCAACTTCAAGCTCTTCAACATCCAAGTCAAGGAGGTCACGACGAATGATGGCGT |
| | CACGACCATCGCTAATAACCTTACCAGCACGGTTCAAGTCTTCTCGGACTCGGAGTACCA |
| | GTTGCCGTACGTCCTCGGCTCTGCGCACCAGGGCTGCCTCCCTCCGTTCCCGGCGGACGTG |
| | TTCATGATTCCGCAGTACGGCTACCTAACGCTCAACAATGGCAGCCAGGCAGTGGGACGG |
| | TCATCCTTTTACTGCCTGGAATATTTCCCATCGCAGATGCTGAGAACGGGCAATAACTTTA |
| | CCTTCAGCTACACCTTCGAGGACGTGCCTTTCCACAGCAGCTACGCGCACAGCCAGAGCC |
| | TGGACCGGCTGATGAATCCTCTCATCGACCAGTACCTGTATTACCTGAACAGAACTCAGA |
| | ATCAGTCCGGAAGTGCCCAAAACAAGGACTTGCTGTTTAGCCGGGGGTCTCCAGCTGGCA |
| | TGTCTGTTCAGCCCAAAAACTGGCTACCTGGACCCTGTTACCGGCAGCAGCGCGTTTCTA |
| | AAACAAAAACAGACAACAACAACAGCAACTTTACCTGGACTGGTGCTTCAAAATATAAC |
| | CTTAATGGGCGTGAATCTATAATCAACCCTGGCACTGCTATGGCCTCACACAAAGACGAC |
| | AAAGACAAGTTCTTTCCCATGAGCGGTGTCATGATTTTTGGAAAGGAGAGCGCCGGAGCT |
| | TCAAACACTGCATTGGACAATGTCATGATCACAGACGAAGAGGAAATCAAAGCCACTAA |
| | CCCCGTGGCCACCGAAAGATTTGGGACTGTGGCAGTCAATCTCCAGAGTGTACATCGATT |
| | GTTAATCAATAAACCGTTTAATTCGTTTCAGTTGAACTTTGGTCTCTGCGTATTTCTTTCTT |
| | ATCTAGTTTCCATGGCTACGTAGATAAGTAGCATGGCGGGTTAATCATTAACTACAAGGA |
| | ACCCCTAGTGATGGAGTTGGCCACTCCCTCTCTGCGCGCTCGCTCGCTCACTGAGGCCGG |
| | GCGACCAAAGGTCGCCCGACGCCCGGGCTTTGCCCGGGCGGCCTCAGTGAGCGAGCGAG |
| | CGCGCAGAGAGGGAGTGGCCAAGCATGCAATTAACTGGCCGTCGTTTTACAACGTCGTGA |
| | CTGGGAAAACCCTGGCGTTACCCAACTTAATCGCCTTGCAGCACATCCCCCTTTCGCCAG |
| | CTGTATCAGCACACAATTGCCCATTATACGCGCGTATAATGGACTATTGTGTGCTGATA |
| TELN- | TATCAGCACACAATAGTCCATTATACGCGCGTATAATGGGCAATTGTGTGCTGATACAGC |
| GFAPG6- | TGGCACGACAGGTTTCCCGACTGGAAAGCGGGCAGTGAGCGCAACGCAATTAATGTGAG |
| BSRGI SEQ ID | TTAGCTCACTCATTAGGCACCCCAGGCTTTACACTTTATGCTTCCGGCTCGTATGTTGTGT |
| NO: 23 | GGAATTGTGAGCGGATAACAATTTCACACAGGAAACAGCTATGACCATGATTACGCCAG |
| | ATTTAATTAAGGCCTTAATTAGGCTAGCTTGGCCACTCCCTCTCTGCGCGCTCGCTCGCTC |
| | ACTGAGGCCGGGCGACCAAAGGTCGCCCGACGCCCGGGCTTTGCCCGGGCGGCCTCAGT |
| | GAGCGAGCGAGCGCGCAGAGAGGGAGTGGCCAACTCCATCACTAGGGGTTCCTGGAGGG |
| | GTGGAGTCGTGACGATATCCATGCGTCGACATAACGCGTGATCTAACATATCCTGGTGTG |
| | GAGTAGCGGACGCTGCTATGACAGAGGCTCGGGGGCCTGAGCTGGCTCTGTGAGCTGGG |
| | GAGGAGGCAGACAGCCAGGCCTTGTCTGCAAGCAGACCTGGCAGCATTGGGCTGGCCGC |
| | CCCCCAGGGCCTCCTCTTCATGCCCAGTGAATGACTCACCTTGGCACAGACACAATGTTC |
| | GGGGTGGGCACAGTGCCTGCTTCCCGCCGCACCCCAGCCCCCCTCAAATGCCTTCCGAGA |
| | AGCCCATTGAGCAGGGGGCTTGCATTGCACCCCAGCCTGACAGCCTGGCATCTTGGGATA |
| | AAAGCAGCACAGCCCCCTAGGGGCTGCCCTTGCTGTGTGGCGCCACCGGCGGTGGAGAA |
| | CAAGGCTCTATTCAGCCTGTGCCCAGGAAAGGGGATCAGGGGATGCCCAGGCATGGACA |
| | GTGGGTGGCAGGGGGGGAGAGGAGGGCTGTCTGCTTCCCAGAAGTCCAAGGACACAAAT |
| | GGGTGAGGGGAGAGCTCTCCCCATAGCTGGGCTGCGGCCCAACCCCACCCCCTCAGGCTA |
| | TGCCAGGGGGTGTTGCCAGGGGCACCCGGGCATCGCCAGTCTAGCCCACTCCTTCATAAA |
| | GCCCTCGCATCCCAGGAGCGAGCAGAGCCAGAGCAGGTTGGAGAGGAGACGCATCACCT |
| | CCGCTGCTCGCGGGGATCCTCTAGAAGCTTCGTTTAGTGAACCGTCAGATCGCCTGGAGA |
| | CGCCATCCACGCTGTTTTGACCTCCATAGAAGACACCGGGACCGATCCAGCCTCCGCGGA |
| | TTCGAATCCCGGCCGGGAACGGTGCATTGGAACGCGGATTCCCCGTGCCAAGAGTGACGT |
| | AAGTACCGCCTATAGAGTCTATAGGCCCACAAAAAATGCTTTCTTCTTTTAATATACTTTT |
| | TTGTTTATCTTATTTCTAATACTTTCCCTAATCTCTTTCTTTCAGGGCAATAATGATACAAT |
| | GTATCATGCCTCTTTGCACCATTCTAAAGAATAACAGTGATAATTTCTGGGTTAAGGCAAT |
| | AGCAATATTTCTGCATATAAATATTTCTGCATATAAATTGTAACTGATGTAAGAGGTTTCA |
| | TATTGCTAATAGCAGCTACAATCCAGCTACCATTCTGCTTTTATTTTATGGTTGGGATAAG |
| | GCTGGATTATTCTGAGTCCAAGCTAGGCCCTTTTGCTAATCATGTTCATACCTCTTATCTTC |
| | CTCCCACAGCTCCTGGGCAACGTGCTGGTCTGTGTGCTGGCCCATCACTTTGGCAAAGAA |
| | TTGGGATTCGAACCGGTCGCCACCGGTCACCAAGCAGGAAGTCAAAGACTTTTTCCGGTG |
| | GGCAAAGGATCACGTGGTTGAGGTGGAGCATGAATTCTACGTCAAAAAGGGTGGAGCCA |
| | AGAAAAGACCCGCCCCCAGTGACGCAGATATAAGTGAGCCCAAACGGGTGCGCGAGTCA |
| | GTTGCGCAGCCATCGACGTCAGACGCGGAAGCTTCGATCAACTACGCGGACAGGTACCA |
| | AAACAAATGTTCTCGTCACGTGGGCATGAATCTGATGCTGTTTCCCTGCAGACAATGCGA |
| | GAGAATGAATCAGAATTCAAATATCTGCTTCACTCACGGACAGAAAGACTGTTTAGAGTG |
| | CTTTCCCGTGTCAGAATCTCAACCCGTTTCTGTCGTCAAAAAGGCGTATCAGAAACTGTGC |
| | TACATTCATCATATCATGGGAAAGGTGCCAGACGCTTGCACTGCCTGCGATCTGGTCAAT |
| | GTGGATTTGGATGACTGCATCTTTGAACAATAAATGATTTAAATCAGGTATGGCTGCCGA |
| | TGGTTATCTTCCAGATTGGCTCGAGGACAACCTCTCTGAGGGCATTCGCGAGTGGTGGGA |
| | CTTGAAACCTGGAGCCCCGAAACCCAAAGCCAACCAGCAAAAGCAGGACGACGGCCGGG |
| | GTCTGGTGCTTCCTGGCTACAAGTACCTCGGACCCTTCAACGGACTCGACAAGGGGGAGC |
| | CCGTCAACGCGGCGGATGCAGCGGCCCTCGAGCACGACAAGGCCTACGACCAGCAGCTC |
| | AAAGCGGGTGACAATCCGTACCTGCGGTATAACCACGCCGACGCCGAGTTTCAGGAGCG |
| | TCTGCAAGAAGATACGTCTTTTGGGGGCAACCTCGGGCGAGCAGTCTTCCAGGCCAAGAA |
| | GAGGGTTCTCGAACCTTTTGGTCTGGTTGAGGAAGGTGCTAAGACGGCTCCTGGAAAGAA |
| | ACGTCCGGTAGAGCAGTCGCCACAAGAGCCAGACTCCTCCTCGGGCATTGGCAAGACAG |
| | GCCAGCAGCCCGCTAAAAAGAGACTCAATTTTGGTCAGACTGGCGACTCAGAGTCAGTCC |
| | CCGACCCACAACCTCTCGGAGAACCTCCAGCAACCCCCGCTGCTGTGGGACCTACTACAA |
| | TGGCTTCAGGCGGTGGCGCACCAATGGCAGACAATAACGAAGGCGCCGACGGAGTGGGT |
| | AATGCCTCAGGAAATTGGCATTGCGATTCCACATGGCTGGGCGACAGAGTCATCACCACC |
| | AGCACCCGAACATGGGCCTTGCCCACCTATAACAACCACCTCTACAAGCAAATCTCCAGT |
| | GCTTCAACGGGGGCCAGCAACGACAACCACTACTTCGGCTACAGCACCCCCTGGGGGTAT |
| | TTTGATTTCAACAGATTCCACTGCCATTTCTCACCACGTGACTGGCAGCGACTCATCAACA |
| | ACAATTGGGGATTCCGGCCCAAGAGACTCAACTTCAAGCTCTTCAACATCCAAGTCAAGG |
| | AGGTCACGACGAATGATGGCGTCACGACCATCGCTAATAACCTTACCAGCACGGTTCAAG |
| | TCTTCTCGGACTCGGAGTACCAGTTGCCGTACGTCCTCGGCTCTGCGCACCAGGGCTGCCT |
| | CCCTCCGTTCCCGGCGGACGTGTTCATGATTCCGCAGTACGGCTACCTAACGCTCAACAA |
| | TGGCAGCCAGGCAGTGGGACGGTCATCCTTTTACTGCCTGGAATATTTCCCATCGCAGAT |
| | GCTGAGAACGGGCAATAACTTTACCTTCAGCTACACCTTCGAGGACGTGCCTTTCCACAG |
| | CAGCTACGCGCACAGCCAGAGCCTGGACCGGCTGATGAATCCTCTCATCGACCAGTACCT |
| | GTATTACCTGAACAGAACTCAGAATCAGTCCGGAAGTGCCCAAAACAAGGACTTGCTGTT |
| | TAGCCGGGGGTCTCCAGCTGGCATGTCTGTTCAGCCCAAAAACTGGCTACCTGGACCCTG |
| | TTACCGGCAGCAGCGCGTTTCTAAAACAAAAACAGACAACAACAACAGCAACTTTACCT |
| | GGACTGGTGCTTCAAAATATAACCTTAATGGGCGTGAATCTATAATCAACCCTGGCACTG |
| | CTATGGCCTCACACAAAGACGACAAAGACAAGTTCTTTCCCATGAGCGGTGTCATGATTT |
| | TTGGAAAGGAGAGCGCCGGAGCTTCAAACACTGCATTGGACAATGTCATGATCACAGAC |
| | GAAGAGGAAATCAAAGCCACTAACCCCGTGGCCACCGAAAGATTTGGGACTGTGGCAGT |
| | CAATCTCCAGAGTGTACATCGATTGTTAATCAATAAACCGTTTAATTCGTTTCAGTTGAAC |
| | TTTGGTCTCTGCGTATTTCTTTCTTATCTAGTTTCCATGGCTACGTAGATAAGTAGCATGGC |
| | GGGTTAATCATTAACTACAAGGAACCCCTAGTGATGGAGTTGGCCACTCCCTCTCTGCGC |
| | GCTCGCTCGCTCACTGAGGCCGGGCGACCAAAGGTCGCCCGACGCCCGGGCTTTGCCCGG |
| | GCGGCCTCAGTGAGCGAGCGAGCGCGCAGAGAGGGAGTGGCCAAGCATGCAATTAACTG |
| | GCCGTCGTTTTACAACGTCGTGACTGGGAAAACCCTGGCGTTACCCAACTTAATCGCCTT |
| | GCAGCACATCCCCCTTTCGCCAGCTGTATCAGCACACAATTGCCCATTATACGCGCGTAT |
| | AATGGACTATTGTGTGCTGATA |
| TELN- | TATCAGCACACAATAGTCCATTATACGCGCGTATAATGGGCAATTGTGTGCTGATACAGC |
| SYNGDJ8- | TGGCACGACAGGTTTCCCGACTGGAAAGCGGGCAGTGAGCGCAACGCAATTAATGTGAG |
| BSRGI SEQ ID | TTAGCTCACTCATTAGGCACCCCAGGCTTTACACTTTATGCTTCCGGCTCGTATGTTGTGT |
| NO: 24 | GGAATTGTGAGCGGATAACAATTTCACACAGGAAACAGCTATGACCATGATTACGCCAG |
| | ATTTAATTAAGGCCTTAATTAGGCTAGCTTGGCCACTCCCTCTCTGCGCGCTCGCTCGCTC |
| | ACTGAGGCCGGGCGACCAAAGGTCGCCCGACGCCCGGGCTTTGCCCGGGCGGCCTCAGT |
| | GAGCGAGCGAGCGCGCAGAGAGGGAGTGGCCAACTCCATCACTAGGGGTTCCTGGAGGG |
| | GTGGAGTCGTGACGATATCCATGCGTCGACATAACGCGTTAGTATCTGCAGAGGGCCCTG |
| | CGTATGAGTGCAAGTGGGTTTTAGGACCAGGATGAGGCGGGGTGGGGGTGCCTACCTGA |
| | CGACCGACCCCGACCCACTGGACAAGCACCCAACCCCCATTCCCCAAATTGCGCATCCCC |
| | TATCAGAGAGGGGGAGGGGAAACAGGATGCGGCGAGGCGCGTGCGCACTGCCAGCTTCA |
| | GCACCGCGGACAGTGCCTTCGCCCCCGCCTGGCGGCGCGCGCCACCGCCGCCTCAGCACT |
| | GAAGGCGCGCTGACGTCACTCGCCGGTCCCCCGCAAACTCCCCTTCCCGGCCACCTTGGT |
| | CGCGTCCGCGCCGCCGCCGGCCCAGCCGGACCGCACCACGCGAGGCGCGAGATAGGGGG |
| | GCACGGGCGCGACCATCTGCGCTGCGGCGCCGGCGACTCAGCGCTGCCTCAGTCTGCGGT |
| | GGGCAGCGGAGGAGTCGTGTCGTGCCTGAGAGCGCAGCTGTGCTCCTGGGCACCGCGCA |
| | GTCCGCCCCCGCGGCTCCTGGCCAGACCACCCCTAGGACCCCCTGCCCCAAGTCGCAGCC |
| | AAGCTTCGTTTAGTGAACCGTCAGATCGCCTGGAGACGCCATCCACGCTGTTTTGACCTCC |
| | ATAGAAGACACCGGGACCGATCCAGCCTCCGCGGATTCGAATCCCGGCCGGGAACGGTG |
| | CATTGGAACGCGGATTCCCCGTGCCAAGAGTGACGTAAGTACCGCCTATAGAGTCTATAG |
| | GCCCACAAAAAATGCTTTCTTCTTTTAATATACTTTTTTGTTTATCTTATTTCTAATACTTTC |
| | CCTAATCTCTTTCTTTCAGGGCAATAATGATACAATGTATCATGCCTCTTTGCACCATTCT |
| | AAAGAATAACAGTGATAATTTCTGGGTTAAGGCAATAGCAATATTTCTGCATATAAATAT |
| | TTCTGCATATAAATTGTAACTGATGTAAGAGGTTTCATATTGCTAATAGCAGCTACAATCC |
| | AGCTACCATTCTGCTTTTATTTTATGGTTGGGATAAGGCTGGATTATTCTGAGTCCAAGCT |
| | AGGCCCTTTTGCTAATCATGTTCATACCTCTTATCTTCCTCCCACAGCTCCTGGGCAACGT |
| | GCTGGTCTGTGTGCTGGCCCATCACTTTGGCAAAGAATTGGGATTCGAACCGGTCGCCAC |
| | CGGTCACAAGCAGGAAGTCAAAGACTTTTTCCGGTGGGCAAAGGATCACGTGGTTGAGG |
| | TGGAGCATGAATTCTACGTCAAAAAGGGTGGAGCCAAGAAAAGACCCGCCCCCAGTGAC |
| | GCAGATATAAGTGAGCCCAAACGGGTGCGCGAGTCAGTTGCGCAGCCATCGACGTCAGA |
| | CGCGGAAGCTTCGATCAACTACGCGGACAGGTACCAAAACAAATGTTCTCGTCACGTGGG |
| | CATGAATCTGATGCTGTTTCCCTGCAGACAATGCGAGAGAATGAATCAGAATTCAAATAT |
| | CTGCTTCACTCACGGACAGAAAGACTGTTTAGAGTGCTTTCCCGTGTCAGAATCTCAACC |
| | CGTTTCTGTCGTCAAAAAGGCGTATCAGAAACTGTGCTACATTCATCATATCATGGGAAA |
| | GGTGCCAGACGCTTGCACTGCCTGCGATCTGGTCAATGTGGATTTGGATGACTGCATCTTT |
| | GAACAATAAATGATTTAAATCAGGTATGGCTGCCGATGGTTATCTTCCAGATTGGCTCGA |
| | GGACACTCTCTCTGAAGGAATAAGACAGTGGTGGAAGCTCAAACCTGGCCCACCACCAC |
| | CAAAGCCCGCAGAGCGGCATAAGGACGACAGCAGGGGTCTTGTGCTTCCTGGGTACAAG |
| | TACCTCGGACCCTTCAACGGACTCGACAAGGGAGAGCCGGTCAACGAGGCAGACGCCGC |
| | GGCCCTCGAGCACGACAAAGCCTACGACCGGCAGCTCGACAGCGGAGACAACCCGTACC |
| | TCAAGTACAACCACGCCGACGCCGAGTTCCAGGAGCGGCTCAAAGAAGATACGTCTTTTG |
| | GGGGCAACCTCGGGCGAGCAGTCTTCCAGGCCAAAAAGAGGCTTCTTGAACCTCTTGGTC |
| | TGGTTGAGGAAGCGGCTAAGACGGCTCCTGGAAAGAAGAGGCCTGTAGAGCACTCTCCT |
| | GTGGAGCCAGACTCCTCCTCGGGAACCGGAAAGGCGGGCCAGCAGCCTGCAAGAAAAAG |
| | ATTGAATTTTGGTCAGACTGGAGACGCAGACTCAGTCCCAGACCCTCAACCAATCGGAGA |
| | ACCTCCCGCAGCCCCCTCAGGTGTGGGATCTCTTACAATGGCTGCAGGCGGTGGCGCACC |
| | AATGGCAGACAATAACGAGGGCGCCGACGGAGTGGGTAATTCCTCGGGAAATTGGCATT |
| | GCGATTCCACATGGATGGGCGACAGAGTCATCACCACCAGCACCCGAACCTGGGCCCTGC |
| | CCACCTACAACAACCACCTCTACAAGCAAATCTCCAACAGCACATCTGGAGGATCTTCAA |
| | ATGACAACGCCTACTTCGGCTACAGCACCCCCTGGGGGTATTTTGACTTTAACAGATTCC |
| | ACTGCCACTTTTCACCACGTGACTGGCAGCGACTCATCAACAACAACTGGGGATTCCGGC |
| | CCAAGAGACTCAGCTTCAAGCTCTTCAACATCCAGGTCAAGGAGGTCACGCAGAATGAA |
| | GGCACCAAGACCATCGCCAATAACCTCACCAGCACCATCCAGGTGTTTACGGACTCGGAG |
| | TACCAGCTGCCGTACGTTCTCGGCTCTGCCCACCAGGGCTGCCTGCCTCCGTTCCCGGCGG |
| | ACGTGTTCATGATTCCCCAGTACGGCTACCTAACACTCAACAACGGTAGTCAGGCCGTGG |
| | GACGCTCCTCCTTCTACTGCCTGGAATACTTTCCTTCGCAGATGCTGAGAACCGGCAACA |
| | ACTTCCAGTTTACTTACACCTTCGAGGACGTGCCTTTCCACAGCAGCTACGCCCACAGCCA |
| | GAGCTTGGACCGGCTGATGAATCCTCTGATTGACCAGTACCTGTACTACTTGTCTCGGACT |
| | CAAACAACAGGAGGCACGACAAATACGCAGACTCTGGGCTTCAGCCAAGGTGGGCCTAA |
| | TACAATGGCCAATCAGGCAAAGAACTGGCTGCCAGGACCCTGTTACCGCCAGCAGCGAG |
| | TATCAAAGACATCTGCGGATAACAACAACAGTGAATACTCGTGGACTGGAGCTACCAAG |
| | TACCACCTCAATGGCAGAGACTCTCTGGTGAATCCGGGCCCGGCCATGGCAAGCCACAAG |
| | GACGATGAAGAAAAGTTTTTTCCTCAGAGCGGGGTTCTCATCTTTGGGAAGCAAGGCTCA |
| | GAGAAAACAAATGTGGACATTGAAAAGGTCATGATTACAGACGAAGAGGAAATCAGGAC |
| | AACCAATCCCGTGGCTACGGAGCAGTATGGTTCTGTATCTACCAACCTCCAGCAAGGTGT |
| | ACATCGATTGTTAATCAATAAACCGTTTAATTCGTTTCAGTTGAACTTTGGTCTCTGCGTA |
| | TTTCTTTCTTATCTAGTTTCCATGGCTACGTAGATAAGTAGCATGGCGGGTTAATCATTAA |
| | CTACAAGGAACCCCTAGTGATGGAGTTGGCCACTCCCTCTCTGCGCGCTCGCTCGCTCACT |
| | GAGGCCGGGCGACCAAAGGTCGCCCGACGCCCGGGCTTTGCCCGGGCGGCCTCAGTGAG |
| | CGAGCGAGCGCGCAGAGAGGGAGTGGCCAAGCATGCAATTAACTGGCCGTCGTTTTACA |
| | ACGTCGTGACTGGGAAAACCCTGGCGTTACCCAACTTAATCGCCTTGCAGCACATCCCCC |
| | TTTCGCCAGCTGTATCAGCACACAATTGCCCATTATACGCGCGTATAATGGACTATTGTGT |
| | GCTGATA |
| TELN-GFAPG- | TATCAGCACACAATAGTCCATTATACGCGCGTATAATGGGCAATTGTGTGCTGATACAGC |
| DJ8-BSRGI | TGGCACGACAGGTTTCCCGACTGGAAAGCGGGCAGTGAGCGCAACGCAATTAATGTGAG |
| SEQ ID NO: 25 | TTAGCTCACTCATTAGGCACCCCAGGCTTTACACTTTATGCTTCCGGCTCGTATGTTGTGT |
| | GGAATTGTGAGCGGATAACAATTTCACACAGGAAACAGCTATGACCATGATTACGCCAG |
| | ATTTAATTAAGGCCTTAATTAGGCTAGCTTGGCCACTCCCTCTCTGCGCGCTCGCTCGCTC |
| | ACTGAGGCCGGGCGACCAAAGGTCGCCCGACGCCCGGGCTTTGCCCGGGCGGCCTCAGT |
| | GAGCGAGCGAGCGCGCAGAGAGGGAGTGGCCAACTCCATCACTAGGGGTTCCTGGAGGG |
| | GTGGAGTCGTGACGATATCCATGCGTCGACATAACGCGTGATCTAACATATCCTGGTGTG |
| | GAGTAGCGGACGCTGCTATGACAGAGGCTCGGGGGCCTGAGCTGGCTCTGTGAGCTGGG |
| | GAGGAGGCAGACAGCCAGGCCTTGTCTGCAAGCAGACCTGGCAGCATTGGGCTGGCCGC |
| | CCCCCAGGGCCTCCTCTTCATGCCCAGTGAATGACTCACCTTGGCACAGACACAATGTTC |
| | GGGGTGGGCACAGTGCCTGCTTCCCGCCGCACCCCAGCCCCCCTCAAATGCCTTCCGAGA |
| | AGCCCATTGAGCAGGGGGCTTGCATTGCACCCCAGCCTGACAGCCTGGCATCTTGGGATA |
| | AAAGCAGCACAGCCCCCTAGGGGCTGCCCTTGCTGTGTGGCGCCACCGGCGGTGGAGAA |
| | CAAGGCTCTATTCAGCCTGTGCCCAGGAAAGGGGATCAGGGGATGCCCAGGCATGGACA |
| | GTGGGTGGCAGGGGGGGAGAGGAGGGCTGTCTGCTTCCCAGAAGTCCAAGGACACAAAT |
| | GGGTGAGGGGAGAGCTCTCCCCATAGCTGGGCTGCGGCCCAACCCCACCCCCTCAGGCTA |
| | TGCCAGGGGGTGTTGCCAGGGGCACCCGGGCATCGCCAGTCTAGCCCACTCCTTCATAAA |
| | GCCCTCGCATCCCAGGAGCGAGCAGAGCCAGAGCAGGTTGGAGAGGAGACGCATCACCT |
| | CCGCTGCTCGCGGGGATCCTCTAGAAGCTTCGTTTAGTGAACCGTCAGATCGCCTGGAGA |
| | CGCCATCCACGCTGTTTTGACCTCCATAGAAGACACCGGGACCGATCCAGCCTCCGCGGA |
| | TTCGAATCCCGGCCGGGAACGGTGCATTGGAACGCGGATTCCCCGTGCCAAGAGTGACGT |
| | AAGTACCGCCTATAGAGTCTATAGGCCCACAAAAAATGCTTTCTTCTTTTAATATACTTTT |
| | TTGTTTATCTTATTTCTAATACTTTCCCTAATCTCTTTCTTTCAGGGCAATAATGATACAAT |
| | GTATCATGCCTCTTTGCACCATTCTAAAGAATAACAGTGATAATTTCTGGGTTAAGGCAAT |
| | AGCAATATTTCTGCATATAAATATTTCTGCATATAAATTGTAACTGATGTAAGAGGTTTCA |
| | TATTGCTAATAGCAGCTACAATCCAGCTACCATTCTGCTTTTATTTTATGGTTGGGATAAG |
| | GCTGGATTATTCTGAGTCCAAGCTAGGCCCTTTTGCTAATCATGTTCATACCTCTTATCTTC |
| | CTCCCACAGCTCCTGGGCAACGTGCTGGTCTGTGTGCTGGCCCATCACTTTGGCAAAGAA |
| | TTGGGATTCGAACCGGTCGCCACCGGTCACCAAGCAGGAAGTCAAAGACTTTTTCCGGTG |
| | GGCAAAGGATCACGTGGTTGAGGTGGAGCATGAATTCTACGTCAAAAAGGGTGGAGCCA |
| | AGAAAAGACCCGCCCCCAGTGACGCAGATATAAGTGAGCCCAAACGGGTGCGCGAGTCA |
| | GTTGCGCAGCCATCGACGTCAGACGCGGAAGCTTCGATCAACTACGCGGACAGGTACCA |
| | AAACAAATGTTCTCGTCACGTGGGCATGAATCTGATGCTGTTTCCCTGCAGACAATGCGA |
| | GAGAATGAATCAGAATTCAAATATCTGCTTCACTCACGGACAGAAAGACTGTTTAGAGTG |
| | CTTTCCCGTGTCAGAATCTCAACCCGTTTCTGTCGTCAAAAAGGCGTATCAGAAACTGTGC |
| | TACATTCATCATATCATGGGAAAGGTGCCAGACGCTTGCACTGCCTGCGATCTGGTCAAT |
| | GTGGATTTGGATGACTGCATCTTTGAACAATAAATGATTTAAATCAGGTATGGCTGCCGA |
| | TGGTTATCTTCCAGATTGGCTCGAGGACACTCTCTCTGAAGGAATAAGACAGTGGTGGAA |
| | GCTCAAACCTGGCCCACCACCACCAAAGCCCGCAGAGCGGCATAAGGACGACAGCAGGG |
| | GTCTTGTGCTTCCTGGGTACAAGTACCTCGGACCCTTCAACGGACTCGACAAGGGAGAGC |
| | CGGTCAACGAGGCAGACGCCGCGGCCCTCGAGCACGACAAAGCCTACGACCGGCAGCTC |
| | GACAGCGGAGACAACCCGTACCTCAAGTACAACCACGCCGACGCCGAGTTCCAGGAGCG |
| | GCTCAAAGAAGATACGTCTTTTGGGGGCAACCTCGGGCGAGCAGTCTTCCAGGCCAAAA |
| | AGAGGCTTCTTGAACCTCTTGGTCTGGTTGAGGAAGCGGCTAAGACGGCTCCTGGAAAGA |
| | AGAGGCCTGTAGAGCACTCTCCTGTGGAGCCAGACTCCTCCTCGGGAACCGGAAAGGCG |
| | GGCCAGCAGCCTGCAAGAAAAAGATTGAATTTTGGTCAGACTGGAGACGCAGACTCAGT |
| | CCCAGACCCTCAACCAATCGGAGAACCTCCCGCAGCCCCCTCAGGTGTGGGATCTCTTAC |
| | AATGGCTGCAGGCGGTGGCGCACCAATGGCAGACAATAACGAGGGCGCCGACGGAGTGG |
| | GTAATTCCTCGGGAAATTGGCATTGCGATTCCACATGGATGGGCGACAGAGTCATCACCA |
| | CCAGCACCCGAACCTGGGCCCTGCCCACCTACAACAACCACCTCTACAAGCAAATCTCCA |
| | ACAGCACATCTGGAGGATCTTCAAATGACAACGCCTACTTCGGCTACAGCACCCCCTGGG |
| | GGTATTTTGACTTTAACAGATTCCACTGCCACTTTTCACCACGTGACTGGCAGCGACTCAT |
| | CAACAACAACTGGGGATTCCGGCCCAAGAGACTCAGCTTCAAGCTCTTCAACATCCAGGT |
| | CAAGGAGGTCACGCAGAATGAAGGCACCAAGACCATCGCCAATAACCTCACCAGCACCA |
| | TCCAGGTGTTTACGGACTCGGAGTACCAGCTGCCGTACGTTCTCGGCTCTGCCCACCAGG |
| | GCTGCCTGCCTCCGTTCCCGGCGGACGTGTTCATGATTCCCCAGTACGGCTACCTAACACT |
| | CAACAACGGTAGTCAGGCCGTGGGACGCTCCTCCTTCTACTGCCTGGAATACTTTCCTTCG |
| | CAGATGCTGAGAACCGGCAACAACTTCCAGTTTACTTACACCTTCGAGGACGTGCCTTTC |
| | CACAGCAGCTACGCCCACAGCCAGAGCTTGGACCGGCTGATGAATCCTCTGATTGACCAG |
| | TACCTGTACTACTTGTCTCGGACTCAAACAACAGGAGGCACGACAAATACGCAGACTCTG |
| | GGCTTCAGCCAAGGTGGGCCTAATACAATGGCCAATCAGGCAAAGAACTGGCTGCCAGG |
| | ACCCTGTTACCGCCAGCAGCGAGTATCAAAGACATCTGCGGATAACAACAACAGTGAAT |
| | ACTCGTGGACTGGAGCTACCAAGTACCACCTCAATGGCAGAGACTCTCTGGTGAATCCGG |
| | GCCCGGCCATGGCAAGCCACAAGGACGATGAAGAAAAGTTTTTTCCTCAGAGCGGGGTT |
| | CTCATCTTTGGGAAGCAAGGCTCAGAGAAAACAAATGTGGACATTGAAAAGGTCATGAT |
| | TACAGACGAAGAGGAAATCAGGACAACCAATCCCGTGGCTACGGAGCAGTATGGTTCTG |
| | TATCTACCAACCTCCAGCAAGGTGTACATCGATTGTTAATCAATAAACCGTTTAATTCGTT |
| | TCAGTTGAACTTTGGTCTCTGCGTATTTCTTTCTTATCTAGTTTCCATGGCTACGTAGATAA |
| | GTAGCATGGCGGGTTAATCATTAACTACAAGGAACCCCTAGTGATGGAGTTGGCCACTCC |
| | CTCTCTGCGCGCTCGCTCGCTCACTGAGGCCGGGCGACCAAAGGTCGCCCGACGCCCGGG |
| | CTTTGCCCGGGCGGCCTCAGTGAGCGAGCGAGCGCGCAGAGAGGGAGTGGCCAAGCATG |
| | CAATTAACTGGCCGTCGTTTTACAACGTCGTGACTGGGAAAACCCTGGCGTTACCCAACT |
| | TAATCGCCTTGCAGCACATCCCCCTTTCGCCAGCTGTATCAGCACACAATTGCCCATTATA |
| | CGCGCGTATAATGGACTATTGTGTGCTGATA |

### LITERATURE CITED

Berns K I, Giraud C. Biology of adeno-associated virus. Curr Top Microbiol Immunol. 1996; 218:1-23.
Chan K Y, Jang M J, Yoo B B, Greenbaum A, Ravi N, Wu W L, Sánchez-Guardado L, Lois C, Mazmanian S K, Deverman B E, Gradinaru V. Engineered AAVs for efficient noninvasive gene delivery to the central and peripheral nervous systems. Nat Neurosci. 2017 August; 20(8):1172-1179.
Heinrich J, Schultz J, Bosse M, Ziegelin G, Lanka E, Moelling K. Linear closed mini DNA generated by the prokaryotic cleaving-joining enzyme TelN is functional in mammalian cells. J Mol Med (Berl). 2002 October; 80(10):648-54.
Hordeaux J, Wang Q, Katz N, Buza E L, Bell P, Wilson J M. The Neurotropic Properties of AAV-PHP.B Are Limited to C57BL/6J Mice. Mol Ther. 2018 Mar. 7; 26(3):664-668.
Huovinen T, Brockmann E C, Akter S, Perez-Gamarra S, Yla-Pelto J, Liu Y, Lamminmäki U. Primer extension mutagenesis powered by selective rolling circle amplification. PLOS One. 2012; 7(2):e31817.
Huovinen T, Julin M, Sanmark H, Lamminmäki U. Enhanced error-prone RCA mutagenesis by concatemer resolution. Plasmid. 2011 October; 66(1):47-51.
Hutchison C A 3rd, Smith H O, Pfannkoch C, Venter J C. Cell-free cloning using phi29 DNA polymerase. Proc Natl Acad Sci USA. 2005 Nov. 29; 102(48):17332-6.
Miyazaki J, Takaki S, Araki K, Tashiro F, Tominaga A, Takatsu K, Yamamura K. Expression vector system based on the chicken beta-actin promoter directs efficient production of interleukin-5. Gene. 1989 Jul. 15; 79(2):269-77.
Mouw M B, Pintel D J. Adeno-associated virus RNAs appear in a temporal order and their splicing is stimulated during coinfection with adenovirus. J Virol. 2000 November; 74(21):9878-88.
Niwa H, Yamamura K, Miyazaki J. Efficient selection for high-expression transfectants with a novel eukaryotic vector. Gene. 1991 Dec. 15; 108(2):193-9.
Nonnenmacher M, van Bakel H, Hajjar R J, Weber T. High capsid-genome correlation facilitates creation of AAV libraries for directed evolution. Mol Ther. 2015 April; 23(4):675-82.
Picher ÁJ, Budeus B, Wafzig O, Krüger C, García-Gómez S, Martínez-Jiménez M I, Díaz-Talavera A, Weber D, Blanco L, Schneider A. TruePrime is a novel method for whole-genome amplification from single cells based on TthPrimPol. Nat Commun. 2016 Nov. 29; 7:13296.
Powell S K, Rivera-Soto R, Gray S J. Viral expression cassette elements to enhance transgene target specificity and expression in gene therapy. Discov Med. 2015 January; 19(102):49-57.
Rybchin V N, Svarchevsky A N. The plasmid prophage N15: a linear DNA with covalently closed ends. Mol Microbiol. 1999 September; 33(5):895-903.
Zolotukhin S, Byrne B J, Mason E, Zolotukhin I, Potter M, Chesnut K, Summerford C, Samulski R J, Muzyczka N. Recombinant adeno-associated virus purification using novel methods improves infectious titer and yield. Gene Ther. 1999 June; 6(6):973-85.

1.-15. (canceled)

16. A variant adeno-associated (AAV) capsid polypeptide which comprises:
(i) the amino acid sequence of any the amino acid sequences provided in Table 2;
(ii) the amino acid sequence of SEQ ID NO: 134; or
(iii) the amino acid sequence of SEQ ID NO: 68.

17. The variant AAV capsid polypeptide of claim 16, which comprises the amino acid sequence of SEQ ID NO: 134.

18. The variant AAV capsid polypeptide of claim 16, which comprises the amino acid sequence of SEQ ID NO: 62.

19. The variant AAV capsid polypeptide of claim 16, wherein the amino acid sequence is present in loop VIII of the variant AAV capsid polypeptide.

20. The variant AAV capsid polypeptide of claim 16, wherein the amino acid sequence is present immediately subsequent to a position selected from 586-592 of the variant AAV capsid polypeptide.

21. The variant AAV capsid polypeptide of claim 16, which further comprises the amino acid sequence of SEQ ID NO: 2, or an amino acid sequence at least 95% identical thereto.

22. The variant AAV capsid polypeptide of claim 16, which further comprises the amino acid sequence of SEQ ID NO: 3, or an amino acid sequence at least 95% identical thereto.

23. An adeno-associated virus (AAV) particle comprising the variant AAV capsid polypeptide of claim 16 and a viral genome.

24. The AAV particle of claim 23, wherein the viral genome encodes one or more siRNA molecules.

25. A pharmaceutical composition comprising the AAV particle of claim 23.

26. A method of delivering a payload to a subject, the method comprising administering to the subject an AAV particle comprising the variant AAV capsid polypeptide of claim 16, thereby delivering the payload.

27. The method of claim 26, wherein the AAV particle is administered intravenously.

28. A method of treating a neurological disease in a subject, the method comprising administering to the subject an AAV particle comprising the variant AAV capsid polypeptide of claim 16, thereby treating the neurological disease in a subject.

29. The method of claim 28, wherein the AAV particle or pharmaceutical composition is administered intravenously.

30. The method of claim 28, wherein the neurological disease is Alzheimer's Disease, Amyotrophic Lateral Sclerosis (ALS), Parkinson's disease, or Huntington's Disease.
